(19) United States
(12) Patent Application Publication
(10) Pub. No.: US 2025/0179079 A1
(43) Pub. Date: Jun. 5, 2025

(54) HEXAHYDROPYRIDO[4,3-B]INDOLYL KETONE DERIVATIVES USEFUL AS CGAS MODULATORS

(71) Applicant: Ventus Therapeutics U.S., Inc., Waltham, MA (US)

(72) Inventors: Ramsay Beveridge, Pointe-Claire (CA); Jason Burch, Pointe-Claire (CA); Patrick Cyr, Saint-Constant (CA)

(73) Assignee: Ventus Therapeutics U.S., Inc., Waltham, MA (US)

(21) Appl. No.: 18/845,854

(22) PCT Filed: Mar. 21, 2023
(86) PCT No.: PCT/US2023/015728

§ 371 Date: Sep. 10, 2024

Related U.S. Application Data
(60) Provisional application No. 63/322,466, filed on Mar. 22, 2022.

(51) Int. Cl. C07D 487/04; A61K 31/437; A61K 31/497; A61K 31/4985; A61K 31/501; A61K 31/506; A61K 31/519; A61K 31/5377; A61K 31/5383; A61K 31/5386; A61K 31/55; C07D 471/04; C07D 471/14; C07D 471/18; C07D 498/18; C07D 519/00

(52) U.S. Cl. CPC C07D 487/04; A61K 31/437; A61K 31/497; A61K 31/4985; A61K 31/501; A61K 31/506; A61K 31/519; A61K 31/5377; A61K 31/5383; A61K 31/5386; A61K 31/55; C07D 471/04; C07D 471/14; C07D 471/18; C07D 498/18; C07D 519/00

(57) ABSTRACT

The present disclosure relates to compound of Formula (I), such as compounds of Formula (II), (III), and (IV), useful for cGAS modulation, wherein L¹, R¹, R², R⁹, X¹, X², X³, X⁴, X⁵, X⁶, X⁷, X⁸, X⁹, X¹⁰, X¹¹, Y, and r are described therein.

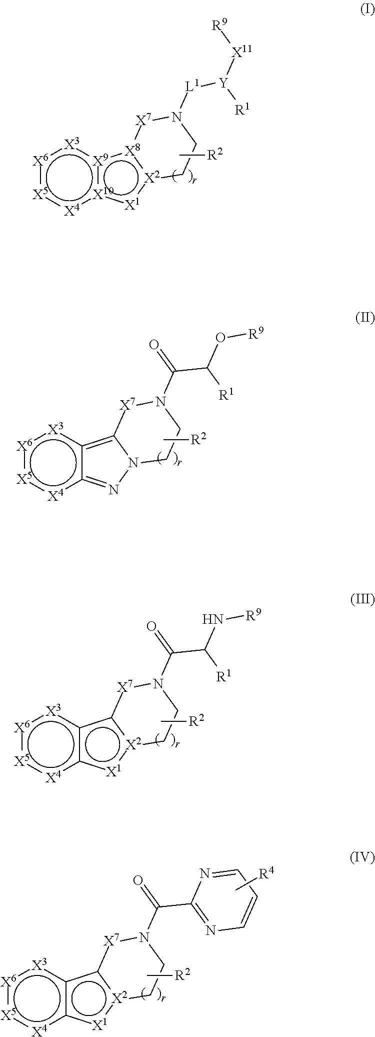

### RELATED APPLICATIONS

The present application claims priority under 35 U.S.C. § 119(e) to United States Provisional Patent Application, U.S. Ser. No. 63/322,466, filed Mar. 22, 2022, the entire contents of which is incorporated herein by reference.

### FIELD OF INVENTION

The present invention is directed to inhibitors of Cyclic GMP-AMP synthase. The inhibitors described herein can be useful in the treatment of diseases or disorders associated with Cyclic GMP-AMP synthase. In particular, the invention is concerned with compounds and pharmaceutical compositions inhibiting Cyclic GMP-AMP synthase, methods of treating diseases or disorders associated with Cyclic GMP-AMP synthase, and methods of synthesizing these compounds.

### BACKGROUND OF THE INVENTION

Introductory cytosolic DNA induces type I interferon and other cytokines that are important for antimicrobial defense but can also induce autoimmunity. This DNA signaling pathway requires the adapter protein STING (Stimulator of Interferon Genes) and the transcription factors NF-κB and IRF3, but the mechanism of DNA sensing was unclear until recently. It is now understood that mammalian cytosolic extracts synthesize cyclic-GMP-AMP (cGAMP) in vitro from ATP and GTP in the presence of DNA rather than RNA. (WO 2014099824). DNA transfection or DNA virus infection of mammalian cells also triggers the production of cGAMP. cGAMP binds to STING, leading to IRF3 activation and induction of interferon-β (IFNβ). Thus, cGAMP is the first cyclic dinucleotide in metazoans, and cGAMP functions as an endogenous secondary messenger that induces interferon production in response to cytosolic DNA.

cGAMP synthase (cGAS) is an enzyme which intervenes in the synthesis of cyclic-GMP-AMP and which belongs to the nucleotidyltransferase family. Overexpression of cGAS activates the transcription factor IRF3 and induces IFNβ in a STING-dependent manner. Knockdown of cGAS inhibits IRF3 activation and IFNβ induction by DNA transfection or DNA virus infection. cGAS binds to DNA in the cytoplasm and catalyzes cGAMP synthesis. These findings indicate that cGAS is a cytosolic DNA sensor that induces interferons by producing the second messenger cGAMP.

The critical role of cGAS in cytosolic DNA sensing has been established in different pathogenic bacteria, viruses, and retroviruses. (US 20210155625) Additionally, cGAS is essential in various other biological processes such as cellular senescence and recognition of ruptured micronuclei in the surveillance of potential cancer cells.

There is a need for therapeutic agents that targets cGAS, Small molecule inhibitors that are specific for cGAS would be of great value in treating diseases that arise from inappropriate cGAS activity and the resulting undesired type I interferon activity. This invention is intended to fill this unmet needs associated with current cGAS inhibition therapy.

### SUMMARY OF THE INVENTION

The present disclosure provides compounds of Formula (I):

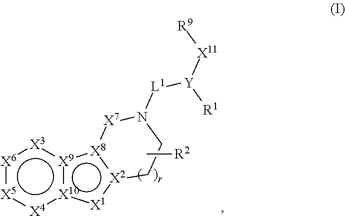

and pharmaceutically acceptable salts, hydrates, solvates, prodrugs, stereoisomers, and tautomers thereof, wherein:
L¹ is —C(O)—, —S(O), —S(O)₂—, or —S(NH)(O)—;
X¹ is independently N, NR⁵, or CH;
X² is independently N or C, provided that at least one of X¹ and X² includes N, wherein X¹ is N or NR⁵ and/or X² is N;
X³, X⁴, X⁵, X⁶, X⁸, X⁹, and X¹⁰ are independently C, CR³ or N, as valency permits, wherein at least one of X³, X⁴, X⁵, and X⁶ is CR³, and wherein X³, X⁴, X⁵, X⁶, X⁸, X⁹, and X¹⁰, independently, are not more than 7 N in total;
X⁷ is independently NH, NCH₃, or C(R²)₂;
X¹¹ is independently O, N or NH;
Y is NH, CH, or C;
R¹ is H or C₁-C₆ alkyl, wherein the alkyl is optionally substituted with one or more R⁴; or
R¹ and R⁹ combine to form a 3- to 8-membered heterocycle or 5- to 10-membered heteroaryl, wherein the heterocycle or heteroaryl is optionally substituted with one or more R⁴;
each R² is independently H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle;
each R³ is independently H, halogen, oxo, —CN, —OR⁵, —SR⁵, —NH₂, —NH(R⁵), —N(R⁵)(R⁶), —NHC(O)R⁵, —CO(OR⁵), —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶;
each R⁴ is independently H, halogen, —CN, —OR⁵, —NH₂, —NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or more R⁷;
each R⁵ is independently H, —C(O)OH, —(CH₂)ₙ—O—(CH₂)ₚ—OR⁸, —(CH₂)ₙ—OR⁸, —(CH₂)ₙ—S(O)₂R⁸, —CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl;
R⁶, R⁷, and R⁸ are independently, at each occurrence, H, halogen, —OH, —CN, —NH₂, C₁-C₆ alkyl, C₁-C₆ hydroxyalkyl, C₁-C₆ alkoxy, C₂-C₆ alkenyl, C₂-C₆ alkynyl, C₃-C₈ cycloalkyl, heterocyclyl, heteroaryl, or aryl;
R⁹ is independently H or C₁-C₄ alkyl, wherein the alkyl is optionally substituted with one or more halogen, —OH, —CN, C₁-C₆ alkoxy, or cycloalkyl;
each n is independently an integer from 0 to 6;
each p is independently an integer from 0 to 6; and
r is an integer from 0 to 2;
provided that:
   (1) when R¹ and R⁹ combine to form a 3- to 8-membered heterocycle or 5- to 10-membered heteroaryl, then X¹¹ is N or NH; and/or
   (2) when X¹¹ is O, then X¹ and X² are both N and X⁷ is —CH(R²)—; and/or
   (3) when X¹¹ is NH and R⁹ is H, then X⁷ is not —CH₂—; and/or
   (4) when X³ is CR³ and R³ is heteroaryl, then R¹ and R⁹ form a heteroaryl;
and further provided that:
   (5) R¹ and R⁹ do not combine to form a pyrazole.

In certain embodiments, when X⁸, X⁹, and/or X¹⁰ are independently CR³, it is understood that R³ is absent to satisfy the valency of these groups. For example, in certain embodiments, X⁸, X⁹, and X¹⁰ are each C. In certain embodiments, X⁸ is C. In certain embodiments, X⁹ is C. In certain embodiments, X¹⁰ is C.

In certain embodiments, R¹ and R⁹ do not combine to form a pyrazole, indole, imidazole, pyridine, or thiazole. In certain embodiments, R¹ and R⁹ do not combine to form a pyrazole. In certain embodiments, R¹ and R⁹ do not combine to form an indole. In certain embodiments, R¹ and R⁹ do not combine to form an imidazole. In certain embodiments, R¹ and R⁹ do not combine to form a pyridine. In certain embodiments, R¹ and R⁹ do not combine to form a thiazole.

In certain embodiments, when R¹ and R⁹ combine to form a pyrimidine, then X⁶ is not C, CO(OR⁵). In certain embodiments, when R¹ and R⁹ combine to form a pyrimidine, then X⁶ is not C, CO(OH). In certain embodiments, when R¹ and R⁹ combine to form a pyrimidine, then R³ is not —CO(OR⁵). In certain embodiments, when R¹ and R⁹ combine to form a pyrimidine, then R³ is not —CO(OH).

In certain embodiments, when R¹ and R⁹ combine to form a pyrimidine, X¹ is NR⁵, and X² is C, then X⁶ is not C—CO(OR⁵). In certain embodiments, when R¹ and R⁹ combine to form a pyrimidine, X¹ is NR⁵, and X² is C, then X⁶ is not C—CO(OH). In certain embodiments, when R¹ and R⁹ combine to form a pyrimidine, X¹ is NR⁵, and X² is C, then R³ is not —CO(OR⁵). In certain embodiments, when R¹ and R⁹ combine to form a pyrimidine, X¹ is NR⁵, and X² is C, then R³ is not —CO(OH).

In certain embodiment, the compound of Formula (I) is not:

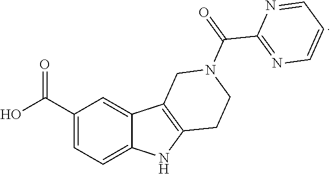 [SMILES: O=C(O)c1ccc2[nH]c3c(c2c1)CN(C(=O)c1ncccn1)CC3]

In certain embodiments, at least one instance of R² is not H. In certain embodiments, X⁷ is —CH(R²)—, wherein R² is not H. In certain embodiments, X⁷ is not —CH₂—.

In certain embodiments, when X¹¹ is O, then X¹ and X² are both N and at least one instance of R² is not H. In certain embodiments, when X¹¹ is O, then X¹ and X² are both N and X⁷ is —CH(R²)—, wherein R² is not H. In certain embodiments, when X¹¹ is 0, then X¹ and X² are both N and X⁷ is not —CH₂—.

In certain embodiments, the compounds of Formula (I) are of Formula (II):

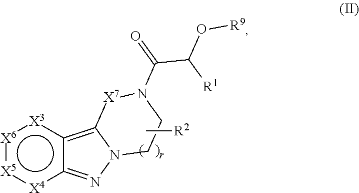

or pharmaceutically acceptable salts, hydrates, solvates, prodrugs, stereoisomers, or tautomers thereof, wherein:
X⁷ is —CH(R²)—, wherein R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl;
the other R² is H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle; and
X³, X⁴, X⁵, and X⁶ are each independently CR³.

In certain embodiments, the compounds of Formula (I) are of Formula (III):

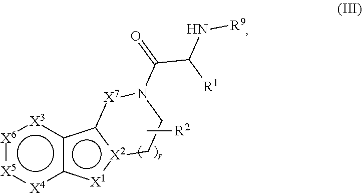

or pharmaceutically acceptable salts, hydrates, solvates, prodrugs, stereoisomers, or tautomers thereof, wherein:
X¹ is N or NR⁵, and X² is N or C;
X³, X⁴, X⁵, and X⁶ are each independently CR³;
X⁷ is —CH(R²)—, wherein R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; and
the other R² is H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle.

In certain embodiments, the compounds of Formula (I) are of Formula (IV):

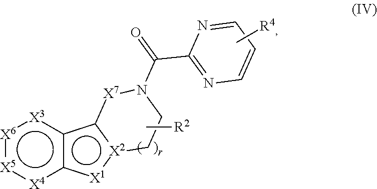

or pharmaceutically acceptable salts, hydrates, solvates, prodrugs, stereoisomers, or tautomers thereof, wherein:
X¹ is N or NR⁵, and X² is N or C;
X⁷ is —CH(R²)—;
X³, X⁴, X⁵, and X⁶ are each independently CR³; and
each R³ is independently H, halogen, —CN, —OR⁵, —SR⁵, —NH₂, —NH(R⁵), —N(R⁵)(R⁶), —NHC(O)R⁵, —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶.

References to a compound or compounds of Formula (I) herein are intended to include compounds of any subgeneric formula or species disclosed herein, and pharmaceutically acceptable salts, hydrates, solvates, prodrugs, stereoisomers, and tautomers thereof. For example, reference to compounds of Formula (I) include compounds of Formulae (II), (III), and (IV), and any subgeneric formulae or species thereof, and pharmaceutically acceptable salts, hydrates, solvates, prodrugs, stereoisomers, and tautomers thereof.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with modulation of cGAS. The method comprises administering to a patient in need of a treatment for diseases or disorders associated with modulation of cGAS an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting cGAS. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the invention is directed to a method of treating or preventing a disease or disorder disclosed herein in a subject in need thereof. The method involves administering to a patient in need thereof an effective amount of a compound of Formula (I), or a pharmaceutically acceptable salt, hydrate, solvate, prodrug, stereoisomer, tautomer, or pharmaceutical composition thereof.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

Another aspect of the present invention relates to compounds of Formula (I), and pharmaceutically acceptable salts, hydrates, solvates, prodrugs, stereoisomers, tautomers, or pharmaceutical compositions thereof, for use in the manufacture of a medicament for inhibiting cGAS.

In some aspects, the present disclosure provides an intermediate as described herein, being suitable for use in a method for preparing a compound as described herein (e.g., the intermediate is selected from the intermediates described in Examples therein.

Another aspect of the present invention relates to compounds of Formula (I), and pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides use of a compound of the present disclosure or a pharmaceutically acceptable salt thereof in the manufacture of a medicament for treating a disease or disorder disclosed herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure relates to compounds and compositions that are capable of inhibiting cGAS. The disclosure features methods of treating, preventing or ameliorating a disease or disorder in which cGAS plays a role by administering to a patient in need thereof a therapeutically effective amount of a compound of Formula (I), or a pharmaceutically acceptable salt, hydrate, solvate, prodrug, stereoisomer, or tautomer thereof. The methods of the present invention can be used in the treatment of a variety of cGAS mediated diseases and disorders by inhibiting the activity of cGAS. The present disclosure also relates to processes for the preparation of these compounds, to pharmaceutical compositions comprising them and to their use in the treatment of disorders in which cGAS is implicated including, but not limited to inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia.

In a first aspect of the invention, the compounds of Formula (I) are described:

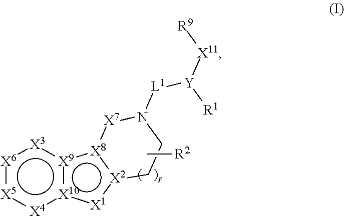

and pharmaceutically acceptable salts, hydrates, solvates, prodrugs, stereoisomers, and tautomers thereof, wherein L¹, R¹, R², R⁹, X¹, X², X³, X⁴, X⁵, X⁶, X⁸, X⁹, X¹⁰, X¹¹, Y, and r are described herein.

The details of the invention are set forth in the accompanying description below. Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus the term “optionally substituted” means that a given chemical moiety has the potential to contain other functional groups, but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆) alkyl, and S(O)N((C₁-C₆) alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆) alkyl, (C₁-C₆) alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆) alkyl, —C(O)(C₁-C₆) alkyl, —OC(O)O(C₁-C₆) alkyl, —NH₂, NH((C₁-C₆) alkyl), N((C₁-C₆) alkyl)₂, —S(O)₂—(C₁-C₆) alkyl, —S(O)NH(C₁-C₆) alkyl, and —S(O)N((C₁-C₆) alkyl)₂. The substituents can themselves be optionally substituted. Furthermore, when containing two fused rings, the aryl groups herein defined may have a saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or polycyclic aromatic radical of 5 to 24 ring atoms, containing one or more ring heteroatoms selected from N, O, S, P, Se, or B, the remaining ring atoms being C. Heteroaryl as herein defined also means a bicyclic heteroaromatic group wherein the heteroatom is selected from N, O, S, P, Se, or B. Heteroaryl as herein defined also means a tricyclic heteroaromatic group containing one or more ring heteroatoms selected from N, O, S, P, Se, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolinyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1λ²-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzoxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with a fully unsaturated aromatic ring, e.g., a 5-membered heteroaromatic ring containing 1 to 3 heteroatoms selected from N, O, S, P, Se, or B, or a 6-membered heteroaromatic ring containing 1 to 3 nitrogens, wherein the saturated or partially unsaturated ring includes 0 to 4 heteroatoms selected from N, O, S, P, Se, or B, and is optionally substituted with one or more oxo. In heteroaryl ring systems containing more than two fused rings, a saturated or partially unsaturated ring may further be fused with a saturated or partially unsaturated ring described herein. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, or benzo[c][1,2]oxaborol-1(3H)-olyl.

“Halogen” or “halo” refers to fluorine, chlorine, bromine, or iodine.

“Alkyl” refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above-mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means a saturated or partially unsaturated hydrocarbon monocyclic or polycyclic (e.g., fused, bridged, or spiro rings) system having 3 to 30 carbon atoms (e.g., C₃-C₁₂, C₃-C₁₀, or C₃-C₈). Examples of cycloalkyl groups include, without limitations, cyclopropyl, cyclobutyl, cyclopentyl, cyclohexyl, cycloheptanyl, cyclooctanyl, norboranyl, norborenyl, bicyclo[2.2.2]octanyl, bicyclo[2.2.2]octenyl, decahydronaphthalenyl, octahydro-1H-indenyl, cyclopentenyl, cyclohexenyl, cyclohexa-1,4-dienyl, cyclohexa-1,3-dienyl, 1,2,3,4-tetrahydronaphthalenyl, octahydropentalenyl, 3a,4,5,6,7,7a-hexahydro-1H-indenyl, 1,2,3,3a-tetrahydropentalenyl, bicyclo[3.1.0]hexanyl, bicyclo[2.1.0]pentanyl, spiro[3.3]heptanyl, bicyclo[2.2.1]heptanyl, bicyclo[2.2.1]hept-2-enyl, bicyclo[2.2.2]octanyl, 6-methylbicyclo[3.1.1]heptanyl, 2,6,6-trimethylbicyclo[3.1.1]heptanyl, adamantyl, and derivatives thereof. In the case of polycyclic cycloalkyl, only one of the rings in the cycloalkyl needs to be non-aromatic.

“Heterocyclyl”, “heterocycle” or “heterocycloalkyl” refers to a saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms (such as O, N, S, P, Se, or B), e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur, unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, oxiranyl, azetidinyl, oxetanyl, thietanyl, 1,2,3,6-tetrahydropyridinyl, tetrahydropyranyl, dihydropyranyl, pyranyl, morpholinyl, tetrahydrothiopyranyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl, 1,4-dioxaspiro[4.5]decanyl, 1-oxaspiro[4.5]decanyl, 1-azaspiro[4.5]decanyl, 3′H-spiro[cyclohexane-1,1′-isobenzofuran]-yl, 7′H-spiro[cyclohexane-1,5′-furo[3,4-b]pyridin]-yl, 3′H-spiro[cyclohexane-1,1′-furo[3,4-c]pyridin]-yl, 3-azabicyclo[3.1.0]hexanyl, 3-azabicyclo[3.1.0]hexan-3-yl, 1,4,5,6-tetrahydropyrrolo[3,4-c]pyrazolyl, 3,4,5,6,7,8-hexahydropyrido[4,3-d]pyrimidinyl, 4,5,6,7-tetrahydro-1H-pyrazolo[3,4-c]pyridinyl, 5,6,7,8-tetrahydropyrido[4,3-d]pyrimidinyl, 2-azaspiro[3.3]heptanyl, 2-methyl-2-azaspiro[3.3]heptanyl, 2-azaspiro[3.5]nonanyl, 2-methyl-2-azaspiro[3.5]nonanyl, 2-azaspiro[4.5]decanyl, 2-methyl-2-azaspiro[4.5]decanyl, 2-oxa-azaspiro[3.4]octanyl, 2-oxa-azaspiro[3.4]octan-6-yl, and the like.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted one or more halogen. Examples of haloalkoxy groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

The term “amine” as used herein refers to primary (R—NH₂, R #H), secondary (R₂—NH, R₂≠H) and tertiary (R₃—N, R #H) amines. A substituted amine is intended to mean an amine where at least one of the hydrogen atoms has been replaced by the substituent.

The term “amino” as used herein means a substituent containing at least one nitrogen atom. Specifically, —NH₂, —NH(alkyl) or alkylamino, —N(alkyl)₂ or dialkylamino, amide-, carbamide-, urea, and sulfamide substituents are included in the term “amino”.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present invention also contemplates isotopically-labelled compounds of Formula I (e.g., those labeled with ²H and ¹⁴C). Deuterated (i.e., ²H or D) and carbon-14 (i.e., ¹⁴C) isotopes are particularly desired for their ease of preparation and detectability. Further, substitution with heavier isotopes such as deuterium may afford certain therapeutic advantages resulting from greater metabolic stability (e.g., increased in vivo half-life or reduced dosage requirements) and hence may be desired in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising an effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumerate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon or rhesus.

An “effective amount” when used in connection with a compound is an amount effective for treating or preventing a disease or disorder in a subject as described herein. “Therapeutically effective” is an effective amount for therapeutic treatment (from a disease that the subject currently suffers from), and “prophylatically effective” is an effective amount for preventative treatment (from a disease that the subject has suffered from in the past, or may suffer from, as a preventative measure).

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

### Compounds

The present disclosure relates to compounds or pharmaceutically acceptable salts, hydrates, solvates, prodrugs, stereoisomers, or tautomers thereof, capable of inhibiting cGAS, which are useful for the treatment of diseases and disorders associated with modulation of cGAS. The invention further relates to compounds, or pharmaceutically acceptable salts, hydrates, solvates, prodrugs, stereoisomers, or tautomers thereof, which can be useful for inhibiting cGAS.

The present disclosure provides compounds of Formula (I):

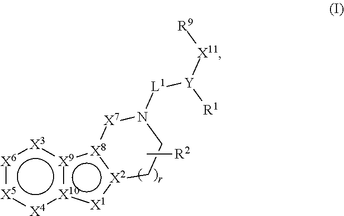

and pharmaceutically acceptable salts, hydrates, solvates, prodrugs, stereoisomers, and tautomers thereof, wherein:
L¹ is —C(O)—, —S(O), —S(O)₂—, or —S(NH)(O)—;
X¹ is independently N, NR⁵, or CH;
X² is independently N or C, provided that at least one of X¹ and X² includes N, wherein X¹ is N or NR⁵ and/or X² is N;
X³, X⁴, X⁵, X⁶, X⁸, X⁹, and X¹⁰ are independently C, CR³ or N, as valency permits, wherein at least one of X³, X⁴, X⁵, and X⁶ is CR³, and wherein X³, X⁴, X⁵, X⁶, X⁸, X⁹, and X¹⁰, independently, are not more than 7 N in total;
X⁷ is independently NH, NCH₃, or C(R²)₂;
X¹¹ is independently O, N or NH;
Y is NH, CH, or C;
R¹ is H or C₁-C₆ alkyl, wherein the alkyl is optionally substituted with one or more R⁴; or
R¹ and R⁹ combine to form a 3- to 8-membered heterocycle or 5- to 10-membered heteroaryl, wherein the heterocycle or heteroaryl is optionally substituted with one or more R⁴;
each R² is independently H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle;
each R³ is independently H, halogen, oxo, —CN, —OR⁵, —SR⁵, —NH₂, —NH(R⁵), —N(R⁵)(R⁶), —NHC(O)R⁵, —CO(OR⁵), —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶;
each R⁴ is independently H, halogen, —CN, —OR⁵, —NH₂, —NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or more R⁷;
each R⁵ is independently H, —C(O)OH, —(CH₂)ₙ—O—(CH₂)ₚ—OR⁸, —(CH₂)ₙ—OR⁸, —(CH₂)ₙ—S(O)₂R⁸, —CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl;
R⁶, R⁷, and R⁸ are independently, at each occurrence, H, halogen, —OH, —CN, —NH₂, C₁-C₆ alkyl, C₁-C₆ hydroxyalkyl, C₁-C₆ alkoxy, C₂-C₆ alkenyl, C₂-C₆ alkynyl, C₃-C₈ cycloalkyl, heterocyclyl, heteroaryl, or aryl;
R⁹ is independently H or C₁-C₄ alkyl, wherein the alkyl is optionally substituted with one or more halogen, —OH, —CN, C₁-C₆ alkoxy, or cycloalkyl;
each n is independently an integer from 0 to 6;
each p is independently an integer from 0 to 6; and
r is an integer from 0 to 2;
provided that:
   (1) when R¹ and R⁹ combine to form a 3- to 8-membered heterocycle or 5- to 10-membered heteroaryl, then X¹¹ is N or NH; and/or
   (2) when X¹¹ is O, then X¹ and X² are both N and X⁷ is —CH(R²)—; and/or
   (3) when X¹¹ is NH and R⁹ is H, then X⁷ is not —CH₂—; and/or
   (4) when X³ is CR³ and R³ is heteroaryl, then R¹ and R⁹ form a heteroaryl;
and further provided that:
   (5) R¹ and R⁹ do not combine to form a pyrazole.

In certain embodiments of Formula (I), X⁷ is of the following formula (b):

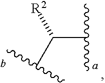

wherein a indicates the point of attachment to the amide nitrogen and b indicates the point of attachment to the heteroaryl ring. In certain embodiments of Formula (I), X⁷ is of the following formula (a):

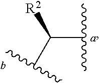

wherein a indicates the point of attachment to the amide nitrogen and b indicates the point of attachment to the heteroaryl ring. In certain embodiments of Formula (I), X⁷ is of the following formula (b-1):

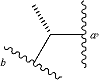

wherein a indicates the point of attachment to the amide nitrogen and b indicates the point of attachment to the heteroaryl ring. In certain embodiments of Formula (I), X⁷ is of the following formula (a-1):

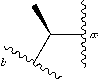

wherein a indicates the point of attachment to the amide nitrogen and b indicates the point of attachment to the heteroaryl ring.

In some embodiments of Formula (I), the carbon atom bonded to R² (at the X⁷ position) is in the (S) configuration. In some embodiments of Formula (I), the carbon atom bonded to R² (at the X⁷ position) is in the (R) configuration.

In certain embodiments, the compounds of Formula (I) are of Formula (II):

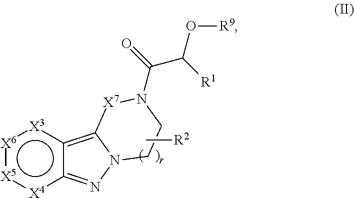

or pharmaceutically acceptable salts, hydrates, solvates, prodrugs, stereoisomers, or tautomers thereof, wherein:
X⁷ is —CH(R²)—, wherein R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl;
the other R² is H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle; and
X³, X⁴, X⁵, and X⁶ are each independently CR³.

In certain embodiments of Formula (II), X⁷ is of the following formula (b):

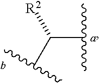

wherein a indicates the point of attachment to the amide nitrogen and b indicates the point of attachment to the heteroaryl ring. In certain embodiments of Formula (II), X⁷ is of the following formula (a):

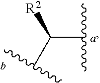

wherein a indicates the point of attachment to the amide nitrogen and b indicates the point of attachment to the heteroaryl ring. In certain embodiments of Formula (II), X⁷ is of the following formula (b-1):

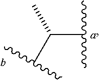

wherein a indicates the point of attachment to the amide nitrogen and b indicates the point of attachment to the heteroaryl ring. In certain embodiments of Formula (II), X⁷ is of the following formula (a-1):

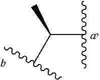

wherein a indicates the point of attachment to the amide nitrogen and b indicates the point of attachment to the heteroaryl ring.

In some embodiments of Formula (II), the carbon atom bonded to R² (at the X⁷ position) is in the (S) configuration. In some embodiments of Formula (II), the carbon atom bonded to R² (at the X⁷ position) is in the (R) configuration.

In some embodiments, the compounds are of Formula (II), or pharmaceutically acceptable salts, hydrates, solvates, prodrugs, stereoisomers, or tautomers thereof, wherein:
r is 1;
R¹ is H;
X⁷ is —CH(R²)—, wherein R² is C₁-C₆ alkyl;
the other R² is H or C₁-C₆ alkyl;
each R³ is independently H, halogen, C₁-C₆ alkyl, or heteroaryl, wherein each alkyl or heteroaryl is optionally substituted with one or more R⁶; and
R⁹ is H.

In certain embodiments, the compounds of Formula (II) are of Formula (II-a):

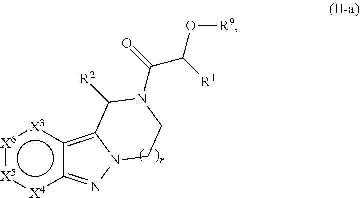

or pharmaceutically acceptable salts, hydrates, solvates, prodrugs, stereoisomers, or tautomers thereof, wherein:
R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; and
X³, X⁴, X⁵, and X⁶ are each independently CR³.

In some embodiments, the compounds are of Formula (II-a), or pharmaceutically acceptable salts, hydrates, solvates, prodrugs, stereoisomers, or tautomers thereof, wherein:
r is 1;
R¹ is H;
R² is C₁-C₆ alkyl;
each R³ is independently H, halogen, C₁-C₆ alkyl, or heteroaryl, wherein each alkyl or heteroaryl is optionally substituted with one or more R⁶; and
R⁹ is H.

In some embodiments of Formula (II-a), the carbon atom bonded to R² is in the (S) configuration. In some embodiments of Formula (II-a), the carbon atom bonded to R² is in the (R) configuration.

In some embodiments, the compounds of Formula (II) are of Formula (II-b):

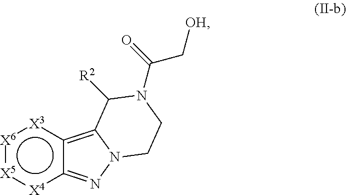

or pharmaceutically acceptable salts, hydrates, solvates, prodrugs, stereoisomers, or tautomers thereof.

In some embodiments, the compounds are of Formula (II-b), or pharmaceutically acceptable salts, hydrates, solvates, prodrugs, stereoisomers, or tautomers thereof, wherein:
R² is C₁-C₆ alkyl; and
each R³ is independently H, halogen, C₁-C₆ alkyl, or heteroaryl, wherein each alkyl or heteroaryl is optionally substituted with one or more R⁶.

In some embodiments of Formula (II-b), the carbon atom bonded to R² is in the (S) configuration. In some embodiments of Formula (II-b), the carbon atom bonded to R² is in the (R) configuration.

In some embodiments, the compounds of Formula (II) are of Formula (II-c):

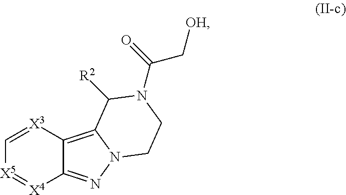

or pharmaceutically acceptable salts, hydrates, solvates, prodrugs, stereoisomers, or tautomers thereof.

In some embodiments, the compounds are of Formula (II-c), or pharmaceutically acceptable salts, hydrates, solvates, prodrugs, stereoisomers, or tautomers thereof, wherein:
R² is C₁-C₆ alkyl;
X³ is CR³, wherein R³ is H or heteroaryl, wherein the heteroaryl is optionally substituted with one or more R⁶;
X⁴ is CR³, wherein R³ is halogen; and
X⁵ is CR³, wherein R³ is halogen.

In some embodiments of Formula (II-c), the carbon atom bonded to R² is in the (S) configuration. In some embodiments of Formula (II-c), the carbon atom bonded to R² is in the (R) configuration.

In some embodiments, the compounds of Formula (II) are of Formula (II-d):

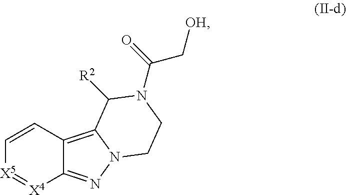

or pharmaceutically acceptable salts, hydrates, solvates, prodrugs, stereoisomers, or tautomers thereof.

In some embodiments, the compounds are of Formula (II-d), or pharmaceutically acceptable salts, hydrates, solvates, prodrugs, stereoisomers, or tautomers thereof, wherein:
R² is C₁-C₆ alkyl;
X⁴ is CR³, wherein R³ is halogen; and
X⁵ is CR³, wherein R³ is halogen.

In some embodiments of Formula (II-d), the carbon atom bonded to R² is in the (S) configuration. In some embodiments of Formula (II-d), the carbon atom bonded to R² is in the (R) configuration.

In some embodiments, the compounds of Formula (I) are compounds of Formula (I-a):

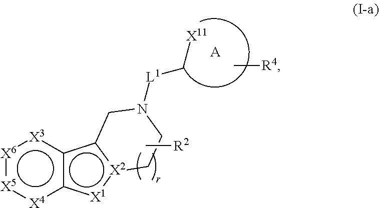

or pharmaceutically acceptable salts, prodrugs, solvates, hydrates, isomers, or tautomers thereof.

In some embodiments of Formula (I-a), the carbon atom bonded to R² is in the (S) configuration. In some embodiments of Formula (I-a), the carbon atom bonded to R² is in the (R) configuration.

In some embodiments, the compounds of Formula (I-a) are compounds of Formula (I-a-1):

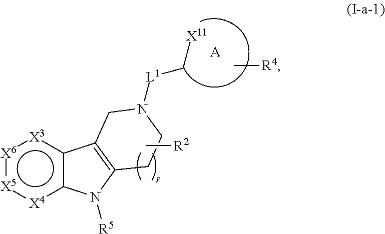

wherein X¹¹ is N or NH,
or pharmaceutically acceptable salts, prodrugs, solvates, hydrates, isomers, or tautomers thereof.

In some embodiments of Formula (I-a-1), the carbon atom bonded to R² is in the (S) configuration. In some embodiments of Formula (I-a-1), the carbon atom bonded to R² is in the (R) configuration.

In certain embodiments, Ring A is not a pyrazole, indole, imidazole, pyridine, or thiazole. In certain embodiments, Ring A is not a pyrazole. In certain embodiments, Ring A is not an indole. In certain embodiments, Ring A is not an imidazole. In certain embodiments, Ring A is not a pyridine. In certain embodiments, Ring A is not a thiazole.

In certain embodiments, when Ring A is a pyrimidine, then X⁶ is not C—CO(OR⁵). In certain embodiments, when Ring A is a pyrimidine, then X⁶ is not C—CO(OH). In certain embodiments, when Ring A is a pyrimidine, then R³ is not —CO(OR⁵). In certain embodiments, when R¹ and R⁹ combine to form a pyrimidine, then R³ is not —CO(OH).

In some embodiments, the compounds of Formula (I-a) are compounds of Formula (I-a-1-i):

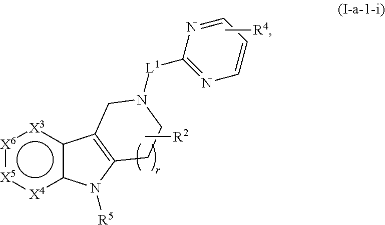

or pharmaceutically acceptable salts, prodrugs, solvates, hydrates, isomers, or tautomers thereof.

In some embodiments of Formula (I-a-1-i), the carbon atom bonded to R² is in the (S) configuration. In some embodiments of Formula (I-a-1-i), the carbon atom bonded to R² is in the (R) configuration.

In certain embodiments, X⁶ is not C—CO(OR⁵). In certain embodiments, X⁶ is not C—CO(OH).

In some embodiments, the compounds of Formula (I-a) are compounds of Formula (I-a-1-ii):

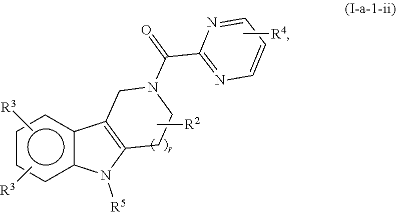

or pharmaceutically acceptable salts, prodrugs, solvates, hydrates, isomers, or tautomers thereof.

In some embodiments of Formula (I-a-1-ii), the carbon atom bonded to R² is in the (S) configuration. In some embodiments of Formula (I-a-1-ii), the carbon atom bonded to R² is in the (R) configuration.

In certain embodiments, when R³ is not —CO(OR⁵). In certain embodiments, R³ is not —CO(OH).

In certain embodiments, the compounds of Formula (I) are of Formula (IV):

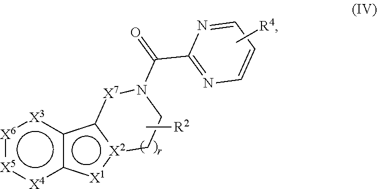

or pharmaceutically acceptable salts, hydrates, solvates, prodrugs, stereoisomers, or tautomers thereof, wherein:
X¹ is N or NR⁵, and X² is N or C;
X⁷ is —CH(R²)—;
X³, X⁴, X⁵, and X⁶ are each independently CR³; and
each R³ is independently H, halogen, —CN, —OR⁵, —SR⁵, —NH₂, —NH(R⁵), —N(R⁵)(R⁶), —NHC(O)R⁵, —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶.

In certain embodiments of Formula (IV), X⁷ is of the following formula (b):

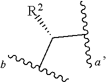

wherein a indicates the point of attachment to the amide nitrogen and b indicates the point of attachment to the heteroaryl ring. In certain embodiments of Formula (IV), X⁷ is of the following formula (a):

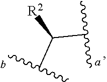

wherein a indicates the point of attachment to the amide nitrogen and b indicates the point of attachment to the heteroaryl ring. In certain embodiments of Formula (IV), X⁷ is of the following formula (b-1):

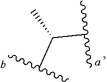

wherein a indicates the point of attachment to the amide nitrogen and b indicates the point of attachment to the heteroaryl ring. In certain embodiments of Formula (IV), X⁷ is of the following formula (a-1):

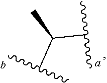

wherein a indicates the point of attachment to the amide nitrogen and b indicates the point of attachment to the heteroaryl ring.

In some embodiments of Formula (IV), the carbon atom bonded to R² (at the X⁷ position) is in the (S) configuration. In some embodiments of Formula (IV), the carbon atom bonded to R² (at the X⁷ position) is in the (R) configuration.

In some embodiments, the compounds are of Formula (IV), or pharmaceutically acceptable salts, hydrates, solvates, prodrugs, stereoisomers, or tautomers thereof, wherein:
X¹ is NH;
X² is N or C;
each R² is independently H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R⁵), or —N(R⁵)(R⁶).

In certain embodiments, the compounds of Formula (I) are of Formula (IV-1):

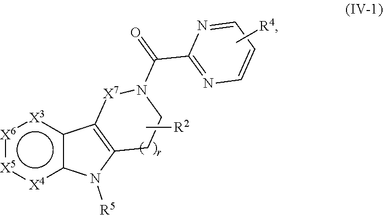

or pharmaceutically acceptable salts, hydrates, solvates, prodrugs, stereoisomers, or tautomers thereof, wherein:
X⁷ is —CH(R²)—;
X³, X⁴, X⁵, and X⁶ are each independently CR³; and
each R³ is independently H, halogen, —CN, —OR⁵, —SR⁵, —NH₂, —NH(R⁵), —N(R⁵)(R⁶), —NHC(O)R⁵, —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶.

In some embodiments, the compounds are of Formula (IV-1), or pharmaceutically acceptable salts, hydrates, solvates, prodrugs, stereoisomers, or tautomers thereof, wherein:
each R² is independently H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R⁵), or —N(R⁵)(R⁶).

In some embodiments of Formula (IV-1), the carbon atom bonded to R² (at the X⁷ position) is in the (S) configuration. In some embodiments of Formula (IV-1), the carbon atom bonded to R² (at the X⁷ position) is in the (R) configuration.

In certain embodiments, the compounds of Formula (I) are of Formula (IV-2):

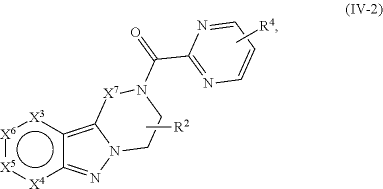

or pharmaceutically acceptable salts, hydrates, solvates, prodrugs, stereoisomers, or tautomers thereof, wherein:
X⁷ is —CH(R²)—;
X³, X⁴, X⁵, and X⁶ are each independently CR³; and
each R³ is independently H, halogen, —CN, —OR⁵, —SR⁵, —NH₂, —NH(R⁵), —N(R⁵)(R⁶), —NHC(O)R⁵, —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶.

In some embodiments, the compounds are of Formula (IV-2), or pharmaceutically acceptable salts, hydrates, solvates, prodrugs, stereoisomers, or tautomers thereof, wherein:
each R² is independently H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R⁵), or —N(R⁵)(R⁶).

In some embodiments of Formula (IV-2), the carbon atom bonded to R² (at the X⁷ position) is in the (S) configuration. In some embodiments of Formula (IV-2), the carbon atom bonded to R² (at the X⁷ position) is in the (R) configuration.

In some embodiments, the compounds of Formula (IV) are of Formula (IV-a):

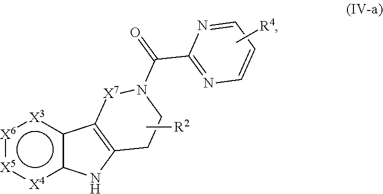

or pharmaceutically acceptable salts, hydrates, solvates, prodrugs, stereoisomers, or tautomers thereof.

In some embodiments, the compounds are of Formula (IV-a), or pharmaceutically acceptable salts, hydrates, solvates, prodrugs, stereoisomers, or tautomers thereof, wherein:
each R² is independently H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R₅), or —N(R⁵)(R⁶).

In some embodiments of Formula (IV-a), the carbon atom bonded to R² (at the X⁷ position) is in the (S) configuration. In some embodiments of Formula (IV-a), the carbon atom bonded to R² (at the X⁷ position) is in the (R) configuration.

In some embodiments, the compounds of Formula (IV) are of Formula (IV-b):

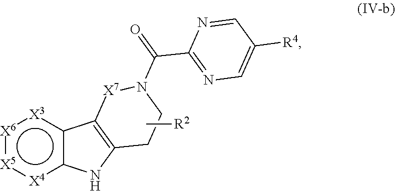

or pharmaceutically acceptable salts, hydrates, solvates, prodrugs, stereoisomers, or tautomers thereof.

In some embodiments, the compounds are of Formula (IV-b), or pharmaceutically acceptable salts, hydrates, solvates, prodrugs, stereoisomers, or tautomers thereof, wherein:
each R² is independently H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R⁵), or —N(R⁵)(R⁶).

In some embodiments of Formula (IV-b), the carbon atom bonded to R² (at the X⁷ position) is in the (S) configuration. In some embodiments of Formula (IV-b), the carbon atom bonded to R² (at the X⁷ position) is in the (R) configuration.

In some embodiments, the compounds of Formula (IV) are of Formula (IV-c):

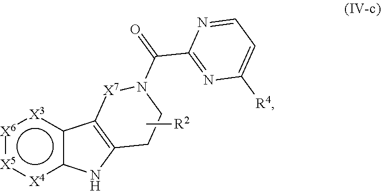

or pharmaceutically acceptable salts, hydrates, solvates, prodrugs, stereoisomers, or tautomers thereof.

In some embodiments, the compounds are of Formula (IV-c), or pharmaceutically acceptable salts, hydrates, solvates, prodrugs, stereoisomers, or tautomers thereof, wherein:
each R² is independently H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R⁵), or —N(R⁵)(R⁶).

In some embodiments of Formula (IV-c), the carbon atom bonded to R² (at the X⁷ position) is in the (S) configuration. In some embodiments of Formula (IV-c), the carbon atom bonded to R² (at the X⁷ position) is in the (R) configuration.

In some embodiments, the compounds of Formula (IV) are of Formula (IV-d):

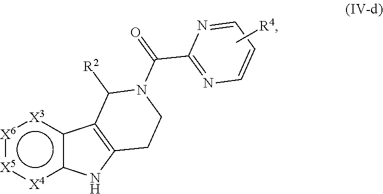

or pharmaceutically acceptable salts, hydrates, solvates, prodrugs, stereoisomers, or tautomers thereof.

In some embodiments, the compounds are of Formula (IV-d), or pharmaceutically acceptable salts, hydrates, solvates, prodrugs, stereoisomers, or tautomers thereof, wherein:
R² is H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R⁵), or —N(R⁵)(R⁶).

In some embodiments of Formula (IV-d), the carbon atom bonded to R² is in the (S) configuration. In some embodiments of Formula (IV-d), the carbon atom bonded to R² is in the (R) configuration.

In some embodiments, the compounds of Formula (IV) are of Formula (IV-e):

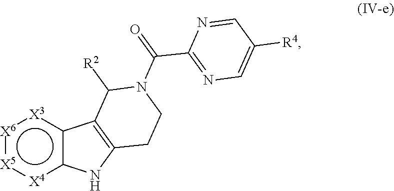

or pharmaceutically acceptable salts, hydrates, solvates, prodrugs, stereoisomers, or tautomers thereof.

In some embodiments, the compounds are of Formula (IV-e), or pharmaceutically acceptable salts, hydrates, solvates, prodrugs, stereoisomers, or tautomers thereof, wherein:
each R² is independently H or C₁-C₆ alkyl;
each R³ is independently H, halogen, C₁-C₆ alkyl, or heteroaryl, wherein each alkyl or heteroaryl is optionally substituted with one or more R⁶; and
R⁴ is H, halogen, C₁-C₆ alkyl, —OR⁵, —NH₂, —NH(R⁵), or —N(R⁵)(R⁶).

In some embodiments of Formula (IV-e), the carbon atom bonded to R² is in the (S) configuration. In some embodiments of Formula (IV-e), the carbon atom bonded to R² is in the (R) configuration.

In some embodiments, the compounds of Formula (IV) are of Formula (IV-f):

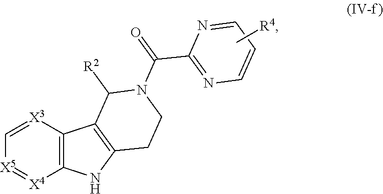

or pharmaceutically acceptable salts, hydrates, solvates, prodrugs, stereoisomers, or tautomers thereof.

In some embodiments, the compounds are of Formula (IV-f), or pharmaceutically acceptable salts, hydrates, solvates, prodrugs, stereoisomers, or tautomers thereof, wherein:
R² is H or C₁-C₆ alkyl;
X³ is CR³, wherein R³ is H or heteroaryl, wherein the heteroaryl is optionally substituted with one or more R⁶;
X⁴ is CR³, wherein R³ is halogen;
X⁵ is CR³, wherein R³ is halogen; and
R⁴ is H, halogen, C₁-C₆ alkyl, —OR⁵, —NH₂, —NH(R⁵), or —N(R⁵)(R⁶).

In some embodiments of Formula (IV-f), the carbon atom bonded to R² is in the (S) configuration. In some embodiments of Formula (IV-f), the carbon atom bonded to R² is in the (R) configuration.

In some embodiments, the compounds of Formula (IV) are of Formula (IV-g):

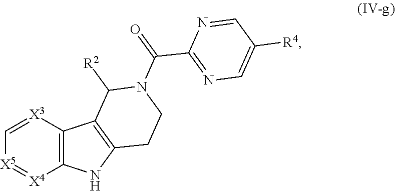

or pharmaceutically acceptable salts, hydrates, solvates, prodrugs, stereoisomers, or tautomers thereof.

In some embodiments, the compounds are of Formula (IV-g), or pharmaceutically acceptable salts, hydrates, solvates, prodrugs, stereoisomers, or tautomers thereof, wherein:
R² is H or C₁-C₆ alkyl;
X³ is CR³, wherein R³ is H or heteroaryl, wherein the heteroaryl is optionally substituted with one or more R⁶.
X⁴ is CR³, wherein R³ is halogen;
X⁵ is CR³, wherein R³ is halogen; and
R⁴ is H, halogen, C₁-C₆ alkyl, —OR⁵, —NH₂, —NH(R⁵), or —N(R⁵)(R⁶).

In some embodiments of Formula (IV-g), the carbon atom bonded to R² is in the (S) configuration. In some embodiments of Formula (IV-g), the carbon atom bonded to R² is in the (R) configuration.

In some embodiments, the compounds of Formula (I-a) are compounds of Formula (I-a-2):

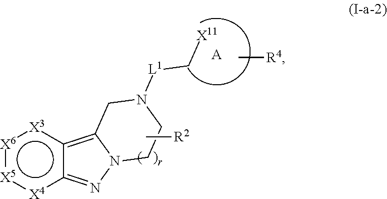

wherein X¹¹ is N or NH, or pharmaceutically acceptable salts, prodrugs, solvates, hydrates, isomers, or tautomers thereof.

In some embodiments of Formula (I-a-2), the carbon atom bonded to R² is in the (S) configuration. In some embodiments of Formula (I-a-2), the carbon atom bonded to R² is in the (R) configuration.

In some embodiments, the compounds of Formula (I-a) are compounds of Formula (I-a-2-i):

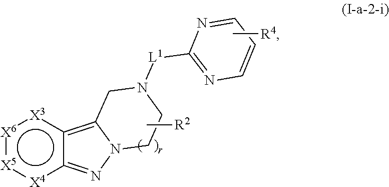

or pharmaceutically acceptable salts, prodrugs, solvates, hydrates, isomers, or tautomers thereof.

In some embodiments of Formula (I-a-2-i), the carbon atom bonded to R² is in the (S) configuration. In some embodiments of Formula (I-a-2-i), the carbon atom bonded to R² is in the (R) configuration.

In some embodiments, the compounds of Formula (I-a) are compounds of Formula (I-a-2-ii):

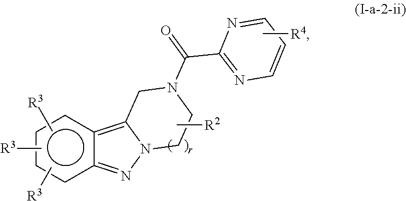

or pharmaceutically acceptable salts, prodrugs, solvates, hydrates, isomers, or tautomers thereof.

In some embodiments of Formula (I-a-2-ii), the carbon atom bonded to R² is in the (S) configuration. In some embodiments of Formula (I-a-2-ii), the carbon atom bonded to R² is in the (R) configuration.

In some embodiments, the compounds of Formula (I-a) are compounds of is Formula (I-a-3) to Formula (I-a-11):

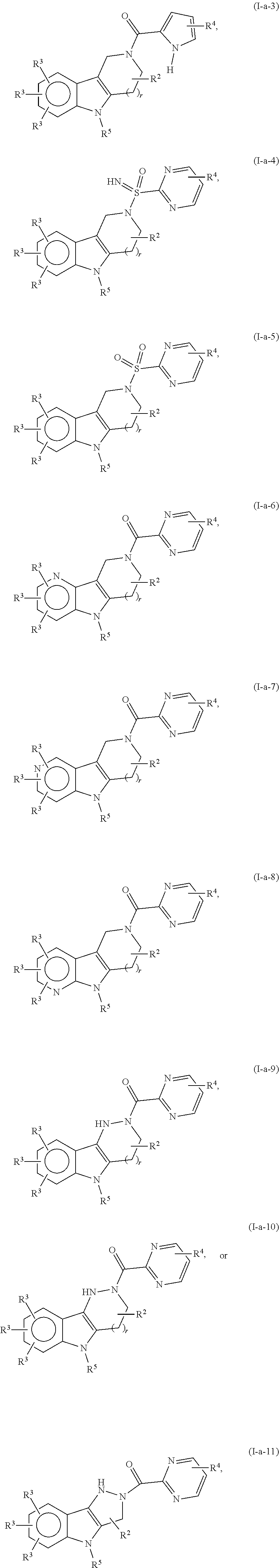

or pharmaceutically acceptable salts, prodrugs, solvates, hydrates, isomers, or tautomers thereof.

In some embodiments of Formulae (I-a-3), (I-a-4), (I-a-5), (I-a-6), (I-a-7), (I-a-8), (I-a-9), (I-a-10), or (I-a-11), the carbon atom bonded to R² is in the (S) configuration. In some embodiments, the carbon atom bonded to R² is in the (R) configuration.

In some embodiments, the compound is of the Formula (I-b):

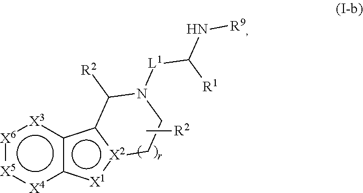

or pharmaceutically acceptable salts, prodrugs, solvates, hydrates, isomers, or tautomers thereof

In some embodiments of Formula (I-b), the carbon atom bonded to R² is in the (S) configuration. In some embodiments of Formula (I-b), the carbon atom bonded to R² is in the (R) configuration.

In some embodiments, the compound is of the Formula (I-b-1):

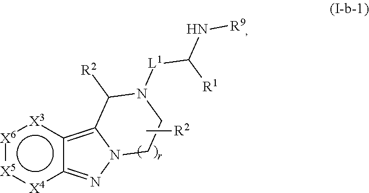

or pharmaceutically acceptable salts, prodrugs, solvates, hydrates, isomers, or tautomers thereof.

In some embodiments of Formula (I-b-1), the carbon atom at position X⁷ bonded to R² is in the (S) configuration. In some embodiments of Formula (I-b-1), the carbon atom at position X⁷ bonded to R² is in the (R) configuration.

In certain embodiments, the compounds of Formula (I) are of Formula (III):

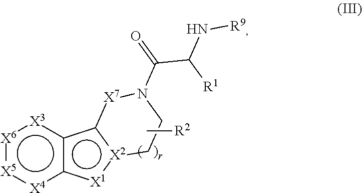

or pharmaceutically acceptable salts, hydrates, solvates, prodrugs, stereoisomers, or tautomers thereof, wherein:
X¹ is N or NR⁵, and X² is N or C;
X³, X⁴, X⁵, and X⁶ are each independently CR³;
X⁷ is —CH(R²)—, wherein R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR, —(CH₂)ₙ—OR, aryl, or heteroaryl; and
the other R² is hydrogen, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle.

In some embodiments, the compounds are of Formula (III), or pharmaceutically acceptable salts, hydrates, solvates, prodrugs, stereoisomers, or tautomers thereof, wherein:
X¹ is NH;
X² is N or C;
R¹ is H;
X⁷ is —CH(R²)—, wherein R² is C₁-C₆ alkyl;
the other R² is H or C₁-C₆ alkyl;
each R³ is independently H, halogen, C₁-C₆ alkyl, or heteroaryl wherein each alkyl or heteroaryl is optionally substituted with one or more R⁶.
R⁹ is H; and
r is 1.

In certain embodiments of Formula (III), X⁷ is of the following formula (b):

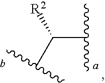

wherein a indicates the point of attachment to the amide nitrogen and b indicates the point of attachment to the heteroaryl ring. In certain embodiments of Formula (III), X⁷ is of the following formula (a):

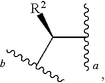

wherein a indicates the point of attachment to the amide nitrogen and b indicates the point of attachment to the heteroaryl ring. In certain embodiments of Formula (III), X⁷ is of the following formula (b-1):

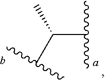

wherein a indicates the point of attachment to the amide nitrogen and b indicates the point of attachment to the heteroaryl ring. In certain embodiments of Formula (III), X⁷ is of the following formula (a-1):

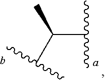

wherein a indicates the point of attachment to the amide nitrogen and b indicates the point of attachment to the heteroaryl ring.

In some embodiments of Formula (III), the carbon atom bonded to R² (at the X⁷ position) is in the (S) configuration. In some embodiments of Formula (III), the carbon atom bonded to R² (at the X⁷ position) is in the (R) configuration.

In some embodiments, the compounds of Formula (III) are of Formula (III-a):

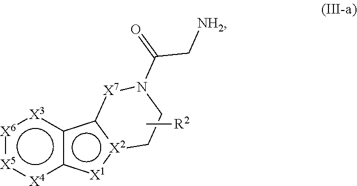

or pharmaceutically acceptable salts, hydrates, solvates, prodrugs, stereoisomers, or tautomers thereof.

In some embodiments, the compounds are of Formula (III-a), or pharmaceutically acceptable salts, hydrates, solvates, prodrugs, stereoisomers, or tautomers thereof, wherein:
X¹ is NH;
X² is N or C; and
X⁷ is —CH(R²)—, wherein R² is C₁-C₆ alkyl;
the other R² is H or C₁-C₆ alkyl; and
each R³ is independently H, halogen, C₁-C₆ alkyl, or heteroaryl wherein each alkyl or heteroaryl is optionally substituted with one or more R⁶.

In some embodiments of Formula (III-a), the carbon atom bonded to R² (at the X⁷ position) is in the (S) configuration. In some embodiments of Formula (III-a), the carbon atom bonded to R² (at the X⁷ position) is in the (R) configuration.

In some embodiments, the compounds of Formula (III) are of Formula (III-b):

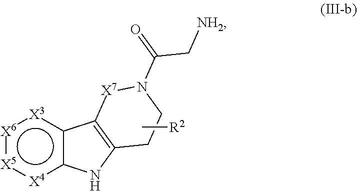

or pharmaceutically acceptable salts, hydrates, solvates, prodrugs, stereoisomers, or tautomers thereof.

In some embodiments, the compounds are of Formula (III-b), or pharmaceutically acceptable salts, hydrates, solvates, prodrugs, stereoisomers, or tautomers thereof, wherein:
X⁷ is —CH(R²)—, wherein R² is C₁-C₆ alkyl;
the other R² is H or C₁-C₆ alkyl; and
each R³ is independently H, halogen, C₁-C₆ alkyl, or heteroaryl wherein each alkyl or heteroaryl is optionally substituted with one or more R⁶.

In some embodiments of Formula (III-b), the carbon atom bonded to R² (at the X⁷ position) is in the (S) configuration. In some embodiments of Formula (III-b), the carbon atom bonded to R² (at the X⁷ position) is in the (R) configuration.

In some embodiments, the compounds of Formula (III) are of Formula (III-c):

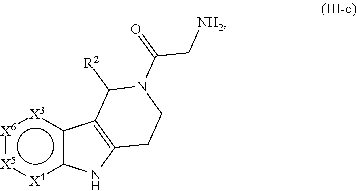

or pharmaceutically acceptable salts, hydrates, solvates, prodrugs, stereoisomers, or tautomers thereof.

In some embodiments, the compounds are of Formula (III-c), or pharmaceutically acceptable salts, hydrates, solvates, prodrugs, stereoisomers, or tautomers thereof, wherein:
R² is C₁-C₆ alkyl; and
each R³ is independently H, halogen, C₁-C₆ alkyl, or heteroaryl, wherein each alkyl or heteroaryl is optionally substituted with one or more R⁶.

In some embodiments of Formula (III-c), the carbon atom bonded to R² is in the (S) configuration. In some embodiments of Formula (III-c), the carbon atom bonded to R² is in the (R) configuration.

In some embodiments, the compounds of Formula (III) are of Formula (III-d):

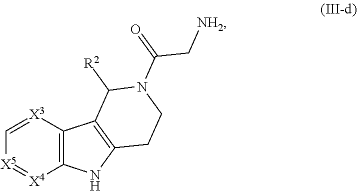

or pharmaceutically acceptable salts, hydrates, solvates, prodrugs, stereoisomers, or tautomers thereof.

In some embodiments, the compounds are of Formula (III-d), or pharmaceutically acceptable salts, hydrates, solvates, prodrugs, stereoisomers, or tautomers thereof, wherein:
R² is C₁-C₆ alkyl;
X³ is CR³, wherein R³ is H or heteroaryl, wherein the heteroaryl is optionally substituted with one or more R⁶;
X⁴ is CR³, wherein R³ is halogen; and
X⁵ is CR³, wherein R³ is halogen.

In some embodiments of Formula (III-d), the carbon atom bonded to R² is in the (S) configuration. In some embodiments of Formula (III-d), the carbon atom bonded to R² is in the (R) configuration.

In some embodiments, the compounds of Formula (III) are of Formula (III-e):

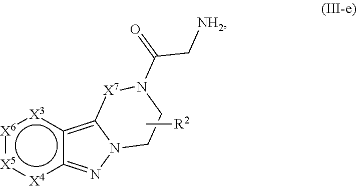

or pharmaceutically acceptable salts, hydrates, solvates, prodrugs, stereoisomers, or tautomers thereof.

In some embodiments, the compounds are of Formula (III-e), or pharmaceutically acceptable salts, hydrates, solvates, prodrugs, stereoisomers, or tautomers thereof, wherein:
X⁷ is —CH(R²)—, wherein R² is C₁-C₆ alkyl;
the other R² is H or C₁-C₆ alkyl; and
each R³ is independently H, halogen, C₁-C₆ alkyl, or heteroaryl wherein each alkyl or heteroaryl is optionally substituted with one or more R⁶.

In some embodiments of Formula (III-e), the carbon atom bonded to R² (at the X⁷ position) is in the (S) configuration. In some embodiments of Formula (III-e), the carbon atom bonded to R² (at the X⁷ position) is in the (R) configuration.

In some embodiments, the compounds of Formula (III) are of Formula (III-f):

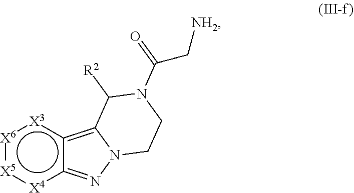

or pharmaceutically acceptable salts, hydrates, solvates, prodrugs, stereoisomers, or tautomers thereof.

In some embodiments, the compounds are of Formula (III-f), or pharmaceutically acceptable salts, hydrates, solvates, prodrugs, stereoisomers, or tautomers thereof, wherein:
R² is C₁-C₆ alkyl; and
each R³ is independently H, halogen, C₁-C₆ alkyl, or heteroaryl, wherein each alkyl or heteroaryl is optionally substituted with one or more R⁶.

In some embodiments of Formula (III-f), the carbon atom bonded to R² is in the (S) configuration. In some embodiments of Formula (III-f), the carbon atom bonded to R² is in the (R) configuration.

In some embodiments, the compounds of Formula (III) are of Formula (III-g):

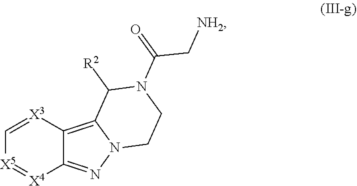

or pharmaceutically acceptable salts, hydrates, solvates, prodrugs, stereoisomers, or tautomers thereof.

In some embodiments, the compounds are of Formula (III-g), or pharmaceutically acceptable salts, hydrates, solvates, prodrugs, stereoisomers, or tautomers thereof, wherein:
R² is C₁-C₆ alkyl;
X³ is CR³, wherein R³ is H or heteroaryl, wherein the heteroaryl is optionally substituted with one or more R⁶.
X⁴ is CR³, wherein R³ is halogen; and
X⁵ is CR³, wherein R³ is halogen.

In some embodiments of Formula (III-g), the carbon atom bonded to R² is in the (S) configuration. In some embodiments of Formula (III-g), the carbon atom bonded to R² is in the (R) configuration.

In some embodiments, the compound is of the Formula (I-b-2):

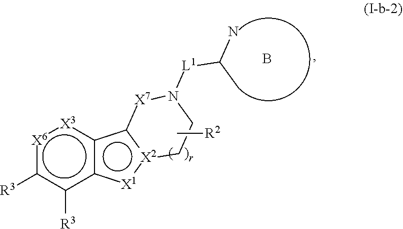

wherein ring B is:

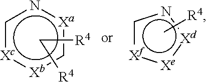

wherein X^{a}, X^{b}, and X^{c} are independently N or CH; X^{d}, X^{e}, and X^{f} are independently N, NH, or CH, or pharmaceutically acceptable salts, prodrugs, solvates, hydrates, isomers, or tautomers thereof.

In some embodiments of Formula (I-b-2), the carbon atom bonded to R² (at the X⁷ position) is in the (S) configuration. In some embodiments of Formula (I-b-2), the carbon atom bonded to R² (at the X⁷ position) is in the (R) configuration.

In some embodiments, the compound is of the Formula (I-c):

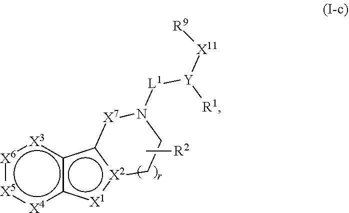

wherein Y is CH or NH; X¹¹ is O, N, or NH; R¹ and R⁹ combine to form a 5- to 10-membered heteroaryl having the formulae:

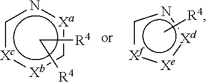

wherein X^{a}, X^{b}, and X^{c} are independently N or CH; X^{d}, X^{e}, and X are independently N, NH, or CH, or pharmaceutically acceptable salts, prodrugs, solvates, hydrates, isomers, or tautomers thereof.

In some embodiments of Formula (I-c), the carbon atom bonded to R² (at the X⁷ position) is in the (S) configuration. In some embodiments of Formula (I-c), the carbon atom bonded to R² (at the X⁷ position) is in the (R) configuration.

In some embodiments, the compound is of Formula (I-d):

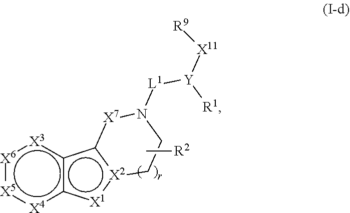

wherein Y is CH or NH; X¹¹ is O, N, or NH; R¹ and R⁹ combine to form a 5- to 10-membered heteroaryl having the formulae:

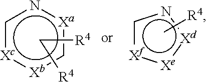

wherein X^{a}, X^{b}, and X^{c} are independently N or CH; X^{d}, X^{e}, and X^{f} are independently N, NH, or CH, or pharmaceutically acceptable salts, prodrugs, solvates, hydrates, isomers, or tautomers thereof.

In some embodiments of Formula (I-d), the carbon atom bonded to R² (at the X⁷ position) is in the (S) configuration. In some embodiments of Formula (I-d), the carbon atom bonded to R² (at the X² position) is in the (R) configuration.

In some embodiments of any of the above Formulae, L¹ is —C(O)—, —S(O)—, —S(O)₂—, —S(NH)(O)—. In some embodiments, L¹ is —C(O)—. In some embodiments, L¹ is —S(O)—. In other embodiments, L¹ is —S(O)₂—. In some embodiments, L¹ is —S(NH)(O)—.

In some embodiments of any of the above Formulae, X¹ is N. In other embodiments, X¹ is NR⁵. In some embodiments, X¹ is CH. In some embodiments, X¹ is N or NR⁵. In some embodiments, X¹ is N or CH.

In some embodiments of any of the above Formulae, X² is N. In other embodiments, X² is C. In some embodiments, X² is N or C.

In some embodiments of any of the above Formulae, X³ is N. In other embodiments, X³ is CR³. In some embodiments, X³ is N or CR³.

In some embodiments of any of the above Formulae, X⁴ is N. In other embodiments, X⁴ is CR³. In some embodiments, X⁴ is N or CR³.

In some embodiments of any of the above Formulae, X⁵ is N. In other embodiments, X⁵ is CR³. In some embodiments, X⁵ is N or CR³.

In some embodiments of any of the above Formulae, X⁶ is N. In other embodiments, X⁶ is CR³. In some embodiments, X⁶ is N or CR³.

In some embodiments of any of the above Formulae, X⁸ is N. In other embodiments, X⁸ is CR³. In other embodiments, X⁸ is C. In some embodiments, X⁸ is N or CR³.

In some embodiments of any of the above Formulae, X⁹ is N. In other embodiments, X⁹ is CR³. In other embodiments, X⁹ is C. In some embodiments, X⁹ is N or CR³.

In some embodiments of any of the above Formulae, X¹⁰ is N. In other embodiments, X¹⁰ is CR³. In other embodiments, X¹⁰ is C. In some embodiments, X¹⁰ is N or CR³.

In some embodiments of any of the above Formulae, X⁷ is NH. In other embodiments, X⁷ is NCH₃. In some embodiments, X⁷ is C(R²)₂.

In some embodiments of any of the above Formulae, X¹¹ is N. In other embodiments, X¹¹ is O. In some embodiments, X¹¹ is NH. In other embodiments, X¹¹ is O or NH. In other embodiments, X¹¹ is O or N. In other embodiments, X¹¹ is NH or N. In other embodiments, X¹¹ is O, N, or NH.

In some embodiments of any of the above Formulae, Y is NH. In other embodiments, Y is CH. In some embodiments, Y is C. In other embodiments, Y is C or CH. In other embodiments, Y is NH or CH. In other embodiments, Y is NH or N. In other embodiments, Y is C or CH.

In some embodiments of any of the above Formulae, R¹ is H. In some embodiments, R¹ is C₁-C₆ alkyl. In some embodiments, R¹ is C₁-C₆ alkyl optionally substituted with one or more R⁴.

In other embodiments of any of the above Formulae, R¹ and R⁹ may combine to form a 3- to 8-membered heterocycle, or 5- to 10-membered heteroaryl. Yet in other embodiments, R¹ and R⁹ may combine to form a 3- to 8-membered heterocycle. In other embodiments, R¹ and R⁹ may combine to form a 5- to 10-membered heteroaryl. Yet in other embodiments, R¹ and R⁹ may combine to form a 3- to 8-membered heterocycle optionally substituted with one or more R⁴. In other embodiments, R¹ and R⁹ may combine to form a 5- to 10-membered heteroaryl optionally substituted with one or more R⁴.

In some embodiments of any of the above Formulae, each instance of R² is independently H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl. In some embodiments, each instance of R² is independently halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl. In some embodiments, each instance of R² is independently H, C₁-C₆ alkyl, C₂-C₆ alkenyl, C₂-C₆ alkynyl, C₁-C₆ haloalkyl, —(CH₂)ₙ—SR⁸, —(CH₂)ₙ—OR⁸, aryl, or heteroaryl. In some embodiments, each instance of R² is independently C₁-C₆ alkyl, C₂-C₆ alkenyl, C₂-C₆ alkynyl, C₁-C₆ haloalkyl, —(CH₂)ₙ—SR⁸, —(CH₂)ₙ—OR⁸, aryl, or heteroaryl.

In some embodiments of any of the above Formulae, at least one instance of R² is H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl. In some embodiments, at least one instance of R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl. In some embodiments, at least one instance of R² is H, C₁-C₆ alkyl, C₂-C₆ alkenyl, C₂-C₆ alkynyl, C₁-C₆ haloalkyl, —(CH₂)ₙ—SR⁸, —(CH₂)ₙ—OR⁸, aryl, or heteroaryl. In some embodiments, at least one instance of R² is C₁-C₆ alkyl, C₂-C₆ alkenyl, C₂-C₆ alkynyl, C₁-C₆ haloalkyl, —(CH₂)ₙ—SR⁸, —(CH₂)ₙ—OR⁸, aryl, or heteroaryl.

In some embodiments of any of the above Formulae, R² is H.

However, in certain embodiments of any of the above Formulae, R² is not hydrogen. For example, in some embodiments, R² is halogen. In some embodiments, R² is CN. In some embodiments, R² is OH. In some embodiments, R² is C₁-C₆ alkyl. In some embodiments, R² is C₁-C₆ alkenyl. In some embodiments, R² is C₂-C₆ alkynyl. In some embodiments, R² is C₁-C₆ haloalkyl. In some embodiments, R² is C₁-C₆ haloalkoxy. In some embodiments, R² is —(CH₂)ₙ—SR⁸. In some embodiments, R² is —(CH₂)ₙ—OR⁸. In some embodiments, R² is aryl. In some embodiments, R² is heteroaryl.

In some embodiments of any of the above Formulae, two R², combined with the carbon to which they are independently attached may form a C₄-C₈ cycloalkyl or 4- to 6-membered heterocycle. In some embodiments, two R², combined with the carbon to which they are independently attached may form a C₄-C₈ cycloalkyl. In some embodiments, two R², combined with the carbon to which they are independently attached may form a 4- to 6-membered heterocycle.

In some embodiments of any of the above Formulae, at least one instance of R² is not H. In some embodiments, at least one instance of R² is C₁-C₆ alkyl. In some embodiments, at least one instance of R² is C₁-C₃ alkyl. In some embodiments, at least one instance of R² is methyl.

In some embodiments of any of the above Formulae, the carbon atom bonded to R² is in the (S) configuration. In some embodiments, the carbon atom bonded to R² is in the (R) configuration.

In some embodiments of any of the above Formulae, X⁷ is —CH(R²)—, wherein R² is not H. In some embodiments, X⁷ is —CH(R²)—, wherein R² is C₁-C₆ alkyl. In some embodiments, X⁷ is —CH(R²)—, wherein R² is C₁-C₃ alkyl. In some embodiments, X⁷ is —CH(R²)—, wherein R² is methyl.

In some embodiments of any of the above Formulae, the carbon atom bonded to R² (at the X⁷ position) is in the (S) configuration. In some embodiments, the carbon atom bonded to R² (at the X⁷ position) is in the (R) configuration.

In certain embodiments of any of the above Formulae, X⁷ is of the following formula (b):

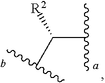

wherein a indicates the point of attachment to the amide nitrogen and b indicates the point of attachment to the heteroaryl ring. In certain embodiments, X⁷ is of the following formula (a):

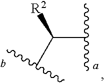

wherein a indicates the point of attachment to the amide nitrogen and b indicates the point of attachment to the heteroaryl ring. In certain embodiments, X⁷ is of the following formula (b-1):

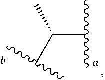

wherein a indicates the point of attachment to the amide nitrogen and b indicates the point of attachment to the heteroaryl ring. In certain embodiments, X⁷ is of the following formula (a-1):

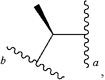

wherein a indicates the point of attachment to the amide nitrogen and b indicates the point of attachment to the heteroaryl ring.

In some embodiments of any of the above Formulae, at least one R³ is H. In other embodiments of any of the above Formulae, at least one R³ is a non-hydrogen group. For example, in certain embodiments, at least one R³ is halogen. In some embodiments, at least one R³ is oxo. In some embodiments, at least one R³ is —CN. In some embodiments, at least one R³ is —OR⁵. In some embodiments, at least one R³ is —SR⁵. In some embodiments, at least one R³ is —NH₂. In some embodiments, at least one R³ is —NH(R⁵). In some embodiments, at least one R³ is —N(R⁵)(R⁶). In some embodiments, at least one R³ is —NHC(O)R⁵. In some embodiments, at least one R³ is —CO(OR⁵). In some embodiments, at least one R³ is —C(O)R⁵. In some embodiments, at least one R³ is —C(O)N(R⁵)₂. In some embodiments, at least one R³ is C₁-C₆ alkyl. In some embodiments, at least one R³ is C₁-C₆ haloalkyl. In some embodiments, at least one R³ is C₁-C₆ haloalkoxy. In some embodiments, at least one R³ is C₂-C₆ alkenyl. In some embodiments, at least one R³ is C₂-C₆ alkynyl. In some embodiments, at least one R³ is C₃-C₈ cycloalkyl. In some embodiments, at least one R³ is heteroaryl. In some embodiments, at least one R³ is heterocyclyl. In some embodiments, at least one R³ is C₁-C₆ alkyl optionally substituted with one or more R⁶. In some embodiments, at least one R³ is C₁-C₆ haloalkyl optionally substituted with one or more R⁶. In some embodiments, at least one R³ is C₁-C₆ haloalkoxy optionally substituted with one or more R⁶. In some embodiments, at least one R³ is C₂-C₆ alkenyl optionally substituted with one or more R⁶. In some embodiments, at least one R³ is C₂-C₆ alkynyl optionally substituted with one or more R⁶. In some embodiments, at least one R³ is C₃-C₈ cycloalkyl optionally substituted with one or more R⁶. In some embodiments, at least one R³ is heteroaryl optionally substituted with one or more R⁶. In some embodiments, at least one R³ is heterocyclyl optionally substituted with one or more R⁶.

In some embodiments of any of the above Formulae, R⁴ is H, halogen, C₁-C₆ alkyl, —OR⁵, —NH₂, —NH(R⁵), or —N(R⁵)(R⁶). In some embodiments, R⁴ is —OR⁵, —NH₂, —NH(R⁵), or —N(R⁵)(R⁶). In some embodiments, R⁴ is H. In some embodiments, R⁴ is halogen. In some embodiments, R⁴ is —CN. In some embodiments, R⁴ is —OR⁵. In some embodiments, R⁴ is —NH₂. In some embodiments, R⁴ is —NH(R⁵). In some embodiments, R⁴ is —N(R⁵)(R⁶). In some embodiments, R⁴ is —NHC(O)R⁵. In some embodiments, R⁴ is —CO(OR⁵). In some embodiments, R⁴ is —C(O)R⁵, In some embodiments, R⁴ is —C(O)N(R⁵)₂. In some embodiments, R⁴ is —(CH₂)ₙ—OR⁸. In some embodiments, R⁴ is C₁-C₆ alkyl. In some embodiments, R⁴ is C₁-C₆ alkoxy. In some embodiments, R⁴ is C₂-C₆ alkenyl. In some embodiments, R⁴ is C₂-C₆ alkynyl. In some embodiments, R⁴ is C₃-C₃ cycloalkyl. In some embodiments, R⁴ is heterocyclyl. In some embodiments, R⁴ is heteroaryl. In some embodiments, R⁴ is aryl. In some embodiments, R⁴ is C₁-C₆ alkyl optionally substituted with one or R₇. In some embodiments, R⁴ is C₁-C₆ alkoxy optionally substituted with one or R₇. In some embodiments, R⁴ is C₂-C₆ alkenyl optionally substituted with one or R₇. In some embodiments, R⁴ is C₂-C₆ alkynyl optionally substituted with one or R₇. In some embodiments, R⁴ is C₃-C₃ cycloalkyl optionally substituted with one or R₇. In some embodiments, R⁴ is heterocyclyl optionally substituted with one or R₇. In some embodiments, R⁴ is heteroaryl optionally substituted with one or R₇. In some embodiments, R⁴ is aryl optionally substituted with one or R₇.

In some embodiments of any of the above Formulae, R⁵ is H. In some embodiments, R⁵ is —C(O)OH. In some embodiments, R⁵ is —(CH₂)ₙ—O—(CH₂)ₚ—OR⁸. In some embodiments, R⁵ is —(CH₂)ₙ—OR⁸. In some embodiments, R⁵ is —(CH₂)ₙ—S(O)₂R⁸. In some embodiments, R⁵ is —CN. In some embodiments, R⁵ is C₁-C₆ alkyl. In some embodiments, R⁵ is C₁-C₆ alkoxy. In some embodiments, R⁵ is C₂-C₆ alkenyl. In some embodiments, R⁵ is C₂-C₆ alkynyl. In some embodiments, R⁵ is C₃-C₈ cycloalkyl. In some embodiments, R⁵ is heterocyclyl. In some embodiments, R⁵ is heteroaryl. In some embodiments, R⁵ is aryl.

In some embodiments of any of the above Formulae, R⁵ is C₁-C₆ alkyl optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁵ is C₁-C₆ alkoxy optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁵ is C₂-C₆ alkenyl optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁵ is C₂-C₆ alkynyl optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁵ is C₃-C₈ cycloalkyl optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁵ is heterocyclyl optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁵ is heteroaryl optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁵ is aryl optionally substituted with one or more halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl.

In some embodiments of any of the above Formulae, R⁶ is H. In some embodiments, R⁶ is halogen. In some embodiments, R⁶ is —OH. In some embodiments, R⁶ is —CN. In some embodiments, R⁶ is —NH₂. In some embodiments, R⁶ is C₁-C₆ alkyl. In some embodiments, R⁶ is C₁-C₆ hydroxyalkyl. In some embodiments, R⁶ is C₁-C₆ alkoxy. In some embodiments, R⁶ is C₂-C₆ alkenyl. In some embodiments, R⁶ is C₂-C₆ alkynyl. In some embodiments, R⁶ is C₃-C₈ cycloalkyl. In some embodiments, R⁶ is heterocyclyl. In some embodiments, R⁶ is heteroaryl. In some embodiments, R⁶ is aryl. In some embodiments, at least one R⁶ is C₁-C₆ alkyl. In some embodiments, at least one R⁶ is C₁-C₃ alkyl. In some embodiments, at least one R⁶ is methyl.

In some embodiments of any of the above Formulae, R⁷ is H. In some embodiments, R⁷ is halogen. In some embodiments, R⁷ is —OH. In some embodiments, R⁷ is —CN. In some embodiments, R⁷ is —NH₂. In some embodiments, R⁷ is C₁-C₆ alkyl. In some embodiments, R⁷ is C₁-C₆ hydroxyalkyl. In some embodiments, R⁷ is C₁-C₆ alkoxy. In some embodiments, R⁷ is C₂-C₆ alkenyl. In some embodiments, R⁷ is C₂-C₆ alkynyl. In some embodiments, R⁷ is C₃-C₈ cycloalkyl. In some embodiments, R⁷ is heterocyclyl. In some embodiments, R⁷ is heteroaryl. In some embodiments, R⁷ is aryl.

In some embodiments of any of the above Formulae, R⁸ is H. In some embodiments, R⁸ is halogen. In some embodiments, R⁸ is —OH. In some embodiments, R⁸ is —CN. In some embodiments, R⁸ is —NH₂. In some embodiments, R⁸ is C₁-C₆ alkyl. In some embodiments, R⁸ is C₁-C₆ hydroxyalkyl. In some embodiments, R⁸ is C₁-C₆ alkoxy. In some embodiments, R⁸ is C₂-C₆ alkenyl. In some embodiments, R⁸ is C₂-C₆ alkynyl. In some embodiments, R⁸ is C₃-C₈ cycloalkyl. In some embodiments, R⁸ is heterocyclyl. In some embodiments, R⁸ is heteroaryl. In some embodiments, R⁸ is aryl.

In some embodiments of any of the above Formulae, R⁹ is H. In some embodiments, R⁹ is C₁-C₄ alkyl. In some embodiments, R⁹ is C₁-C₄ alkyl optionally substituted with one or more —OH, halogen, —CN, C₁-C₆ alkoxy, or cycloalkyl.

In some embodiments of any of the above Formulae, n is an integer selected from 0 to 6. In some embodiments, n is an integer selected from 0 to 5. In some embodiments, n is an integer selected from 0 to 4. In some embodiments, n is an integer selected from 0 to 3. In some embodiments, n is an integer selected from 0 to 2. In some embodiments, n is an integer selected from 0 and 1. In some embodiments, n is an integer selected from 1 to 6. In some embodiments, n is an integer selected from 1 to 5. In some embodiments, n is an integer selected from 1 to 4. In some embodiments, n is an integer selected from 1 to 3. In some embodiments, n is an integer selected from 1 and 2. In some embodiments, n is an integer selected from 2 to 6. In some embodiments, n is an integer selected from 2 to 5. In some embodiments, n is an integer selected from 2 to 4. In some embodiments, n is an integer selected from 2 and 3. In some embodiments, n is an integer selected from 3 to 6. In some embodiments, n is an integer selected from 3 to 5. In some embodiments, n is an integer selected from 3 and 4. In some embodiments, n is an integer selected from 4 to 6. In some embodiments, n is an integer selected from 4 and 5.

In some embodiments of any of the above Formulae, p is an integer selected from 0 to 6. In some embodiments, p is an integer selected from 0 to 5. In some embodiments, n is an integer selected from 0 to 4. In some embodiments, p is an integer selected from 0 to 3. In some embodiments, p is an integer selected from 0 to 2. In some embodiments, p is an integer selected from 0 and 1. In some embodiments, p is an integer selected from 1 to 6. In some embodiments, p is an integer selected from 1 to 5. In some embodiments, p is an integer selected from 1 to 4. In some embodiments, p is an integer selected from 1 to 3. In some embodiments, p is an integer selected from 1 and 2. In some embodiments, p is an integer selected from 2 to 6. In some embodiments, p is an integer selected from 2 to 5. In some embodiments, p is an integer selected from 2 to 4. In some embodiments, p is an integer selected from 2 and 3. In some embodiments, p is an integer selected from 3 to 6. In some embodiments, p is an integer selected from 3 to 5. In some embodiments, p is an integer selected from 3 and 4. In some embodiments, p is an integer selected from 4 to 6. In some embodiments, p is an integer selected from 4 and 5. In some embodiments, p is 0. In some embodiments, p is 1. In some embodiments, p is 2. In some embodiments, p is 3. In some embodiments, p is 4. In some embodiments, p is 5. In some embodiments, p is 6.

In some embodiments of any of the above Formulae, r is 0, 1, or 2. In some embodiments, r is 0. In some embodiments, r is 1. In some embodiments, r is 2. In some embodiments, r is 1 or 2. In some embodiments, r is 0 or 1. In some embodiments, r is 0 or 2.

In some embodiments, the compound is selected from any one of the compounds of Tables 1-5, or a pharmaceutically acceptable salt, hydrate, solvate, prodrug, stereoisomer, or tautomer thereof.

In certain embodiments, the compound is selected from any one of the compounds of Tables 1-5, or a pharmaceutically acceptable salt thereof.

In some embodiments, the compound is selected from a pharmaceutically acceptable salt of any one of the compounds of Tables 1-5.

In some embodiments, the compound is a free base selected from any one of the compounds of Tables 1-5.

The below Tables 1-5 also provide the location of the compound in the Examples, i.e., by Example Number (Ex) or as provided in Table A (TA) of the Examples. The asterix (*) next to the Compound Number (#) signifies that arbitrary stereochemistry has been assigned.

Compounds of Formula (I)

| # | Ex | Name/Structure |
|---|---|---|
| Rac-60 | 9 | 1-(7,8-dichloro-1-methyl-3,4-dihydropyrazino[1,2-b]indazol-2(1H)-yl)-2- hydroxyethan-1-one 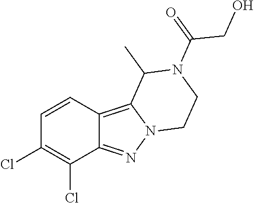 [SMILES: CC1c2c3ccc(Cl)c(Cl)c3nn2CCN1C(=O)CO] |
| 60A | 9 | (R)-1-(7,8-dichloro-1-methyl-3,4-dihydropyrazino[1,2-b]indazol-2(1H)-yl)-2- hydroxyethan-1-one 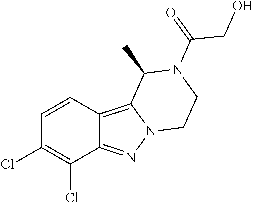 [SMILES: C[C@@H]1c2c3ccc(Cl)c(Cl)c3nn2CCN1C(=O)CO] |
| 60B | 9 | (S)-1-(7,8-dichloro-1-methyl-3,4-dihydropyrazino[1,2-b]indazol-2(1H)-yl)-2- hydroxyethan-1-one 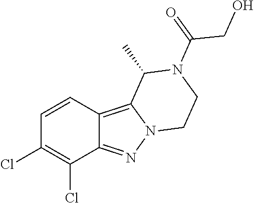 [SMILES: C[C@H]1c2c3ccc(Cl)c(Cl)c3nn2CCN1C(=O)CO] |
| Rac-61 | 11 | 1-(7,8-dichloro-1-methyl-10-(1-methyl-1H-pyrazol-3-yl)-3,4- dihydropyrazino[1,2-b]indazol-2(1H)-yl)-2-hydroxyethan-1-one 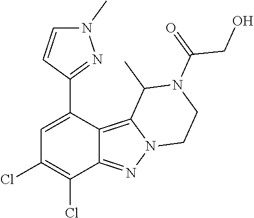 [SMILES: CC1c2c3c(-c4ccn(C)n4)cc(Cl)c(Cl)c3nn2CCN1C(=O)CO] |
| 61A* (75) | 11 | (R)-1-(7,8-dichloro-1-methyl-10-(1-methyl-1H-pyrazol-3-yl)-3,4- dihydropyrazino[1,2-b]indazol-2(1H)-yl)-2-hydroxyethan-1-one 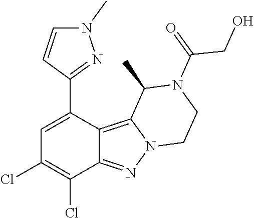 [SMILES: C[C@@H]1c2c3c(-c4ccn(C)n4)cc(Cl)c(Cl)c3nn2CCN1C(=O)CO] |
| 61B* (67) | 11 | (S)-1-(7,8-dichloro-1-methyl-10-(1-methyl-1H-pyrazol-3-yl)-3,4- dihydropyrazino[1,2-b]indazol-2(1H)-yl)-2-hydroxyethan-1-one 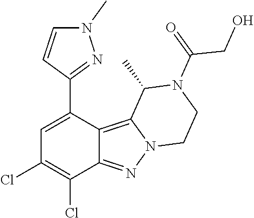 [SMILES: C[C@H]1c2c3c(-c4ccn(C)n4)cc(Cl)c(Cl)c3nn2CCN1C(=O)CO] |

TABLE 2
Compounds of Formula (I)

| # | Ex | Name/Structure |
|---|---|---|
| Rac-51 | 8 | 2-amino-1-(6,7-dichloro-1-methyl-1,3,4,5-tetrahydro-2H-pyrido[4,3- b]indol-2-yl)ethan-1-one 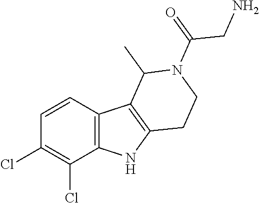 [SMILES: CC1c2c([nH]c3c(Cl)c(Cl)ccc23)CCN1C(=O)CN] |
| 51A* | 8 | (R)-2-amino-1-(6,7-dichloro-1-methyl-1,3,4,5-tetrahydro-2H-pyrido[4,3- b]indol-2-yl)ethan-1-one 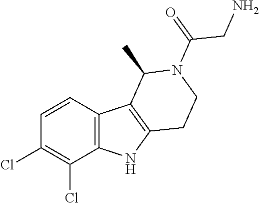 [SMILES: C[C@@H]1c2c([nH]c3c(Cl)c(Cl)ccc23)CCN1C(=O)CN] |
| 51B* (63) | 8 | (S)-2-amino-1-(6,7-dichloro-1-methyl-1,3,4,5-tetrahydro-2H-pyrido[4,3- b]indol-2-yl)ethan-1-one 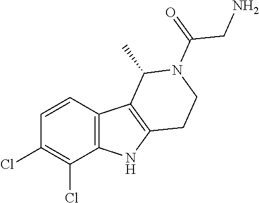 [SMILES: C[C@H]1c2c([nH]c3c(Cl)c(Cl)ccc23)CCN1C(=O)CN] |
| 66 | TA | 2-amino-1-(8,9-dichloro-1,2,4,5,6,7-hexahydro-1,5-epiminooxocino[5,4- b]indol-12-yl)ethan-1-one 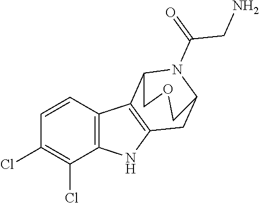 [SMILES: NCC(=O)N1C2COCC1c1c([nH]c3c(Cl)c(Cl)ccc13)C2] |
| Rac-120 | 21 | 2-amino-1-(7,8-dichloro-1-methyl-3,4-dihydropyrazino[1,2-b]indazol- 2(1H)-yl)ethan-1-one 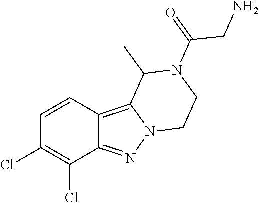 [SMILES: CC1c2c3ccc(Cl)c(Cl)c3nn2CCN1C(=O)CN] |
| 120A* | 21 | (R)-2-amino-1-(7,8-dichloro-1-methyl-3,4-dihydropyrazino[1,2-b]indazol- 2(1H)-yl)ethan-1-one 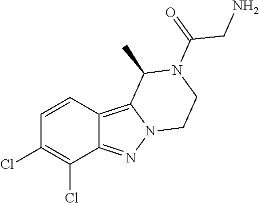 [SMILES: C[C@@H]1c2c3ccc(Cl)c(Cl)c3nn2CCN1C(=O)CN] |
| 120B* | 21 | (S)-2-amino-1-(7,8-dichloro-1-methyl-3,4-dihydropyrazino[1,2-b]indazol- 2(1H)-yl)ethan-1-one 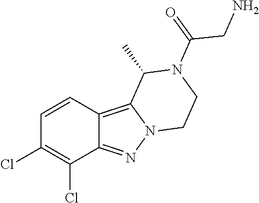 [SMILES: C[C@H]1c2c3ccc(Cl)c(Cl)c3nn2CCN1C(=O)CN] |
| 64 | TA | 1-(6,7-dichloro-1,3,4,5-tetrahydro-2H-pyrido[4,3-b]indol-2-yl)-2- (methylamino)ethan-1-one 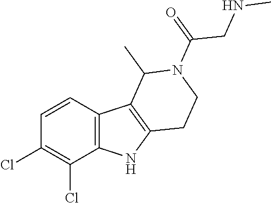 [SMILES: CNCC(=O)N1CCc2[nH]c3c(Cl)c(Cl)ccc3c2C1C] |
| 69 | TA | 1-(6,7-dichloro-1,3,4,5-tetrahydro-2H-pyrido[4,3-b]indol-2-yl)-2-((2- methoxyethyl)amino)ethan-1-one 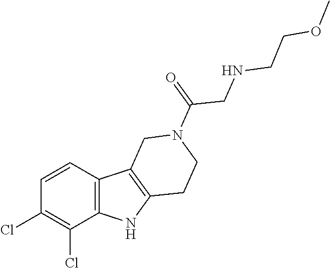 [SMILES: COCCNCC(=O)N1CCc2[nH]c3c(Cl)c(Cl)ccc3c2C1] |

TABLE 3
Compound of Formula (I)

| # | Ex | Name/Structure |
|---|---|---|
| 1 | 1 | (6,7-dichloro-1,3,4,5-tetrahydropyrido[4,3-b]indol-2-yl)-(5-methoxypyrimidin- 2-yl)methanone; Also referred to as (6,7-dichloro-1,3,4,5-tetrahydro-2H- pyrido[4,3-b]indol-2-yl)(5-methoxypyrimidin-2-yl)methanone 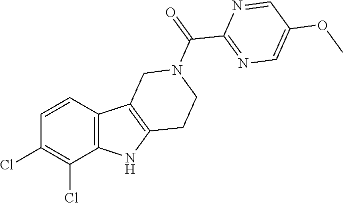 [SMILES: COc1cnc(C(=O)N2CCc3[nH]c4c(Cl)c(Cl)ccc4c3C2)nc1] |
| 2 | TA | (6,7-dichloro-1,3,4,5-tetrahydro-2H-pyrido[4,3-b]indol-2-yl)(pyrimidin-2- yl)methanone 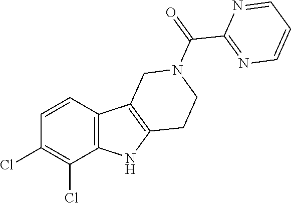 [SMILES: O=C(c1ncccn1)N1CCc2[nH]c3c(Cl)c(Cl)ccc3c2C1] |
| 4 | TA | (6,7-dichloro-1,3,4,5-tetrahydro-2H-pyrido[4,3-b]indol-2-yl)(4- methoxypyrimidin-2-yl)methanone 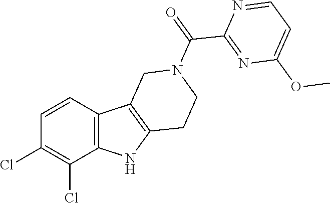 [SMILES: COc1ccnc(C(=O)N2CCc3[nH]c4c(Cl)c(Cl)ccc4c3C2)n1] |
| 8 | TA | (6,7-dichloro-1,3,4,5-tetrahydro-2H-pyrido[4,3-b]indol-2-yl)(4- methylpyrimidin-2-yl)methanone 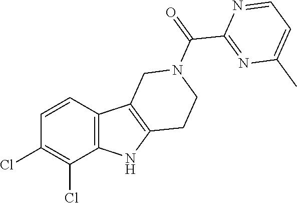 [SMILES: Cc1ccnc(C(=O)N2CCc3[nH]c4c(Cl)c(Cl)ccc4c3C2)n1] |
| 12 | TA | (6,7-dichloro-1,3,4,5-tetrahydro-2H-pyrido[4,3-b]indol-2-yl)(5- methylpyrimidin-2-yl)methanone 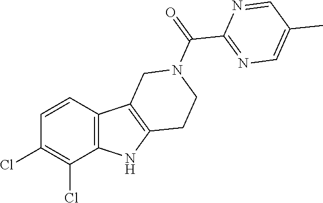 [SMILES: Cc1cnc(C(=O)N2CCc3[nH]c4c(Cl)c(Cl)ccc4c3C2)nc1] |
| 14 | TA | (4-aminopyrimidin-2-yl)(6,7-dichloro-1,3,4,5-tetrahydro-2H-pyrido[4,3- b]indol-2-yl)methanone 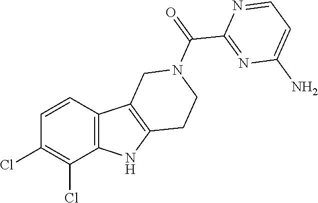 [SMILES: Nc1ccnc(C(=O)N2CCc3[nH]c4c(Cl)c(Cl)ccc4c3C2)n1] |
| 15 | 5 | (6,7-dichloro-1,3,4,5-tetrahydro-2H-pyrido[4,3-b]indol-2-yl)(5-fluoropyrimidin- 2-yl)methanone 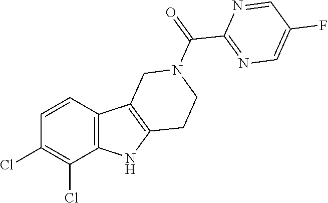 [SMILES: O=C(c1ncc(F)cn1)N1CCc2[nH]c3c(Cl)c(Cl)ccc3c2C1] |
| 16 | TA | (6,7-dichloro-1,3,4,5-tetrahydro-2H-pyrido[4,3-b]indol-2-yl)(5- hydroxypyrimidin-2-yl)methanone 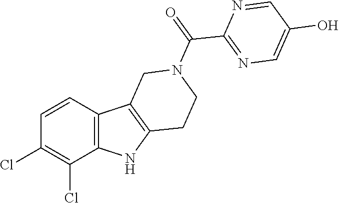 [SMILES: O=C(c1ncc(O)cn1)N1CCc2[nH]c3c(Cl)c(Cl)ccc3c2C1] |
| 17 | 4 | 2-(6,7-dichloro-2-(5-methoxypyrimidine-2-carbonyl)-1,2,3,4-tetrahydro-5H- pyrido[4,3-b]indol-5-yl)acetic acid 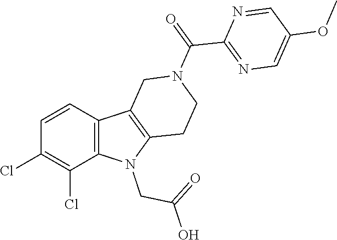 [SMILES: COc1cnc(C(=O)N2CCc3c(c4ccc(Cl)c(Cl)c4n3CC(=O)O)C2)nc1] |
| 18 | TA | (6,7-dichloro-5-(2-(methylsulfonyl)ethyl)-1,3,4,5-tetrahydro-2H-pyrido[4,3- b]indol-2-yl)(5-methoxypyrimidin-2-yl)methanone 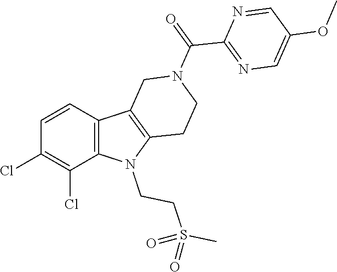 [SMILES: COc1cnc(C(=O)N2CCc3c(c4ccc(Cl)c(Cl)c4n3CCS(C)(=O)=O)C2)nc1] |
| 19 | 3 | (6,7-dichloro-5-(3-methoxypropyl)-1,3,4,5-tetrahydro-2H-pyrido[4,3-b]indol-2- yl)(5-methoxypyrimidin-2-yl)methanone 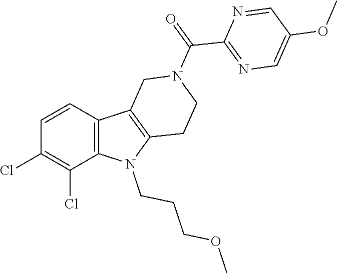 [SMILES: COCCCn1c2c(c3ccc(Cl)c(Cl)c31)CN(C(=O)c1ncc(OC)cn1)CC2] |
| 20 | TA | (6,7-dichloro-5-(methylsulfonyl)-1,3,4,5-tetrahydro-2H-pyrido[4,3-b]indol-2- yl)(5-methoxypyrimidin-2-yl)methanone 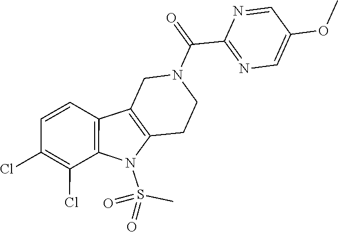 [SMILES: COc1cnc(C(=O)N2CCc3c(c4ccc(Cl)c(Cl)c4n3S(C)(=O)=O)C2)nc1] |
| 21 | TA | 3-(6,7-dichloro-2-(5-methoxypyrimidine-2-carbonyl)-1,2,3,4-tetrahydro-5H- pyrido[4,3-b]indol-5-yl)propanoic acid 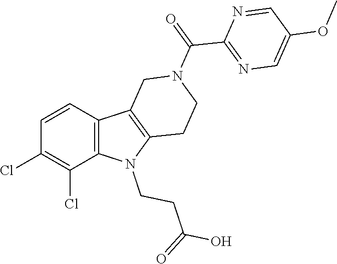 [SMILES: COc1cnc(C(=O)N2CCc3c(c4ccc(Cl)c(Cl)c4n3CCC(=O)O)C2)nc1] |
| 22 | TA | (6,7-dichloro-5-(2-hydroxyethyl)-1,3,4,5-tetrahydro-2H-pyrido[4,3-b]indol-2- yl)(5-methoxypyrimidin-2-yl)methanone 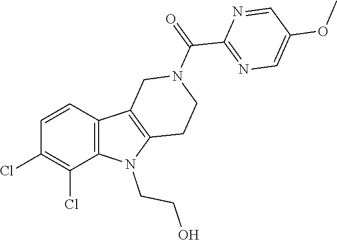 [SMILES: COc1cnc(C(=O)N2CCc3c(c4ccc(Cl)c(Cl)c4n3CCO)C2)nc1] |
| 23 | TA | (6,7-dichloro-5-((2-methoxyethoxy)methyl)-1,3,4,5-tetrahydro-2H-pyrido[4,3- b]indol-2-yl)(5-methoxypyrimidin-2-yl)methanone 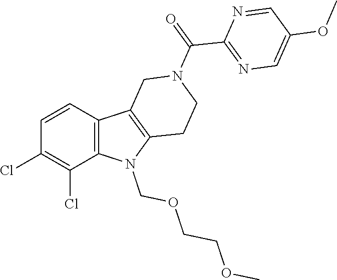 [SMILES: COCCOCn1c2c(c3ccc(Cl)c(Cl)c31)CN(C(=O)c1ncc(OC)cn1)CC2] |
| 24 | TA | (6,7-dichloro-5-(3-hydroxypropyl)-1,3,4,5-tetrahydro-2H-pyrido[4,3-b]indol-2- yl)(5-methoxypyrimidin-2-yl)methanone 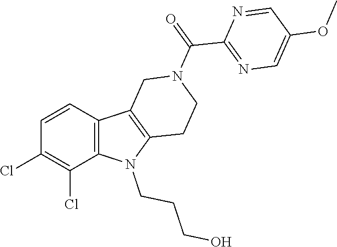 [SMILES: COc1cnc(C(=O)N2CCc3c(c4ccc(Cl)c(Cl)c4n3CCCO)C2)nc1] |
| 25 | TA | 4-(6,7-dichloro-2-(5-methoxypyrimidine-2-carbonyl)-1,2,3,4-tetrahydro-5H- pyrido[4,3-b]indol-5-yl)butanoic acid 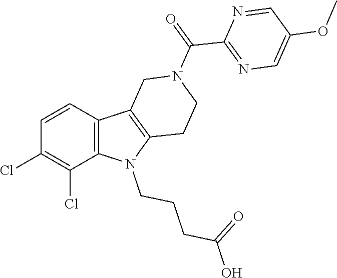 [SMILES: COc1cnc(C(=O)N2CCc3c(c4ccc(Cl)c(Cl)c4n3CCCC(=O)O)C2)nc1] |
| 26 | TA | methyl 4-(6,7-dichloro-2-(5-methoxypyrimidine-2-carbonyl)-1,2,3,4-tetrahydro- 5H-pyrido[4,3-b]indol-5-yl)butanoate 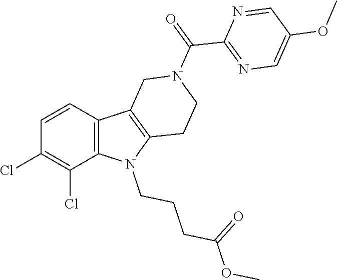 [SMILES: COC(=O)CCCn1c2c(c3ccc(Cl)c(Cl)c31)CN(C(=O)c1ncc(OC)cn1)CC2] |
| 28 | TA | 2-(6,7-dichloro-2-(5-methoxypyrimidine-2-carbonyl)-1,2,3,4-tetrahydro-5H- pyrido[4,3-b]indol-5-yl)acetonitrile 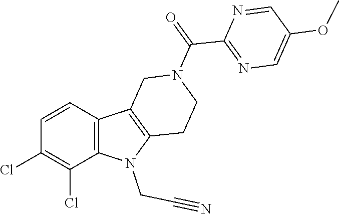 [SMILES: COc1cnc(C(=O)N2CCc3c(c4ccc(Cl)c(Cl)c4n3CC#N)C2)nc1] |
| 29 | 5 | (6,7-dichloro-1,3,4,5-tetrahydro-2H-pyrido[4,3-b]indol-2-yl)(5-((2- hydroxyethyl)amino)pyrimidin-2-yl)methanone 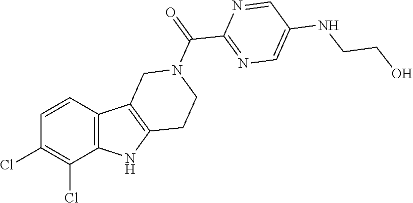 [SMILES: O=C(c1ncc(NCCO)cn1)N1CCc2[nH]c3c(Cl)c(Cl)ccc3c2C1] |
| 30 | TA | (5-aminopyrimidin-2-yl)(6,7-dichloro-1,3,4,5-tetrahydro-2H-pyrido[4,3- b]indol-2-yl)methanone 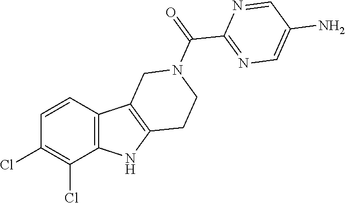 [SMILES: Nc1cnc(C(=O)N2CCc3[nH]c4c(Cl)c(Cl)ccc4c3C2)nc1] |
| 31 | TA | (6,7-dichloro-1,3,4,5-tetrahydro-2H-pyrido[4,3-b]indol-2-yl)(5- morpholinopyrimidin-2-yl)methanone 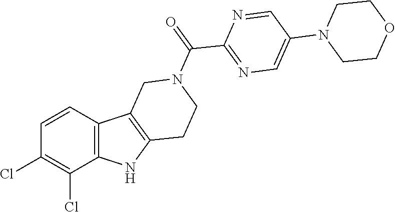 [SMILES: O=C(c1ncc(N2CCOCC2)cn1)N1CCc2[nH]c3c(Cl)c(Cl)ccc3c2C1] |
| 32 | TA | (6,7-dichloro-1,3,4,5-tetrahydro-2H-pyrido[4,3-b]indol-2-yl)(5-((2- hydroxyethyl)(methyl)amino)pyrimidin-2-yl)methanone 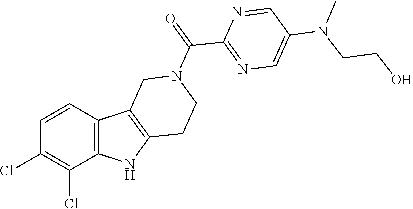 [SMILES: CN(CCO)c1cnc(C(=O)N2CCc3[nH]c4c(Cl)c(Cl)ccc4c3C2)nc1] |
| 33 | TA | (6,7-dichloro-1,3,4,5-tetrahydro-2H-pyrido[4,3-b]indol-2-yl)(5-((2- (dimethylamino)ethyl)(methyl)amino)pyrimidin-2-yl)methanone 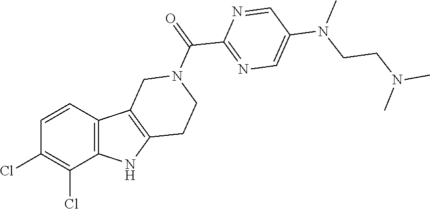 [SMILES: CN(C)CCN(C)c1cnc(C(=O)N2CCc3[nH]c4c(Cl)c(Cl)ccc4c3C2)nc1] |
| 34 | TA | (6,7-dichloro-1,3,4,5-tetrahydro-2H-pyrido[4,3-b]indol-2-yl)(5-(4-(2- hydroxyethyl)piperazin-1-yl)pyrimidin-2-yl)methanone 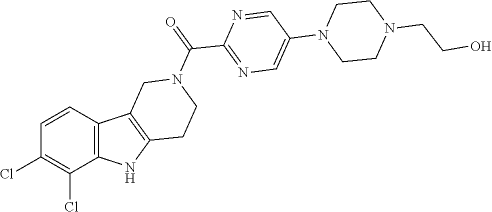 [SMILES: O=C(c1ncc(N2CCN(CCO)CC2)cn1)N1CCc2[nH]c3c(Cl)c(Cl)ccc3c2C1] |
| 35 | TA | (R)-(6,7-dichloro-1,3,4,5-tetrahydro-2H-pyrido[4,3-b]indol-2-yl)(5-(2- (hydroxymethyl)pyrrolidin-1-yl)pyrimidin-2-yl)methanone 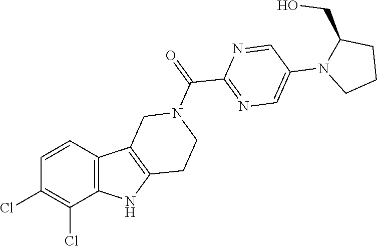 [SMILES: O=C(c1ncc(N2CCC[C@@H]2CO)cn1)N1CCc2[nH]c3c(Cl)c(Cl)ccc3c2C1] |
| 36 | TA | (S)-(6,7-dichloro-1,3,4,5-tetrahydro-2H-pyrido[4,3-b]indol-2-yl)(5-(2- (hydroxymethyl)pyrrolidin-1-yl)pyrimidin-2-yl)methanone 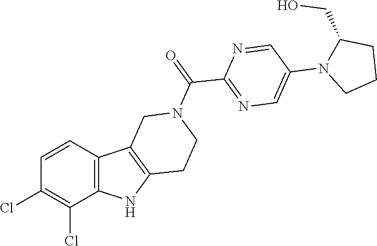 [SMILES: O=C(c1ncc(N2CCC[C@H]2CO)cn1)N1CCc2[nH]c3c(Cl)c(Cl)ccc3c2C1] |
| 37 | TA | (6,7-dichloro-1,3,4,5-tetrahydro-2H-pyrido[4,3-b]indol-2-yl)(5- (dimethylamino)pyrimidin-2-yl)methanone 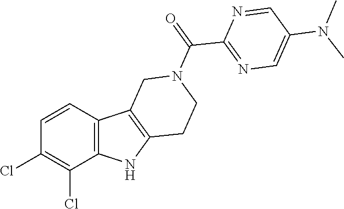 [SMILES: CN(C)c1cnc(C(=O)N2CCc3[nH]c4c(Cl)c(Cl)ccc4c3C2)nc1] |
| 38 | TA | (6,7-dichloro-1,3,4,5-tetrahydro-2H-pyrido[4,3-b]indol-2-yl)(5- (methylamino)pyrimidin-2-yl)methanone 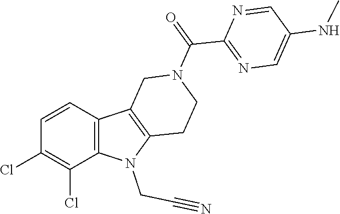 [SMILES: CNc1cnc(C(=O)N2CCc3c(c4ccc(Cl)c(Cl)c4n3CC#N)C2)nc1] |
| 39 | TA | (S)-(6,7-dichloro-1,3,4,5-tetrahydro-2H-pyrido[4,3-b]indol-2-yl)(5-(3- hydroxypyrrolidin-1-yl)pyrimidin-2-yl)methanone 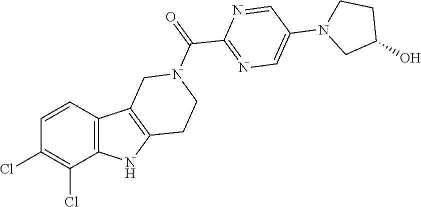 [SMILES: O=C(c1ncc(N2CC[C@H](O)C2)cn1)N1CCc2[nH]c3c(Cl)c(Cl)ccc3c2C1] |
| 40 | TA | (R)-(6,7-dichloro-1,3,4,5-tetrahydro-2H-pyrido[4,3-b]indol-2-yl)(5-(3- hydroxypyrrolidin-1-yl)pyrimidin-2-yl)methanone 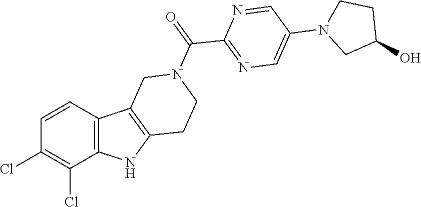 [SMILES: O=C(c1ncc(N2CC[C@@H](O)C2)cn1)N1CCc2[nH]c3c(Cl)c(Cl)ccc3c2C1] |
| 41 | TA | (6,7-dichloro-1,3,4,5-tetrahydro-2H-pyrido[4,3-b]indol-2-yl)(5-(pyrrolidin-1- yl)pyrimidin-2-yl)methanone 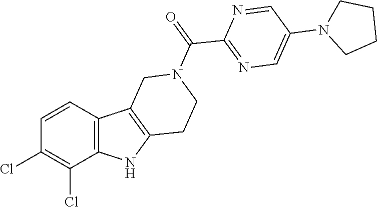 [SMILES: O=C(c1ncc(N2CCCC2)cn1)N1CCc2[nH]c3c(Cl)c(Cl)ccc3c2C1] |
| 42 | TA | (6,7-dichloro-1,3,4,5-tetrahydro-2H-pyrido[4,3-b]indol-2-yl)(5-(3- hydroxyazetidin-1-yl)pyrimidin-2-yl)methanone 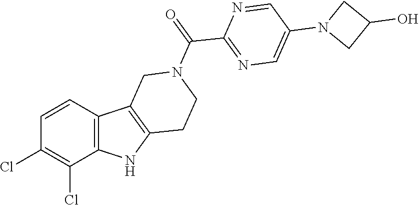 [SMILES: O=C(c1ncc(N2CC(O)C2)cn1)N1CCc2[nH]c3c(Cl)c(Cl)ccc3c2C1] |
| 43 | TA | (6,7-dichloro-1,3,4,5-tetrahydro-2H-pyrido[4,3-b]indol-2-yl)(5-((2- methoxyethyl)amino)pyrimidin-2-yl)methanone 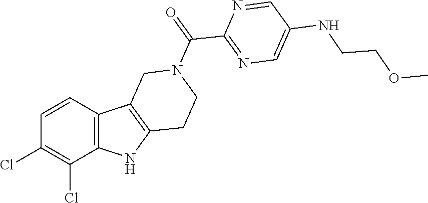 [SMILES: COCCNc1cnc(C(=O)N2CCc3[nH]c4c(Cl)c(Cl)ccc4c3C2)nc1] |
| Rac-44 | 6 | (6,7-dichloro-1-methyl-1,3,4,5-tetrahydro-2H-pyrido[4,3-b]indol-2-yl)(5- morpholinopyrimidin-2-yl)methanone 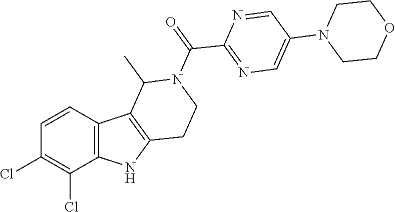 [SMILES: CC1c2c([nH]c3c(Cl)c(Cl)ccc23)CCN1C(=O)c1ncc(N2CCOCC2)cn1] |
| 44A* | 6 | (R)-(6,7-dichloro-1-methyl-1,3,4,5-tetrahydro-2H-pyrido[4,3-b]indol-2-yl)(5- morpholinopyrimidin-2-yl)methanone 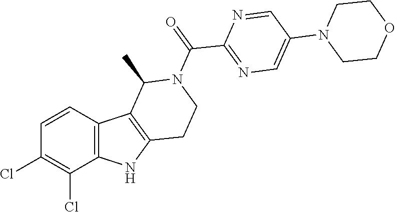 [SMILES: C[C@@H]1c2c([nH]c3c(Cl)c(Cl)ccc23)CCN1C(=O)c1ncc(N2CCOCC2)cn1] |
| 44B* | 6 | (S)-(6,7-dichloro-1-methyl-1,3,4,5-tetrahydro-2H-pyrido[4,3-b]indol-2-yl)(5- morpholinopyrimidin-2-yl)methanone 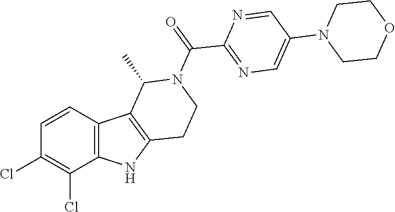 [SMILES: C[C@H]1c2c([nH]c3c(Cl)c(Cl)ccc23)CCN1C(=O)c1ncc(N2CCOCC2)cn1] |
| Rac-45 | 7 | (6,7-dichloro-1-methyl-1,3,4,5-tetrahydro-2H-pyrido[4,3-b]indol-2-yl)(5- methoxypyrimidin-2-yl)methanone 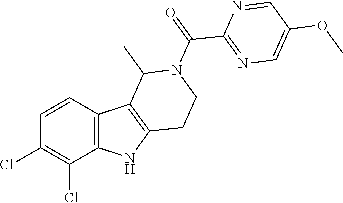 [SMILES: COc1cnc(C(=O)N2CCc3[nH]c4c(Cl)c(Cl)ccc4c3C2C)nc1] |
| 45A* | 7 | (R)-(6,7-dichloro-1-methyl-1,3,4,5-tetrahydro-2H-pyrido[4,3-b]indol-2-yl)(5- methoxypyrimidin-2-yl)methanone 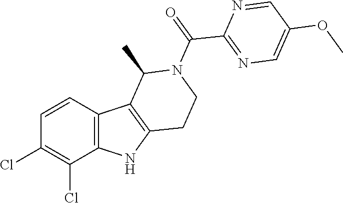 [SMILES: COc1cnc(C(=O)N2CCc3[nH]c4c(Cl)c(Cl)ccc4c3[C@H]2C)nc1] |
| 45B* (62) | 7 | (S)-(6,7-dichloro-1-methyl-1,3,4,5-tetrahydro-2H-pyrido[4,3-b]indol-2-yl)(5- methoxypyrimidin-2-yl)methanone 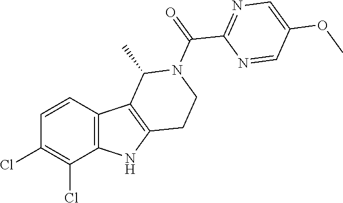 [SMILES: COc1cnc(C(=O)N2CCc3[nH]c4c(Cl)c(Cl)ccc4c3[C@@H]2C)nc1] |
| Rac-46 | TA | (6,7-dichloro-1-methyl-1,3,4,5-tetrahydro-2H-pyrido[4,3-b]indol-2-yl)(5- (methylamino)pyrimidin-2-yl)methanone 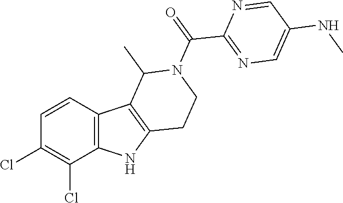 [SMILES: CNc1cnc(C(=O)N2CCc3[nH]c4c(Cl)c(Cl)ccc4c3C2C)nc1] |
| 46A* | TA | (R)-(6,7-dichloro-1-methyl-1,3,4,5-tetrahydro-2H-pyrido[4,3-b]indol-2-yl)(5- (methylamino)pyrimidin-2-yl)methanone 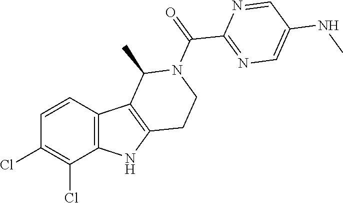 [SMILES: CNc1cnc(C(=O)N2CCc3[nH]c4c(Cl)c(Cl)ccc4c3[C@H]2C)nc1] |
| 46B* | TA | (S)-(6,7-dichloro-1-methyl-1,3,4,5-tetrahydro-2H-pyrido[4,3-b]indol-2-yl)(5- (methylamino)pyrimidin-2-yl)methanone 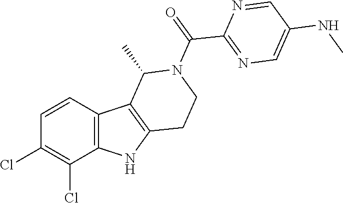 [SMILES: CNc1cnc(C(=O)N2CCc3[nH]c4c(Cl)c(Cl)ccc4c3[C@@H]2C)nc1] |
| Rac-47 | TA | (6,7-dichloro-1-methyl-1,3,4,5-tetrahydro-2H-pyrido[4,3-b]indol-2-yl)(5-((2- (dimethylamino)ethyl)(methyl)amino)pyrimidin-2-yl)methanone 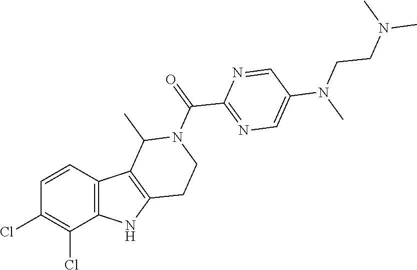 [SMILES: CC1c2c([nH]c3c(Cl)c(Cl)ccc23)CCN1C(=O)c1ncc(N(C)CCN(C)C)cn1] |
| 47A* | TA | (R)-(6,7-dichloro-1-methyl-1,3,4,5-tetrahydro-2H-pyrido[4,3-b]indol-2-yl)(5- ((2-(dimethylamino)ethyl)(methyl)amino)pyrimidin-2-yl)methanone 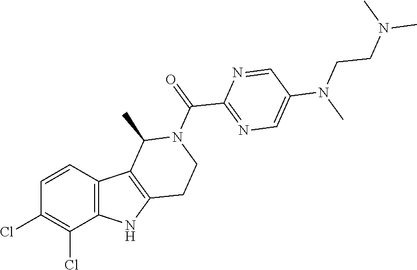 [SMILES: C[C@@H]1c2c([nH]c3c(Cl)c(Cl)ccc23)CCN1C(=O)c1ncc(N(C)CCN(C)C)cn1] |
| 47B* | TA | (S)-(6,7-dichloro-1-methyl-1,3,4,5-tetrahydro-2H-pyrido[4,3-b]indol-2-yl)(5- ((2-(dimethylamino)ethyl)(methyl)amino)pyrimidin-2-yl)methanone 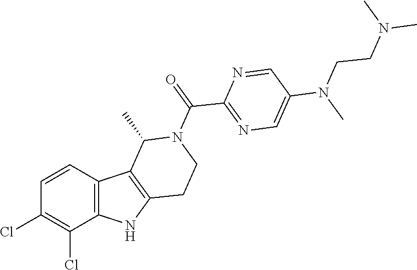 [SMILES: C[C@H]1c2c([nH]c3c(Cl)c(Cl)ccc23)CCN1C(=O)c1ncc(N(C)CCN(C)C)cn1] |
| Rac-48 | TA | (6,7-dichloro-1-methyl-1,3,4,5-tetrahydro-2H-pyrido[4,3-b]indol-2-yl)(5-(4- methylpiperazin-1-yl)pyrimidin-2-yl)methanone 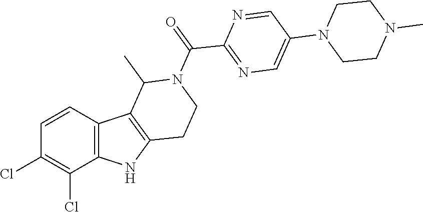 [SMILES: CC1c2c([nH]c3c(Cl)c(Cl)ccc23)CCN1C(=O)c1ncc(N2CCN(C)CC2)cn1] |
| 48A* | TA | (R)-(6,7-dichloro-1-methyl-1,3,4,5-tetrahydro-2H-pyrido[4,3-b]indol-2-yl)(5-(4 methylpiperazin-1-yl)pyrimidin-2-yl)methanone 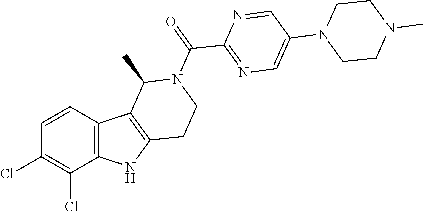 [SMILES: C[C@@H]1c2c([nH]c3c(Cl)c(Cl)ccc23)CCN1C(=O)c1ncc(N2CCN(C)CC2)cn1] |
| 48B* | TA | (S)-(6,7-dichloro-1-methyl-1,3,4,5-tetrahydro-2H-pyrido[4,3-b]indol-2-yl)(5-(4- methylpiperazin-1-yl)pyrimidin-2-yl)methanone 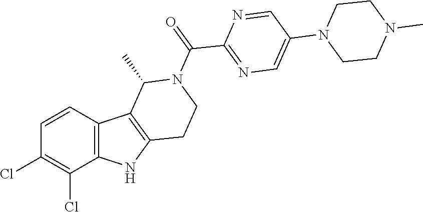 [SMILES: C[C@H]1c2c([nH]c3c(Cl)c(Cl)ccc23)CCN1C(=O)c1ncc(N2CCN(C)CC2)cn1] |
| Rac-49 | TA | (6,7-dichloro-1-methyl-1,3,4,5-tetrahydro-2H-pyrido[4,3-b]indol-2-yl)(5-(4- (oxetan-3-yl)piperazin-1-yl)pyrimidin-2-yl)methanone 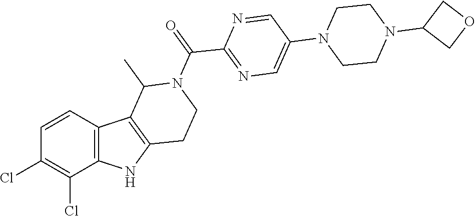 [SMILES: CC1c2c([nH]c3c(Cl)c(Cl)ccc23)CCN1C(=O)c1ncc(N2CCN(C3COC3)CC2)cn1] |
| 49A* | TA | (R)-(6,7-dichloro-1-methyl-1,3,4,5-tetrahydro-2H-pyrido[4,3-b]indol-2-yl)(5-(4- (oxetan-3-yl)piperazin-1-yl)pyrimidin-2-yl)methanone 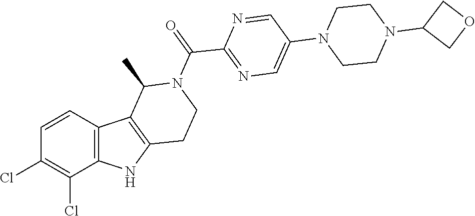 [SMILES: C[C@@H]1c2c([nH]c3c(Cl)c(Cl)ccc23)CCN1C(=O)c1ncc(N2CCN(C3COC3)CC2)cn1] |
| 49B* | TA | (S)-(6,7-dichloro-1-methyl-1,3,4,5-tetrahydro-2H-pyrido[4,3-b]indol-2-yl)(5-(4- (oxetan-3-yl)piperazin-1-yl)pyrimidin-2-yl)methanone 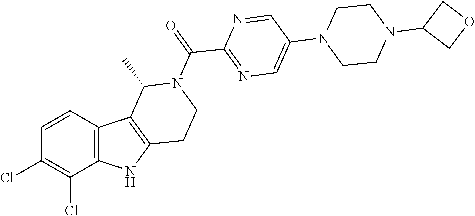 [SMILES: C[C@H]1c2c([nH]c3c(Cl)c(Cl)ccc23)CCN1C(=O)c1ncc(N2CCN(C3COC3)CC2)cn1] |
| Rac-50 | TA | (6,7-dichloro-1-methyl-1,3,4,5-tetrahydro-2H-pyrido[4,3-b]indol-2-yl)(5-((S)- 7,7-difluorohexahydropyrrolo[1,2-a]pyrazin-2(1H)-yl)pyrimidin-2- yl)methanone 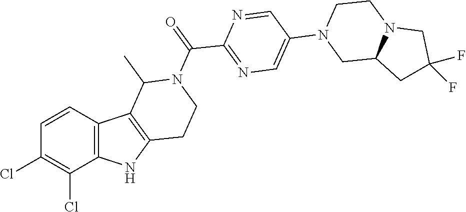 [SMILES: CC1c2c([nH]c3c(Cl)c(Cl)ccc23)CCN1C(=O)c1ncc(N2CCN3CC(F)(F)C[C@H]3C2)cn1] |
| 50A* | TA | ((R)-6,7-dichloro-1-methyl-1,3,4,5-tetrahydro-2H-pyrido[4,3-b]indol-2-yl)(5- ((S)-7,7-difluorohexahydropyrrolo[1,2-a]pyrazin-2(1H)-yl)pyrimidin-2- yl)methanone 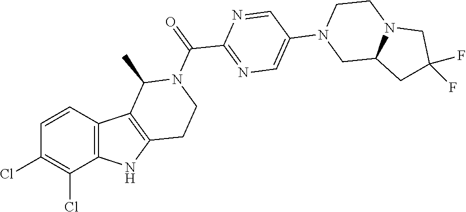 [SMILES: C[C@@H]1c2c([nH]c3c(Cl)c(Cl)ccc23)CCN1C(=O)c1ncc(N2CCN3CC(F)(F)C[C@H]3C2)cn1] |
| 50B* | TA | ((S)-6,7-dichloro-1-methyl-1,3,4,5-tetrahydro-2H-pyrido[4,3-b]indol-2-yl)(5- ((S)-7,7-difluorohexahydropyrrolo[1,2-a]pyrazin-2(1H)-yl)pyrimidin-2- yl)methanone 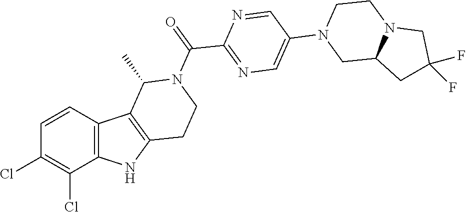 [SMILES: C[C@H]1c2c([nH]c3c(Cl)c(Cl)ccc23)CCN1C(=O)c1ncc(N2CCN3CC(F)(F)C[C@H]3C2)cn1] |
| Rac-52 | 10 | (7,8-dichloro-1-methyl-3,4-dihydropyrazino[1,2-b]indazol-2(1H)-yl)(5- methoxypyrimidin-2-yl)methanone 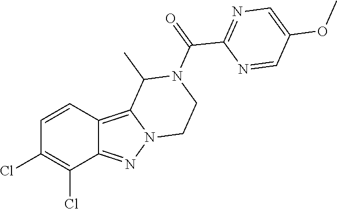 [SMILES: COc1cnc(C(=O)N2CCn3nc4c(Cl)c(Cl)ccc4c3C2C)nc1] |
| 52A* | 10 | (R)-(7,8-dichloro-1-methyl-3,4-dihydropyrazino[1,2-b]indazol-2(1H)-yl)(5- methoxypyrimidin-2-yl)methanone 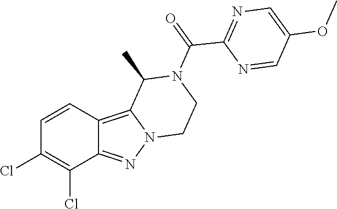 [SMILES: COc1cnc(C(=O)N2CCn3nc4c(Cl)c(Cl)ccc4c3[C@H]2C)nc1] |
| 52B* | 10 | (S)-(7,8-dichloro-1-methyl-3,4-dihydropyrazino[1,2-b]indazol-2(1H)-yl)(5- methoxypyrimidin-2-yl)methanone 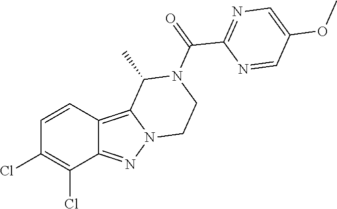 [SMILES: COc1cnc(C(=O)N2CCn3nc4c(Cl)c(Cl)ccc4c3[C@@H]2C)nc1] |
| Rac-67 | TA | (6,7-dichloro-1-methyl-1,3,4,5-tetrahydro-2H-pyrido[4,3-b]indol-2-yl)(4-(2- methoxyethoxy)pyrimidin-2-yl)methanone 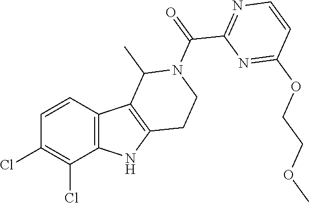 [SMILES: COCCOc1ccnc(C(=O)N2CCc3[nH]c4c(Cl)c(Cl)ccc4c3C2C)n1] |
| 67A* | TA | (R)-(6,7-dichloro-1-methyl-1,3,4,5-tetrahydro-2H-pyrido[4,3-b]indol-2-yl)(4-(2- methoxyethoxy)pyrimidin-2-yl)methanone 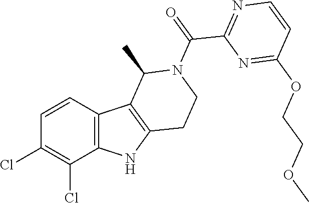 [SMILES: COCCOc1ccnc(C(=O)N2CCc3[nH]c4c(Cl)c(Cl)ccc4c3[C@H]2C)n1] |
| 67B* | TA | (S)-(6,7-dichloro-1-methyl-1,3,4,5-tetrahydro-2H-pyrido[4,3-b]indol-2-yl)(4-(2- methoxyethoxy)pyrimidin-2-yl)methanone 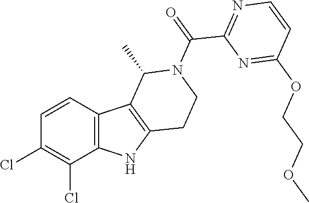 [SMILES: COCCOc1ccnc(C(=O)N2CCc3[nH]c4c(Cl)c(Cl)ccc4c3[C@@H]2C)n1] |
| Rac-68 | TA | (6,7-dichloro-1-methyl-1,3,4,5-tetrahydro-2H-pyrido[4,3-b]indol-2-yl)(4- methoxypyrimidin-2-yl)methanone 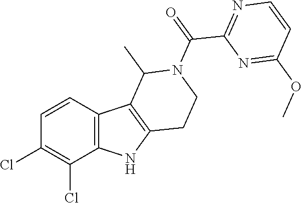 [SMILES: COc1ccnc(C(=O)N2CCc3[nH]c4c(Cl)c(Cl)ccc4c3C2C)n1] |
| 68A* | TA | (R)-(6,7-dichloro-1-methyl-1,3,4,5-tetrahydro-2H-pyrido[4,3-b]indol-2-yl)(4- methoxypyrimidin-2-yl)methanone 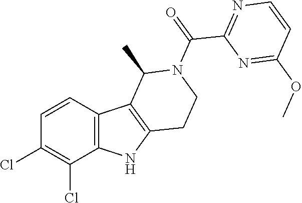 [SMILES: COc1ccnc(C(=O)N2CCc3[nH]c4c(Cl)c(Cl)ccc4c3[C@H]2C)n1] |
| 68B* | TA | (S)-(6,7-dichloro-1-methyl-1,3,4,5-tetrahydro-2H-pyrido[4,3-b]indol-2-yl)(4- methoxypyrimidin-2-yl)methanone 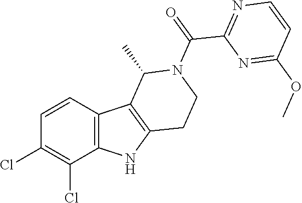 [SMILES: COc1ccnc(C(=O)N2CCc3[nH]c4c(Cl)c(Cl)ccc4c3[C@@H]2C)n1] |
| Rac-70 | TA | (6,7-dichloro-1-methyl-1,3,4,5-tetrahydro-2H-pyrido[4,3-b]indol-2-yl)(4-(2- hydroxyethoxy)pyrimidin-2-yl)methanone 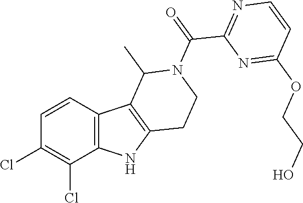 [SMILES: CC1c2c([nH]c3c(Cl)c(Cl)ccc23)CCN1C(=O)c1nccc(OCCO)n1] |
| 70A* | TA | (R)-(6,7-dichloro-1-methyl-1,3,4,5-tetrahydro-2H-pyrido[4,3-b]indol-2-yl)(4- (2-hydroxyethoxy)pyrimidin-2-yl)methanone 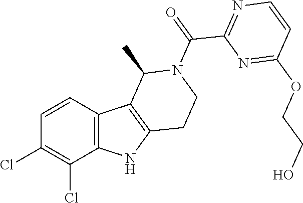 [SMILES: C[C@@H]1c2c([nH]c3c(Cl)c(Cl)ccc23)CCN1C(=O)c1nccc(OCCO)n1] |
| 70B* | TA | (S)-(6,7-dichloro-1-methyl-1,3,4,5-tetrahydro-2H-pyrido[4,3-b]indol-2-yl)(4-(2- hydroxyethoxy)pyrimidin-2-yl)methanone 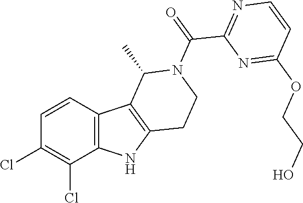 [SMILES: C[C@H]1c2c([nH]c3c(Cl)c(Cl)ccc23)CCN1C(=O)c1nccc(OCCO)n1] |
| 71 | 13 | (6,7-dichloro-9-(1-methyl-1H-pyrazol-3-yl)-1,3,4,5-tetrahydro-2H-pyrido[4,3- b]indol-2-yl)(5-methoxypyrimidin-2-yl)methanone 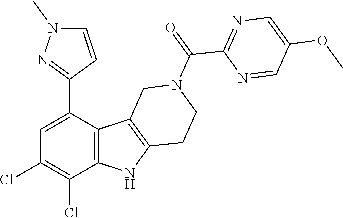 [SMILES: COc1cnc(C(=O)N2CCc3[nH]c4c(Cl)c(Cl)cc(-c5ccn(C)n5)c4c3C2)nc1] |
| 72 | TA | (6,7-dichloro-9-(pyrimidin-5-yl)-1,3,4,5-tetrahydro-2H-pyrido[4,3-b]indol-2- yl)(5-methoxypyrimidin-2-yl)methanone 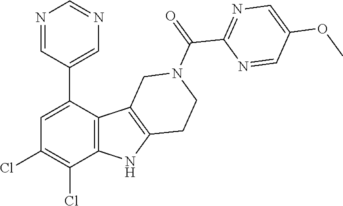 [SMILES: COc1cnc(C(=O)N2CCc3[nH]c4c(Cl)c(Cl)cc(-c5cncnc5)c4c3C2)nc1] |
| 73 | TA | (6,7-dichloro-9-(pyridin-3-yl)-1,3,4,5-tetrahydro-2H-pyrido[4,3-b]indol-2-yl)(5- methoxypyrimidin-2-yl)methanone 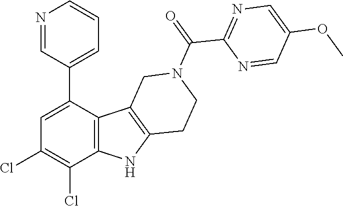 [SMILES: COc1cnc(C(=O)N2CCc3[nH]c4c(Cl)c(Cl)cc(-c5cccnc5)c4c3C2)nc1] |
| Rac-76 | TA | (6,7-dichloro-1-methyl-1,3,4,5-tetrahydro-2H-pyrido[4,3-b]indol-2-yl)(5-(2- (methylamino)ethoxy)pyrimidin-2-yl)methanone 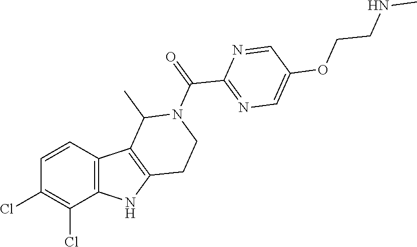 [SMILES: CNCCOc1cnc(C(=O)N2CCc3[nH]c4c(Cl)c(Cl)ccc4c3C2C)nc1] |
| 76A* | TA | (R)-(6,7-dichloro-1-methyl-1,3,4,5-tetrahydro-2H-pyrido[4,3-b]indol-2-yl)(5-(2- (methylamino)ethoxy)pyrimidin-2-yl)methanone 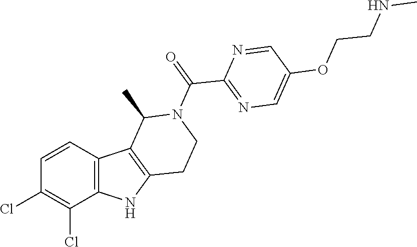 [SMILES: CNCCOc1cnc(C(=O)N2CCc3[nH]c4c(Cl)c(Cl)ccc4c3[C@H]2C)nc1] |
| 76B* | TA | (S)-(6,7-dichloro-1-methyl-1,3,4,5-tetrahydro-2H-pyrido[4,3-b]indol-2-yl)(5-(2- (methylamino)ethoxy)pyrimidin-2-yl)methanone 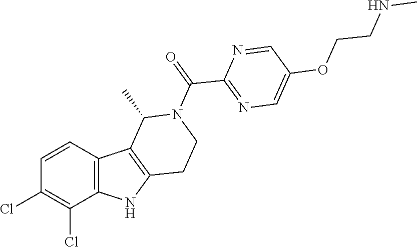 [SMILES: CNCCOc1cnc(C(=O)N2CCc3[nH]c4c(Cl)c(Cl)ccc4c3[C@@H]2C)nc1] |
| Rac-77 | 14 | (6,7-dichloro-1-methyl-1,3,4,5-tetrahydro-2H-pyrido[4,3-b]indol-2-yl)(5-(2- hydroxyethoxy)pyrimidin-2-yl)methanone 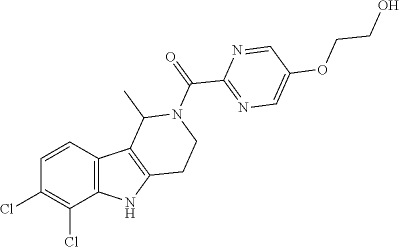 [SMILES: CC1c2c([nH]c3c(Cl)c(Cl)ccc23)CCN1C(=O)c1ncc(OCCO)cn1] |
| 77A* | 14 | (R)-(6,7-dichloro-1-methyl-1,3,4,5-tetrahydro-2H-pyrido[4,3-b]indol-2-yl)(5-(2- hydroxyethoxy)pyrimidin-2-yl)methanone 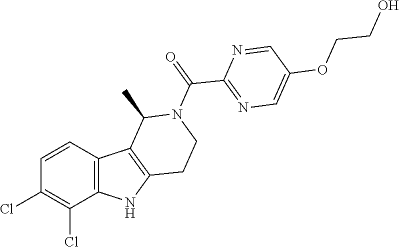 [SMILES: C[C@@H]1c2c([nH]c3c(Cl)c(Cl)ccc23)CCN1C(=O)c1ncc(OCCO)cn1] |
| 77B* | 14 | (S)-(6,7-dichloro-1-methyl-1,3,4,5-tetrahydro-2H-pyrido[4,3-b]indol-2-yl)(5-(2- hydroxyethoxy)pyrimidin-2-yl)methanone 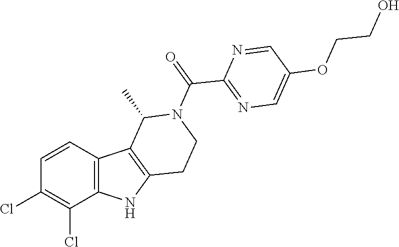 [SMILES: C[C@H]1c2c([nH]c3c(Cl)c(Cl)ccc23)CCN1C(=O)c1ncc(OCCO)cn1] |
| Rac-78 | TA | (6,7-dichloro-1-methyl-1,3,4,5-tetrahydro-2H-pyrido[4,3-b]indol-2-yl)(5-(2- methoxyethoxy)pyrimidin-2-yl)methanone 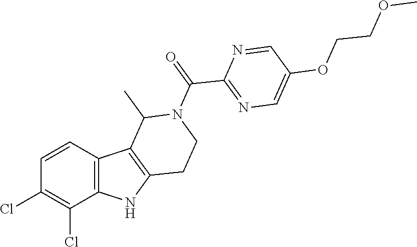 [SMILES: COCCOc1cnc(C(=O)N2CCc3[nH]c4c(Cl)c(Cl)ccc4c3C2C)nc1] |
| 78A* | TA | (R)-(6,7-dichloro-1-methyl-1,3,4,5-tetrahydro-2H-pyrido[4,3-b]indol-2-yl)(5-(2- methoxyethoxy)pyrimidin-2-yl)methanone 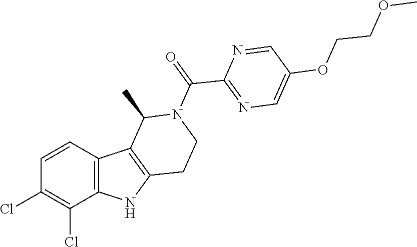 [SMILES: COCCOc1cnc(C(=O)N2CCc3[nH]c4c(Cl)c(Cl)ccc4c3[C@H]2C)nc1] |
| 78B* | TA | (S)-(6,7-dichloro-1-methyl-1,3,4,5-tetrahydro-2H-pyrido[4,3-b]indol-2-yl)(5-(2- methoxyethoxy)pyrimidin-2-yl)methanone 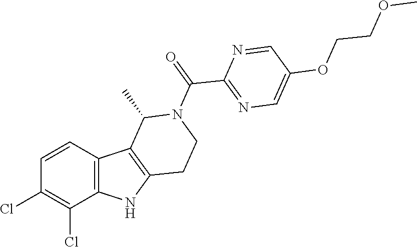 [SMILES: COCCOc1cnc(C(=O)N2CCc3[nH]c4c(Cl)c(Cl)ccc4c3[C@@H]2C)nc1] |
| Rac-79 | TA | 3-((2-(6,7-dichloro-1-methyl-2,3,4,5-tetrahydro-1H-pyrido[4,3-b]indole-2- carbonyl)pyrimidin-5-yl)oxy)propanenitrile 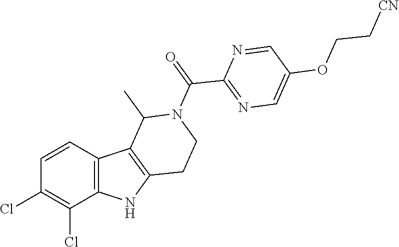 [SMILES: CC1c2c([nH]c3c(Cl)c(Cl)ccc23)CCN1C(=O)c1ncc(OCCC#N)cn1] |
| 79A* | TA | (R)-3-((2-(6,7-dichloro-1-methyl-2,3,4,5-tetrahydro-1H-pyrido[4,3-b]indole-2- carbonyl)pyrimidin-5-yl)oxy)propanenitrile 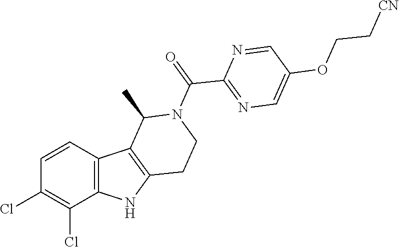 [SMILES: C[C@@H]1c2c([nH]c3c(Cl)c(Cl)ccc23)CCN1C(=O)c1ncc(OCCC#N)cn1] |
| 79B* | TA | (S)-3-((2-(6,7-dichloro-1-methyl-2,3,4,5-tetrahydro-1H-pyrido[4,3-b]indole-2- carbonyl)pyrimidin-5-yl)oxy)propanenitrile 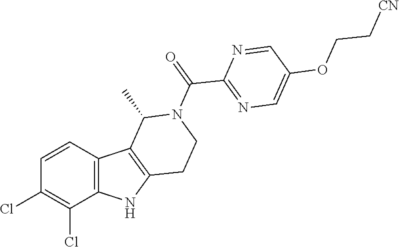 [SMILES: C[C@H]1c2c([nH]c3c(Cl)c(Cl)ccc23)CCN1C(=O)c1ncc(OCCC#N)cn1] |
| Rac-80 | TA | (6,7-dichloro-1-methyl-1,3,4,5-tetrahydro-2H-pyrido[4,3-b]indol-2-yl)(5- hydroxypyrimidin-2-yl)methanone 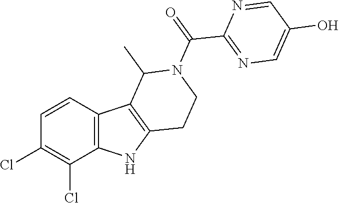 [SMILES: CC1c2c([nH]c3c(Cl)c(Cl)ccc23)CCN1C(=O)c1ncc(O)cn1] |
| 80A* | TA | (R)-(6,7-dichloro-1-methyl-1,3,4,5-tetrahydro-2H-pyrido[4,3-b]indol-2-yl)(5- hydroxypyrimidin-2-yl)methanone 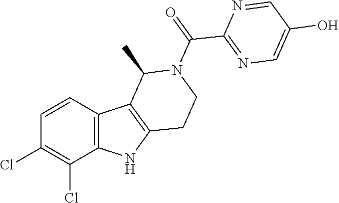 [SMILES: C[C@@H]1c2c([nH]c3c(Cl)c(Cl)ccc23)CCN1C(=O)c1ncc(O)cn1] |
| 80B* | TA | (S)-(6,7-dichloro-1-methyl-1,3,4,5-tetrahydro-2H-pyrido[4,3-b]indol-2-yl)(5- hydroxypyrimidin-2-yl)methanone 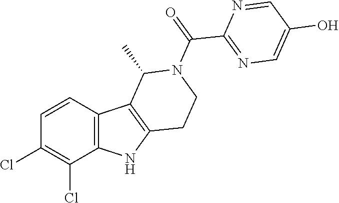 [SMILES: C[C@H]1c2c([nH]c3c(Cl)c(Cl)ccc23)CCN1C(=O)c1ncc(O)cn1] |
| Rac-81 | TA | (6,7-dichloro-1-methyl-1,3,4,5-tetrahydro-2H-pyrido[4,3-b]indol-2-yl)(4- hydroxypyrimidin-2-yl)methanone 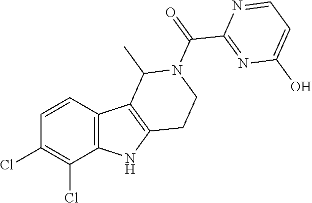 [SMILES: CC1c2c([nH]c3c(Cl)c(Cl)ccc23)CCN1C(=O)c1nccc(O)n1] |
| 81A* | TA | (R)-(6,7-dichloro-1-methyl-1,3,4,5-tetrahydro-2H-pyrido[4,3-b]indol-2-yl)(4- hydroxypyrimidin-2-yl)methanone 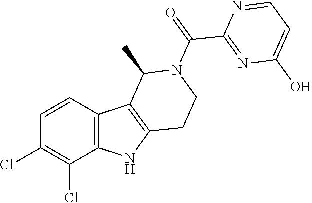 [SMILES: C[C@@H]1c2c([nH]c3c(Cl)c(Cl)ccc23)CCN1C(=O)c1nccc(O)n1] |
| 81B* | TA | (S)-(6,7-dichloro-1-methyl-1,3,4,5-tetrahydro-2H-pyrido[4,3-b]indol-2-yl)(4- hydroxypyrimidin-2-yl)methanone 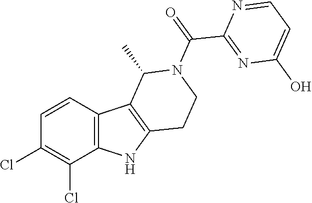 [SMILES: C[C@H]1c2c([nH]c3c(Cl)c(Cl)ccc23)CCN1C(=O)c1nccc(O)n1] |
| Rac-86 | TA | (9-(6-aminopyridin-3-yl)-6,7-dichloro-1-methyl-1,3,4,5-tetrahydro-2H- pyrido[4,3-b]indol-2-yl)(5-methoxypyrimidin-2-yl)methanone 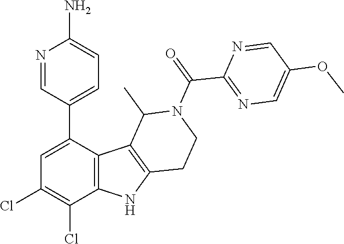 [SMILES: COc1cnc(C(=O)N2CCc3[nH]c4c(Cl)c(Cl)cc(-c5ccc(N)nc5)c4c3C2C)nc1] |
| 86A* | TA | (R)-(9-(6-aminopyridin-3-yl)-6,7-dichloro-1-methyl-1,3,4,5-tetrahydro-2H- pyrido[4,3-b]indol-2-yl)(5-methoxypyrimidin-2-yl)methanone 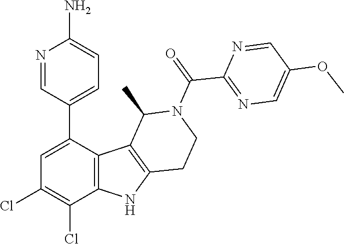 [SMILES: COc1cnc(C(=O)N2CCc3[nH]c4c(Cl)c(Cl)cc(-c5ccc(N)nc5)c4c3[C@H]2C)nc1] |
| 86B* | TA | (S)-(9-(6-aminopyridin-3-yl)-6,7-dichloro-1-methyl-1,3,4,5-tetrahydro-2H- pyrido[4,3-b]indol-2-yl)(5-methoxypyrimidin-2-yl)methanone 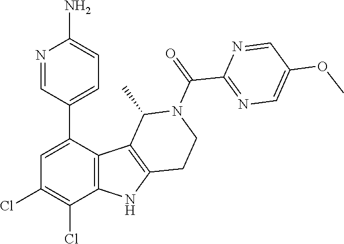 [SMILES: COc1cnc(C(=O)N2CCc3[nH]c4c(Cl)c(Cl)cc(-c5ccc(N)nc5)c4c3[C@@H]2C)nc1] |
| Rac-87 | TA | (10-(6-aminopyridin-3-yl)-7,8-dichloro-1-methyl-3,4-dihydropyrazino[1,2- b]indazol-2(1H)-yl)(5-methoxypyrimidin-2-yl)methanone 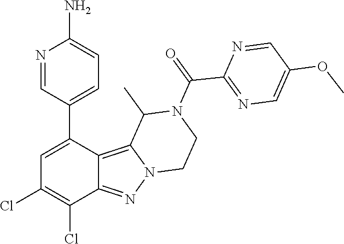 [SMILES: COc1cnc(C(=O)N2CCn3nc4c(Cl)c(Cl)cc(-c5ccc(N)nc5)c4c3C2C)nc1] |
| 87A* | TA | (R)-(10-(6-aminopyridin-3-yl)-7,8-dichloro-1-methyl-3,4-dihydropyrazino[1,2- b]indazol-2(1H)-yl)(5-methoxypyrimidin-2-yl)methanone 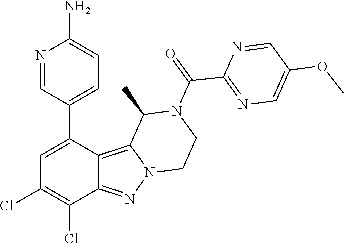 [SMILES: COc1cnc(C(=O)N2CCn3nc4c(Cl)c(Cl)cc(-c5ccc(N)nc5)c4c3[C@H]2C)nc1] |
| 87B* | TA | (S)-(10-(6-aminopyridin-3-yl)-7,8-dichloro-1-methyl-3,4-dihydropyrazino[1,2- b]indazol-2(1H)-yl)(5-methoxypyrimidin-2-yl)methanone 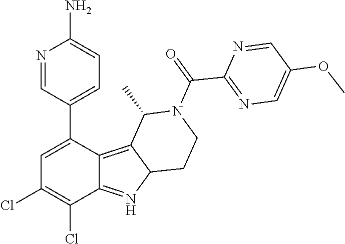 [SMILES: COc1cnc(C(=O)N2CCC3N=c4c(Cl)c(Cl)cc(-c5ccc(N)nc5)c4=C3[C@@H]2C)nc1] |
| Rac-88 | 16 | (6,7-dichloro-1-methyl-1,3,4,5-tetrahydro-2H-pyrido[4,3-b]indol-2-yl)(4-(2- (dimethylamino)ethoxy)pyrimidin-2-yl)methanone 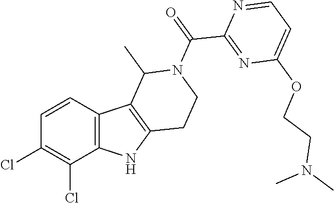 [SMILES: CC1c2c([nH]c3c(Cl)c(Cl)ccc23)CCN1C(=O)c1nccc(OCCN(C)C)n1] |
| 88A* | 16 | (R)-(6,7-dichloro-1-methyl-1,3,4,5-tetrahydro-2H-pyrido[4,3-b]indol-2-yl)(4-(2- (dimethylamino)ethoxy)pyrimidin-2-yl)methanone 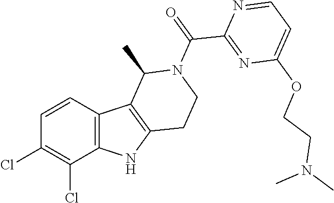 [SMILES: C[C@@H]1c2c([nH]c3c(Cl)c(Cl)ccc23)CCN1C(=O)c1nccc(OCCN(C)C)n1] |
| 88B* | 16 | (S)-(6,7-dichloro-1-methyl-1,3,4,5-tetrahydro-2H-pyrido[4,3-b]indol-2-yl)(4-(2- (dimethylamino)ethoxy)pyrimidin-2-yl)methanone 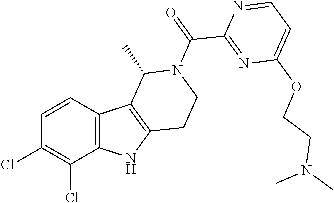 [SMILES: C[C@H]1c2c([nH]c3c(Cl)c(Cl)ccc23)CCN1C(=O)c1nccc(OCCN(C)C)n1] |
| Rac-89 | TA | 3-((2-(6,7-dichloro-1-methyl-2,3,4,5-tetrahydro-1H-pyrido[4,3-b]indole-2- carbonyl)pyrimidin-4-yl)oxy)propanenitrile 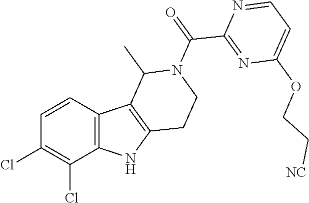 [SMILES: CC1c2c([nH]c3c(Cl)c(Cl)ccc23)CCN1C(=O)c1nccc(OCCC#N)n1] |
| 89A* | TA | (R)-3-((2-(6,7-dichloro-1-methyl-2,3,4,5-tetrahydro-1H-pyrido[4,3-b]indole-2- carbonyl)pyrimidin-4-yl)oxy)propanenitrile 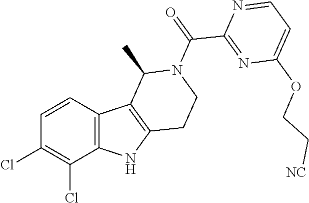 [SMILES: C[C@@H]1c2c([nH]c3c(Cl)c(Cl)ccc23)CCN1C(=O)c1nccc(OCCC#N)n1] |
| 89B* | TA | (S)-3-((2-(6,7-dichloro-1-methyl-2,3,4,5-tetrahydro-1H-pyrido[4,3-b]indole-2- carbonyl)pyrimidin-4-yl)oxy)propanenitrile 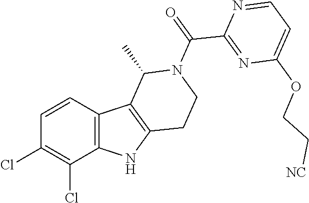 [SMILES: C[C@H]1c2c([nH]c3c(Cl)c(Cl)ccc23)CCN1C(=O)c1nccc(OCCC#N)n1] |
| Rac-96 | TA | (7-chloro-6-hydroxy-1-methyl-1,3,4,5-tetrahydro-2H-pyrido[4,3-b]indol-2- yl)(5-methoxypyrimidin-2-yl)methanone 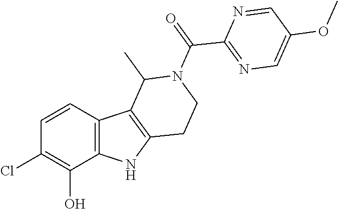 [SMILES: COc1cnc(C(=O)N2CCc3[nH]c4c(O)c(Cl)ccc4c3C2C)nc1] |
| 96A* | TA | (R)-(7-chloro-6-hydroxy-1-methyl-1,3,4,5-tetrahydro-2H-pyrido[4,3-b]indol-2- yl)(5-methoxypyrimidin-2-yl)methanone 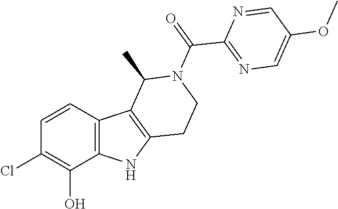 [SMILES: COc1cnc(C(=O)N2CCc3[nH]c4c(O)c(Cl)ccc4c3[C@H]2C)nc1] |
| 96B* | TA | (S)-(7-chloro-6-hydroxy-1-methyl-1,3,4,5-tetrahydro-2H-pyrido[4,3-b]indol-2- yl)(5-methoxypyrimidin-2-yl)methanone 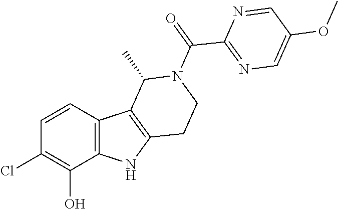 [SMILES: COc1cnc(C(=O)N2CCc3[nH]c4c(O)c(Cl)ccc4c3[C@@H]2C)nc1] |
| Rac-101 | 19 | (6,7-dichloro-1-methyl-9-(methylthio)-1,3,4,5-tetrahydro-2H-pyrido[4,3- b]indol-2-yl)(5-methoxypyrimidin-2-yl)methanone 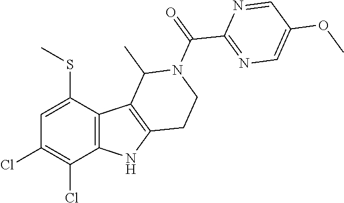 [SMILES: COc1cnc(C(=O)N2CCc3[nH]c4c(Cl)c(Cl)cc(SC)c4c3C2C)nc1] |
| 101A* | 19 | 2-[(1R)-6,7-dichloro-1-methyl-9-(methylsulfanyl)-1H,3H,4H,5H-pyrido[4,3- b]indole-2-carbonyl]-5-methoxypyrimidine; also referred to as (R)-(6,7- dichloro-1-methyl-9-(methylthio)-1,3,4,5-tetrahydro-2H-pyrido[4,3-b]indol-2- yl)(5-methoxypyrimidin-2-yl)methanone 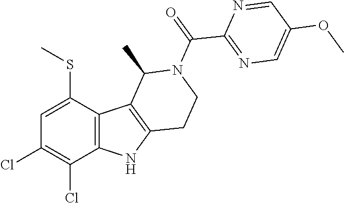 [SMILES: COc1cnc(C(=O)N2CCc3[nH]c4c(Cl)c(Cl)cc(SC)c4c3[C@H]2C)nc1] |
| 101B* | 19 | 2-[(1S)-6,7-dichloro-1-methyl-9-(methylsulfanyl)-1H,3H,4H,5H-pyrido[4,3- b]indole-2-carbonyl]-5-methoxypyrimidine; Also referred to as: (S)-(6,7- dichloro-1-methyl-9-(methylthio)-1,3,4,5-tetrahydro-2H-pyrido[4,3-b]indol-2- yl)(5-methoxypyrimidin-2-yl)methanone 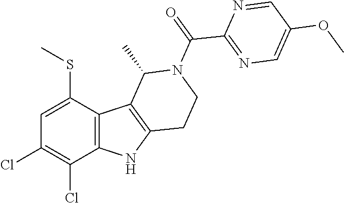 [SMILES: COc1cnc(C(=O)N2CCc3[nH]c4c(Cl)c(Cl)cc(SC)c4c3[C@@H]2C)nc1] |
| Rac-102 | TA | (6,7-dichloro-9-ethynyl-1-methyl-1,3,4,5-tetrahydro-2H-pyrido[4,3-b]indol-2- yl)(5-methoxypyrimidin-2-yl)methanone 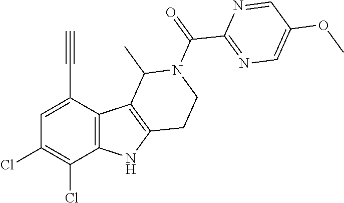 [SMILES: C#Cc1cc(Cl)c(Cl)c2[nH]c3c(c12)C(C)N(C(=O)c1ncc(OC)cn1)CC3] |
| 102A* | TA | (R)-(6,7-dichloro-9-ethynyl-1-methyl-1,3,4,5-tetrahydro-2H-pyrido[4,3-b]indol- 2-yl)(5-methoxypyrimidin-2-yl)methanone 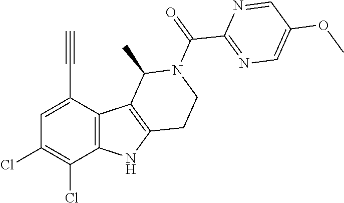 [SMILES: C#Cc1cc(Cl)c(Cl)c2[nH]c3c(c12)[C@@H](C)N(C(=O)c1ncc(OC)cn1)CC3] |
| 102B* | TA | (S)-(6,7-dichloro-9-ethynyl-1-methyl-1,3,4,5-tetrahydro-2H-pyrido[4,3-b]indol- 2-yl)(5-methoxypyrimidin-2-yl)methanone 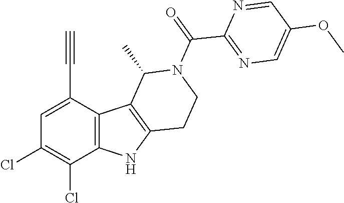 [SMILES: C#Cc1cc(Cl)c(Cl)c2[nH]c3c(c12)[C@H](C)N(C(=O)c1ncc(OC)cn1)CC3] |
| Rac-103 | 20 | (6,7-dichloro-1,9-dimethyl-1,3,4,5-tetrahydro-2H-pyrido[4,3-b]indol-2-yl)(5- methoxypyrimidin-2-yl)methanone 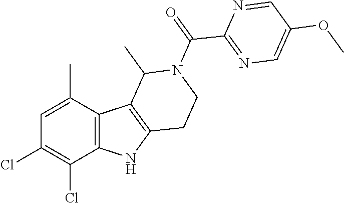 [SMILES: COc1cnc(C(=O)N2CCc3[nH]c4c(Cl)c(Cl)cc(C)c4c3C2C)nc1] |
| 103A* | 20 | (R)-(6,7-dichloro-1,9-dimethyl-1,3,4,5-tetrahydro-2H-pyrido[4,3-b]indol-2- yl)(5-methoxypyrimidin-2-yl)methanone 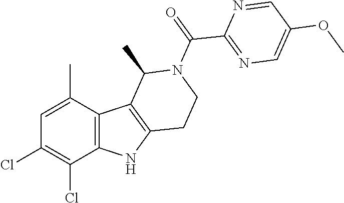 [SMILES: COc1cnc(C(=O)N2CCc3[nH]c4c(Cl)c(Cl)cc(C)c4c3[C@H]2C)nc1] |
| 103B* | 20 | (S)-(6,7-dichloro-1,9-dimethyl-1,3,4,5-tetrahydro-2H-pyrido[4,3-b]indol-2- yl)(5-methoxypyrimidin-2-yl)methanone 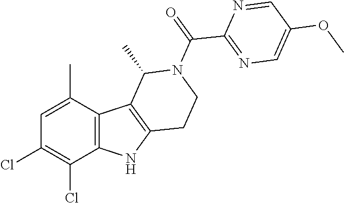 [SMILES: COc1cnc(C(=O)N2CCc3[nH]c4c(Cl)c(Cl)cc(C)c4c3[C@@H]2C)nc1] |
| Rac-104 | TA | (6,7-dichloro-1-methyl-9-(prop-1-yn-1-yl)-1,3,4,5-tetrahydro-2H-pyrido[4,3- b]indol-2-yl)(5-methoxypyrimidin-2-yl)methanone 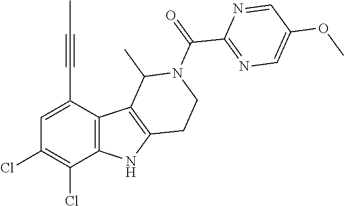 [SMILES: CC#Cc1cc(Cl)c(Cl)c2[nH]c3c(c12)C(C)N(C(=O)c1ncc(OC)cn1)CC3] |
| 104A* | TA | (R)-(6,7-dichloro-1-methyl-9-(prop-1-yn-1-yl)-1,3,4,5-tetrahydro-2H- pyrido[4,3-b]indol-2-yl)(5-methoxypyrimidin-2-yl)methanone 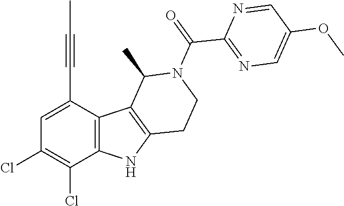 [SMILES: CC#Cc1cc(Cl)c(Cl)c2[nH]c3c(c12)[C@@H](C)N(C(=O)c1ncc(OC)cn1)CC3] |
| 104B* | TA | (S)-(6,7-dichloro-1-methyl-9-(prop-1-yn-1-yl)-1,3,4,5-tetrahydro-2H- pyrido[4,3-b]indol-2-yl)(5-methoxypyrimidin-2-yl)methanone 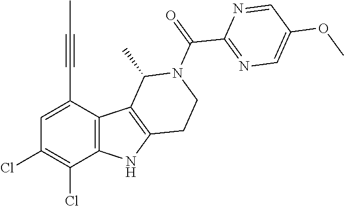 [SMILES: CC#Cc1cc(Cl)c(Cl)c2[nH]c3c(c12)[C@H](C)N(C(=O)c1ncc(OC)cn1)CC3] |
| Rac-105 | TA | (6,7-dichloro-9-(3-hydroxyprop-1-yn-1-yl)-1-methyl-1,3,4,5-tetrahydro-2H- pyrido[4,3-b]indol-2-yl)(5-methoxypyrimidin-2-yl)methanone 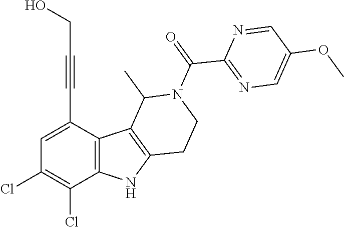 [SMILES: COc1cnc(C(=O)N2CCc3[nH]c4c(Cl)c(Cl)cc(C#CCO)c4c3C2C)nc1] |
| 105A* | TA | (R)-(6,7-dichloro-9-(3-hydroxyprop-1-yn-1-yl)-1-methyl-1,3,4,5-tetrahydro-2H- pyrido[4,3-b]indol-2-yl)(5-methoxypyrimidin-2-yl)methanone 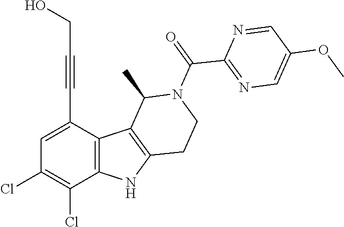 [SMILES: COc1cnc(C(=O)N2CCc3[nH]c4c(Cl)c(Cl)cc(C#CCO)c4c3[C@H]2C)nc1] |
| 105B* | TA | (S)-(6,7-dichloro-9-(3-hydroxyprop-1-yn-1-yl)-1-methyl-1,3,4,5-tetrahydro-2H- pyrido[4,3-b]indol-2-yl)(5-methoxypyrimidin-2-yl)methanone 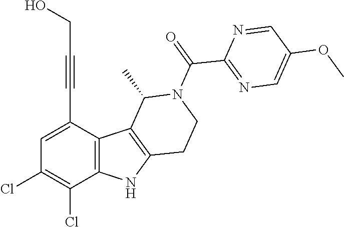 [SMILES: COc1cnc(C(=O)N2CCc3[nH]c4c(Cl)c(Cl)cc(C#CCO)c4c3[C@@H]2C)nc1] |
| Rac-107 | TA | (6,7-dichloro-1-methyl-5-(oxetan-3-ylmethyl)-1,3,4,5-tetrahydro-2H- pyrido[4,3-b]indol-2-yl)(5-methoxypyrimidin-2-yl)methanone 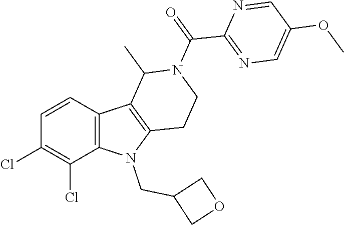 [SMILES: COc1cnc(C(=O)N2CCc3c(c4ccc(Cl)c(Cl)c4n3CC3COC3)C2C)nc1] |
| 107A* | TA | (R)-(6,7-dichloro-1-methyl-5-(oxetan-3-ylmethyl)-1,3,4,5-tetrahydro-2H- pyrido[4,3-b]indol-2-yl)(5-methoxypyrimidin-2-yl)methanone 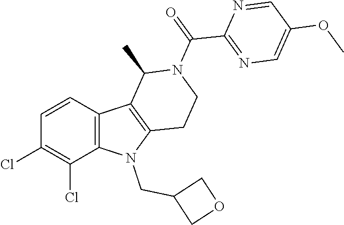 [SMILES: COc1cnc(C(=O)N2CCc3c(c4ccc(Cl)c(Cl)c4n3CC3COC3)[C@H]2C)nc1] |
| 107B* | TA | (S)-(6,7-dichloro-1-methyl-5-(oxetan-3-ylmethyl)-1,3,4,5-tetrahydro-2H- pyrido[4,3-b]indol-2-yl)(5-methoxypyrimidin-2-yl)methanone 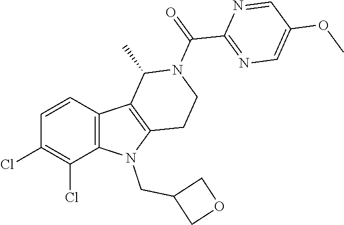 [SMILES: COc1cnc(C(=O)N2CCc3c(c4ccc(Cl)c(Cl)c4n3CC3COC3)[C@@H]2C)nc1] |
| Rac-108 | TA | 2-(6,7-dichloro-2-(5-methoxypyrimidine-2-carbonyl)-1-methyl-1,2,3,4- tetrahydro-5H-pyrido[4,3-b]indol-5-yl)acetonitrile 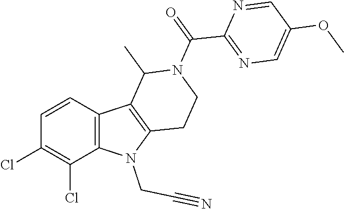 [SMILES: COc1cnc(C(=O)N2CCc3c(c4ccc(Cl)c(Cl)c4n3CC#N)C2C)nc1] |
| 108A* | TA | (R)-2-(6,7-dichloro-2-(5-methoxypyrimidine-2-carbonyl)-1-methyl-1,2,3,4- tetrahydro-5H-pyrido[4,3-b]indol-5-yl)acetonitrile 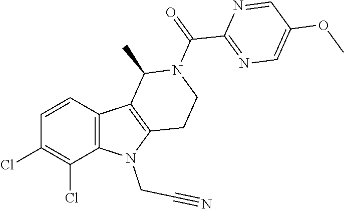 [SMILES: COc1cnc(C(=O)N2CCc3c(c4ccc(Cl)c(Cl)c4n3CC#N)[C@H]2C)nc1] |
| 108B* | TA | (S)-2-(6,7-dichloro-2-(5-methoxypyrimidine-2-carbonyl)-1-methyl-1,2,3,4- tetrahydro-5H-pyrido[4,3-b]indol-5-yl)acetonitrile 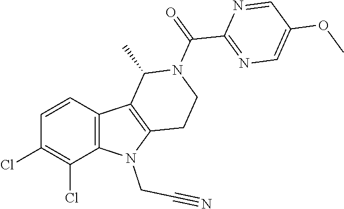 [SMILES: COc1cnc(C(=O)N2CCc3c(c4ccc(Cl)c(Cl)c4n3CC#N)[C@@H]2C)nc1] |
| Rac-109 | TA | (6,7-dichloro-5-((2-methoxyethoxy)methyl)-1-methyl-1,3,4,5-tetrahydro-2H- pyrido[4,3-b]indol-2-yl)(5-methoxypyrimidin-2-yl)methanone 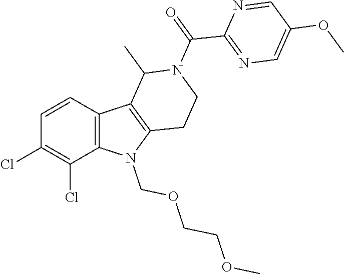 [SMILES: COCCOCn1c2c(c3ccc(Cl)c(Cl)c31)C(C)N(C(=O)c1ncc(OC)cn1)CC2] |
| 109A* | TA | (R)-(6,7-dichloro-5-((2-methoxyethoxy)methyl)-1-methyl-1,3,4,5-tetrahydro- 2H-pyrido[4,3-b]indol-2-yl)(5-methoxypyrimidin-2-yl)methanone 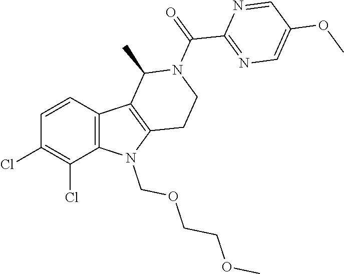 [SMILES: COCCOCn1c2c(c3ccc(Cl)c(Cl)c31)[C@@H](C)N(C(=O)c1ncc(OC)cn1)CC2] |
| 109B* | TA | (S)-(6,7-dichloro-5-((2-methoxyethoxy)methyl)-1-methyl-1,3,4,5-tetrahydro- 2H-pyrido[4,3-b]indol-2-yl)(5-methoxypyrimidin-2-yl)methanone 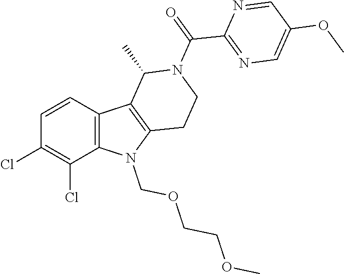 [SMILES: COCCOCn1c2c(c3ccc(Cl)c(Cl)c31)[C@H](C)N(C(=O)c1ncc(OC)cn1)CC2] |
| Rac-112 | TA | (5,6-dichloro-1-methyl-3,4-dihydropyrrolo[3,4-b]indol-2(1H)-yl)(5- methoxypyrimidin-2-yl)methanone 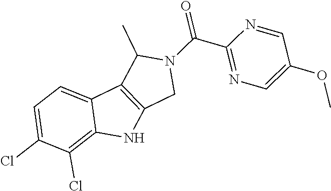 [SMILES: COc1cnc(C(=O)N2Cc3[nH]c4c(Cl)c(Cl)ccc4c3C2C)nc1] |
| 112A* | TA | (R)-(5,6-dichloro-1-methyl-3,4-dihydropyrrolo[3,4-b]indol-2(1H)-yl)(5- methoxypyrimidin-2-yl)methanone 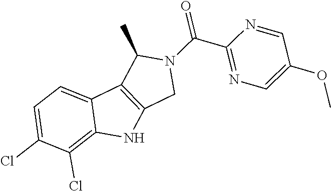 [SMILES: COc1cnc(C(=O)N2Cc3[nH]c4c(Cl)c(Cl)ccc4c3[C@H]2C)nc1] |
| 112B* | TA | (S)-(5,6-dichloro-1-methyl-3,4-dihydropyrrolo[3,4-b]indol-2(1H)-yl)(5- methoxypyrimidin-2-yl)methanone 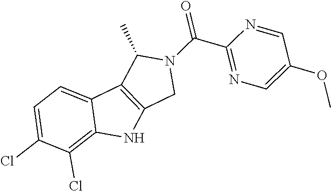 [SMILES: COc1cnc(C(=O)N2Cc3[nH]c4c(Cl)c(Cl)ccc4c3[C@@H]2C)nc1] |
| Rac-113 | TA | (6,7-dichloro-1-methyl-9-(1-methyl-1H-pyrazol-3-yl)-1,3,4,5-tetrahydro-2H- pyrido[4,3-b]indol-2-yl)(5-methoxypyrimidin-2-yl)methanone 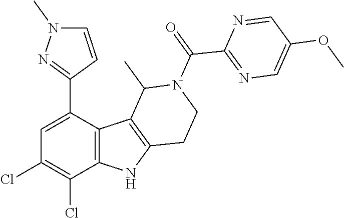 [SMILES: COc1cnc(C(=O)N2CCc3[nH]c4c(Cl)c(Cl)cc(-c5ccn(C)n5)c4c3C2C)nc1] |
| 113A* | TA | (R)-(6,7-dichloro-1-methyl-9-(1-methyl-1H-pyrazol-3-yl)-1,3,4,5-tetrahydro- 2H-pyrido[4,3-b]indol-2-yl)(5-methoxypyrimidin-2-yl)methanone 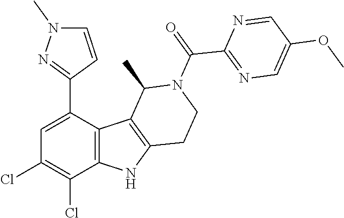 [SMILES: COc1cnc(C(=O)N2CCc3[nH]c4c(Cl)c(Cl)cc(-c5ccn(C)n5)c4c3[C@H]2C)nc1] |
| 113B* | TA | (S)-(6,7-dichloro-1-methyl-9-(1-methyl-1H-pyrazol-3-yl)-1,3,4,5-tetrahydro- 2H-pyrido[4,3-b]indol-2-yl)(5-methoxypyrimidin-2-yl)methanone 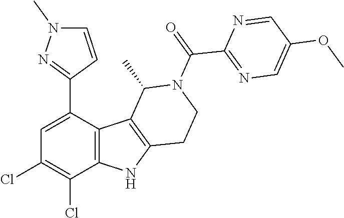 [SMILES: COc1cnc(C(=O)N2CCc3[nH]c4c(Cl)c(Cl)cc(-c5ccn(C)n5)c4c3[C@@H]2C)nc1] |
| Rac-115 | TA | (6,7-dichloro-8-methoxy-1-methyl-1,3,4,5-tetrahydro-2H-pyrido[4,3-b]indol-2- yl)(5-methoxypyrimidin-2-yl)methanone 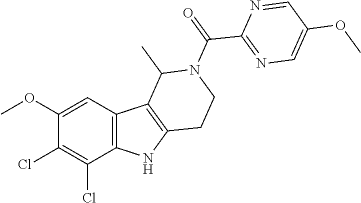 |
| 115A* | TA | (R)-(6,7-dichloro-8-methoxy-1-methyl-1,3,4,5-tetrahydro-2H-pyrido[4,3- b]indol-2-yl)(5-methoxypyrimidin-2-yl)methanone 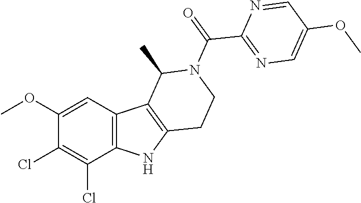 |
| 115B* | TA | (S)-(6,7-dichloro-8-methoxy-1-methyl-1,3,4,5-tetrahydro-2H-pyrido[4,3- b]indol-2-yl)(5-methoxypyrimidin-2-yl)methanone 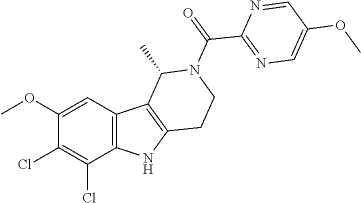 |
| Rac-116 | TA | (6,7-dichloro-8-hydroxy-1-methyl-1,3,4,5-tetrahydro-2H-pyrido[4,3-b]indol-2- yl)(5-methoxypyrimidin-2-yl)methanone 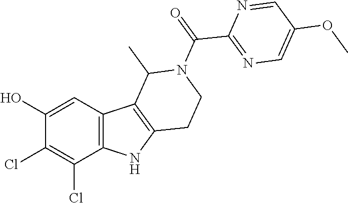 [SMILES: COc1cnc(C(=O)N2CCc3[nH]c4c(Cl)c(Cl)c(O)cc4c3C2C)nc1] |
| 116A* | TA | (R)-(6,7-dichloro-8-hydroxy-1-methyl-1,3,4,5-tetrahydro-2H-pyrido[4,3- b]indol-2-yl)(5-methoxypyrimidin-2-yl)methanone 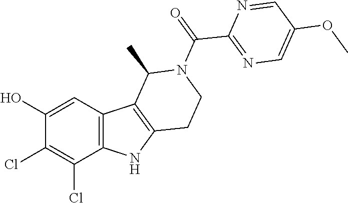 [SMILES: COc1cnc(C(=O)N2CCc3[nH]c4c(Cl)c(Cl)c(O)cc4c3[C@H]2C)nc1] |
| 116B* | TA | (S)-(6,7-dichloro-8-hydroxy-1-methyl-1,3,4,5-tetrahydro-2H-pyrido[4,3- b]indol-2-yl)(5-methoxypyrimidin-2-yl)methanone 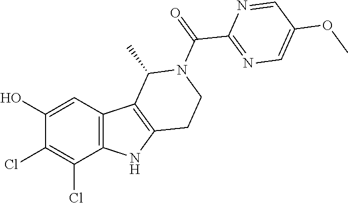 [SMILES: COc1cnc(C(=O)N2CCc3[nH]c4c(Cl)c(Cl)c(O)cc4c3[C@@H]2C)nc1] |
| Rac-117 | TA | (6,7-dichloro-1-methyl-8-(methylthio)-1,3,4,5-tetrahydro-2H-pyrido[4,3- b]indol-2-yl)(5-methoxypyrimidin-2-yl)methanone 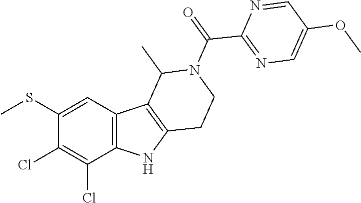 |
| 117A* | TA | (R)-(6,7-dichloro-1-methyl-8-(methylthio)-1,3,4,5-tetrahydro-2H-pyrido[4,3- b]indol-2-yl)(5-methoxypyrimidin-2-yl)methanone 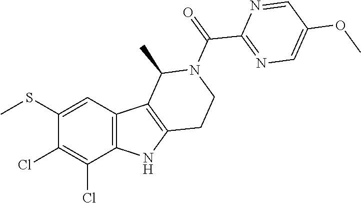 |
| 117B* | TA | (S)-(6,7-dichloro-1-methyl-8-(methylthio)-1,3,4,5-tetrahydro-2H-pyrido[4,3- b]indol-2-yl)(5-methoxypyrimidin-2-yl)methanone 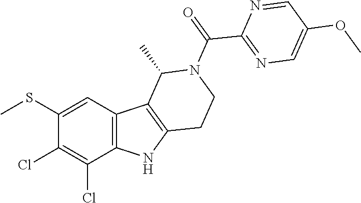 |
| Rac-118 | TA | (7-chloro-6-fluoro-1-methyl-1,3,4,5-tetrahydro-2H-pyrido[4,3-b]indol-2-yl)(5- methoxypyrimidin-2-yl)methanone 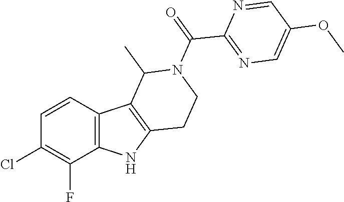 [SMILES: COc1cnc(C(=O)N2CCc3[nH]c4c(F)c(Cl)ccc4c3C2C)nc1] |
| 118A* | TA | (R)-(7-chloro-6-fluoro-1-methyl-1,3,4,5-tetrahydro-2H-pyrido[4,3-b]indol-2- yl)(5-methoxypyrimidin-2-yl)methanone 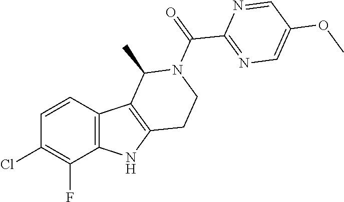 [SMILES: COc1cnc(C(=O)N2CCc3[nH]c4c(F)c(Cl)ccc4c3[C@H]2C)nc1] |
| 118B* | TA | (S)-(7-chloro-6-fluoro-1-methyl-1,3,4,5-tetrahydro-2H-pyrido[4,3-b]indol-2- yl)(5-methoxypyrimidin-2-yl)methanone 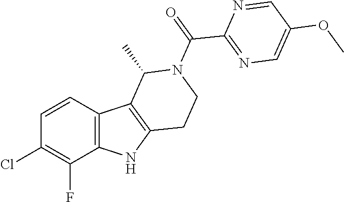 [SMILES: COc1cnc(C(=O)N2CCc3[nH]c4c(F)c(Cl)ccc4c3[C@@H]2C)nc1] |
| Rac-121 | TA | (7-bromo-6-chloro-1-methyl-1,3,4,5-tetrahydro-2H-pyrido[4,3-b]indol-2-yl)(5- methoxypyrimidin-2-yl)methanone 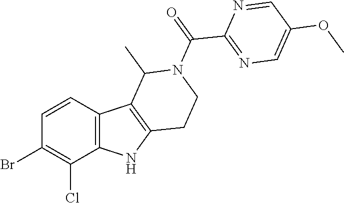 [SMILES: COc1cnc(C(=O)N2CCc3[nH]c4c(Cl)c(Br)ccc4c3C2C)nc1] |
| 121A* | TA | (R)-(7-bromo-6-chloro-1-methyl-1,3,4,5-tetrahydro-2H-pyrido[4,3-b]indol-2- yl)(5-methoxypyrimidin-2-yl)methanone 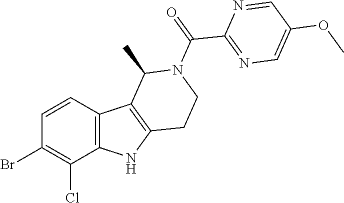 [SMILES: COc1cnc(C(=O)N2CCc3[nH]c4c(Cl)c(Br)ccc4c3[C@H]2C)nc1] |
| 121B* | TA | (S)-(7-bromo-6-chloro-1-methyl-1,3,4,5-tetrahydro-2H-pyrido[4,3-b]indol-2- yl)(5-methoxypyrimidin-2-yl)methanone 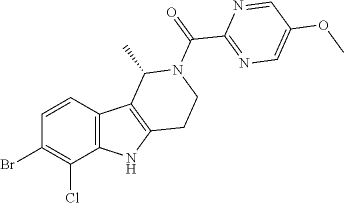 [SMILES: COc1cnc(C(=O)N2CCc3[nH]c4c(Cl)c(Br)ccc4c3[C@@H]2C)nc1] |
| Rac-123 | TA | 7-chloro-2-(5-methoxypyrimidine-2-carbonyl)-1-methyl-2,3,4,5-tetrahydro-1H- pyrido[4,3-b]indole-6-carbonitrile 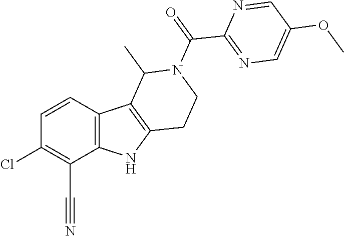 [SMILES: COc1cnc(C(=O)N2CCc3[nH]c4c(C#N)c(Cl)ccc4c3C2C)nc1] |
| 123A* | TA | (R)-7-chloro-2-(5-methoxypyrimidine-2-carbonyl)-1-methyl-2,3,4,5-tetrahydro- 1H-pyrido[4,3-b]indole-6-carbonitrile 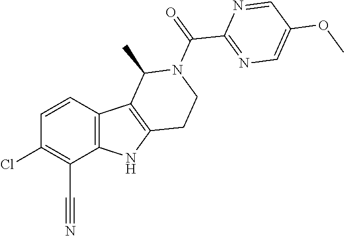 [SMILES: COc1cnc(C(=O)N2CCc3[nH]c4c(C#N)c(Cl)ccc4c3[C@H]2C)nc1] |
| 123B* | TA | (S)-7-chloro-2-(5-methoxypyrimidine-2-carbonyl)-1-methyl-2,3,4,5-tetrahydro- 1H-pyrido[4,3-b]indole-6-carbonitrile 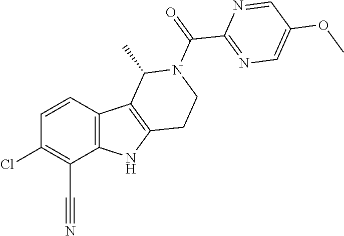 [SMILES: COc1cnc(C(=O)N2CCc3[nH]c4c(C#N)c(Cl)ccc4c3[C@@H]2C)nc1] |
| Rac-125 | TA | 3-(6,7-dichloro-2-(5-methoxypyrimidine-2-carbonyl)-1-methyl-2,3,4,5- tetrahydro-1H-pyrido[4,3-b]indol-9-yl)propanenitrile 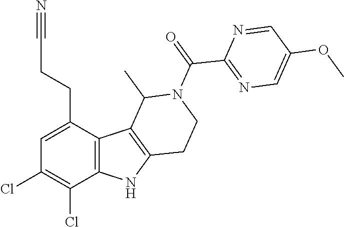 [SMILES: COc1cnc(C(=O)N2CCc3[nH]c4c(Cl)c(Cl)cc(CCC#N)c4c3C2C)nc1] |
| 125A* | TA | (R)-3-(6,7-dichloro-2-(5-methoxypyrimidine-2-carbonyl)-1-methyl-2,3,4,5- tetrahydro-1H-pyrido[4,3-b]indol-9-yl)propanenitrile 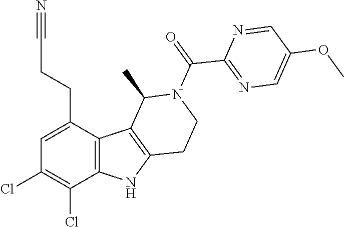 [SMILES: COc1cnc(C(=O)N2CCc3[nH]c4c(Cl)c(Cl)cc(CCC#N)c4c3[C@H]2C)nc1] |
| 125B* | TA | (S)-3-(6,7-dichloro-2-(5-methoxypyrimidine-2-carbonyl)-1-methyl-2,3,4,5- tetrahydro-1H-pyrido[4,3-b]indol-9-yl)propanenitrile 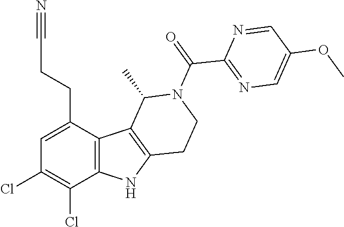 [SMILES: COc1cnc(C(=O)N2CCc3[nH]c4c(Cl)c(Cl)cc(CCC#N)c4c3[C@@H]2C)nc1] |
| Rac-126 | TA | 2-(6,7-dichloro-2-(5-methoxypyrimidine-2-carbonyl)-1-methyl-2,3,4,5- tetrahydro-1H-pyrido[4,3-b]indol-9-yl)acetonitrile 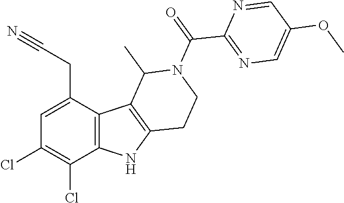 [SMILES: COc1cnc(C(=O)N2CCc3[nH]c4c(Cl)c(Cl)cc(CC#N)c4c3C2C)nc1] |
| 126A* | TA | (R)-2-(6,7-dichloro-2-(5-methoxypyrimidine-2-carbonyl)-1-methyl-2,3,4,5- tetrahydro-1H-pyrido[4,3-b]indol-9-yl)acetonitrile 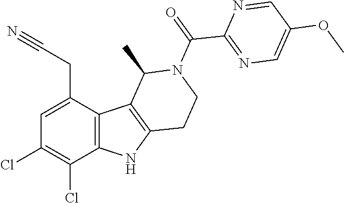 [SMILES: COc1cnc(C(=O)N2CCc3[nH]c4c(Cl)c(Cl)cc(CC#N)c4c3[C@H]2C)nc1] |
| 126B* | TA | (S)-2-(6,7-dichloro-2-(5-methoxypyrimidine-2-carbonyl)-1-methyl-2,3,4,5- tetrahydro-1H-pyrido[4,3-b]indol-9-yl)acetonitrile 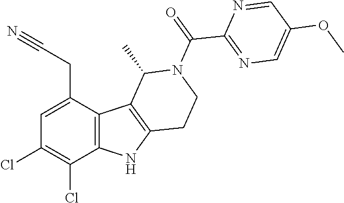 [SMILES: COc1cnc(C(=O)N2CCc3[nH]c4c(Cl)c(Cl)cc(CC#N)c4c3[C@@H]2C)nc1] |
| Rac-127 | TA | (6,7-dichloro-9-(difluoromethyl)-1-methyl-1,3,4,5-tetrahydro-2H-pyrido[4,3- b]indol-2-yl)(5-methoxypyrimidin-2-yl)methanone 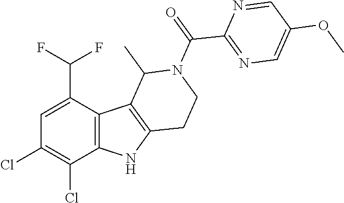 [SMILES: COc1cnc(C(=O)N2CCc3[nH]c4c(Cl)c(Cl)cc(C(F)F)c4c3C2C)nc1] |
| 127A* | TA | (R)-(6,7-dichloro-9-(difluoromethyl)-1-methyl-1,3,4,5-tetrahydro-2H- pyrido[4,3-b]indol-2-yl)(5-methoxypyrimidin-2-yl)methanone 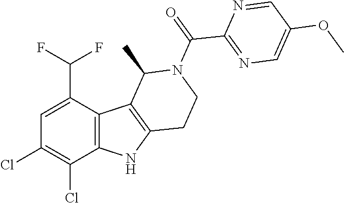 [SMILES: COc1cnc(C(=O)N2CCc3[nH]c4c(Cl)c(Cl)cc(C(F)F)c4c3[C@H]2C)nc1] |
| 127B* | TA | (S)-(6,7-dichloro-9-(difluoromethyl)-1-methyl-1,3,4,5-tetrahydro-2H- pyrido[4,3-b]indol-2-yl)(5-methoxypyrimidin-2-yl)methanone 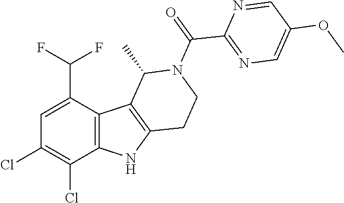 [SMILES: COc1cnc(C(=O)N2CCc3[nH]c4c(Cl)c(Cl)cc(C(F)F)c4c3[C@@H]2C)nc1] |
| Rac-128 | TA | (6,7-dichloro-9-ethyl-1-methyl-1,3,4,5-tetrahydro-2H-pyrido[4,3-b]indol-2- yl)(5-methoxypyrimidin-2-yl)methanone 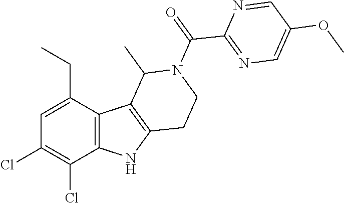 [SMILES: CCc1cc(Cl)c(Cl)c2[nH]c3c(c12)C(C)N(C(=O)c1ncc(OC)cn1)CC3] |
| 128A* | TA | (R)-(6,7-dichloro-9-ethyl-1-methyl-1,3,4,5-tetrahydro-2H-pyrido[4,3-b]indol-2- yl)(5-methoxypyrimidin-2-yl)methanone 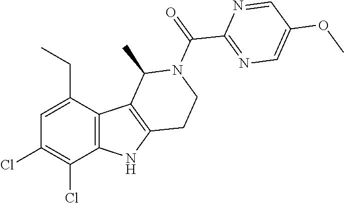 [SMILES: CCc1cc(Cl)c(Cl)c2[nH]c3c(c12)[C@@H](C)N(C(=O)c1ncc(OC)cn1)CC3] |
| 128B* | TA | (S)-(6,7-dichloro-9-ethyl-1-methyl-1,3,4,5-tetrahydro-2H-pyrido[4,3-b]indol-2- yl)(5-methoxypyrimidin-2-yl)methanone 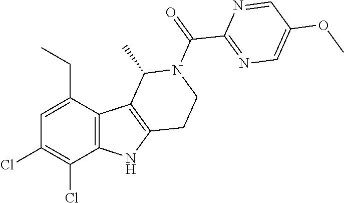 [SMILES: CCc1cc(Cl)c(Cl)c2[nH]c3c(c12)[C@H](C)N(C(=O)c1ncc(OC)cn1)CC3] |
| Rac-129 | TA | (6,7-dichloro-9-(difluoromethoxy)-1-methyl-1,3,4,5-tetrahydro-2H-pyrido[4,3- b]indol-2-yl)(5-methoxypyrimidin-2-yl)methanone 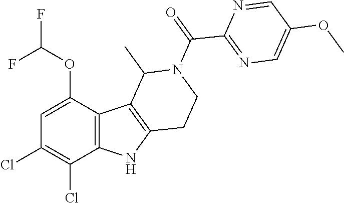 [SMILES: COc1cnc(C(=O)N2CCc3[nH]c4c(Cl)c(Cl)cc(OC(F)F)c4c3C2C)nc1] |
| 129A* | TA | (R)-(6,7-dichloro-9-(difluoromethoxy)-1-methyl-1,3,4,5-tetrahydro-2H- pyrido[4,3-b]indol-2-yl)(5-methoxypyrimidin-2-yl)methanone 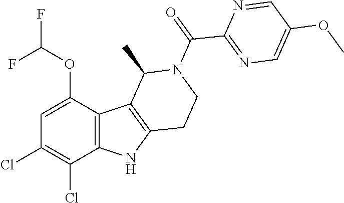 [SMILES: COc1cnc(C(=O)N2CCc3[nH]c4c(Cl)c(Cl)cc(OC(F)F)c4c3[C@H]2C)nc1] |
| 129B* | TA | (S)-(6,7-dichloro-9-(difluoromethoxy)-1-methyl-1,3,4,5-tetrahydro-2H- pyrido[4,3-b]indol-2-yl)(5-methoxypyrimidin-2-yl)methanone 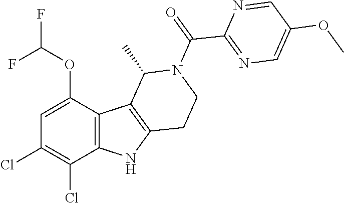 [SMILES: COc1cnc(C(=O)N2CCc3[nH]c4c(Cl)c(Cl)cc(OC(F)F)c4c3[C@@H]2C)nc1] |
| Rac-131 | TA | (6,7-dichloro-1-methyl-9-vinyl-1,3,4,5-tetrahydro-2H-pyrido[4,3-b]indol-2- yl)(5-methoxypyrimidin-2-yl)methanone 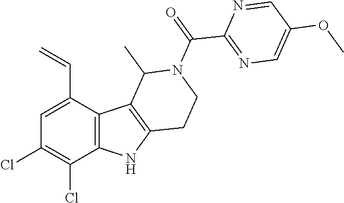 [SMILES: C=Cc1cc(Cl)c(Cl)c2[nH]c3c(c12)C(C)N(C(=O)c1ncc(OC)cn1)CC3] |
| 131A* | TA | (R)-(6,7-dichloro-1-methyl-9-vinyl-1,3,4,5-tetrahydro-2H-pyrido[4,3-b]indol-2- yl)(5-methoxypyrimidin-2-yl)methanone 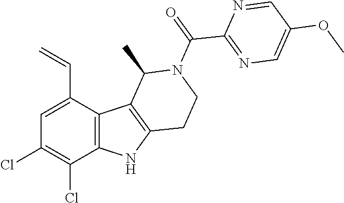 [SMILES: C=Cc1cc(Cl)c(Cl)c2[nH]c3c(c12)[C@@H](C)N(C(=O)c1ncc(OC)cn1)CC3] |
| 131B* | TA | (S)-(6,7-dichloro-1-methyl-9-vinyl-1,3,4,5-tetrahydro-2H-pyrido[4,3-b]indol-2- yl)(5-methoxypyrimidin-2-yl)methanone 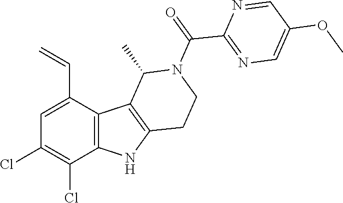 [SMILES: C=Cc1cc(Cl)c(Cl)c2[nH]c3c(c12)[C@H](C)N(C(=O)c1ncc(OC)cn1)CC3] |
| Rac-132 | 22 | (6,7-dichloro-9-methoxy-1-methyl-1,3,4,5-tetrahydro-2H-pyrido[4,3-b]indol-2- yl)(5-methoxypyrimidin-2-yl)methanone 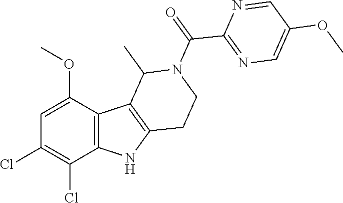 [SMILES: COc1cnc(C(=O)N2CCc3[nH]c4c(Cl)c(Cl)cc(OC)c4c3C2C)nc1] |
| 132A* | 22 | (R)-(6,7-dichloro-9-methoxy-1-methyl-1,3,4,5-tetrahydro-2H-pyrido[4,3- b]indol-2-yl)(5-methoxypyrimidin-2-yl)methanone 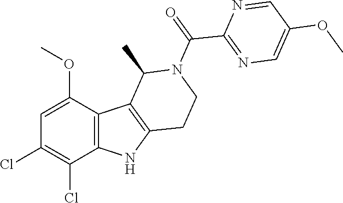 [SMILES: COc1cnc(C(=O)N2CCc3[nH]c4c(Cl)c(Cl)cc(OC)c4c3[C@H]2C)nc1] |
| 132B* | 22 | (S)-(6,7-dichloro-9-methoxy-1-methyl-1,3,4,5-tetrahydro-2H-pyrido[4,3- b]indol-2-yl)(5-methoxypyrimidin-2-yl)methanone 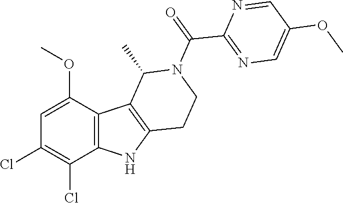 [SMILES: COc1cnc(C(=O)N2CCc3[nH]c4c(Cl)c(Cl)cc(OC)c4c3[C@@H]2C)nc1] |
| Rac-133 | TA | 7,8-dichloro-2,3,4,9-tetrahydro-1H-2,4-epiminocarbazol-10-yl)(5- methoxypyrimidin-2-yl)methanone 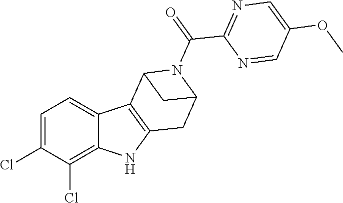 [SMILES: COc1cnc(C(=O)N2C3Cc4[nH]c5c(Cl)c(Cl)ccc5c4C2C3)nc1] |
| 133A* | TA | ((2S,4R)-7,8-dichloro-2,3,4,9-tetrahydro-1H-2,4-epiminocarbazol-10-yl)(5- methoxypyrimidin-2-yl)methanone 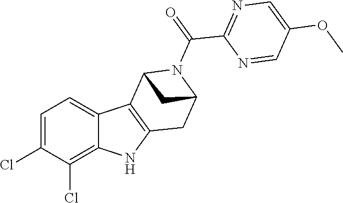 [SMILES: COc1cnc(C(=O)N2[C@@H]3Cc4[nH]c5c(Cl)c(Cl)ccc5c4[C@H]2C3)nc1] |
| 133B* | TA | ((2R,4S)-7,8-dichloro-2,3,4,9-tetrahydro-1H-2,4-epiminocarbazol-10-yl)(5- methoxypyrimidin-2-yl)methanone 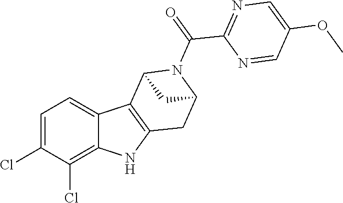 [SMILES: COc1cnc(C(=O)N2[C@H]3Cc4[nH]c5c(Cl)c(Cl)ccc5c4[C@@H]2C3)nc1] |
| Rac-137 | TA | (6,7-dichloro-4-phenyl-1,3,4,5-tetrahydro-2H-pyrido[4,3-b]indol-2-yl)(5- methoxypyrimidin-2-yl)methanone 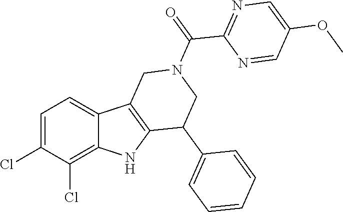 [SMILES: COc1cnc(C(=O)N2Cc3c([nH]c4c(Cl)c(Cl)ccc34)C(c3ccccc3)C2)nc1] |
| 137A* | TA | (R)-(6,7-dichloro-4-phenyl-1,3,4,5-tetrahydro-2H-pyrido[4,3-b]indol-2-yl)(5- methoxypyrimidin-2-yl)methanone 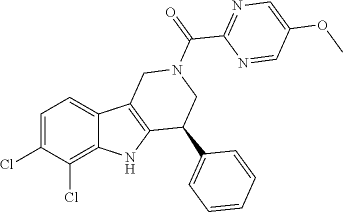 [SMILES: COc1cnc(C(=O)N2Cc3c([nH]c4c(Cl)c(Cl)ccc34)[C@H](c3ccccc3)C2)nc1] |
| 137B* | TA | (S)-(6,7-dichloro-4-phenyl-1,3,4,5-tetrahydro-2H-pyrido[4,3-b]indol-2-yl)(5- methoxypyrimidin-2-yl)methanone 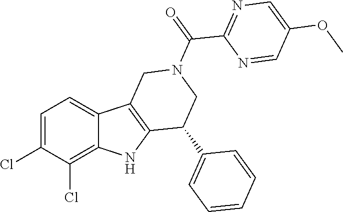 [SMILES: COc1cnc(C(=O)N2Cc3c([nH]c4c(Cl)c(Cl)ccc34)[C@@H](c3ccccc3)C2)nc1] |
| Rac-138 | TA | (6,7-dichloro-4-(pyridin-3-yl)-1,3,4,5-tetrahydro-2H-pyrido[4,3-b]indol-2-yl)(5- methoxypyrimidin-2-yl)methanone 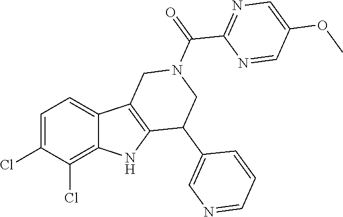 [SMILES: COc1cnc(C(=O)N2Cc3c([nH]c4c(Cl)c(Cl)ccc34)C(c3cccnc3)C2)nc1] |
| 138A* | TA | (R)-(6,7-dichloro-4-(pyridin-3-yl)-1,3,4,5-tetrahydro-2H-pyrido[4,3-b]indol-2- yl)(5-methoxypyrimidin-2-yl)methanone 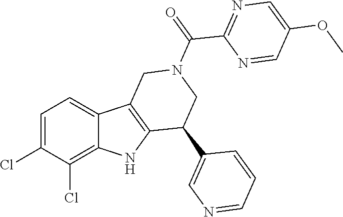 [SMILES: COc1cnc(C(=O)N2Cc3c([nH]c4c(Cl)c(Cl)ccc34)[C@H](c3cccnc3)C2)nc1] |
| 138B* | TA | (S)-(6,7-dichloro-4-(pyridin-3-yl)-1,3,4,5-tetrahydro-2H-pyrido[4,3-b]indol-2- yl)(5-methoxypyrimidin-2-yl)methanone 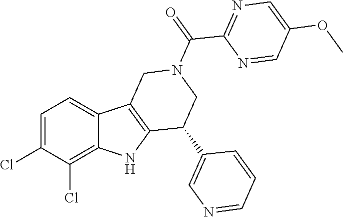 [SMILES: COc1cnc(C(=O)N2Cc3c([nH]c4c(Cl)c(Cl)ccc34)[C@@H](c3cccnc3)C2)nc1] |
| Rac-139 | TA | 6,7-dichloro-2-(5-methoxypyrimidine-2-carbonyl)-2,3,4,5-tetrahydro-1H- pyrido[4,3-b]indole-4-carbonitrile 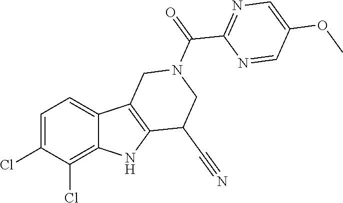 [SMILES: COc1cnc(C(=O)N2Cc3c([nH]c4c(Cl)c(Cl)ccc34)C(C#N)C2)nc1] |
| 139A* | TA | (R)-6,7-dichloro-2-(5-methoxypyrimidine-2-carbonyl)-2,3,4,5-tetrahydro-1H- pyrido[4,3-b]indole-4-carbonitrile 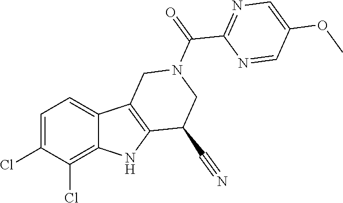 [SMILES: COc1cnc(C(=O)N2Cc3c([nH]c4c(Cl)c(Cl)ccc34)[C@H](C#N)C2)nc1] |
| 139B* | TA | (S)-6,7-dichloro-2-(5-methoxypyrimidine-2-carbonyl)-2,3,4,5-tetrahydro-1H- pyrido[4,3-b]indole-4-carbonitrile 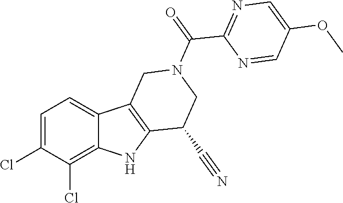 [SMILES: COc1cnc(C(=O)N2Cc3c([nH]c4c(Cl)c(Cl)ccc34)[C@@H](C#N)C2)nc1] |
| Rac-140 | TA | (7,8-dichloro-1-methyl-10-(1-methyl-1H-pyrazol-3-yl)-3,4- dihydropyrazino[1,2-b]indazol-2(1H)-yl)(5-methoxypyrimidin-2-yl)methanone 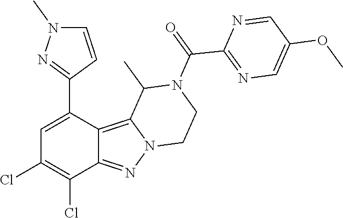 [SMILES: COc1cnc(C(=O)N2CCn3nc4c(Cl)c(Cl)cc(-c5ccn(C)n5)c4c3C2C)nc1] |
| 140A* | TA | (R)-(7,8-dichloro-1-methyl-10-(1-methyl-1H-pyrazol-3-yl)-3,4- dihydropyrazino[1,2-b]indazol-2(1H)-yl)(5-methoxypyrimidin-2-yl)methanone 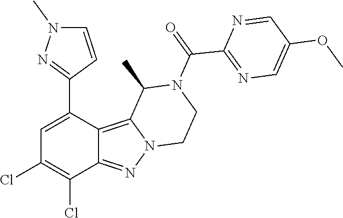 [SMILES: COc1cnc(C(=O)N2CCn3nc4c(Cl)c(Cl)cc(-c5ccn(C)n5)c4c3[C@H]2C)nc1] |
| 140B* | TA | (S)-(7,8-dichloro-1-methyl-10-(1-methyl-1H-pyrazol-3-yl)-3,4- dihydropyrazino[1,2-b]indazol-2(1H)-yl)(5-methoxypyrimidin-2-yl)methanone 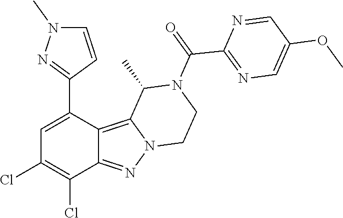 [SMILES: COc1cnc(C(=O)N2CCn3nc4c(Cl)c(Cl)cc(-c5ccn(C)n5)c4c3[C@@H]2C)nc1] |
| 141 | TA | (6,7-dichloro-5-methyl-1,3,4,5-tetrahydro-2H-pyrido[4,3-b]indol-2-yl)(5- methoxypyrimidin-2-yl)methanone 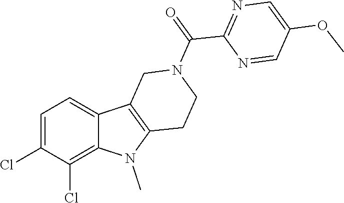 [SMILES: COc1cnc(C(=O)N2CCc3c(c4ccc(Cl)c(Cl)c4n3C)C2)nc1] |
| Rac-142 | TA | (7-bromo-6-fluoro-1-methyl-1,3,4,5-tetrahydro-2H-pyrido[4,3-b]indol-2-yl)(5- methoxypyrimidin-2-yl)methanone 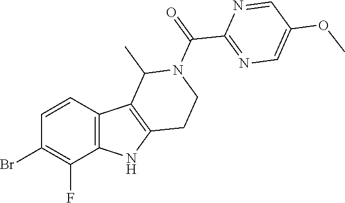 [SMILES: COc1cnc(C(=O)N2CCc3[nH]c4c(F)c(Br)ccc4c3C2C)nc1] |
| 142A* | TA | (R)-(7-bromo-6-fluoro-1-methyl-1,3,4,5-tetrahydro-2H-pyrido[4,3-b]indol-2- yl)(5-methoxypyrimidin-2-yl)methanone 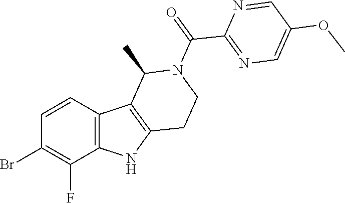 [SMILES: COc1cnc(C(=O)N2CCc3[nH]c4c(F)c(Br)ccc4c3[C@H]2C)nc1] |
| 142B* | TA | (S)-(7-bromo-6-fluoro-1-methyl-1,3,4,5-tetrahydro-2H-pyrido[4,3-b]indol-2- yl)(5-methoxypyrimidin-2-yl)methanone 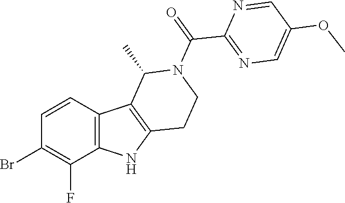 [SMILES: COc1cnc(C(=O)N2CCc3[nH]c4c(F)c(Br)ccc4c3[C@@H]2C)nc1] |
| Rac-144 | TA | (6-chloro-7-ethynyl-1-methyl-1,3,4,5-tetrahydro-2H-pyrido[4,3-b]indol-2-yl)(5- methoxypyrimidin-2-yl)methanone 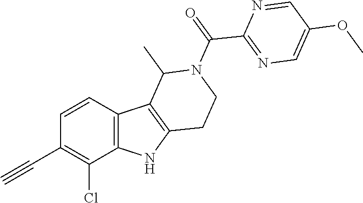 [SMILES: C#Cc1ccc2c3c([nH]c2c1Cl)CCN(C(=O)c1ncc(OC)cn1)C3C] |
| 144A* | TA | (R)-(6-chloro-7-ethynyl-1-methyl-1,3,4,5-tetrahydro-2H-pyrido[4,3-b]indol-2- yl)(5-methoxypyrimidin-2-yl)methanone 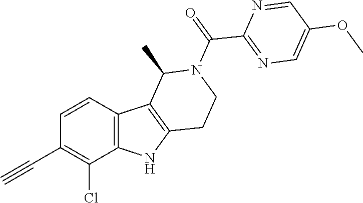 [SMILES: C#Cc1ccc2c3c([nH]c2c1Cl)CCN(C(=O)c1ncc(OC)cn1)[C@@H]3C] |
| 144B* | TA | (S)-(6-chloro-7-ethynyl-1-methyl-1,3,4,5-tetrahydro-2H-pyrido[4,3-b]indol-2- yl)(5-methoxypyrimidin-2-yl)methanone 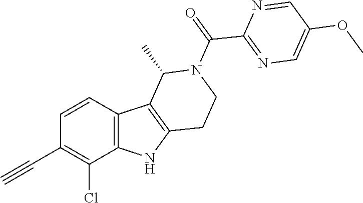 [SMILES: C#Cc1ccc2c3c([nH]c2c1Cl)CCN(C(=O)c1ncc(OC)cn1)[C@H]3C] |
| Rac-147 | TA | (6,7-dichloro-1-(fluoromethyl)-1,3,4,5-tetrahydro-2H-pyrido[4,3-b]indol-2- yl)(5-methoxypyrimidin-2-yl)methanone 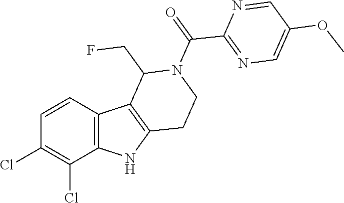 [SMILES: COc1cnc(C(=O)N2CCc3[nH]c4c(Cl)c(Cl)ccc4c3C2CF)nc1] |
| 147A* | TA | (S)-(6,7-dichloro-1-(fluoromethyl)-1,3,4,5-tetrahydro-2H-pyrido[4,3-b]indol-2- yl)(5-methoxypyrimidin-2-yl)methanone 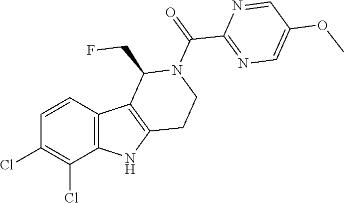 [SMILES: COc1cnc(C(=O)N2CCc3[nH]c4c(Cl)c(Cl)ccc4c3[C@H]2CF)nc1] |
| 147B* | TA | (R)-(6,7-dichloro-1-(fluoromethyl)-1,3,4,5-tetrahydro-2H-pyrido[4,3-b]indol-2- yl)(5-methoxypyrimidin-2-yl)methanone 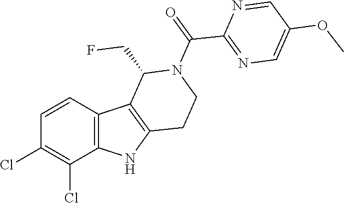 [SMILES: COc1cnc(C(=O)N2CCc3[nH]c4c(Cl)c(Cl)ccc4c3[C@@H]2CF)nc1] |
| Rac-148 | TA | (6,7-dichloro-1-((methylthio)methyl)-1,3,4,5-tetrahydro-2H-pyrido[4,3-b]indol- 2-yl)(5-methoxypyrimidin-2-yl)methanone 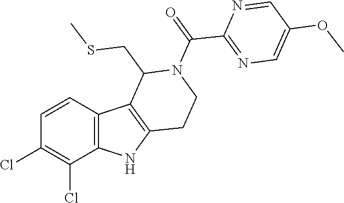 [SMILES: COc1cnc(C(=O)N2CCc3[nH]c4c(Cl)c(Cl)ccc4c3C2CSC)nc1] |
| 148A* | TA | (S)-(6,7-dichloro-1-((methylthio)methyl)-1,3,4,5-tetrahydro-2H-pyrido[4,3- b]indol-2-yl)(5-methoxypyrimidin-2-yl)methanone 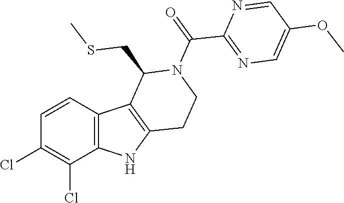 [SMILES: COc1cnc(C(=O)N2CCc3[nH]c4c(Cl)c(Cl)ccc4c3[C@H]2CSC)nc1] |
| 148B* | TA | (R)-(6,7-dichloro-1-((methylthio)methyl)-1,3,4,5-tetrahydro-2H-pyrido[4,3- b]indol-2-yl)(5-methoxypyrimidin-2-yl)methanone 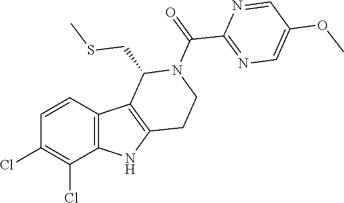 [SMILES: COc1cnc(C(=O)N2CCc3[nH]c4c(Cl)c(Cl)ccc4c3[C@@H]2CSC)nc1] |
| Rac-149 | TA | (6,7-dichloro-1-(difluoromethyl)-1,3,4,5-tetrahydro-2H-pyrido[4,3-b]indol-2- yl)(5-methoxypyrimidin-2-yl)methanone 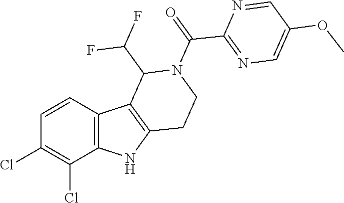 [SMILES: COc1cnc(C(=O)N2CCc3[nH]c4c(Cl)c(Cl)ccc4c3C2C(F)F)nc1] |
| 149A* | TA | (S)-(6,7-dichloro-1-(difluoromethyl)-1,3,4,5-tetrahydro-2H-pyrido[4,3-b]indol- 2-yl)(5-methoxypyrimidin-2-yl)methanone 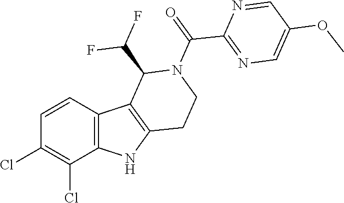 [SMILES: COc1cnc(C(=O)N2CCc3[nH]c4c(Cl)c(Cl)ccc4c3[C@H]2C(F)F)nc1] |
| 149B* | TA | (R)-(6,7-dichloro-1-(difluoromethyl)-1,3,4,5-tetrahydro-2H-pyrido[4,3-b]indol- 2-yl)(5-methoxypyrimidin-2-yl)methanone 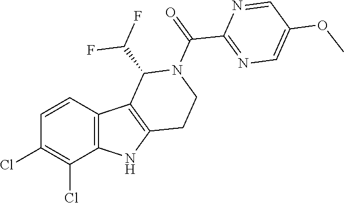 [SMILES: COc1cnc(C(=O)N2CCc3[nH]c4c(Cl)c(Cl)ccc4c3[C@@H]2C(F)F)nc1] |
| Rac-150 | TA | (6,7-dichloro-1-(methoxymethyl)-1,3,4,5-tetrahydro-2H-pyrido[4,3-b]indol-2- yl)(5-methoxypyrimidin-2-yl)methanone 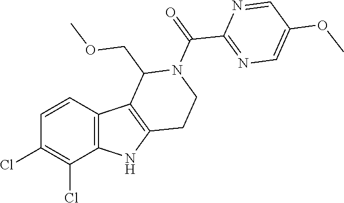 [SMILES: COCC1c2c([nH]c3c(Cl)c(Cl)ccc23)CCN1C(=O)c1ncc(OC)cn1] |
| 150A* | TA | (S)-(6,7-dichloro-1-(methoxymethyl)-1,3,4,5-tetrahydro-2H-pyrido[4,3-b]indol- 2-yl)(5-methoxypyrimidin-2-yl)methanone 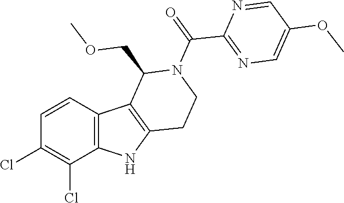 [SMILES: COC[C@@H]1c2c([nH]c3c(Cl)c(Cl)ccc23)CCN1C(=O)c1ncc(OC)cn1] |
| 150B* | TA | (R)-(6,7-dichloro-1-(methoxymethyl)-1,3,4,5-tetrahydro-2H-pyrido[4,3-b]indol- 2-yl)(5-methoxypyrimidin-2-yl)methanone 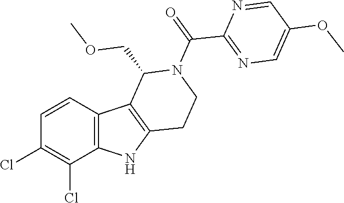 [SMILES: COC[C@H]1c2c([nH]c3c(Cl)c(Cl)ccc23)CCN1C(=O)c1ncc(OC)cn1] |
| Rac-155 | 6 | (6,7-dichloro-1-methyl-1,3,4,5-tetrahydro-2H-pyrido[4,3-b]indol-2-yl)(5- fluoropyrimidin-2-yl)methanone 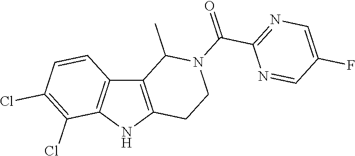 [SMILES: CC1c2c([nH]c3c(Cl)c(Cl)ccc23)CCN1C(=O)c1ncc(F)cn1] |
| 155A* | 6 | (R)-(6,7-dichloro-1-methyl-1,3,4,5-tetrahydro-2H-pyrido[4,3-b]indol-2-yl)(5- fluoropyrimidin-2-yl)methanone 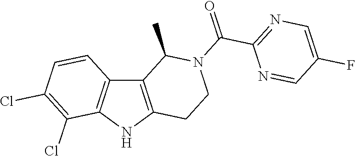 [SMILES: C[C@@H]1c2c([nH]c3c(Cl)c(Cl)ccc23)CCN1C(=O)c1ncc(F)cn1] |
| 155B* | 6 | (S)-(6,7-dichloro-1-methyl-1,3,4,5-tetrahydro-2H-pyrido[4,3-b]indol-2-yl)(5- fluoropyrimidin-2-yl)methanone 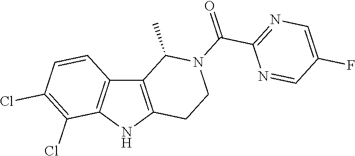 [SMILES: C[C@H]1c2c([nH]c3c(Cl)c(Cl)ccc23)CCN1C(=O)c1ncc(F)cn1] |
| Rac-156 | 19 | (9-bromo-6,7-dichloro-1-methyl-1,3,4,5-tetrahydro-2H-pyrido[4,3-b]indol-2- yl)(5-methoxypyrimidin-2-yl)methanone 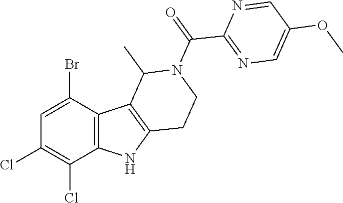 [SMILES: COc1cnc(C(=O)N2CCc3[nH]c4c(Cl)c(Cl)cc(Br)c4c3C2C)nc1] |
| 156A* | 19 | (R)-(9-bromo-6,7-dichloro-1-methyl-1,3,4,5-tetrahydro-2H-pyrido[4,3-b]indol- 2-yl)(5-methoxypyrimidin-2-yl)methanone 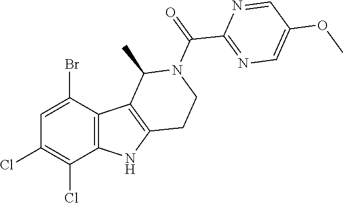 [SMILES: COc1cnc(C(=O)N2CCc3[nH]c4c(Cl)c(Cl)cc(Br)c4c3[C@H]2C)nc1] |
| 156B* | 19 | (S)-(9-bromo-6,7-dichloro-1-methyl-1,3,4,5-tetrahydro-2H-pyrido[4,3-b]indol- 2-yl)(5-methoxypyrimidin-2-yl)methanone 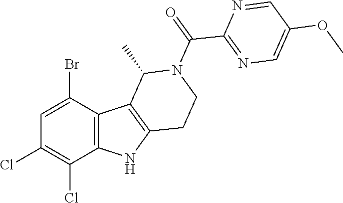 [SMILES: COc1cnc(C(=O)N2CCc3[nH]c4c(Cl)c(Cl)cc(Br)c4c3[C@@H]2C)nc1] |
| Rac-100 | TA | (6,7-dichloro-1-methyl-3,4-dihydroimidazo[1,2-a:5,4-c′]dipyridin-2(1H)-yl)(5- methoxypyrimidin-2-yl)methanone 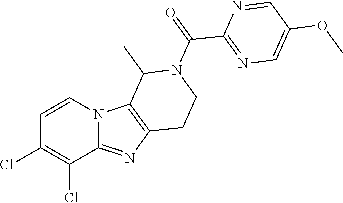 [SMILES: COc1cnc(C(=O)N2CCc3nc4c(Cl)c(Cl)ccn4c3C2C)nc1] |
| 100A* | TA | (R)-(6,7-dichloro-1-methyl-3,4-dihydroimidazo[1,2-a:5,4-c′]dipyridin-2(1H)- yl)(5-methoxypyrimidin-2-yl)methanone 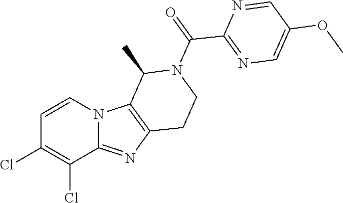 [SMILES: COc1cnc(C(=O)N2CCc3nc4c(Cl)c(Cl)ccn4c3[C@H]2C)nc1] |
| 100B* | TA | (S)-(6,7-dichloro-1-methyl-3,4-dihydroimidazo[1,2-a:5,4-c′]dipyridin-2(1H)- yl)(5-methoxypyrimidin-2-yl)methanone 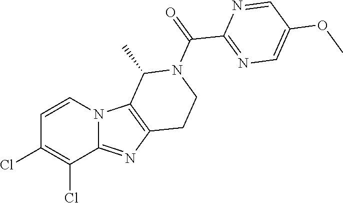 [SMILES: COc1cnc(C(=O)N2CCc3nc4c(Cl)c(Cl)ccn4c3[C@@H]2C)nc1] |
| Rac-111 | TA | (3,4-dichloro-9-methyl-6,9-dihydropyrido[4′,3′:4,5]imidazo[1,2-c]pyrimidin- 8(7H)-yl)(5-methoxypyrimidin-2-yl)methanone 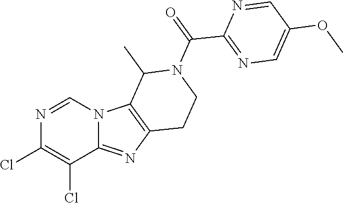 [SMILES: COc1cnc(C(=O)N2CCc3nc4c(Cl)c(Cl)ncn4c3C2C)nc1] |
| 111A* | TA | (R)-(3,4-dichloro-9-methyl-6,9-dihydropyrido[4′,3′:4,5]imidazo[1,2- c]pyrimidin-8(7H)-yl)(5-methoxypyrimidin-2-yl)methanone 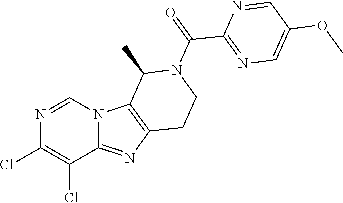 [SMILES: COc1cnc(C(=O)N2CCc3nc4c(Cl)c(Cl)ncn4c3[C@H]2C)nc1] |
| 111B* | TA | (S)-(3,4-dichloro-9-methyl-6,9-dihydropyrido[4′,3′:4,5]imidazo[1,2- c]pyrimidin-8(7H)-yl)(5-methoxypyrimidin-2-yl)methanone 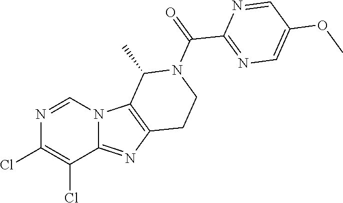 [SMILES: COc1cnc(C(=O)N2CCc3nc4c(Cl)c(Cl)ncn4c3[C@@H]2C)nc1] |
| Rac-114 | TA | (6,7-dichloro-1-methyl-1,3,4,5-tetrahydro-2H-pyrrolo[3,2-c:4,5-c′]dipyridin-2- yl)(5-methoxypyrimidin-2-yl)methanone 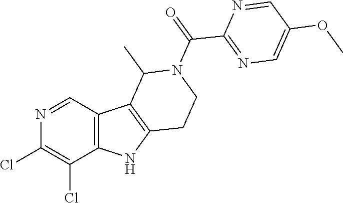 [SMILES: COc1cnc(C(=O)N2CCc3[nH]c4c(Cl)c(Cl)ncc4c3C2C)nc1] |
| 114A* | TA | (R)-(6,7-dichloro-1-methyl-1,3,4,5-tetrahydro-2H-pyrrolo[3,2-c:4,5- c′]dipyridin-2-yl)(5-methoxypyrimidin-2-yl)methanone 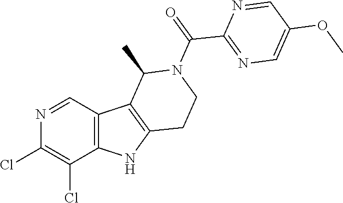 [SMILES: COc1cnc(C(=O)N2CCc3[nH]c4c(Cl)c(Cl)ncc4c3[C@H]2C)nc1] |
| 114B* | TA | (S)-(6,7-dichloro-1-methyl-1,3,4,5-tetrahydro-2H-pyrrolo[3,2-c:4,5- c′]dipyridin-2-yl)(5-methoxypyrimidin-2-yl)methanone 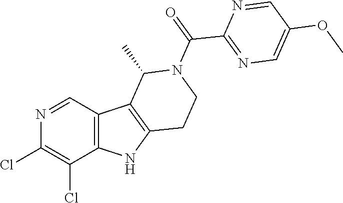 [SMILES: COc1cnc(C(=O)N2CCc3[nH]c4c(Cl)c(Cl)ncc4c3[C@@H]2C)nc1] |
| Rac-134 | TA | (3,4-dichloro-10-methyl-7,8-dihydropyrido[4′,3′:3,4]pyrazolo[1,5-a]pyrazin- 9(10H)-yl)(5-methoxypyrimidin-2-yl)methanone 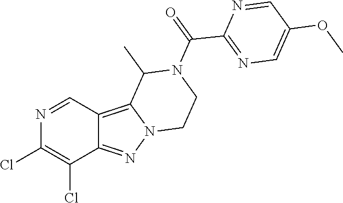 [SMILES: COc1cnc(C(=O)N2CCn3nc4c(Cl)c(Cl)ncc4c3C2C)nc1] |
| 134A* | TA | (R)-(3,4-dichloro-10-methyl-7,8-dihydropyrido[4′,3′:3,4]pyrazolo[1,5- a]pyrazin-9(10H)-yl)(5-methoxypyrimidin-2-yl)methanone 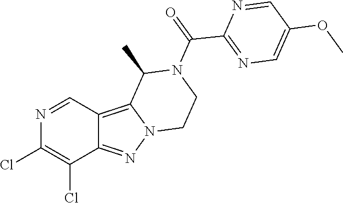 [SMILES: COc1cnc(C(=O)N2CCn3nc4c(Cl)c(Cl)ncc4c3[C@H]2C)nc1] |
| 134B* | TA | (S)-(3,4-dichloro-10-methyl-7,8-dihydropyrido[4′,3′:3,4]pyrazolo[1,5-a]pyrazin- 9(10H)-yl)(5-methoxypyrimidin-2-yl)methanone 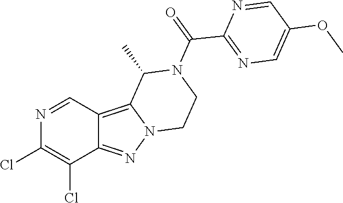 [SMILES: COc1cnc(C(=O)N2CCn3nc4c(Cl)c(Cl)ncc4c3[C@@H]2C)nc1] |
| 135 | TA | (5,6-dichloro-1-methyl-3,4-dihydropyrazolo[4,3-b]indol-2(1H)-yl)(5- methoxypyrimidin-2-yl)methanone 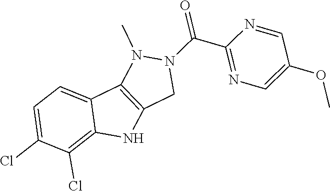 [SMILES: COc1cnc(C(=O)N2Cc3[nH]c4c(Cl)c(Cl)ccc4c3N2C)nc1] |
| 136 | TA | (6,7-dichloro-1-methyl-1,3,4,5-tetrahydro-2H-pyridazino[4,3-b]indol-2-yl)(5- methoxypyrimidin-2-yl)methanone 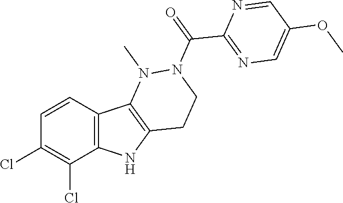 [SMILES: COc1cnc(C(=O)N2CCc3[nH]c4c(Cl)c(Cl)ccc4c3N2C)nc1] |
| Rac-143 | TA | (3,4-dichloro-2-methoxy-9-methyl-5,6,7,9-tetrahydro-8H-pyrrolo[3,2-b:4,5- c′]dipyridin-8-yl)(5-methoxypyrimidin-2-yl)methanone 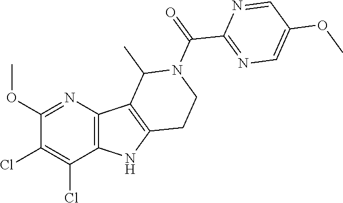 [SMILES: COc1cnc(C(=O)N2CCc3[nH]c4c(Cl)c(Cl)c(OC)nc4c3C2C)nc1] |
| 143A* | TA | (R)-(3,4-dichloro-2-methoxy-9-methyl-5,6,7,9-tetrahydro-8H-pyrrolo[3,2-b:4,5- c′]dipyridin-8-yl)(5-methoxypyrimidin-2-yl)methanone 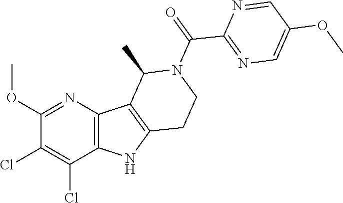 [SMILES: COc1cnc(C(=O)N2CCc3[nH]c4c(Cl)c(Cl)c(OC)nc4c3[C@H]2C)nc1] |
| 143B* | TA | (S)-(3,4-dichloro-2-methoxy-9-methyl-5,6,7,9-tetrahydro-8H-pyrrolo[3,2-b:4,5- c′]dipyridin-8-yl)(5-methoxypyrimidin-2-yl)methanone 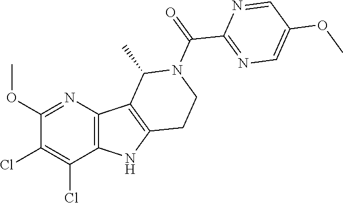 [SMILES: COc1cnc(C(=O)N2CCc3[nH]c4c(Cl)c(Cl)c(OC)nc4c3[C@@H]2C)nc1] |
| Rac-145 | TA | (3-bromo-4-chloro-2-methoxy-9-methyl-5,6,7,9-tetrahydro-8H-pyrrolo[3,2- b:4,5-c′]dipyridin-8-yl)(5-methoxypyrimidin-2-yl)methanone 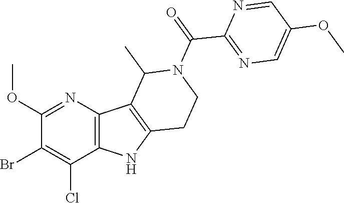 [SMILES: COc1cnc(C(=O)N2CCc3[nH]c4c(Cl)c(Br)c(OC)nc4c3C2C)nc1] |
| 145A* | TA | (R)-(3-bromo-4-chloro-2-methoxy-9-methyl-5,6,7,9-tetrahydro-8H-pyrrolo[3,2- b:4,5-c′]dipyridin-8-yl)(5-methoxypyrimidin-2-yl)methanone 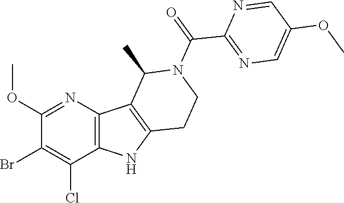 [SMILES: COc1cnc(C(=O)N2CCc3[nH]c4c(Cl)c(Br)c(OC)nc4c3[C@H]2C)nc1] |
| 145B* | TA | (S)-(3-bromo-4-chloro-2-methoxy-9-methyl-5,6,7,9-tetrahydro-8H-pyrrolo[3,2- b:4,5-c′]dipyridin-8-yl)(5-methoxypyrimidin-2-yl)methanone 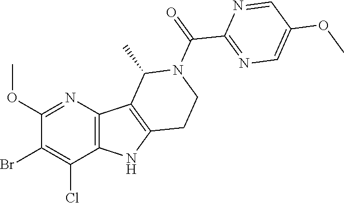 [SMILES: COc1cnc(C(=O)N2CCc3[nH]c4c(Cl)c(Br)c(OC)nc4c3[C@@H]2C)nc1] |
| Rac-146 | TA | (6,7-dichloro-9-methoxy-1-methyl-1,3,4,5-tetrahydro-2H-pyrrolo[3,2-c:4,5- c′]dipyridin-2-yl)(5-methoxypyrimidin-2-yl)methanone 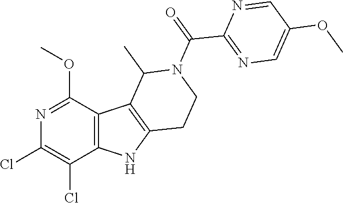 [SMILES: COc1cnc(C(=O)N2CCc3[nH]c4c(Cl)c(Cl)nc(OC)c4c3C2C)nc1] |
| 146A* | TA | (R)-(6,7-dichloro-9-methoxy-1-methyl-1,3,4,5-tetrahydro-2H-pyrrolo[3,2-c:4,5- c′]dipyridin-2-yl)(5-methoxypyrimidin-2-yl)methanone 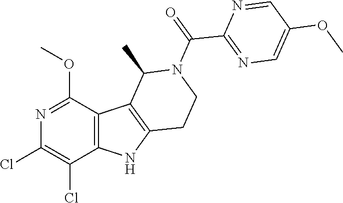 [SMILES: COc1cnc(C(=O)N2CCc3[nH]c4c(Cl)c(Cl)nc(OC)c4c3[C@H]2C)nc1] |
| 146B* | TA | (S)-(6,7-dichloro-9-methoxy-1-methyl-1,3,4,5-tetrahydro-2H-pyrrolo[3,2-c:4,5- c′]dipyridin-2-yl)(5-methoxypyrimidin-2-yl)methanone 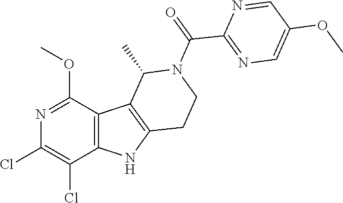 [SMILES: COc1cnc(C(=O)N2CCc3[nH]c4c(Cl)c(Cl)nc(OC)c4c3[C@@H]2C)nc1] |
| Rac-153 | TA | (7,8-dichloro-1-methyl-3,4-dihydropyrazino[2,1-a]isoindol-2(1H)-yl)(5- methoxypyrimidin-2-yl)methanone 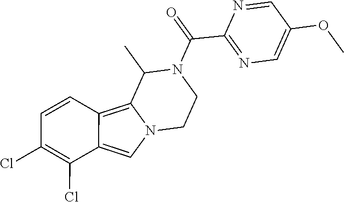 [SMILES: COc1cnc(C(=O)N2CCn3cc4c(Cl)c(Cl)ccc4c3C2C)nc1] |
| 153A* | TA | (R)-(7,8-dichloro-1-methyl-3,4-dihydropyrazino[2,1-a]isoindol-2(1H)-yl)(5- methoxypyrimidin-2-yl)methanone 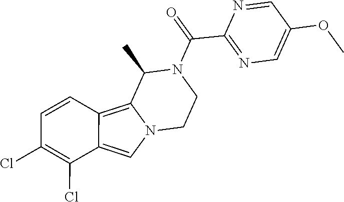 [SMILES: COc1cnc(C(=O)N2CCn3cc4c(Cl)c(Cl)ccc4c3[C@H]2C)nc1] |
| 153B* | TA | (S)-(7,8-dichloro-1-methyl-3,4-dihydropyrazino[2,1-a]isoindol-2(1H)-yl)(5- methoxypyrimidin-2-yl)methanone 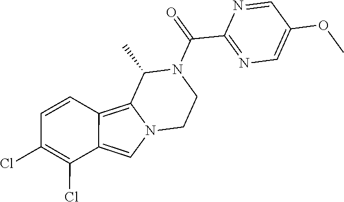 [SMILES: COc1cnc(C(=O)N2CCn3cc4c(Cl)c(Cl)ccc4c3[C@@H]2C)nc1] |
| Rac-110 | TA | 3,4-dichloro-8-(5-methoxypyrimidine-2-carbonyl)-2,9-dimethyl-2,5,6,7,8,9- hexahydro-1H-pyrrolo[3,2-c:4,5-c′]dipyridin-1-one 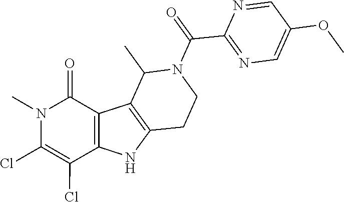 [SMILES: COc1cnc(C(=O)N2CCc3[nH]c4c(Cl)c(Cl)n(C)c(=O)c4c3C2C)nc1] |
| 110A* | TA | (R)-3,4-dichloro-8-(5-methoxypyrimidine-2-carbonyl)-2,9-dimethyl-2,5,6,7,8,9- hexahydro-1H-pyrrolo[3,2-c:4,5-c′]dipyridin-1-one 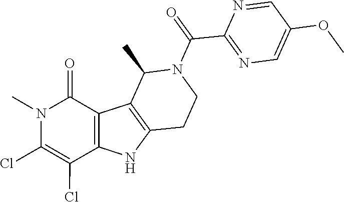 [SMILES: COc1cnc(C(=O)N2CCc3[nH]c4c(Cl)c(Cl)n(C)c(=O)c4c3[C@H]2C)nc1] |
| 110B* | TA | (S)-3,4-dichloro-8-(5-methoxypyrimidine-2-carbonyl)-2,9-dimethyl-2,5,6,7,8,9- hexahydro-1H-pyrrolo[3,2-c:4,5-c′]dipyridin-1-one 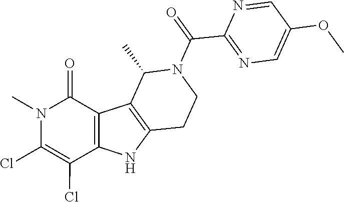 [SMILES: COc1cnc(C(=O)N2CCc3[nH]c4c(Cl)c(Cl)n(C)c(=O)c4c3[C@@H]2C)nc1] |
| 119 | TA | 6-chloro-2-(5-methoxypyrimidine-2-carbonyl)-7-methyl-1,2,3,4,5,7-hexahydro- 8H-pyrrolo[2,3-c:4,5-c′]dipyridin-8-one 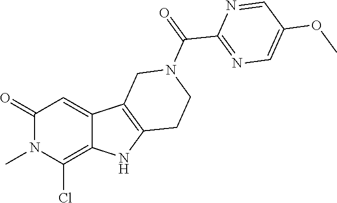 [SMILES: COc1cnc(C(=O)N2CCc3[nH]c4c(Cl)n(C)c(=O)cc4c3C2)nc1] |
| 122 | TA | 3-chloro-8-(5-methoxypyrimidine-2-carbonyl)-1-methyl-2-oxo-2,5,6,7,8,9- hexahydro-1H-pyrrolo[3,2-b:4,5-c′]dipyridine-4-carbonitrile 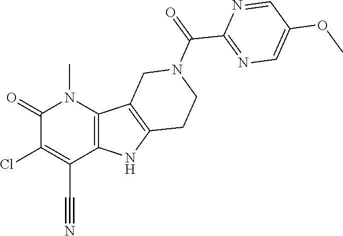 [SMILES: COc1cnc(C(=O)N2CCc3[nH]c4c(C#N)c(Cl)c(=O)n(C)c4c3C2)nc1] |

TABLE 4
Compounds of Formula (I)

| # | Ex | Name/Structure |
|---|---|---|
| 3 | TA | (6,7-dichloro-1,3,4,5-tetrahydro-2H-pyrido[4,3-b]indol-2-yl)(1-methyl-1H- imidazol-2-yl)methanone 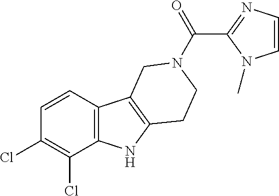 [SMILES: Cn1ccnc1C(=O)N1CCc2[nH]c3c(Cl)c(Cl)ccc3c2C1] |
| 5 | TA | (6,7-dichloro-1,3,4,5-tetrahydro-2H-pyrido[4,3-b]indol-2-yl)(1H-imidazol-2- yl)methanone 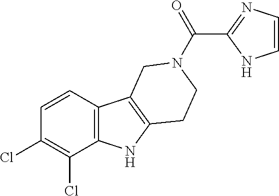 [SMILES: O=C(c1ncc[nH]1)N1CCc2[nH]c3c(Cl)c(Cl)ccc3c2C1] |
| 6 | TA | (6,7-dichloro-1,3,4,5-tetrahydro-2H-pyrido[4,3-b]indol-2-yl)(1H-1,2,4-triazol- 3-yl)methanone 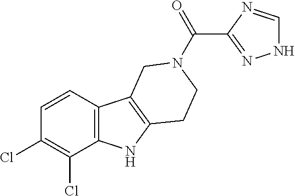 [SMILES: O=C(c1nc[nH]n1)N1CCc2[nH]c3c(Cl)c(Cl)ccc3c2C1] |
| 7 | TA | (6,7-dichloro-1,3,4,5-tetrahydro-2H-pyrido[4,3-b]indol-2-yl)(1H-tetrazol-5- yl)methanone (tautomer 1); Also referred to herein as (6,7-dichloro-1,3,4,5- tetrahydro-2H-pyrido[4,3-b]indol-2-yl)(2H-tetrazol-5-yl)methanone (tautomer 2) 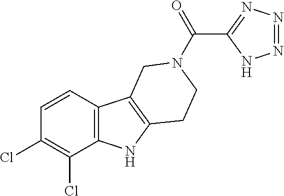 [SMILES: O=C(c1nnn[nH]1)N1CCc2[nH]c3c(Cl)c(Cl)ccc3c2C1] tautomer 1 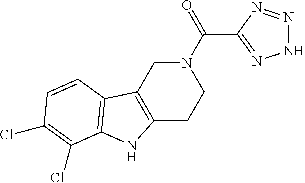 [SMILES: O=C(c1nn[nH]n1)N1CCc2[nH]c3c(Cl)c(Cl)ccc3c2C1] tautomer 2 |
| 9 | TA | (6,7-dichloro-1,3,4,5-tetrahydro-2H-pyrido[4,3-b]indol-2-yl)(1-methyl-1H- 1,2,4-triazol-3-yl)methanone 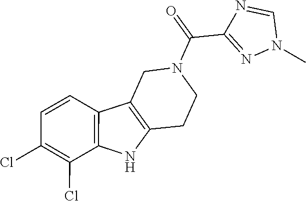 [SMILES: Cn1cnc(C(=O)N2CCc3[nH]c4c(Cl)c(Cl)ccc4c3C2)n1] |
| 10 | TA | (6,7-dichloro-1,3,4,5-tetrahydro-2H-pyrido[4,3-b]indol-2-yl)(5-methyl-1H- imidazol-2-yl)methanone 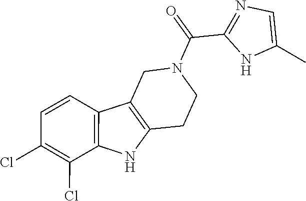 [SMILES: Cc1cnc(C(=O)N2CCc3[nH]c4c(Cl)c(Cl)ccc4c3C2)[nH]1] |
| 11 | TA | (6,7-dichloro-1,3,4,5-tetrahydro-2H-pyrido[4,3-b]indol-2-yl)(oxazol-2- yl)methanone 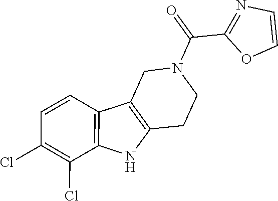 [SMILES: O=C(c1ncco1)N1CCc2[nH]c3c(Cl)c(Cl)ccc3c2C1] |
| 13 | TA | 5-(6,7-dichloro-2,3,4,5-tetrahydro-1H-pyrido[4,3-b]indole-2-carbonyl)-2,4- dihydro-3H-1,2,4-triazol-3-one 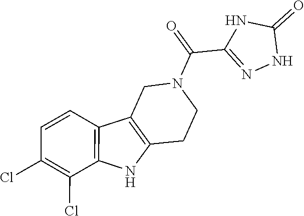 [SMILES: O=C(c1n[nH]c(=O)[nH]1)N1CCc2[nH]c3c(Cl)c(Cl)ccc3c2C1] |
| Rac-90 | TA | (5-amino-1H-imidazol-2-yl)(6,7-dichloro-1-methyl-1,3,4,5-tetrahydro-2H- pyrido[4,3-b]indol-2-yl)methanone 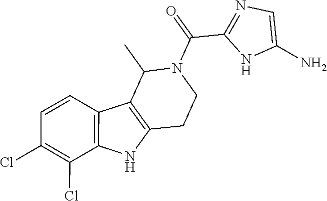 [SMILES: CC1c2c([nH]c3c(Cl)c(Cl)ccc23)CCN1C(=O)c1ncc(N)[nH]1] |
| 90A* | TA | (R)-(5-amino-1H-imidazol-2-yl)(6,7-dichloro-1-methyl-1,3,4,5-tetrahydro-2H- pyrido[4,3-b]indol-2-yl)methanone 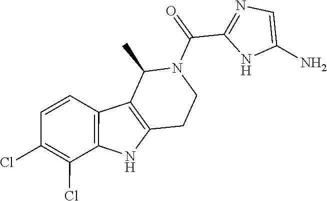 [SMILES: C[C@@H]1c2c([nH]c3c(Cl)c(Cl)ccc23)CCN1C(=O)c1ncc(N)[nH]1] |
| 90B* | TA | (S)-(5-amino-1H-imidazol-2-yl)(6,7-dichloro-1-methyl-1,3,4,5-tetrahydro-2H- pyrido[4,3-b]indol-2-yl)methanone 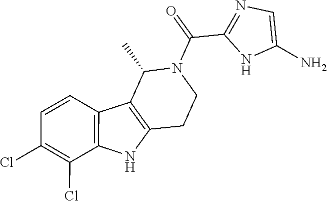 [SMILES: C[C@H]1c2c([nH]c3c(Cl)c(Cl)ccc23)CCN1C(=O)c1ncc(N)[nH]1] |
| Rac-91 | TA | (6,7-dichloro-1-methyl-1,3,4,5-tetrahydro-2H-pyrido[4,3-b]indol-2-yl)(5- (methylamino)-1H-imidazol-2-yl)methanone 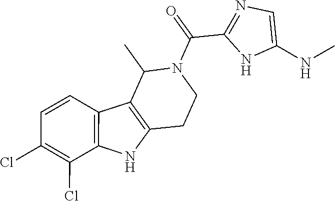 [SMILES: CNc1cnc(C(=O)N2CCc3[nH]c4c(Cl)c(Cl)ccc4c3C2C)[nH]1] |
| 91A* | TA | (R)-(6,7-dichloro-1-methyl-1,3,4,5-tetrahydro-2H-pyrido[4,3-b]indol-2-yl)(5- (methylamino)-1H-imidazol-2-yl)methanone 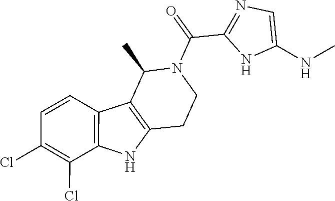 [SMILES: CNc1cnc(C(=O)N2CCc3[nH]c4c(Cl)c(Cl)ccc4c3[C@H]2C)[nH]1] |
| 91B* | TA | (S)-(6,7-dichloro-1-methyl-1,3,4,5-tetrahydro-2H-pyrido[4,3-b]indol-2-yl)(5- (methylamino)-1H-imidazol-2-yl)methanone 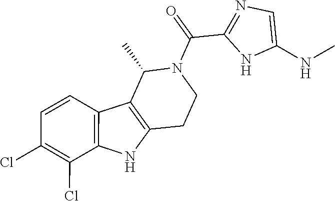 [SMILES: CNc1cnc(C(=O)N2CCc3[nH]c4c(Cl)c(Cl)ccc4c3[C@@H]2C)[nH]1] |
| Rac-92 | TA | (6,7-dichloro-1-methyl-1,3,4,5-tetrahydro-2H-pyrido[4,3-b]indol-2-yl)(5- (dimethylamino)-1H-imidazol-2-yl)methanone 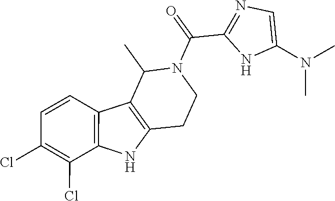 [SMILES: CC1c2c([nH]c3c(Cl)c(Cl)ccc23)CCN1C(=O)c1ncc(N(C)C)[nH]1] |
| 92A* | TA | (R)-(6,7-dichloro-1-methyl-1,3,4,5-tetrahydro-2H-pyrido[4,3-b]indol-2-yl)(5- (dimethylamino)-1H-imidazol-2-yl)methanone 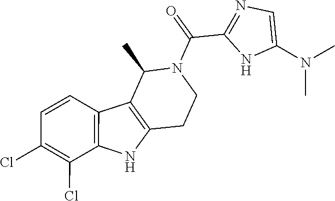 [SMILES: C[C@@H]1c2c([nH]c3c(Cl)c(Cl)ccc23)CCN1C(=O)c1ncc(N(C)C)[nH]1] |
| 92B* | TA | (S)-(6,7-dichloro-1-methyl-1,3,4,5-tetrahydro-2H-pyrido[4,3-b]indol-2-yl)(5- (dimethylamino)-1H-imidazol-2-yl)methanone 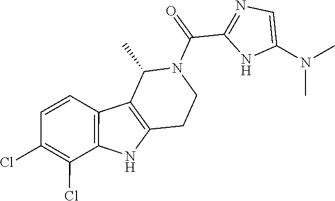 [SMILES: C[C@H]1c2c([nH]c3c(Cl)c(Cl)ccc23)CCN1C(=O)c1ncc(N(C)C)[nH]1] |
| Rac-93 | TA | (6,7-dichloro-1-methyl-1,3,4,5-tetrahydro-2H-pyrido[4,3-b]indol-2-yl)(5- hydroxy-1H-imidazol-2-yl)methanone 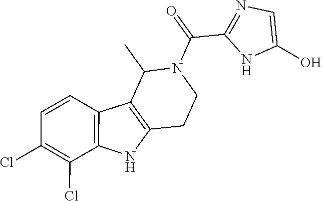 [SMILES: CC1c2c([nH]c3c(Cl)c(Cl)ccc23)CCN1C(=O)c1ncc(O)[nH]1] |
| 93A* | TA | (R)-(6,7-dichloro-1-methyl-1,3,4,5-tetrahydro-2H-pyrido[4,3-b]indol-2-yl)(5- hydroxy-1H-imidazol-2-yl)methanone 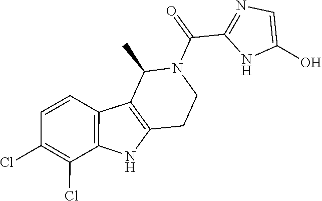 [SMILES: C[C@@H]1c2c([nH]c3c(Cl)c(Cl)ccc23)CCN1C(=O)c1ncc(O)[nH]1] |
| 93B* | TA | (S)-(6,7-dichloro-1-methyl-1,3,4,5-tetrahydro-2H-pyrido[4,3-b]indol-2-yl)(5- hydroxy-1H-imidazol-2-yl)methanone 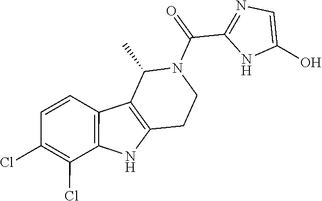 [SMILES: C[C@H]1c2c([nH]c3c(Cl)c(Cl)ccc23)CCN1C(=O)c1ncc(O)[nH]1] |
| Rac-94 | TA | (6,7-dichloro-1-methyl-1,3,4,5-tetrahydro-2H-pyrido[4,3-b]indol-2-yl)(5- methoxy-1H-imidazol-2-yl)methanone 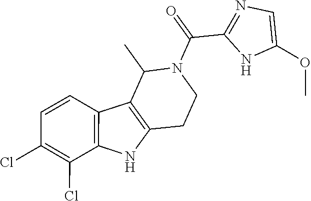 [SMILES: COc1cnc(C(=O)N2CCc3[nH]c4c(Cl)c(Cl)ccc4c3C2C)[nH]1] |
| 94A* | TA | (R)-(6,7-dichloro-1-methyl-1,3,4,5-tetrahydro-2H-pyrido[4,3-b]indol-2-yl)(5- methoxy-1H-imidazol-2-yl)methanone 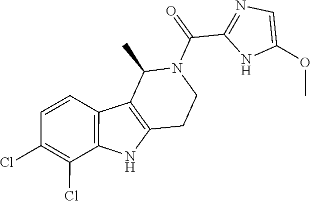 [SMILES: COc1cnc(C(=O)N2CCc3[nH]c4c(Cl)c(Cl)ccc4c3[C@H]2C)[nH]1] |
| 94B* | TA | (S)-(6,7-dichloro-1-methyl-1,3,4,5-tetrahydro-2H-pyrido[4,3-b]indol-2-yl)(5- methoxy-1H-imidazol-2-yl)methanone 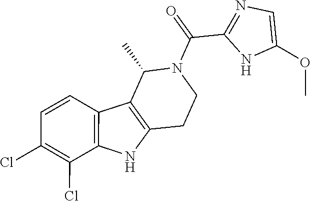 [SMILES: COc1cnc(C(=O)N2CCc3[nH]c4c(Cl)c(Cl)ccc4c3[C@@H]2C)[nH]1] |
| Rac-95 | 17 | 1-[6,7-dichloro-1-methyl-1H,3H,4H,5H-pyrido[4,3-b]indole-2-carbonyl]-3H- imidazole-4-carbonitrile; Also referred to as: 2-(6,7-dichloro-1-methyl-2,3,4,5- tetrahydro-1H-pyrido[4,3-b]indole-2-carbonyl)-1H-imidazole-5-carbonitrile 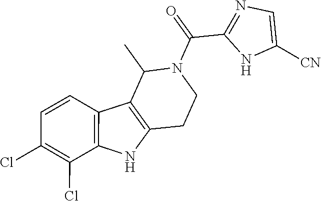 [SMILES: CC1c2c([nH]c3c(Cl)c(Cl)ccc23)CCN1C(=O)c1ncc(C#N)[nH]1] |
| 95A* | 17 | 1-[(1R)-6,7-dichloro-1-methyl-1H,3H,4H,5H-pyrido[4,3-b]indole-2-carbonyl]- 3H-imidazole-4-carbonitrile 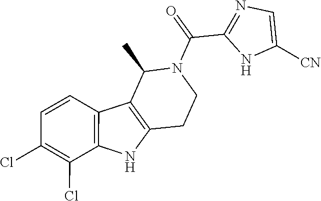 [SMILES: C[C@@H]1c2c([nH]c3c(Cl)c(Cl)ccc23)CCN1C(=O)c1ncc(C#N)[nH]1] |
| 95B* | 17 | 1-[(1S)-6,7-dichloro-1-methyl-1H,3H,4H,5H-pyrido[4,3-b]indole-2-carbonyl]- 3H-imidazole-4-carbonitrile; Also referred to as: (S)-2-(6,7-dichloro-1-methyl- 2,3,4,5-tetrahydro-1H-pyrido[4,3-b]indole-2-carbonyl)-1H-imidazole-5- carbonitrile 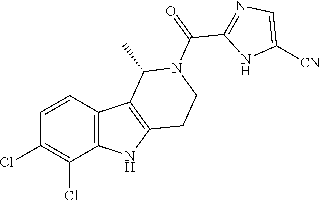 [SMILES: C[C@H]1c2c([nH]c3c(Cl)c(Cl)ccc23)CCN1C(=O)c1ncc(C#N)[nH]1] |
| 154 | TA | (6,7-dichloro-1,3,4,5-tetrahydro-2H-pyrido[4,3-b]indol-2-yl)(1-methyl-4,5- dihydro-1H-1,2,4-triazol-3-yl)methanone 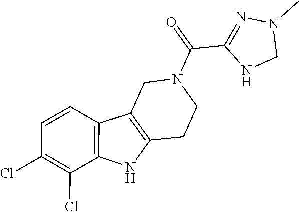 [SMILES: CN1CNC(C(=O)N2CCc3[nH]c4c(Cl)c(Cl)ccc4c3C2)=N1] |

TABLE 5
Additional Compounds

| # | Ex | Name/Structure |
|---|---|---|
| 59 | 12 | 6,7-dichloro-1,3,4,5-tetrahydro-2H-pyrido[4,3-b]indole-2-carbohydrazide 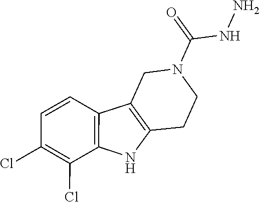 [SMILES: NNC(=O)N1CCc2[nH]c3c(Cl)c(Cl)ccc3c2C1] |
| Rac-97 | 18 | 6,7-dichloro-N-hydroxy-1-methyl-1,3,4,5-tetrahydro-2H-pyrido[4,3- b]indole-2-carboxamide 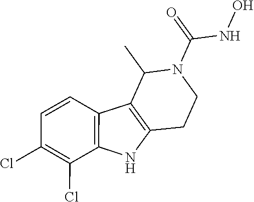 [SMILES: CC1c2c([nH]c3c(Cl)c(Cl)ccc23)CCN1C(=O)NO] |
| 97A* | 18 | (R)-6,7-dichloro-N-hydroxy-1-methyl-1,3,4,5-tetrahydro-2H-pyrido[4,3- b]indole-2-carboxamide 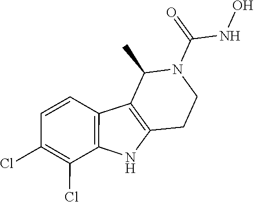 [SMILES: C[C@@H]1c2c([nH]c3c(Cl)c(Cl)ccc23)CCN1C(=O)NO] |
| 97B* | 18 | (S)-6,7-dichloro-N-hydroxy-1-methyl-1,3,4,5-tetrahydro-2H-pyrido[4,3- b]indole-2-carboxamide 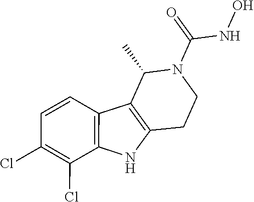 [SMILES: C[C@H]1c2c([nH]c3c(Cl)c(Cl)ccc23)CCN1C(=O)NO] |
| Rac-98 | TA | 6,7-dichloro-N-methoxy-1-methyl-1,3,4,5-tetrahydro-2H-pyrido[4,3- b]indole-2-carboxamide 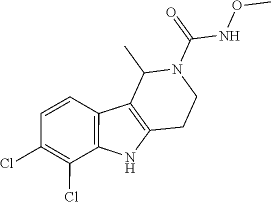 [SMILES: CONC(=O)N1CCc2[nH]c3c(Cl)c(Cl)ccc3c2C1C] |
| 98A* | TA | (R)-6,7-dichloro-N-methoxy-1-methyl-1,3,4,5-tetrahydro-2H-pyrido[4,3- b]indole-2-carboxamide 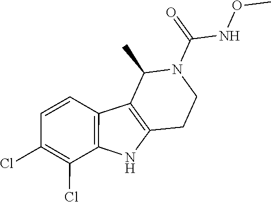 [SMILES: CONC(=O)N1CCc2[nH]c3c(Cl)c(Cl)ccc3c2[C@H]1C] |
| 98B* | TA | (S)-6,7-dichloro-N-methoxy-1-methyl-1,3,4,5-tetrahydro-2H-pyrido[4,3- b]indole-2-carboxamide 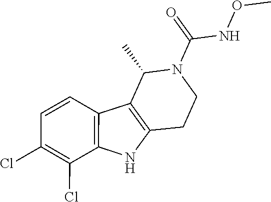 [SMILES: CONC(=O)N1CCc2[nH]c3c(Cl)c(Cl)ccc3c2[C@@H]1C] |
| 27 | TA | 1-(6,7-dichloro-2-(5-methoxypyrimidine-2-carbonyl)-1,2,3,4-tetrahydro-5H- pyrido[4,3-b]indol-5-yl)ethan-1-one 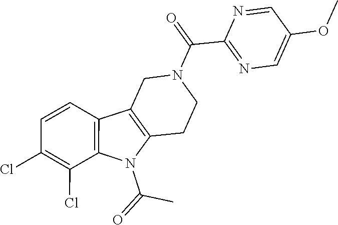 [SMILES: COc1cnc(C(=O)N2CCc3c(c4ccc(Cl)c(Cl)c4n3C(C)=O)C2)nc1] |
| Rac-82 | TA | (6,7-dichloro-1-methyl-1,3,4,5-tetrahydro-2H-pyrido[4,3-b]indol-2-yl)(4- methoxy-5-methylpyrimidin-2-yl)methanone 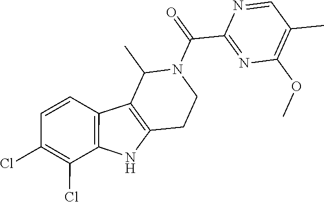 [SMILES: COc1nc(C(=O)N2CCc3[nH]c4c(Cl)c(Cl)ccc4c3C2C)ncc1C] |
| 82A* | TA | (R)-(6,7-dichloro-1-methyl-1,3,4,5-tetrahydro-2H-pyrido[4,3-b]indol-2- yl)(4-methoxy-5-methylpyrimidin-2-yl)methanone 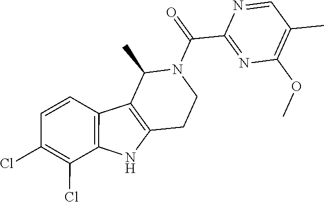 [SMILES: COc1nc(C(=O)N2CCc3[nH]c4c(Cl)c(Cl)ccc4c3[C@H]2C)ncc1C] |
| 82B* | TA | (S)-(6,7-dichloro-1-methyl-1,3,4,5-tetrahydro-2H-pyrido[4,3-b]indol-2-yl)(4- methoxy-5-methylpyrimidin-2-yl)methanone 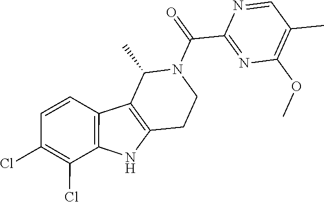 [SMILES: COc1nc(C(=O)N2CCc3[nH]c4c(Cl)c(Cl)ccc4c3[C@@H]2C)ncc1C] |
| Rac-83 | 15 | (6,7-dichloro-1-methyl-1,3,4,5-tetrahydro-2H-pyrido[4,3-b]indol-2-yl)(4,5- dimethoxypyrimidin-2-yl)methanone 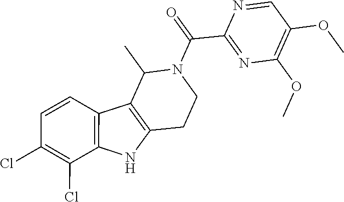 [SMILES: COc1cnc(C(=O)N2CCc3[nH]c4c(Cl)c(Cl)ccc4c3C2C)nc1OC] |
| 83A* | 15 | (R)-(6,7-dichloro-1-methyl-1,3,4,5-tetrahydro-2H-pyrido[4,3-b]indol-2- yl)(4,5-dimethoxypyrimidin-2-yl)methanone 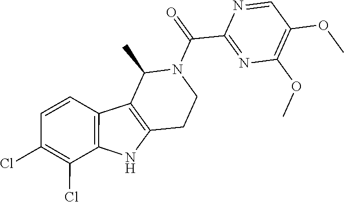 [SMILES: COc1cnc(C(=O)N2CCc3[nH]c4c(Cl)c(Cl)ccc4c3[C@H]2C)nc1OC] |
| 83B* | 15 | (S)-(6,7-dichloro-1-methyl-1,3,4,5-tetrahydro-2H-pyrido[4,3-b]indol-2- yl)(4,5-dimethoxypyrimidin-2-yl)methanone 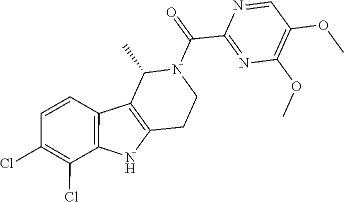 [SMILES: COc1cnc(C(=O)N2CCc3[nH]c4c(Cl)c(Cl)ccc4c3[C@@H]2C)nc1OC] |
| Rac-84 | TA | (6,7-dichloro-1-methyl-1,3,4,5-tetrahydro-2H-pyrido[4,3-b]indol-2-yl)(4- methoxy-5-(methylamino)pyrimidin-2-yl)methanone 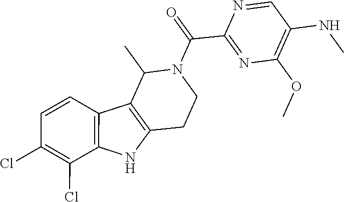 [SMILES: CNc1cnc(C(=O)N2CCc3[nH]c4c(Cl)c(Cl)ccc4c3C2C)nc1OC] |
| 84A* | TA | (R)-(6,7-dichloro-1-methyl-1,3,4,5-tetrahydro-2H-pyrido[4,3-b]indol-2- yl)(4-methoxy-5-(methylamino)pyrimidin-2-yl)methanone 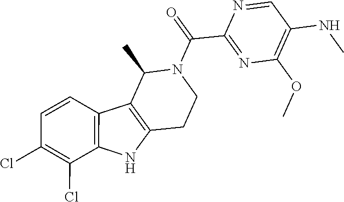 [SMILES: CNc1cnc(C(=O)N2CCc3[nH]c4c(Cl)c(Cl)ccc4c3[C@H]2C)nc1OC] |
| 84B* | TA | (S)-(6,7-dichloro-1-methyl-1,3,4,5-tetrahydro-2H-pyrido[4,3-b]indol-2-yl)(4- methoxy-5-(methylamino)pyrimidin-2-yl)methanone 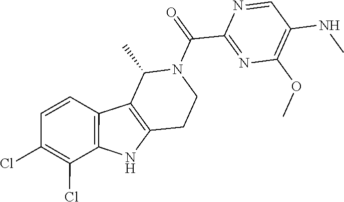 [SMILES: CNc1cnc(C(=O)N2CCc3[nH]c4c(Cl)c(Cl)ccc4c3[C@@H]2C)nc1OC] |
| Rac-85 | TA | (5-amino-4-methoxypyrimidin-2-yl)(6,7-dichloro-1-methyl-1,3,4,5- tetrahydro-2H-pyrido[4,3-b]indol-2-yl)methanone 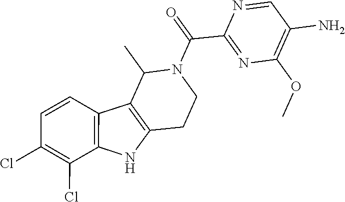 [SMILES: COc1nc(C(=O)N2CCc3[nH]c4c(Cl)c(Cl)ccc4c3C2C)ncc1N] |
| 85A* | TA | (R)-(5-amino-4-methoxypyrimidin-2-yl)(6,7-dichloro-1-methyl-1,3,4,5- tetrahydro-2H-pyrido[4,3-b]indol-2-yl)methanone 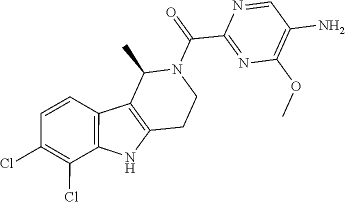 [SMILES: COc1nc(C(=O)N2CCc3[nH]c4c(Cl)c(Cl)ccc4c3[C@H]2C)ncc1N] |
| 85B* | TA | (S)-(5-amino-4-methoxypyrimidin-2-yl)(6,7-dichloro-1-methyl-1,3,4,5- tetrahydro-2H-pyrido[4,3-b]indol-2-yl)methanone 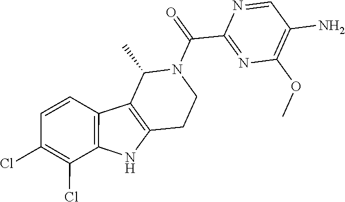 [SMILES: COc1nc(C(=O)N2CCc3[nH]c4c(Cl)c(Cl)ccc4c3[C@@H]2C)ncc1N] |
| Rac-106 | TA | (6,7-dichloro-1-methyl-1,3,4,5-tetrahydro-2H-pyrido[4,3-b]indol-2-yl)(6,7- dihydro-5H-pyrimido[4,5-b][1,4]oxazin-2-yl)methanone 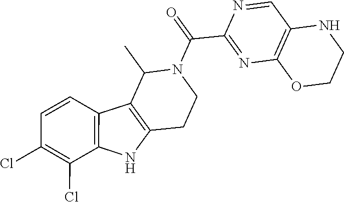 [SMILES: CC1c2c([nH]c3c(Cl)c(Cl)ccc23)CCN1C(=O)c1ncc2c(n1)OCCN2] |
| 106A* | TA | (R)-(6,7-dichloro-1-methyl-1,3,4,5-tetrahydro-2H-pyrido[4,3-b]indol-2- yl)(6,7-dihydro-5H-pyrimido[4,5-b][1,4]oxazin-2-yl)methanone 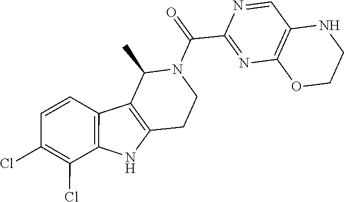 [SMILES: C[C@@H]1c2c([nH]c3c(Cl)c(Cl)ccc23)CCN1C(=O)c1ncc2c(n1)OCCN2] |
| 106B* | TA | (S)-(6,7-dichloro-1-methyl-1,3,4,5-tetrahydro-2H-pyrido[4,3-b]indol-2- yl)(6,7-dihydro-5H-pyrimido[4,5-b][1,4]oxazin-2-yl)methanone 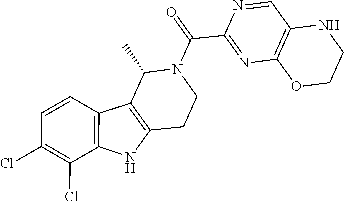 [SMILES: C[C@H]1c2c([nH]c3c(Cl)c(Cl)ccc23)CCN1C(=O)c1ncc2c(n1)OCCN2] |
| 124 | TA | (7,8-dichloro-1,4,5,6-tetrahydroazepino[4,5-b]indol-3(2H)-yl)(5- methoxypyrimidin-2-yl)methanone 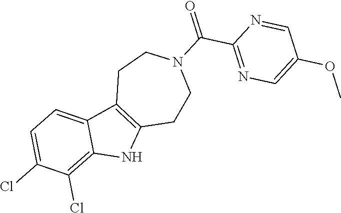 [SMILES: COc1cnc(C(=O)N2CCc3[nH]c4c(Cl)c(Cl)ccc4c3CC2)nc1] |
| 53 | TA | (R)-2-amino-1-(6,7-dichloro-1,3,4,5-tetrahydro-2H-pyrido[4,3-b]indol-2-yl)- 3-methoxypropan-1-one 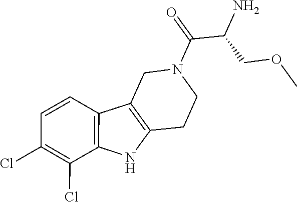 [SMILES: COC[C@@H](N)C(=O)N1CCc2[nH]c3c(Cl)c(Cl)ccc3c2C1] |
| 54 | TA | (R)-2-amino-1-(6,7-dichloro-1,3,4,5-tetrahydro-2H-pyrido[4,3-b]indol-2-yl)- 3-hydroxypropan-1-one 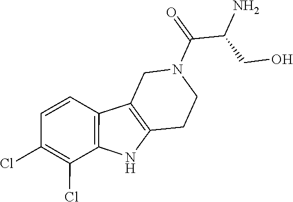 [SMILES: N[C@H](CO)C(=O)N1CCc2[nH]c3c(Cl)c(Cl)ccc3c2C1] |
| 57 | TA | 2-(6,7-dichloro-1,3,4,5-tetrahydro-2H-pyrido[4,3-b]indol-2-yl)-2- oxoacetamide 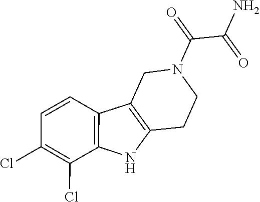 [SMILES: NC(=O)C(=O)N1CCc2[nH]c3c(Cl)c(Cl)ccc3c2C1] |
| 58 | TA | (R)-2-amino-1-(6,7-dichloro-1,3,4,5-tetrahydro-2H-pyrido[4,3-b]indol-2- yl)propan-1-one 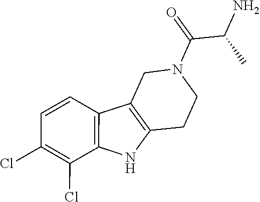 [SMILES: C[C@@H](N)C(=O)N1CCc2[nH]c3c(Cl)c(Cl)ccc3c2C1] |
| 65 | TA | 2-amino-1-(5,6-dichloro-3,4-dihydropyrrolo[3,4-b]indol-2(1H)-yl)ethan-1- one 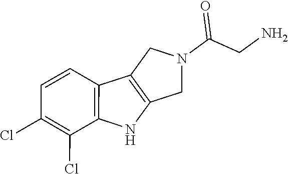 [SMILES: NCC(=O)N1Cc2[nH]c3c(Cl)c(Cl)ccc3c2C1] |
| Rac-99 | TA | 6,7-dichloro-2-(5-methoxypyrimidine-2-sulfonimidoyl)-1-methyl-2,3,4,5- tetrahydro-1H-pyrido[4,3-b]indole 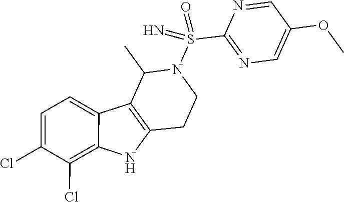 [SMILES: COc1cnc(S(=N)(=O)N2CCc3[nH]c4c(Cl)c(Cl)ccc4c3C2C)nc1] |
| 99A* | TA | (1R)-6,7-dichloro-2-(5-methoxypyrimidine-2-sulfonimidoyl)-1-methyl- 2,3,4,5-tetrahydro-1H-pyrido[4,3-b]indole 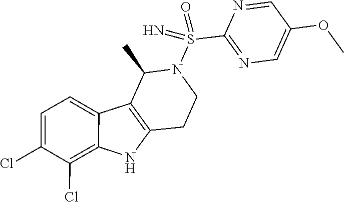 [SMILES: COc1cnc(S(=N)(=O)N2CCc3[nH]c4c(Cl)c(Cl)ccc4c3[C@H]2C)nc1] |
| 99B* | TA | (1S)-6,7-dichloro-2-(5-methoxypyrimidine-2-sulfonimidoyl)-1-methyl- 2,3,4,5-tetrahydro-1H-pyrido[4,3-b]indole 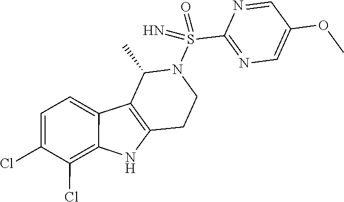 [SMILES: COc1cnc(S(=N)(=O)N2CCc3[nH]c4c(Cl)c(Cl)ccc4c3[C@@H]2C)nc1] |
| 55 | TA | (6,7-dichloro-1,3,4,5-tetrahydro-2H-pyrido[4,3-b]indol-2-yl)(5- methoxypyrazin-2-yl)methanone 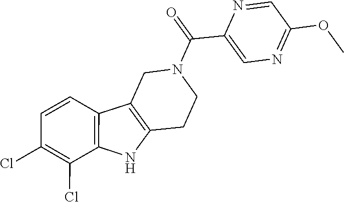 [SMILES: COc1cnc(C(=O)N2CCc3[nH]c4c(Cl)c(Cl)ccc4c3C2)cn1] |
| 56 | TA | (6,7-dichloro-1,3,4,5-tetrahydro-2H-pyrido[4,3-b]indol-2-yl)(6- methoxypyridazin-3-yl)methanone 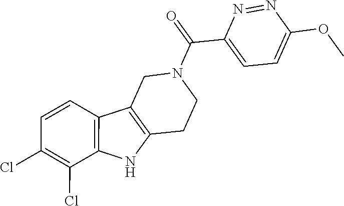 [SMILES: COc1ccc(C(=O)N2CCc3[nH]c4c(Cl)c(Cl)ccc4c3C2)nn1] |
| 157 | TA | (S)-1-(6,7-dichloro-1-methyl-1,3,4,5-tetrahydro-2H-pyrido[4,3-b]indol-2-yl)- 2-hydroxyethan-1-one - 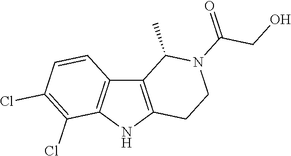 [SMILES: C[C@H]1c2c([nH]c3c(Cl)c(Cl)ccc23)CCN1C(=O)CO] |
| 158 | TA | 2-amino-1-(6,7-dichloro-1,3,4,5-tetrahydro-2H-pyrido[4,3-b]indol-2- yl)ethan-1-one 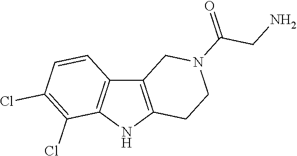 [SMILES: NCC(=O)N1CCc2[nH]c3c(Cl)c(Cl)ccc3c2C1] |
| Rac-130 | TA | (6,7-dichloro-9-((difluoromethyl)thio)-1-methyl-1,3,4,5-tetrahydro-2H- pyrido[4,3-b]indol-2-yl)(5-methoxypyrimidin-2-yl)methanone 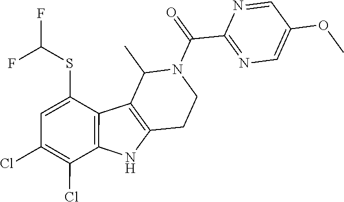 [SMILES: COc1cnc(C(=O)N2CCc3[nH]c4c(Cl)c(Cl)cc(SC(F)F)c4c3C2C)nc1] |
| 130A* | TA | (R)-(6,7-dichloro-9-((difluoromethyl)thio)-1-methyl-1,3,4,5-tetrahydro-2H- pyrido[4,3-b]indol-2-yl)(5-methoxypyrimidin-2-yl)methanone 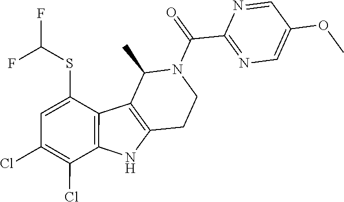 [SMILES: COc1cnc(C(=O)N2CCc3[nH]c4c(Cl)c(Cl)cc(SC(F)F)c4c3[C@H]2C)nc1] |
| 130B* | TA | (S)-(6,7-dichloro-9-((difluoromethyl)thio)-1-methyl-1,3,4,5-tetrahydro-2H- pyrido[4,3-b]indol-2-yl)(5-methoxypyrimidin-2-yl)methanone 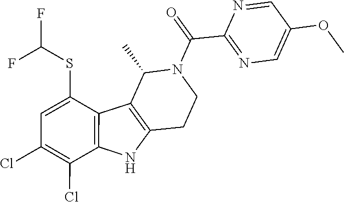 [SMILES: COc1cnc(C(=O)N2CCc3[nH]c4c(Cl)c(Cl)cc(SC(F)F)c4c3[C@@H]2C)nc1] |
| Rac-151 | TA | (6,7-dichloro-1-methyl-3,4-dihydropyrimido[1,6-a]indol-2(1H)-yl)(5- methoxypyrimidin-2-yl)methanone 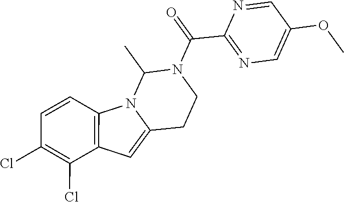 [SMILES: COc1cnc(C(=O)N2CCc3cc4c(Cl)c(Cl)ccc4n3C2C)nc1] |
| 151A* | TA | (R)-(6,7-dichloro-1-methyl-3,4-dihydropyrimido[1,6-a]indol-2(1H)-yl)(5- methoxypyrimidin-2-yl)methanone 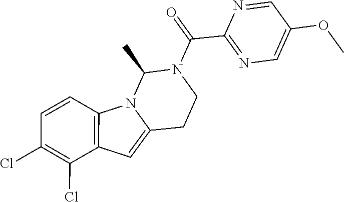 [SMILES: COc1cnc(C(=O)N2CCc3cc4c(Cl)c(Cl)ccc4n3[C@H]2C)nc1] |
| 151B* | TA | (S)-(6,7-dichloro-1-methyl-3,4-dihydropyrimido[1,6-a]indol-2(1H)-yl)(5- methoxypyrimidin-2-yl)methanone 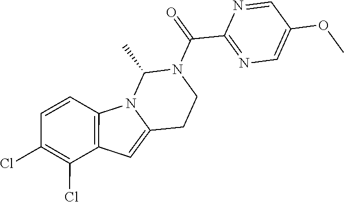 [SMILES: COc1cnc(C(=O)N2CCc3cc4c(Cl)c(Cl)ccc4n3[C@@H]2C)nc1] |
| Rac-152 | TA | (6,7-dichloro-1-methyl-3,4-dihydropyrido[4,3-b]indolizin-2(1H)-yl)(5- methoxypyrimidin-2-yl)methanone 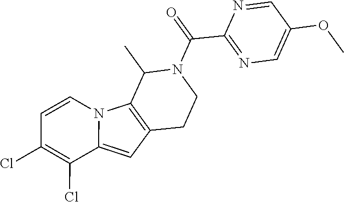 [SMILES: COc1cnc(C(=O)N2CCc3cc4c(Cl)c(Cl)ccn4c3C2C)nc1] |
| 152A* | TA | (R)-(6,7-dichloro-1-methyl-3,4-dihydropyrido[4,3-b]indolizin-2(1H)-yl)(5- methoxypyrimidin-2-yl)methanone 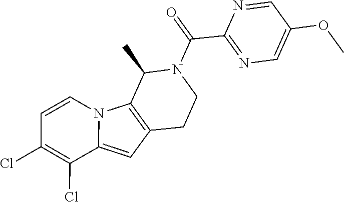 [SMILES: COc1cnc(C(=O)N2CCc3cc4c(Cl)c(Cl)ccn4c3[C@H]2C)nc1] |
| 152B* | TA | (S)-(6,7-dichloro-1-methyl-3,4-dihydropyrido[4,3-b]indolizin-2(1H)-yl)(5- methoxypyrimidin-2-yl)methanone 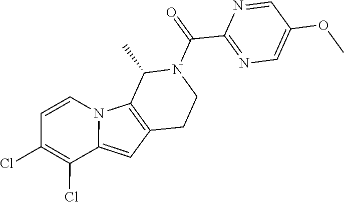 [SMILES: COc1cnc(C(=O)N2CCc3cc4c(Cl)c(Cl)ccn4c3[C@@H]2C)nc1] |

In some embodiments, the compound is an isotopic derivative of said compound.

In some embodiments, the compound is a pharmaceutically acceptable salt. In some embodiments, the compound is a hydrochloride salt.

It should be understood that all isomeric forms are included within the present invention, including mixtures thereof. If the compound contains a double bond, the substituent may be in the E or Z configuration. If the compound contains a disubstituted cycloalkyl, the cycloalkyl substituent may have a cis- or trans configuration. All tautomeric forms are also intended to be included.

Compounds of the invention, and pharmaceutically acceptable salts, hydrates, solvates, stereoisomers and prodrugs thereof may exist in their tautomeric form (for example, as an amide or imino ether). All such tautomeric forms are contemplated herein as part of the present invention.

The compounds of the invention may contain asymmetric or chiral centers, and, therefore, exist in different stereoisomeric forms. It is intended that all stereoisomeric forms of the compounds of the invention as well as mixtures thereof, including racemic mixtures, form part of the present invention. In addition, the present invention embraces all geometric and positional isomers. For example, if a compound of the invention incorporates a double bond or a fused ring, both the cis- and trans-forms, as well as mixtures, are embraced within the scope of the invention. each compound herein disclosed includes all the enantiomers that conform to the general structure of the compound. The compounds may be in a racemic or enantiomerically pure form, or any other form in terms of stereochemistry. The assay results may reflect the data collected for the racemic form, the enantiomerically pure form, or any other form in terms of stereochemistry.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of the invention may be atropisomers (e.g., substituted biaryls) and are considered as part of this invention. Enantiomers can also be separated by use of a chiral HPLC or SFC column.

It is also possible that the compounds of the invention may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound of Formula (I) incorporates a double bond or a fused ring, both the cis- and trans-forms, as well as mixtures, are embraced within the scope of the invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester,” “prodrug” and the like, is intended to equally apply to the salt, solvate, ester and prodrug of enantiomers, stereoisomers, rotamers, tautomers, positional isomers, racemates or prodrugs of the inventive compounds.

The compounds as described herein may form salts which are also within the scope of this invention. Reference to a compound of the Formula herein is understood to include reference to salts thereof, unless otherwise indicated.

The present invention relates to compounds which are modulators of cGAS. In one embodiment, the compounds of the present invention are inhibitors of cGAS. In another embodiment, the cGAS is Isoform 1. In another embodiment, the cGAS is Isoform 2.

In some embodiments, the compounds as described herein are selective inhibitors of cGAS.

The invention is directed to compounds as described herein and pharmaceutically acceptable salts, hydrates, solvates, prodrugs, stereoisomers, or tautomers thereof, and pharmaceutical compositions comprising one or more compounds as described herein, or pharmaceutically acceptable salts, hydrates, solvates, prodrugs, stereoisomers, or tautomers thereof.

### Methods of Synthesizing the Compounds

The compounds of the present invention may be made by a variety of methods, including standard chemistry. Suitable synthetic routes are depicted in the Examples.

The compounds as described herein may be prepared by methods known in the art of organic synthesis as set forth in part by the following synthetic schemes. In the Examples, it is well understood that protecting groups for sensitive or reactive groups are employed where necessary in accordance with general principles or chemistry. Protecting groups are manipulated according to standard methods of organic synthesis. See, e.g., T. W. Greene and P. G. M. Wuts, “Protective Groups in Organic Synthesis”, Third edition, Wiley, New York 1999. These groups are removed at a convenient stage of the compound synthesis using methods that are readily apparent to those skilled in the art. The selection processes, as well as the reaction conditions and order of those steps, as well as if a stereocenter exists, will be recognized by one skilled in the art. The present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Methods of Using the Disclosed Compounds

Another aspect of the invention relates to a method of treating a disease or disorder associated with modulation of cGAS. The method comprises administering to a patient in need of a treatment for diseases or disorders associated with modulation of cGAS an effective amount of the composition and/or compound, as described herein, or a pharmaceutically acceptable salt, hydrate, solvate, prodrug, stereoisomer, or tautomer thereof.

In another aspect, the present invention is directed to a method of inhibiting cGAS. The method involves administering to a patient in need thereof an effective amount of a compound, as described herein, or a pharmaceutically acceptable salt, hydrate, solvate, prodrug, stereoisomer, or tautomer thereof.

Another aspect of the present invention relates to a method of treating, preventing, inhibiting or eliminating a disease or disorder in a patient associated with the inhibition of cGAS, the method comprising administering to a patient in need thereof an effective amount of a compound, as described herein, or a pharmaceutically acceptable salt, hydrate, solvate, prodrug, stereoisomer, or tautomer thereof. In one embodiment, the disease may be, but not limited to, cancer and metastasis.

The present invention also relates to the use of an inhibitor of cGAS for the preparation of a medicament used in the treatment, prevention, inhibition or elimination of a disease or condition mediated by cGAS, wherein the medicament comprises a compound, as described herein, or a pharmaceutically acceptable salt, hydrate, solvate, prodrug, stereoisomer, or tautomer thereof.

In another aspect, the present invention relates to a method for the manufacture of a medicament for treating, preventing, inhibiting, or eliminating a disease or condition mediated by cGAS, wherein the medicament comprises a compound, as described herein, or a pharmaceutically acceptable salt, hydrate, solvate, prodrug, stereoisomer, or tautomer thereof.

Another aspect of the present invention relates to a compound, as described herein, or a pharmaceutically acceptable salt, hydrate, solvate, prodrug, stereoisomer, or tautomer thereof, for use in the manufacture of a medicament for treating a disease associated with inhibiting cGAS.

In another aspect, the present invention relates to the use of a compound, as described herein, or a pharmaceutically acceptable salt, hydrate, solvate, prodrug, stereoisomer, or tautomer thereof, in the treatment of a disease associated with inhibiting cGAS.

Another aspect of the invention relates to a method of treating cancer. The method comprises administering to a patient in need thereof an effective amount of a compound, as described herein, or a pharmaceutically acceptable salt, hydrate, solvate, prodrug, stereoisomer, or tautomer thereof.

Another aspect of the invention relates to a method of treating or preventing cancer. The method comprises administering to a patient in need thereof an effective amount of a compound, as described herein, or a pharmaceutically acceptable salt, hydrate, solvate, prodrug, stereoisomer, or tautomer thereof.

In one embodiment, the present invention relates to the use of an inhibitor of cGAS for the preparation of a medicament used in treatment, prevention, inhibition or elimination of a disease or disorder associated with cancer.

In another embodiment, the present invention relates to a compound, as described herein, or a pharmaceutically acceptable salt, hydrate, solvate, prodrug, stereoisomer, or tautomer thereof, or a pharmaceutical composition comprising a compound of the present invention and a pharmaceutically acceptable carrier used for the treatment of diseases or disorders including, but not limited to, systemic lupus erythematosus (SLE), cutaneous lupus erythematosus (CLE), scleroderma, psoriasis, Aicardi Goutieres syndrome, Sjogren's syndrome, rheumatoid arthritis, inflammatory bowel diseases, multiple sclerosis, diabetes, cardiovascular, and neurodegenerative diseases.

In one embodiment, the compounds of the instant disclosure are used for the treatment of cancer including, but not limited to, bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, pancreatic cancer, penile cancer, testicular germ cell cancer, thymoma carcinoma, thymic carcinoma, lung cancer, ovarian cancer, and prostate cancer.

In one aspect, the disease or condition is an inflammatory, allergic or autoimmune diseases such as systemic lupus erythematosus (SLE), cutaneous lupus erythematosus (CLE), psoriasis, insulin-dependent diabetes mellitus (IDDM), scleroderma, Aicardi Gourtiers syndrome, dermatomyositis, inflammatory bowel diseases, multiple sclerosis, rheumatoid arthritis, or Sjogren's syndrome (SS).

In one embodiment, the compounds of the invention may be used to treat inflammation of any tissue and organs of the body, including musculoskeletal inflammation, vascular inflammation, neural inflammation, digestive system inflammation, ocular inflammation, inflammation of the reproductive system, and other inflammation, as exemplified below.

Musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation which may be treated with compounds of the invention include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic). Ocular inflammation refers to inflammation of any structure of the eye, including the eye lids. Examples of ocular inflammation which may be treated with the compounds of the invention include blepharitis, blepharochalasis, conjunctivitis, dacryoadenitis, keratitis, keratoconjunctivitis sicca (dry eye), scleritis, trichiasis, and uveitis. Examples of inflammation of the nervous system which may be treated with the compounds of the invention include encephalitis, Guillain-Barre syndrome, meningitis, neuromyotonia, narcolepsy, multiple sclerosis, myelitis and schizophrenia.

Examples of inflammation of the vasculature or lymphatic system which may be treated with the compounds of the invention include arthrosclerosis, arthritis, phlebitis, vasculitis, and lymphangitis.

Examples of inflammatory conditions of the digestive system which may be treated with the compounds of the invention include cholangitis, cholecystitis, enteritis, enterocolitis. gastritis, gastroenteritis, inflammatory bowel disease (such as Crohn's disease and ulcerative colitis), ileitis, and proctitis.

Examples of inflammatory conditions of the reproductive system which may be treated with the compounds of the invention include cervicitis, chorioamnionitis, endometritis, epididymitis, omphalitis, oophoritis, orchitis, salpingitis, tubo-ovarian abscess, urethritis, vaginitis, vulvitis, and vulvodynia.

The agents may be used to treat autoimmune conditions having an inflammatory component. Such conditions include systemic lupus erythematosus, cutaneous lupus erythematosus, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome. Grave's disease, Guillain-Barre syndrome, Hashimoto's disease, Henoch-Schonlein purpura, Kawasaki's disease, microscopic colitis, microscopic polyarteritis, mixed connective tissue disease, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, Aicardi Gourtiers syndrome, temporal arteritis, Wegener's granulomatosis, warm autoimmune haemolytic anemia, interstitial cystitis, lyme disease, morphea, psoriasis, sarcoidosis, scleroderma, ulcerative colitis, and vitiligo.

The compounds of the invention may be used to treat T-cell mediated hypersensitivity diseases having an inflammatory component. Such conditions include contact hypersensitivity, contact dermatitis (including that due to poison ivy), uticaria, skin allergies, respiratory allergies (hayfever, allergic rhinitis) and gluten-sensitive enteropathy (Celliac disease).

Other inflammatory conditions which may be treated with the agents include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome. Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, astopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autroimmine) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Suitable treatments include treatment of transplant rejection, rheumatoid arthritis, psoriatic arthritis, multiple sclerosis. Type 1 diabetes, asthma, inflammatory bowel disease, systemic lupus erythematosis, psoriasis, chronic pulmonary disease, and inflammation accompanying infectious conditions (e.g., sepsis).

The compounds of the instant disclosure and pharmaceutically acceptable salts thereof may also be used in combination with one or more other agents in the prevention or treatment of an allergic inflammatory autoimmune disease, wherein such other agents can include: antigen immunotherapy agents; anti-histamines; steroids, NSAIDs; bronchodilators (e.g. beta 2 agonists, adrenergic agonists, anticholinergic agents, theophylline); methotrexate; leukotriene modulators; monoclonal antibody agents such as anti-lgE, anti-TNF, anti-IL-5, anti-IL-6, anti-IL-12, anti-IL-1 and similar agents; receptor therapies agents such as entanercept; and antigen non-specific immunotherapeutic agents such interferon or other cytokines/chemokines, cytokine/chemokine receptor modulators, cytokine agonists or antagonists, and TLR antagonist.

Another aspect of the invention is directed to pharmaceutical compositions comprising a compound of Formula (I) and a pharmaceutically acceptable carrier. The pharmaceutical acceptable carrier may further include an excipient, diluent, or surfactant.

In one embodiment, are provided methods of treating a disease or disorder associated with modulation of cGAS including, cancer or cell proliferative disorder, comprising administering to a patient suffering from at least one of said diseases or disorder a compound, as described herein, or a pharmaceutically acceptable salt, hydrate, solvate, prodrug, stereoisomer, or tautomer thereof.

One therapeutic use of the compounds or compositions of the present invention which inhibit cGAS is to provide treatment to patients or subjects suffering from a cancer or cell proliferative disorder.

The disclosed compounds of the invention can be administered in effective amounts to treat or prevent a disorder and/or prevent the development thereof in subjects.

Administration of the disclosed compounds can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Illustrative pharmaceutical compositions are tablets and gelatin capsules comprising a Compound of the Invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564 which is hereby incorporated by reference in its entirety.

Disclosed compounds can also be delivered by the use of monoclonal antibodies as individual carriers to which the disclosed compounds are coupled. The disclosed compounds can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the Disclosed compounds can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, disclosed compounds are not covalently bound to a polymer, e.g., a polycarboxylic acid polymer, or a polyacrylate.

Parenteral injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention is directed to pharmaceutical compositions comprising a compound, as described herein, or a pharmaceutically acceptable salt, hydrate, solvate, prodrug, stereoisomer, or tautomer thereof, and a pharmaceutically acceptable carrier. The pharmaceutical acceptable carrier may further include an excipient, diluent, or surfactant. In some embodiments, the pharmaceutical composition can further comprise an additional pharmaceutically active agent.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EXAMPLES

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Synthesis of Compounds

Nuclear magnetic resonance (NMR) spectra were recorded at 400 MHz as stated and at 300.3 K unless otherwise stated; the chemical shifts (6) are reported in parts per million (ppm). Spectra were recorded using a Bruker Avance 400 instrument with 8, 16 or 32 scans.

LC-MS chromatograms and spectra were recorded using a Shimadzu LCMS-2020. Injection volumes were 0.7-8.0 μl and the flow rates were typically 0.8 or 1.2 ml/min. Detection methods were diode array (DAD) or evaporative light scattering (ELSD) as well as positive ion electrospray ionisation. MS range was 100-1000 Da. Solvents were gradients of water and acetonitrile both containing a modifier (typically 0.01-0.04%) such as trifluoroacetic acid or ammonium carbonate.

The Examples set forth herein below provide syntheses and experimental results obtained for certain exemplary compounds. Unless otherwise indicated, all numbers expressing quantities of ingredients, reaction conditions, concentrations, properties, stabilities, and so forth used in the specification and claims are to be understood as being modified in all instances by the term “about.” At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contain certain errors resulting from variations in experiments, testing measurements, statistical analyses and such. The following is to be construed as merely illustrative, and not limitations of the preceding disclosure in any way whatsoever. Those skilled in the art will promptly recognize appropriate variations from the procedures both as to reactants and as to reaction and purification conditions and techniques. Unless otherwise indicated, starting materials or intermediates are commercially available or are known in the chemical literature.

As used herein, the following abbreviations may have the following meanings:

| Abbreviation | Term |
|---|---|
| AcOH or HOAc | Acetic acid |
| ACN or MeCN | Acetonitrile |
| AIBN | Azobisisobutyronitrile |
| ATP | Adenosine triphosphate |
| nBuLi | n-Butyl-lithium |
| mCPBA | meta-Chloroperoxybenzoic acid |
| DBAD | Di-tert-butyl azodicarboxylate |
| DBU | 1,8-Diazabicyclo[5.4.0]undec-7-ene |
| DCE | Dichloroethane |
| DCM | Dichloromethane |
| DIPEA or DIEA | Diisopropylethylamine |
| DMAP | 4-Dimethylaminopyridine |
| DMF | N,N-dimethylformamide |
| DMSO | Dimethylsulfoxide |
| dsDNA | Double stranded DNA |
| DTT | Dithiothreitol |
| EDCI | 1-Ethyl-3-(3-dimethylaminopropyl)carbodiimide |
| Equiv. or eq. | Equivalent |
| EtOAc or EA | Ethyl acetate |
| EtOH | Ethanol |
| FA | Formic acid |
| GTP | Guanosine triphosphate |
| h | Hour(s) |
| h-cGAS | Human-cGAS |
| HATU | 1-[Bis(dimethylamino)methylene]-1H-1,2,3-triazolo |
| | [4,5-b]pyridinium 3-oxidhexafluorophosphate |
| HOBt | Hydroxybenzotriazole |
| Prep-HPLC | Preparative High-performance liquid chromatography |
| LCMS (ES) | Liquid Chromatography-Mass Spectrometry |
| | (Electrospray Ionization) |
| LHMDS | Lithium bis(trimethylsilyl)amide |
| min | Minute(s) |
| NMI | N-Methylimidazole |
| NMR | Nuclear magnetic resonance |
| NBS | N-bromosuccinimide |
| NCS | N-chlorosuccinimide |
| NMM | N-Methylmorpholine |
| NMO | N-Methylmorpholine-N-oxide |
| PE | Petroleum ether |
| Pd(dppf)Cl₂ | [1,1′-Bis(diphenylphosphino)ferrocene] |
| | dichloropalladium(II) |
| PMB | 4-methoxybenzyl |
| rt | Room temperature |
| TBAB | Tetra-n-butylammonioum bromide |
| TBAF | Tetra-n-butylammonioum fluoride |
| TCFH | Chloro-N,N,N′,N′-tetramethylformamidinium |
| | hexafluorophosphate |
| TEA | Triethylamine |
| TFA | Trifluoroacetic acid |
| THF | Tetrahydrofuran |
| Tris-HCl | Tris(hydroxymethyl)aminomethane hydrochloride |
| T3P | Propylphosphonic acid |

### Example 1: (6,7-dichloro-1,3,4,5-tetrahydropyrido[4,3-b]indol-2-yl)-(5-methoxypyrimidin-2-yl)methanone (Compound 1)

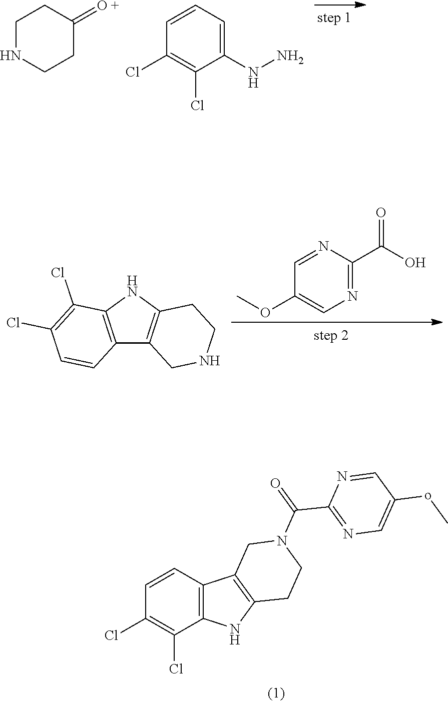

Step 1: A solution of piperidin-4-one hydrochloride (1.00 eq, 1867 mg, 13.8 mmol) and (2,3-dichlorophenyl)hydrazine hydrochloride (1.00 eq, 3000 mg, 13.8 mmol) in 1,4-dioxane (60 mL) was treated with sulfuric acid (3.00 eq, 2.3 mL, 41.3 mmol) and stirred at 100° C. for 4 h. The crude mixture was cooled down to room temperature, filtered, rinsed with 40% EtOH in EtOAc (30 mL) and dried in vacuo to give 6,7-dichloro-2,3,4,5-tetrahydro-1H-pyrido[4,3-b]indole sulfuric acid (1:0.5) (6.65 g, 83%) as a beige solid. LCMS (ES, m/z)=241.1 [M+H]⁺. ¹HNMR (400 MHz, DMSO-d₆) δ 11.65 (s, 1H), 7.92 (br. s, 1H), 7.47 (d, J=8.4 Hz, 1H), 7.22 (d, J=8.4 Hz, 1H), 4.25 (s, 2H), 3.42 (t, J=6.1 Hz, 2H), 2.99 (t, J=6.1 Hz, 2H).

Step 2: 6,7-dichloro-2,3,4,5-tetrahydro-1H-pyrido[4,3-b]indole sulfuric acid (1:0.5) (1.00 eq, 678 mg, 1.17 mmol), diisopropylethylamine (4.00 eq, 1.80 mL, 10.4 mmol) and 5-methoxypyrimidine-2-carboxylic acid (1.00 eq, 400 mg, 2.60 mmol) were combined in DMF (8.6 mL), followed by O-(7-azabenzotriazol-1-yl)-N,N,N,N-tetramethyluronium hexafluorophosphate (1.00 eq, 1.02 g, 2.60 mmol) added in one portion. The reaction mixture was stirred at room temperature for 3 h. The crude mixture was purified by prep-HPLC using a C18 column and a 20-70% MeCN/0.1% aqueous formic acid gradient to afford (6,7-dichloro-1,3,4,5-tetrahydropyrido[4,3-b]indol-2-yl)-(5-methoxypyrimidin-2-yl)methanone (Compound 1) (211 mg, 22% yield) as an off-white solid. LCMS (ES, m/z)=377.0 [M+H]⁺. ¹HNMR (400 MHz, DMSO-d₆) δ 11.58-11.50 (m, 1H), 8.69-8.60 (m, 2H), 7.56-7.23 (m, 1H), 7.22-7.05 (m, 1H), 4.85-4.38 (m, 2H), 4.00-3.94 (m, 3H), 4.06-3.49 (m, 2H), 2.94-2.79 (m, 2H).

### Example 2: (R)-2-amino-1-(6,7-dichloro-1,3,4,5-tetrahydro-2H-pyrido[4,3-b]indol-2-yl)-3-methoxypropan-1-one (Compound 53)

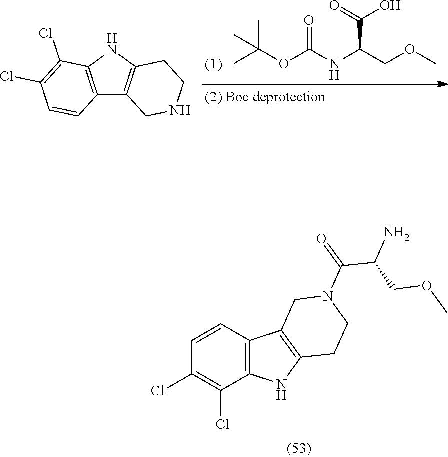 [SMILES: Clc1ccc2c3c([nH]c2c1Cl)CCNC3 ; COC[C@@H](NC(=O)OC(C)(C)C)C(=O)O ; COC[C@@H](N)C(=O)N1CCc2[nH]c3c(Cl)c(Cl)ccc3c2C1]

6,7-dichloro-2,3,4,5-tetrahydro-1H-pyrido[4,3-b]indole sulfuric acid (Step 1 product of Example 1) (1:0.5) (1.00 eq, 34.9 mg, 0.120 mmol), diisopropylethylamine (4.4 eq, 93 μL, 0.54 mmol) and (2R)-2-(tert-butoxycarbonylamino)-3-methoxy-propanoic acid (1.11 eq, 29.3 mg, 0.134 mmol) were combined in DMF (0.97 mL), followed by O-(7-azabenzotriazol-1-yl)-N,N,N,N-tetramethyluronium hexafluorophosphate (1.11 eq, 52.4 mg, 0.134 mmol) in one portion. The reaction mixture was stirred at room temperature for 2 h. Then, the crude mixture was concentrated in vacuo. DCM (1.0 mL) and TFA (1.0 mL) were added, and the reaction mixture was stirred at room temperature for 2 h. The crude mixture was concentrated in vacuo, neutralized with Et₃N and purified by prep-HPLC using a C18 column and a 15-60% MeCN/0.1% aqueous formic acid gradient to afford (R)-2-amino-1-(6,7-dichloro-1,3,4,5-tetrahydro-2H-pyrido[4,3-b]indol-2-yl)-3-methoxypropan-1-one (Compound 53) (18.5 mg, 40% yield) as a beige solid. LCMS (ES, m/z)=342.1 [M+H]⁺. ¹HNMR (400 MHz, DMSO-d₆) δ 11.55-11.48 (m, 1H), 8.21 (s, 2H), 7.50-7.42 (m, 1H), 7.23-7.13 (m, 1H), 4.90-4.71 (m, 1H), 4.71-4.58 (m, 1H), 4.42-4.33 (m, 1H), 4.14-3.64 (m, 2H), 3.59-3.44 (m, 2H), 3.31-3.19 (m, 3H), 2.99-2.78 (m, 2H).

### Example 3: (6,7-dichloro-5-(3-methoxypropyl)-1,3,4,5-tetrahydro-2H-pyrido[4,3-b]indol-2-yl)(5-methoxypyrimidin-2-yl)methanone (Compound 19)

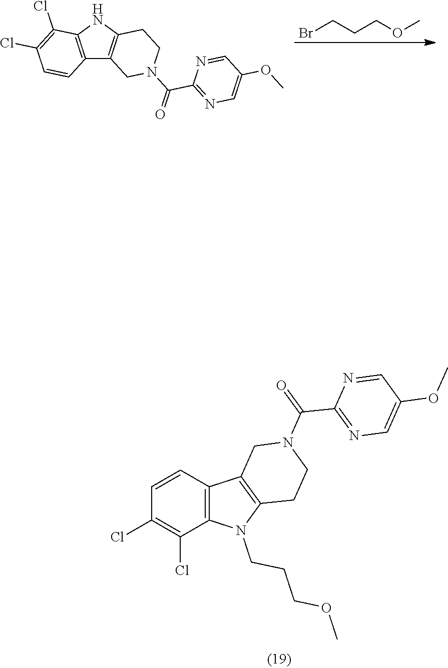

To a solution of (6,7-dichloro-1,3,4,5-tetrahydropyrido[4,3-b]indol-2-yl)-(5-methoxypyrimidin-2-yl)methanone (Compound 1 of Example 1) (1.00 eq, 35.0 mg, 0.0928 mmol) and 1-bromo-3-methoxy-propane (2.00 eq, 28.4 mg, 0.186 mmol) in DMF (0.88 mL) at 0° C. was added sodium hydride 60% in mineral oil (2.0 eq, 7.4 mg, 0.19 mmol) at 0° C. and the reaction mixture was stirred at rt overnight. The crude mixture was quenched with formic acid (30 L), filtered and purified by prep-HPLC using a C18 column and a 20-70% MeCN/0.1% aqueous formic acid gradient to afford (6,7-dichloro-5-(3-methoxypropyl)-1,3,4,5-tetrahydro-2H-pyrido[4,3-b]indol-2-yl)(5-methoxypyrimidin-2-yl)methanone (Compound 19) (32.2 mg, 77% yield) as a beige solid. LCMS (ES, m/z)=449.1 [M+H]⁺. ¹HNMR (400 MHz, DMSO-d₆) δ 8.69-8.59 (m, 2H), 7.58-7.27 (m, 1H), 7.27-7.12 (m, 1H), 4.85-4.43 (m, 2H), 4.01-3.94 (m, 3H), 4.11-3.54 (m, 2H), 3.31-3.14 (m, 4H), 3.25-3.20 (m, 3H), 2.97-2.81 (m, 2H), 1.96-1.87 (m, 2H).

### Example 4: 2-(6,7-dichloro-2-(5-methoxypyrimidine-2-carbonyl)-1,2,3,4-tetrahydro-5H-pyrido[4,3-b]indol-5-yl)acetic acid (Compound 17)

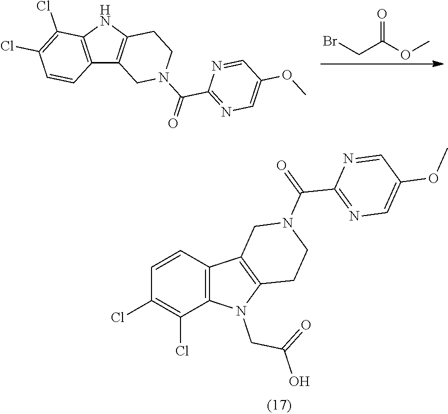

To a solution of (6,7-dichloro-1,3,4,5-tetrahydropyrido[4,3-b]indol-2-yl)-(5-methoxypyrimidin-2-yl)methanone (Compound 1 of Example 1) (1.00 eq, 25.0 mg, 0.0662 mmol) and methyl 2-bromoacetate (2.00 eq, 20.3 mg, 0.132 mmol) in DMF (0.44 mL) at 0° C. was added sodium hydride 60% in mineral oil (2.0 eq, 3.2 mg, 0.13 mmol) at 0° C. and the reaction mixture was stirred at overnight. To the mixture, lithium hydroxide monohydrate (4.00 eq, 11.4 mg, 0.265 mmol) and water (0.30 mL) were added. The mixture was stirred at room temperature for 1 h. The crude mixture was quenched with formic acid (50 L), filtered and purified by prep-HPLC using a C18 column and a 10-50% MeCN/0.1% aqueous formic acid gradient to afford 2-(6,7-dichloro-2-(5-methoxypyrimidine-2-carbonyl)-1,2,3,4-tetrahydro-5H-pyrido[4,3-b]indol-5-yl)acetic acid (Compound 17) (26.9 mg, 93%) as a white solid. LCMS (ES, m/z)=435.2 [M+H]⁺. ¹HNMR (400 MHz, DMSO-d₆) δ 13.87 (br. s, 1H), 8.68-8.60 (m, 2H), 7.58-7.27 (m, 1H), 7.27-7.10 (m, 1H), 5.06 (s, 2H), 4.88-4.39 (m, 2H), 3.97 (s, 3H), 4.07-3.51 (m, 2H), 2.87-2.72 (m, 2H).

### Example 5: (6,7-dichloro-1,3,4,5-tetrahydro-2H-pyrido[4,3-b]indol-2-yl)(5-fluoropyrimidin-2-yl)methanone (Compound 15) and (6,7-dichloro-1,3,4,5-tetrahydro-2H-pyrido[4,3-b]indol-2-yl)(5-((2-hydroxyethyl)amino)pyrimidin-2-yl)methanone (Compound 29)

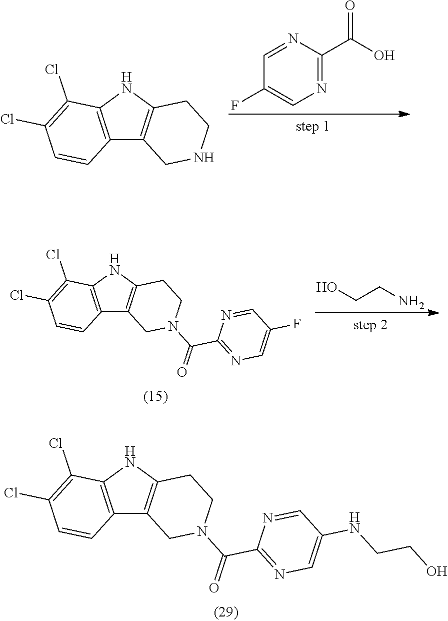

Step 1: 6,7-dichloro-2,3,4,5-tetrahydro-1H-pyrido[4,3-b]indole sulfuric acid (1:0.5) (Step 1 product of Example 1) (1.00 eq, 1.00 g, 1.72 mmol), diisopropylethylamine (4.50 eq, 3.00 mL, 17.2 mmol) and 5-fluoropyrimidine-2-carboxylic acid (1.00 eq, 544 mg, 3.83 mmol) were combined in DMF (19 mL), followed by O-(7-azabenzotriazol-1-yl)-N,N,N,N-tetramethyluronium hexafluorophosphate (1.00 eq, 1.50 g, 3.83 mmol) added in one portion. The reaction mixture was stirred at rt for 3 h. To the crude mixture was added water (20 mL) and the precipitate formed was filtered, washed with water (20 mL), EtOH (5 mL) and dried in vacuo to give (6,7-dichloro-1,3,4,5-tetrahydro-2H-pyrido[4,3-b]indol-2-yl)(5-fluoropyrimidin-2-yl)methanone (Compound 15) (568 mg, 41% yield) as a beige solid. LCMS (ES, m/z)=365.2 [M+H]⁺. ¹HNMR (400 MHz, DMSO-d₆) δ 11.59-11.51 (m, 1H), 9.07-8.98 (m, 2H), 7.53-7.25 (m, 1H), 7.22-7.06 (m, 1H), 4.88-4.37 (m, 2H), 4.08-3.49 (m, 2H), 2.97-2.77 (m, 2H).

Step 2: (6,7-dichloro-1,3,4,5-tetrahydropyrido[4,3-b]indol-2-yl)-(5-fluoropyrimidin-2-yl)methanone (Compound 15) (1.00 eq, 30.0 mg, 0.0821 mmol), ethanolamine (3.00 eq, 14.9 μL, 0.246 mmol) and diisopropylethylamine (3.00 eq, 42.9 μL, 0.246 mmol) were combined in DMSO (0.21 mL) and the reaction mixture was stirred at 120° C. for 2 h. The crude mixture was purified by reverse-phase chromatography using a C18 column and a 20-60% MeCN/0.1% aqueous formic acid gradient to afford (6,7-dichloro-1,3,4,5-tetrahydro-2H-pyrido[4,3-b]indol-2-yl)(5-((2-hydroxyethyl)amino)pyrimidin-2-yl)methanone (Compound 29) (25.6 mg, 71% yield) as a white solid. LCMS (ES, m/z)=406.2 [M+H]⁺. ¹HNMR (400 MHz, DMSO-d₆) δ 11.56-11.49 (m, 1H), 8.24-8.15 (m, 2H), 7.54-7.24 (m, 1H), 7.22-7.04 (m, 1H), 6.47 (q, J=5.5 Hz, 1H), 4.81-4.45 (m, 2H), 4.36 (s, 1H), 3.99 (t, J=5.8 Hz, 1H), 3.63-3.55 (m, 3H), 3.27-3.14 (m, 2H), 2.94-2.81 (m, 2H).

### Example 6: (6,7-dichloro-1-methyl-1,3,4,5-tetrahydro-2H-pyrido[4,3-b]indol-2-yl)(5-fluoropyrimidin-2-yl)methanone (Compound 155, rac-155), (R)-(6,7-dichloro-1-methyl-1,3,4,5-tetrahydro-2H-pyrido[4,3-b]indol-2-yl)(5-fluoropyrimidin-2-yl)methanone (Compound 155A*), (S)-(6,7-dichloro-1-methyl-1,3,4,5-tetrahydro-2H-pyrido[4,3-b]indol-2-yl)(5-fluoropyrimidin-2-yl)methanone (Compound 155B*), (6,7-dichloro-1-methyl-1,3,4,5-tetrahydro-2H-pyrido[4,3-b]indol-2-yl)(5-morpholinopyrimidin-2-yl)methanone (Compound 44, Rac-44), (R)-(6,7-dichloro-1-methyl-1,3,4,5-tetrahydro-2H-pyrido[4,3-b]indol-2-yl)(5-morpholinopyrimidin-2-yl)methanone (Compound 44A*) and (S)-(6,7-dichloro-1-methyl-1,3,4,5-tetrahydro-2H-pyrido[4,3-b]indol-2-yl)(5-morpholinopyrimidin-2-yl)methanone (Compound 44B*)

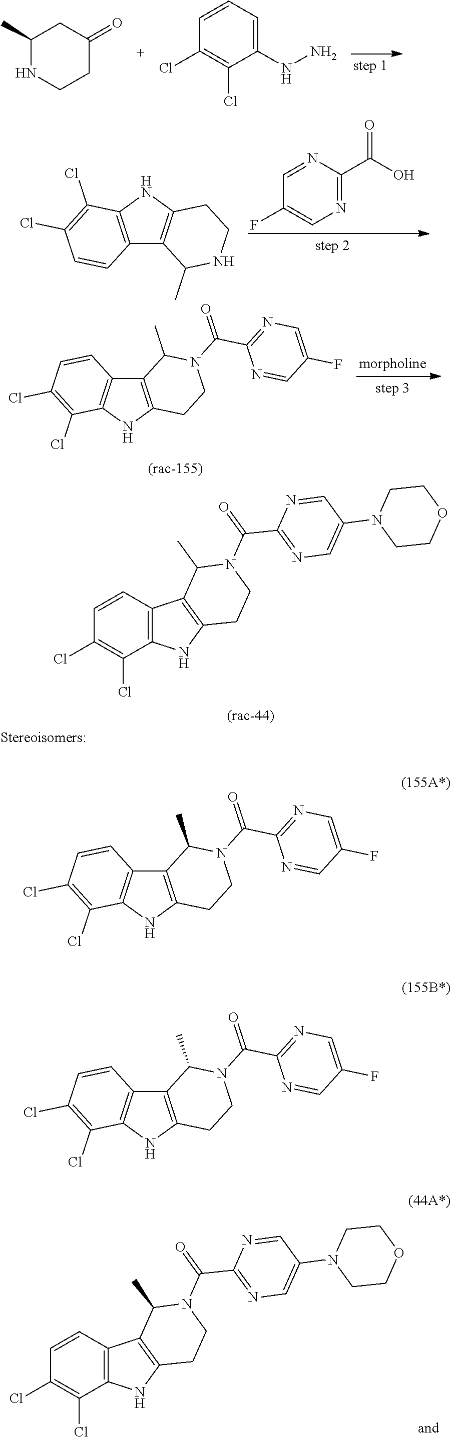

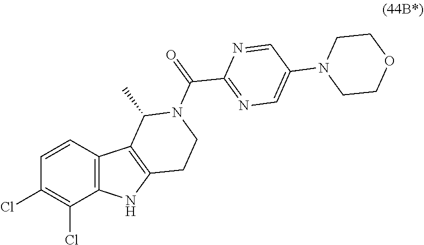 [SMILES: C[C@H]1c2c([nH]c3c(Cl)c(Cl)ccc23)CCN1C(=O)c1ncc(N2CCOCC2)cn1]

Step 1: Into a solution of (2,3-dichlorophenyl)hydrazine hydrochloride (1.00 eq, 100 g, 468 mmol) and (2S)-2-methylpiperidin-4-one hydrochloride (1.00 eq, 70.1 g, 468 mmol) in EtOH (500 mL) was added conc. H₂SO₄ (10.0 eq, 459 g, 4.68 mol) at room temperature. The mixture was stirred for overnight at 80° C. The resulting mixture was cooled to room temperature and poured into ice water dropwise. The mixture was extracted with EtOAc (5×1000 mL). The combined organic layers were washed with water (3×1000 mL) and dried over anhydrous Na₂SO₄. After filtration, the filtrate was concentrated under reduced pressure. The residue was dissolved in ethyl acetate (40 mL). The precipitated solids were collected by filtration and washed with ethyl acetate (3×5 mL). The residue was purified by reverse flash chromatography using a C18 column and a 45% MeOH/0.5% aqueous formic acid gradient to give 6,7-dichloro-1-methyl-1H,2H,3H,4H,5H-pyrido[4,3-b]indole;trifluoroacetic acid (also referred to herein as 6,7-dichloro-1-methyl-2,3,4,5-tetrahydro-1H-pyrido[4,3-b]indole;2,2,2-trifluoroacetic acid) (10.1 g, 6% yield) as a light brown solid, which was a 60:40 S/R mixture of enantiomers. LCMS (ES, m/z)=255.0 [M+H]⁺. ¹H NMR (400 MHz, DMSO-d₆) δ 7.54 (d, J=8.5 Hz, 1H), 7.22 (d, J=8.4 Hz, 1H), 4.77 (q, J=6.7 Hz, 1H), 3.62-3.53 (m, 1H), 3.46-3.40 (m, 1H), 3.04 (q, J=5.7 Hz, 2H), 1.64 (d, J=6.7 Hz, 3H).

Step 2: 6,7-dichloro-1-methyl-2,3,4,5-tetrahydro-1H-pyrido[4,3-b]indole;2,2,2-trifluoroacetic acid as a 60:40 S/R mixture of enantiomers (1.00 eq, 1.04 g, 2.82 mmol), 5-fluoropyrimidine-2-carboxylic acid (1.10 eq, 440 mg, 3.10 mmol) and diisopropylethylamine (3.50 eq, 1.72 mL, 9.86 mmol) were combined in DMF (9.0 mL), followed by O-(7-azabenzotriazol-1-yl)-N,N,N,N-tetramethyluronium hexafluorophosphate (1.10 eq, 1.22 g, 3.10 mmol) in one portion. The reaction mixture was stirred at room temperature overnight. To the crude mixture was added water (30 mL) and the precipitate formed was filtered, washed with water (50 mL) and dried in vacuo to give [6,7-dichloro-1-methyl-1,3,4,5-tetrahydropyrido[4,3-b]indol-2-yl]-(5-fluoropyrimidin-2-yl)methanone (Compound 155, rac-155) (985 mg, 92% yield) as a beige solid as a mixture of 60:40 S/R mixture of enantiomers, (R)-(6,7-dichloro-1-methyl-1,3,4,5-tetrahydro-2H-pyrido[4,3-b]indol-2-yl)(5-fluoropyrimidin-2-yl)methanone (Compound 155A*), (S)-(6,7-dichloro-1-methyl-1,3,4,5-tetrahydro-2H-pyrido[4,3-b]indol-2-yl)(5-fluoropyrimidin-2-yl)methanone (Compound 155B*), stereochemistry arbitrarily assigned. LCMS (ES, m/z)=379.1 [M+H]⁺. ¹HNMR (400 MHz, DMSO-d₆) δ 11.57-11.48 (m, 1H), 9.06-8.98 (m, 2H), 7.59-7.27 (m, 1H), 7.22-7.06 (m, 1H), 5.82-4.62 (m, 1H), 3.63-3.33 (m, 2H), 2.91-2.79 (m, 1H), 2.75-2.65 (m, 1H), 1.57-1.42 (m, 3H).

Step 3: A solution of [6,7-dichloro-1-methyl-1,3,4,5-tetrahydropyrido[4,3-b]indol-2-yl]-(5-fluoropyrimidin-2-yl)methanone (Compound 155, rac-155) (1.00 eq, 30.0 mg, 0.0791 mmol), diisopropylethylamine (3.0 eq, 41 μL, 0.24 mmol), morpholine (3.0 eq, 21 μL, 0.24 mmol) and in DMSO (0.40 mL) was stirred at 120° C. for 2 h. The crude mixture was directly purified by reverse-phase chromatography using a C18 column and a 20-70% MeCN/0.1% aqueous formic acid gradient to afford (6,7-dichloro-1-methyl-1,3,4,5-tetrahydro-2H-pyrido[4,3-b]indol-2-yl)(5-morpholinopyrimidin-2-yl)methanone (Compound 44, Rac-44) (32.0 mg, 91%) as a white solid, provided as a mixture of 60:40 S/R mixture of enantiomers, (R)-(6,7-dichloro-1-methyl-1,3,4,5-tetrahydro-2H-pyrido[4,3-b]indol-2-yl)(5-morpholinopyrimidin-2-yl)methanone (Compound 44A*) and (S)-(6,7-dichloro-1-methyl-1,3,4,5-tetrahydro-2H-pyrido[4,3-b]indol-2-yl)(5-morpholinopyrimidin-2-yl)methanone (Compound 44B*), stereochemistry arbitrarily assigned. LCMS (ES, m/z)=446.2 [M+H]⁺. ¹HNMR (400 MHz, DMSO-d₆) δ 11.57-11.48 (m, 2H), 8.59-8.51 (m, 3H), 7.57-7.27 (m, 1H), 7.23-7.05 (m, 1H), 5.74 (q, J=6.5 Hz, 1H), 4.81-4.71 (m, 1H), 3.81-3.72 (m, 4H), 3.61-3.41 (m, 2H), 2.92-2.83 (m, 2H), 2.75-2.66 (m, 1H), 1.54-1.45 (m, 3H).

### Example 7: (6,7-dichloro-1-methyl-1,3,4,5-tetrahydro-2H-pyrido[4,3-b]indol-2-yl)(5-methoxypyrimidin-2-yl)methanone (Compound 45, rac-45), (R)-(6,7-dichloro-1-methyl-1,3,4,5-tetrahydro-2H-pyrido[4,3-b]indol-2-yl)(5-methoxypyrimidin-2-yl)methanone (Compound 45A*) and (S)-(6,7-dichloro-1-methyl-1,3,4,5-tetrahydro-2H-pyrido[4,3-b]indol-2-yl)(5-methoxypyrimidin-2-yl)methanone (Compound 45B*)

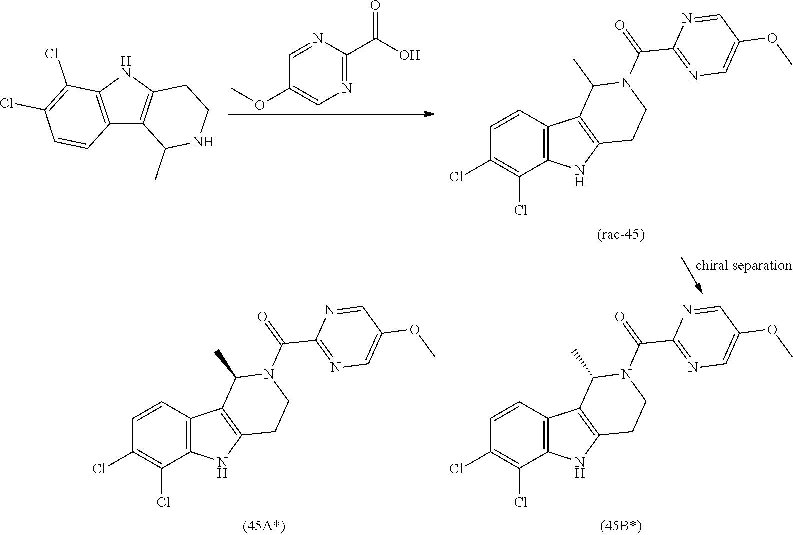

Into a 20 mL vial were added 6,7-dichloro-1-methyl-2,3,4,5-tetrahydro-1H-pyrido[4,3-b]indole;2,2,2-trifluoroacetic acid (Step 1 product, Example 6) as a 60:40 S/R mixture of enantiomers (1.00 eq, 100 mg, 0.390 mmol), 5-methoxypyrimidine-2-carboxylic acid (1.10 eq, 66.4 mg, 0.43 mmol), DMF (5.0 mL) and NMM (2.00 eq, 79.3 mg, 0.78 mmol) at room temperature. Then HATU (1.20 eq, 179 mg, 0.47 mmol) was added. The reaction progress was monitored by LCMS. The reaction was quenched by the addition of water (20 mL) at room temperature. The resulting mixture was extracted with ethyl acetate (3×30 mL), washed with saturate brine (3×20 mL). Dried over anhydrous Na₂SO₄. After filtration, the filtrate was concentrated under reduced pressure. The crude product was purified by Prep-HPLC using XBridge Shield RP18 OBD Column (30*150 mm, 5 μm) and 44% to 54% ACN in water (10 mmol/L NH₄HCO₃), to afford 2-[6,7-dichloro-1-methyl-1H,3H,4H,5H-pyrido[4,3-b]indole-2-carbonyl]-5-methoxypyrimidine, also referred to herein as (6,7-dichloro-1-methyl-1,3,4,5-tetrahydro-2H-pyrido[4,3-b]indol-2-yl)(5-methoxypyrimidin-2-yl)methanone (Compound 45, rac-45) (26.1 mg, 8% yield) as a white solid, which was provided as a mixture of enantiomers, Compound 45A* and Compound 45B*, stereochemistry arbitrarily assigned. The enantiomers were separated by Chiral Prep-HPLC using CHIRAL ART Cellulose-SB (4.6*100 mm, 3 μm) and 70% Hexanes (0.2% formic acid) in MeOH:DCM (1:1) as the eluent to afford 2-[(1S)-6,7-dichloro-1-methyl-1H,3H,4H,5H-pyrido[4,3-b]indole-2-carbonyl]-5-methoxypyrimidine, also referred to herein as (S)-(6,7-dichloro-1-methyl-1,3,4,5-tetrahydro-2H-pyrido[4,3-b]indol-2-yl)(5-methoxypyrimidin-2-yl)methanone (Compound 45B*) (chiral RT(min): 11.56, 12.0 mg) as a white solid and (R)-(6,7-dichloro-1-methyl-1,3,4,5-tetrahydro-2H-pyrido[4,3-b]indol-2-yl)(5-methoxypyrimidin-2-yl)methanone (Compound 45A*) (chiral RT(min): 9.92, 4 mg) as a white solid.

Compound 45B*: LCMS (ES, m/z)=391.0 [M+H]⁺. ¹HNMR (400 MHz, DMSO-d₆) δ 11.52 (d, J=16.5 Hz, 1H), 8.64 (m, J=13.0, 1.1 Hz, 2H), 7.55-7.09 (d, J=8.4 Hz, 2H), 5.76 (m, J=6.6 Hz, 1H), 4.79-4.68 (d, J=13.2 Hz, 1H), 3.98 (s, 3H), 3.54-3.46 (m, 1H), 2.87 (t, J=11.2 Hz, 1H), 2.72 (d, 1H) 1.50-1.48 (m, 3H).

Compound 45A*: LCMS (ES, m/z)=391.0 [M+H]⁺. ¹HNMR (400 MHz, DMSO-d₆) δ 11.52 (s, 1H), 8.68-8.61 (d, J=2.8 Hz, 2H), 8.57 (s, 1H), 7.62 (s, 1H), 7.55-7.00 (d, J=8.4 Hz, 2H), 5.80 (s, 1H), 4.79 (s, 1H), 4.01-3.95 (m, 5H), 3.61-3.50 (t, J=8.5 Hz, 3H), 2.99-2.95 (t, J=1.6 Hz, 3H), 2.89 (s, 1H), 1.50-1.45 (t, J=6.6 Hz, 3H), 1.45-1.40 (d, J=2.0 Hz, 2H).

### Example 8: (R)-2-amino-1-(6,7-dichloro-1-methyl-1,3,4,5-tetrahydro-2H-pyrido[4,3-b]indol-2-yl)ethan-1-one (Compound 51A*), and (S)-2-amino-1-(6,7-dichloro-1-methyl-1,3,4,5-tetrahydro-2H-pyrido[4,3-b]indol-2-yl)ethan-1-one (Compound 51B*)

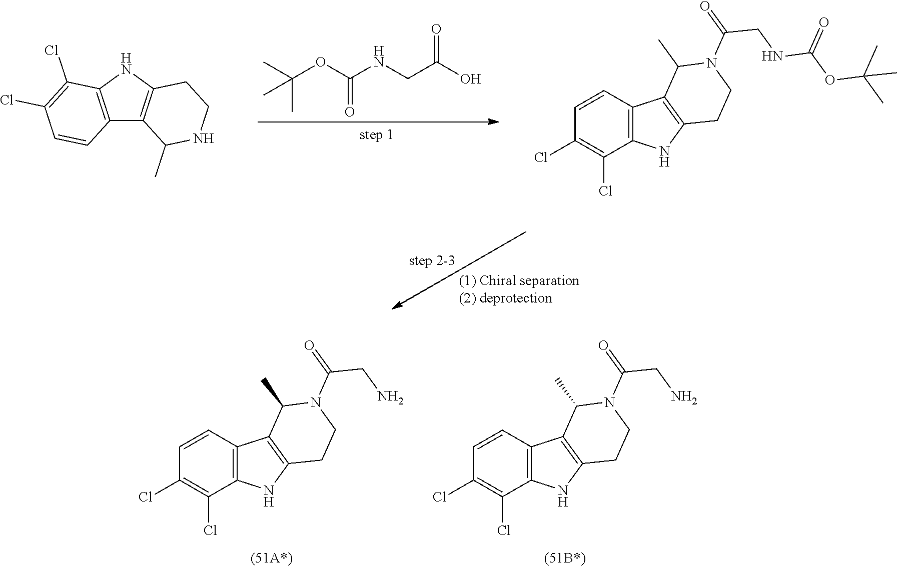 [SMILES: CC1NCCc2[nH]c3c(Cl)c(Cl)ccc3c21 ; CC(C)(C)OC(=O)NCC(=O)O ; CC1c2c([nH]c3c(Cl)c(Cl)ccc23)CCN1C(=O)CNC(=O)OC(C)(C)C ; C[C@@H]1c2c([nH]c3c(Cl)c(Cl)ccc23)CCN1C(=O)CN ; C[C@H]1c2c([nH]c3c(Cl)c(Cl)ccc23)CCN1C(=O)CN]

Step 1: Into a 40 mL vial were added 6,7-dichloro-1-methyl-2,3,4,5-tetrahydro-1H-pyrido[4,3-b]indole;2,2,2-trifluoroacetic acid (Step 1 product, Example 6) as a 60:40 S/R mixture of enantiomers (1.00 eq, 550 mg, 2.15 mmol), [(tert-butoxycarbonyl)amino]acetic acid (1.20 eq, 453 mg, 2.58 mmol), NMM (2.00 eq, 436 mg, 4.31 mmol) and dimethylformamide (10 mL), then HATU (1.20 eq, 984 mg, 2.58 mmol) was added at room temperature. The resulting mixture was stirred for 2 h at room temperature. The reaction progress was monitored by LCMS. The reaction was quenched by the addition of water (30 mL) at room temperature. The resulting mixture was extracted with ethyl acetate (3×50 mL). The combined organic layers were washed with brine (2×20 mL), dried over anhydrous MgSO₄. After filtration, the filtrate was concentrated under reduced pressure. The residue was purified by silica gel column chromatography, eluted with petroleum ether/ethyl acetate (1:1) to afford tert-butyl N-(2-{6,7-dichloro-1-methyl-1H,3H,4H,5H-pyrido[4,3-b]indol-2-yl}-2-oxoethyl)carbamate (740 mg, 75% yield) as a white solid mixture of enantiomers. LCMS (ES, m/z)=412.0 [M+H]⁺. ¹HNMR (400 MHz, DMSO-d₆) δ 11.46 (d, J=10.2 Hz, 1H), 7.47 (m, J=8.4, 4.6 Hz, 1H), 7.18 (m, J=13.5, 8.4 Hz, 1H), 6.79 (d, J=6.1 Hz, 1H), 5.61 (d, J=6.9 Hz, 1H), 5.32-4.60 (m, 1H), 3.91 (t, J=6.5 Hz, 2H), 3.43 (t, J=13.6 Hz, 1H), 3.15-2.61 (m, 2H), 1.39 (d, J=1.9 Hz, 9H), 1.35 (d, J=21.3 Hz, 3H).

Step 2: 250 mg of the mixture of enantiomers were separated by Chiral Prep-HPLC using Chiral Prep-HPLC using CHIRAL ART Cellulose-SB (4.6*100 mm, 3 μm) and 45% Hexanes (0.2% formic acid) in MeOH:DCM (1:1) as the eluent to as the eluent to afford tert-butyl (S)-(2-(6,7-dichloro-1-methyl-1,3,4,5-tetrahydro-2H-pyrido[4,3-b]indol-2-yl)-2-oxoethyl)carbamate (chiral RT(min)=10.49, 132 mg) as a white solid and tert-butyl (R)-(2-(6,7-dichloro-1-methyl-1,3,4,5-tetrahydro-2H-pyrido[4,3-b]indol-2-yl)-2-oxoethyl)carbamate (chiral RT(min)=9.83, 260 mg) as a white solid. LCMS (ES, m/z)=412.1 [M+H]⁺. ¹HNMR (400 MHz, DMSO-d₆) δ 11.46 (d, J=10.2 Hz, 1H), 7.47 (m, J=8.4, 4.6 Hz, 1H), 7.18 (m, J=13.5, 8.4 Hz, 1H), 6.79 (d, J=6.1 Hz, 1H), 5.61 (d, J=6.9 Hz, 1H), 5.32-4.60 (m, 1H), 3.91 (t, J=6.5 Hz, 2H), 3.43 (t, J=13.6 Hz, 1H), 3.15-2.61 (m, 2H), 1.39 (d, J=1.9 Hz, 9H), 1.35 (d, J=21.3 Hz, 3H). Stereochemistry arbitrarily assigned.

Step 3A: Into a mL vial were added tert-butyl N-{2-[(1R)-6,7-dichloro-1-methyl-1H,3H,4H,5H-pyrido[4,3-b]indol-2-yl]-2-oxoethyl}carbamate (132 mg, 0.32 mmol, 1.0 equiv) and HCl(g) (58 mg, 1.6 mmol, 5.0 equiv), DCM (5.00 mL) at room temperature. The resulting mixture was stirred for 2 h at room temperature. The reaction was monitored by LCMS. The resulting mixture was concentrated under reduced pressure. The product was lyophilized to afford 2-amino-1-[(1R)-6,7-dichloro-1-methyl-1H,3H,4H,5H-pyrido[4,3-b]indol-2-yl]ethanone hydrochloride (Compound 51A*, 74.1 mg) as a light green solid. LCMS (ES, m/z)=311.9 [M+H]⁺. ¹H NMR (400 MHz, DMSO-d₆) δ 11.53 (d, J=11.5 Hz, 1H), 8.09 (s, 2H), 7.50 (m, J=20.1, 8.4 Hz, 1H), 7.20 (m, J=15.4, 8.4 Hz, 1H), 5.61-4.60 (m, 1H), 4.20-3.75 (m, 3H), 3.61-3.46 (m, 1H), 3.21-2.93 (m, 1H), 2.80 (m, J=16.3, 4.8 Hz, 1H), 1.48 (m, J=34.6, 6.5 Hz, 3H). Stereochemistry arbitrarily assigned.

Step 3B: Into a 50 mL round-bottom flask were added tert-butyl (S)-(2-(6,7-dichloro-1-methyl-1,3,4,5-tetrahydro-2H-pyrido[4,3-b]indol-2-yl)-2-oxoethyl)carbamate (1.0 eq, 260 mg, 0.62 mmol) and TFA (0.5 mL) in DCM (2 mL) at room temperature. The resulting mixture was stirred for 2 h at room temperature. The reaction was monitored by LCMS. The crude mixture was concentrated in vacuo, neutralized with DIPEA and purified by reverse-phase chromatography using a C18 column and a 20-60% MeCN/0.1% aqueous formic acid gradient to afford (S)-2-amino-1-(6,7-dichloro-1-methyl-1,3,4,5-tetrahydro-2H-pyrido[4,3-b]indol-2-yl)ethan-1-one (Compound 51B*) (24.4 mg, 65% yield) as a pale yellow solid. LCMS (ES, m/z)=312.2 [M+H]⁺. ¹HNMR (400 MHz, DMSO-d₆) δ 11.54 (d, J=10.5 Hz, 1H), 8.31-7.99 (m, 3H), 7.50 (m, J=17.6, 8.4 Hz, 1H), 7.20 (m, J=15.0, 8.4 Hz, 1H), 5.77-5.08 (m, 1H), 4.19-3.81 (m, 3H), 3.50 (t, J=12.0 Hz, 1H), 3.23-2.94 (m, 1H), 2.88-2.69 (m, 1H), 1.48 (m, J=34.9, 6.5 Hz, 3H). Stereochemistry arbitrarily assigned.

### Example 9: 1-(7,8-dichloro-1-methyl-3,4-dihydropyrazino[1,2-b]indazol-2(1H)-yl)-2-hydroxyethan-1-one (Compound 60, rac-60), (R)-1-(7,8-dichloro-1-methyl-3,4-dihydropyrazino[1,2-b]indazol-2(1H)-yl)-2-hydroxyethan-1-one (Compound 60A), and (S)-1-(7,8-dichloro-1-methyl-3,4-dihydropyrazino[1,2-b]indazol-2(1H)-yl)-2-hydroxyethan-1-one (Compound 60B)

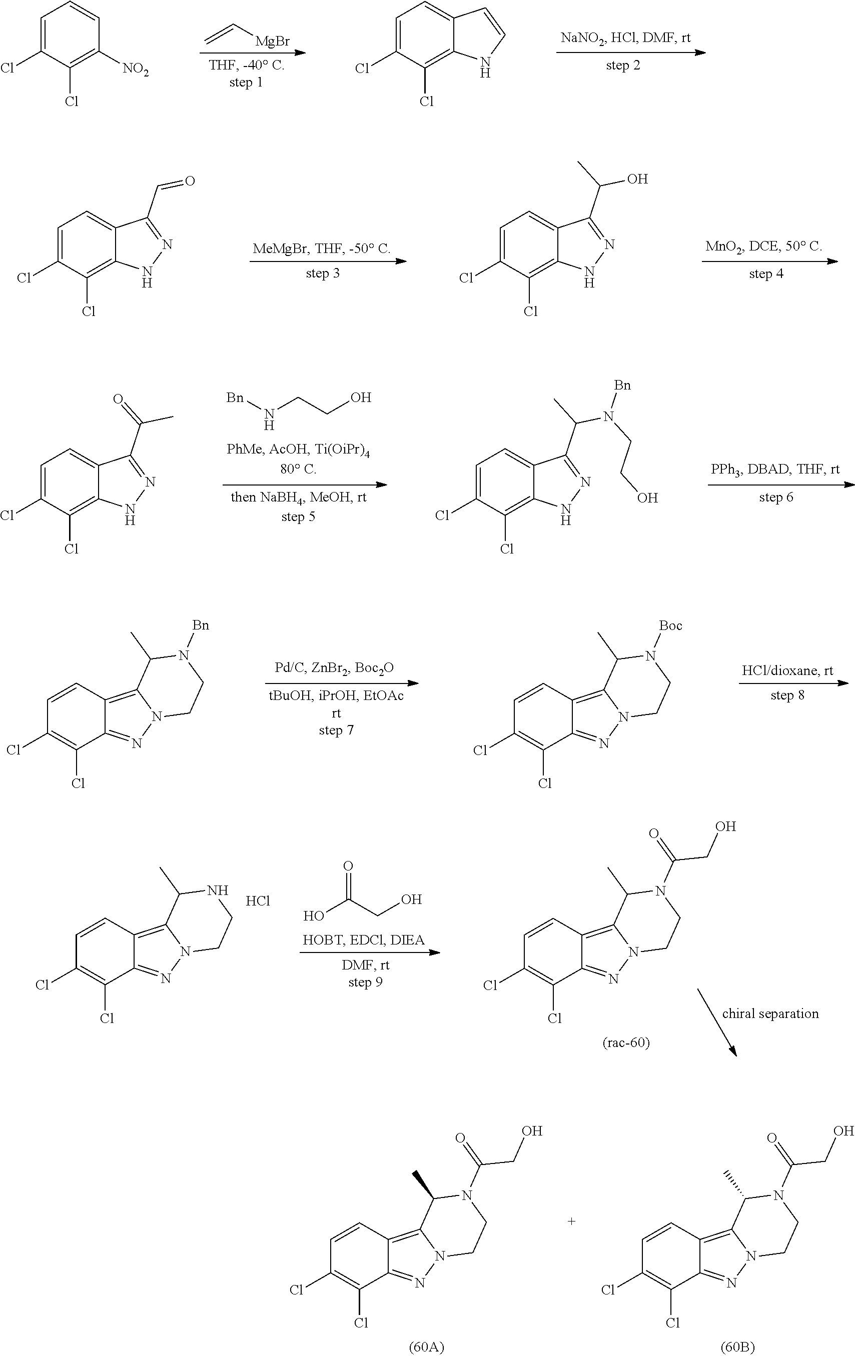

Step 1: To a stirred solution of 2,3-dichloronitrobenzene (1.00 eq, 10.0 g, 52.1 mmol) in THF (500 mL) was added bromo(ethenyl)magnesium 1 M (3.00 eq, 157 mL, 157 mmol) dropwise at −40° C. under nitrogen atmosphere. The resulting mixture was stirred for 40 min at −40° C. under nitrogen atmosphere. The reaction was quenched by the addition of sat. NH4Cl (aq.) (400 mL) at room temperature. The resulting mixture was extracted with EtOAc (3×500 mL), the combined organic layers were washed with brine (2×200 mL), dried over anhydrous Na₂SO₄. After filtration, the filtrate was concentrated under reduced pressure. The residue was purified by silica gel column chromatography, eluted with PE/EA (92:8) to afford 6,7-dichloro-1H-indole (5.5 g, 54%) as an off-white solid. LCMS (ES, m/z)=186 [M+H]⁺.

Step 2: Under nitrogen, to a stirred solution of NaNO₂ (8.04 eq, 16.4 g, 238 mmol) in H₂O (110 mL) and DMF (83 mL) were added HCl 2 M (2.71 eq, 40.0 mL, 80.0 mmol) dropwise at 0° C., the resulting mixture was stirred for 10 min at 0° C., and then to the above mixture solution was added 6,7-dichloro-1H-indole (1.0 eq, 5.5 g, 30 mmol) in DMF (83 mL) dropwise at 0° C. And then the resulting mixture was stirred for 12 h at room temperature. The resulting mixture was diluted with EtOAc (3.0 L), washed with water (3×300 mL) and brine (3×300 mL), dried over anhydrous Na₂SO₄. After filtration, the filtrate was concentrated under reduced pressure. The residue was purified by silica gel column chromatography, eluted with PE/EA (4:1) to afford 6,7-dichloro-1H-indazole-3-carbaldehyde (2.5 g, 40%) as a purple solid. LCMS (ES, m/z)=215 [M+H]⁺.

Step 3: Under nitrogen, to a stirred solution of 6,7-dichloro-1H-indazole-3-carbaldehyde (1.0 eq, 3.0 g, 14 mmol) in THF (90 mL) was added MeMgBr 1 M (3.0 eq, 42 mL, 42 mmol) dropwise at −50° C. The resulting mixture was stirred for 1 h at −50° C. under nitrogen atmosphere. The reaction was then quenched by the addition of sat. NH₄Cl (aq.) (400 mL) at 0° C., extracted with EtOAc (3×600 mL). The combined organic layers were washed with brine (2×200 mL), dried over anhydrous Na₂SO₄. After filtration, the filtrate was concentrated under reduced pressure to afford 1-(6,7-dichloro-1H-indazol-3-yl)ethanol (2.1 g, 62%) as a light yellow solid. LCMS (ES, m/z)=231 [M+H]⁺.

Step 4: To a solution of 1-(6,7-dichloro-1H-indazol-3-yl)ethanol (1.0 eq, 2.1 g, 9.1 mmol) in DCE (80 mL) was added MnO₂ (210 g) at room temperature, and then the resulting mixture was stirred for 1 h at 50° C. The resulting mixture was filtered, the filter cake was washed with MeOH (3×10 mL), and the filtration was concentrated under reduced pressure to afford 1-(6,7-dichloro-1H-indazol-3-yl)ethanone (1.8 g, 85%) as a white solid. LCMS (ES, m/z)=229 [M+H]⁺.

Step 5: To a solution of 1-(6,7-dichloro-1H-indazol-3-yl)ethanone (1.00 eq, 1.00 g, 4.37 mmol) in toluene (70 mL) was added 2-(benzylamino)ethanol (2.00 eq, 1.32 g, 8.73 mmol), AcOH (2.00 eq, 524 mg, 8.73 mmol) and Ti(O-iPr)₄ (1.53 eq, 1.90 g, 6.68 mmol) at room temperature, and then the resulting mixture was stirred for 5 h at 80° C. To the above mixture was then added NaBH₄ (3.00 eq, 495 mg, 13.1 mmol) in portions over 2 min at 0° C. And then the resulting mixture was stirred for another 12 h at room temperature. The reaction was quenched with sat. NH₄Cl (aq.)(50 ml) at 0° C., extracted with EtOAc (3×200 mL), the combined organic layers were dried over anhydrous Na₂SO₄. After filtration, the filtrate was concentrated under reduced pressure. The residue was purified by silica gel column chromatography, eluted with PE/EA (3:2) to afford 2-{benzyl[1-(6,7-dichloro-1H-indazol-3-yl)ethyl]amino}ethanol (600 mg, 37%) as an off-white oil. LCMS (ES, m/z)=364 [M+H]⁺.

Step 6: Under nitrogen, to a solution of 2-{benzyl[1-(6,7-dichloro-1H-indazol-3-yl)ethyl]amino}ethanol (1.00 eq, 600 mg, 1.65 mmol) in THF (6.0 mL) was added DBAD (3.00 eq, 1.14 g, 4.94 mmol) and PPh₃ (3.00 eq, 1.30 g, 4.94 mmol) at room temperature. The resulting mixture was stirred for 2 h at room temperature. The reaction was quenched by the addition of water (100 mL), extracted with EtOAc (3×200 mL), the combined organic layers were washed with brine (1×100 mL), dried over anhydrous Na₂SO₄. After filtration, the filtrate was concentrated under reduced pressure. The residue was purified by silica gel column chromatography, eluted with PE/EA (3:2) to afford 2-benzyl-7,8-dichloro-1-methyl-1H,3H,4H-pyrazino[1,2-b]indazole (410 mg, 70%) as a white solid. LCMS (ES, m/z)=346 [M+H]⁺.

Step 7: Under hydrogen, to a solution of 2-benzyl-7,8-dichloro-1-methyl-1H,3H,4H-pyrazino[1,2-b]indazole (1.00 eq, 300 mg, 0.866 mmol) in t-BuOH (7.0 mL), 2-Propanol (7.0 mL) and EA (7.0 mL) was added ZnBr₂ (3.00 eq, 195 mg, 0.866 mmol) and (Boc)₂O (79.3 eq, 15.0 g, 68.7 mmol) and Pd/C 10% (30 mg), and then the resulting mixture solution was stirred for 5 h at room temperature under hydrogen, and then the resulting solution was filtered through a Celite pad, and the filtrate was concentrated under reduced pressure. The residue was purified by silica gel column chromatography, eluted with PE/EA (4:1) to afford tert-butyl 7,8-dichloro-1-methyl-1H,3H,4H-pyrazino[1,2-b]indazole-2-carboxylate (110 mg, 35%) as a white solid. LCMS (ES, m/z)=356 [M+H]⁺.

Step 8: A solution of tert-butyl 7,8-dichloro-1-methyl-1H,3H,4H-pyrazino[1,2-b]indazole-2-carboxylate (1.00 eq, 110 mg, 0.309 mmol) in HCl(gas) in 1,4-dioxane (3.0 mL) was stirred for 1 h at room temperature. The resulting mixture was concentrated under vacuum to 7,8-dichloro-1-methyl-1,2,3,4-tetrahydropyrazino[1,2-b]indazole hydrochloride (110 mg) as a crude white solid. LCMS (ES, m/z)=256 [M+H]⁺.

Step 9: To a solution of glycolic acid (1.20 eq, 31.2 mg, 0.410 mmol) in DMF (2.0 mL) was added EDCI (3.01 eq, 197 mg, 1.03 mmol), HOBT (1.49 eq, 69.0 mg, 0.511 mmol), DIEA (3.01 eq, 133 mg, 1.03 mmol) and 7,8-dichloro-1-methyl-1H,2H,3H,4H-pyrazino[1,2-b]indazole hydrochloride (1.00 eq, 100 mg, 0.342 mmol) at room temperature. The resulting mixture was stirred for 5 h at 55° C. The reaction was quenched by the addition of water (5.0 mL) at room temperature. The resulting mixture was extracted with EtOAc (3×20 mL). The combined organic layers were washed with brine (lx 30 mL), dried over anhydrous Na₂SO₄. After filtration, the filtrate was concentrated under reduced pressure. The residue was purified by reverse flash chromatography eluting MeCN/water to afford 40 mg of 1-{7,8-dichloro-1-methyl-1H,3H,4H-pyrazino[1,2-b]indazol-2-yl}-2-hydroxyethanone) as a crude yellow oil. The crude product was purified by Prep-CHIRAL-HPLC using CHIRAL ART Cellulose-SB (2*25 cm, 5 m) and 50% Hexanes (0.2% TFA) in MeOH:EtOH (1:1) to afford both enantiomers, 1-[(1R)-7,8-dichloro-1-methyl-1H,3H,4H-pyrazino[1,2-b]indazol-2-yl]-2-hydroxyethanone (Compound 60A) (chiral RT(min)=10.17, 6.5 mg, 6% yield) as a white solid, and 1-[(1S)-7,8-dichloro-1-methyl-1H,3H,4H-pyrazino[1,2-b]indazol-2-yl]-2-hydroxyethanone (Compound 60B) (chiral RT(min)=8.08, 8.5 mg, 8% yield) as a white solid. Stereochemistry confirmed by X-ray crystallography.

Compound 60A: (LCMS (ES, m/z)=313.98 [M+H]⁺. ¹HNMR (400 MHz, DMSO-d₆) δ 7.88-7.78 (m, 1H), 7.19-7.17 (m, 1H), 6.04-5.72 (m, 1H), 4.83-4.51 (m, 2H), 4.33-4.18 (m, 3H), 3.79 (t, J=10.0 Hz, 2H), 1.61-1.52 (m, 3H).

Compound 60B: LCMS (ES, m/z)=313.98 [M+H]⁺. ¹HNMR (400 MHz, DMSO-d₆) δ 7.88-7.78 (m, 1H), 7.19-7.17 (m, 1H), 6.04-5.72 (m, 1H), 4.83-4.51 (m, 2H), 4.33-4.18 (m, 3H), 3.79 (t, J=10.0 Hz, 2H), 1.61-1.52 (m, 3H).

### Example 10: (7,8-dichloro-1-methyl-3,4-dihydropyrazino[1,2-b]indazol-2(1H)-yl)(5-methoxypyrimidin-2-yl)methanone (Compound 52, rac-52), (R)-(7,8-dichloro-1-methyl-3,4-dihydropyrazino[1,2-b]indazol-2(1H)yl)(5-methoxypyrimidin-2-yl)methanone (Compound 52A*), and (S)-(7,8-dichloro-1-methyl-3,4-dihydropyrazino[1,2-b]indazol-2(1H)yl)(5-methoxypyrimidin-2-yl)methanone (Compound 52B*)

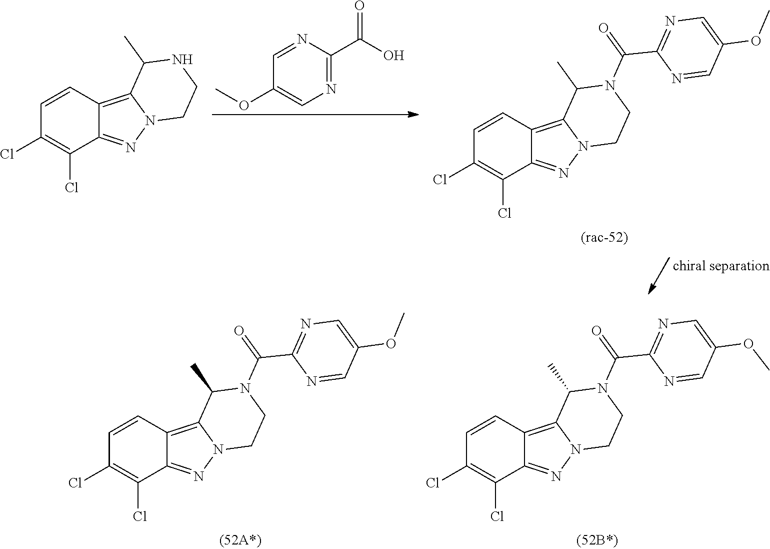

To a solution of 5-methoxypyrimidine-2-carboxylic acid (1.21 eq, 51.0 mg, 0.331 mmol) in DMF (2.0 mL) was added HOBt (2.00 eq, 74.0 mg, 0.548 mmol), EDCI (2.00 eq, 105 mg, 0.548 mmol), 7,8-dichloro-1-methyl-1,2,3,4-tetrahydropyrazino[1,2-b]indazole hydrochloride (Step 8 product, Example 9) (1.00 eq, 70.0 mg, 0.273 mmol) and DIEA (3.00 eq, 106 mg, 0.820 mmol) at room temperature. The resulting mixture was stirred for 1 h at room temperature. The reaction was quenched with water (20 mL), extracted with EtOAc (3×50 mL). The combined organic layers were washed with brine (lx 20 mL), dried over anhydrous Na₂SO₄. After filtration, the filtrate was concentrated under reduced pressure. The crude product was purified by Prep-HPLC using Xselect CSH C18 OBD Column (30*150 mm 5 m) and 34-66% of acetonitrile in water (0.05% TFA) to afford 2-{7,8-dichloro-1-methyl-1H,3H,4H-pyrazino[1,2-b]indazole-2-carbonyl}-5-methoxypyrimidine (20 mg) as a white solid, and then the product was further purified by Prep-CHIRAL-HPLC using CHIRAL ART Cellulose-SB (2*25 cm, 5 m) and 50% Hexanes (0.5% 2 M NH₃-MeOH) in MeOH:DCM (1:1) to afford (R)-(7,8-dichloro-1-methyl-3,4-dihydropyrazino[1,2-b]indazol-2(1H)-yl)(5-methoxypyrimidin-2-yl)methanone (Compound 52A*) (chiral RT(min)=7.23, 4.0 mg, 4% yield) as a white solid, and (S)-(7,8-dichloro-1-methyl-3,4-dihydropyrazino[1,2-b]indazol-2(1H)-yl)(5-methoxypyrimidin-2-yl)methanone (Compound 52B*) (chiral RT(min)=6.13, 4.2 mg, 4% yield) as a white solid. Stereochemistry arbitrarily assigned.

Compound 52A*: LCMS (ES, m/z)=392.00 [M+H]⁺. ¹H NMR (400 MHz, DMSO-d₆) δ 8.69 (s, 2H), 7.94-7.67 (m, 1H), 7.23-7.12 (m, 1H), 6.21-4.95 (m, 1H), 4.66-4.38 (m, 2H), 4.00 (s, 3H), 3.95-3.69 (m, 2H), 1.68-1.62 (m, 3H).

Compound 52B*: LCMS (ES, m/z)=392.00 [M+H]⁺. ¹H NMR (400 MHz, DMSO-d₆) δ 8.69 (s, 2H), 7.94-7.67 (m, 1H), 7.23-7.12 (m, 1H), 6.21-4.95 (m, 1H), 4.66-4.38 (m, 2H), 4.00 (s, 3H), 3.95-3.69 (m, 2H), 1.68-1.62 (m, 3H).

### Example 11: 1-(7,8-dichloro-1-methyl-10-(1-methyl-1H-pyrazol-3-yl)-3,4-dihydropyrazino[1,2-b]indazol-2(1H)-yl)-2-hydroxyethan-1-one (Compound 61, rac-61), (R)-1-(7,8-dichloro-1-methyl-10-(1-methyl-1H-pyrazol-3-yl)-3,4-dihydropyrazino[1,2-b]indazol-2(1H)-yl)-2-hydroxyethan-1-one (Compound 61A*) and (S)-1-(7,8-dichloro-1-methyl-10-(1-methyl-1H-pyrazol-3-yl)-3,4-dihydropyrazino[1,2-b]indazol-2(1H)-yl)-2-hydroxyethan-1-one (Compound 61B*)

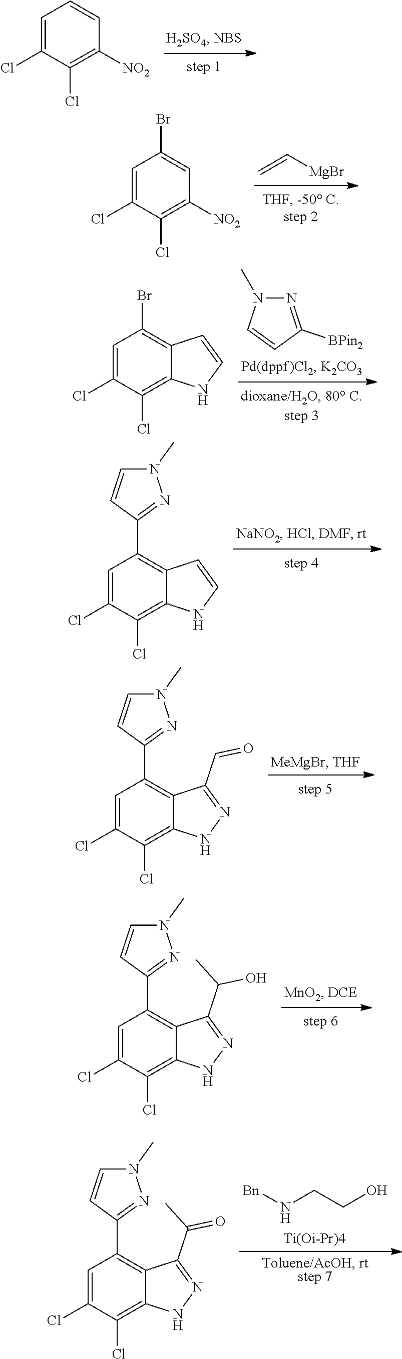

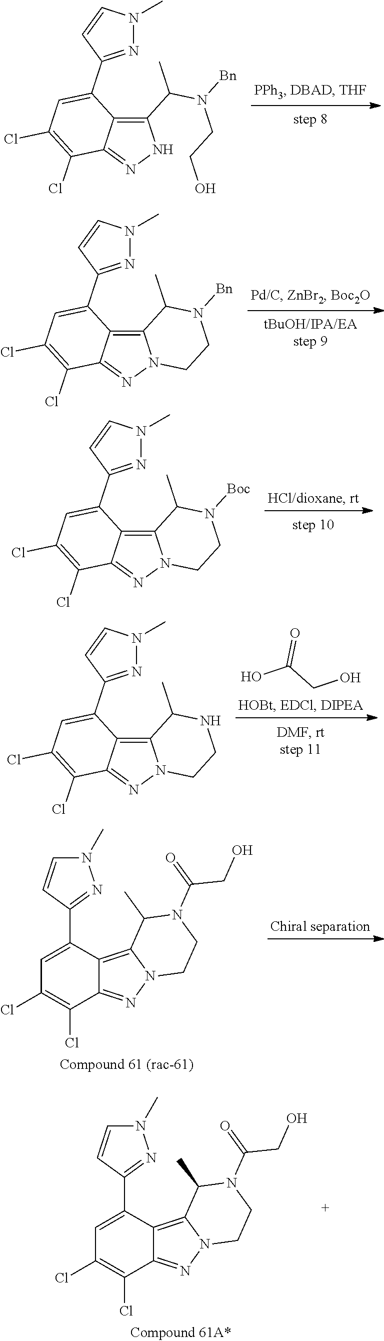

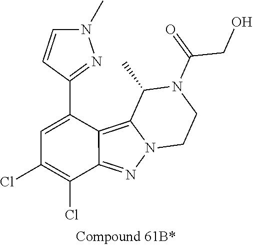 [SMILES: C[C@H]1c2c3c(-c4ccn(C)n4)cc(Cl)c(Cl)c3nn2CCN1C(=O)CO]

Step 1: Into a 500 mL round-bottom flask were added 2,3-dichloronitrobenzene (1.00 eq, 20.0 g, 104 mmol) and H₂SO₄ (27.0 eq, 200 mL, 2814 mmol) at room temperature. To the above mixture was added NBS (1.19 eq, 22.1 g, 124 mmol) dropwise at room temperature. The resulting mixture was stirred for overnight at 60° C. The reaction was added into water/ice (2000 mL) at room temperature. The resulting mixture was extracted with EtOAc (3×2000 mL) and dried over anhydrous Na₂SO₄. After filtration, the filtrate was concentrated under reduced pressure. The residue was purified by silica gel column chromatography, eluted with PE/EA (10:1) to afford 5-bromo-1,2-dichloro-3-nitrobenzene (24 g, 85% yield) as a light yellow oil.

Step 2: Into a 500 mL 3-necked round-bottom flask were added 5-bromo-1,2-dichloro-3-nitrobenzene (1.00 eq, 8.00 g, 29.5 mmol) and THF (100 mL) at room temperature. To the above mixture was added bromo(ethenyl)magnesium (7.00 eq, 207 mL, 207 mmol) dropwise over 2 h at −50° C. The resulting mixture was stirred for additional 1 h at −50° C. The reaction was quenched with sat. NH₄Cl (aq.) at 0° C. The aqueous layer was extracted with EtOAc (3×100 mL). The residue was purified by silica gel column chromatography, eluted with PE/EA (10:1) to afford 4-bromo-6,7-dichloro-1H-indole (2.0 g, 18% yield) as a dark yellow solid. LCMS (ES, m/z)=263.85 [M+H]⁻.

Step 3: Into a 40 mL vial were added 4-bromo-6,7-dichloro-1H-indole (1.00 eq, 1.50 g, 5.7 mmol), 1-methyl-3-(4,4,5,5-tetramethyl-1,3,2-dioxaborolan-2-yl)pyrazole (1.00 eq, 1178 mg, 5.66 mmol), dioxane (15 mL), Pd(dppf)Cl₂ (0.15 eq, 621 mg, 0.849 mmol), K₂CO₃ (3.00 eq, 2347 mg, 17.0 mmol) and H₂O (3.0 mL) at room temperature. The resulting mixture was stirred for 4 h at 80° C. under nitrogen atmosphere. The reaction was quenched by the addition of water (20 mL) at room temperature. The aqueous layer was extracted with EtOAc (3×100 mL). The resulting mixture was concentrated under reduced pressure. The residue was purified by silica gel column chromatography, eluted with PE/EA (5:1) to afford 6,7-dichloro-4-(1-methylpyrazol-3-yl)-1H-indole (1.0 g, 53% yield) as an off-white solid. LCMS (ES, m/z)=266.00 [M+H]⁻.

Step 4: Into a 100 mL 3-necked round-bottom flask were added NaNO₂ (8.00 eq, 6.22 g, 90.2 mmol) and DMF (30 mL) at room temperature. To the above mixture was added conc. HCl (2.76 eq, 15.2 mL, 30.4 mmol) dropwise at 0° C. The resulting mixture was stirred for additional 10 min at 0° C. To the above mixture was added 6,7-dichloro-4-(1-methylpyrazol-3-yl)-1H-indole (1.0 eq, 3.0 g, 11 mmol) dropwise over 10 min at 0° C. The resulting mixture was stirred for additional overnight at room temperature. The reaction was quenched with water at room temperature. The resulting mixture was extracted with EtOAc (3×30 mL). The combined organic layers were washed with water (3×30 mL). The filtrate was concentrated under reduced pressure. The residue was purified by silica gel column chromatography, eluted with CH₂Cl₂/MeOH (5:1) to afford 6,7-dichloro-4-(1-methylpyrazol-3-yl)-1H-indazole-3-carbaldehyde (1.0 g, 22% yield) as a brown yellow solid. LCMS (ES, m/z)=294.85 [M+H]⁻.

Step 5: Into a 100 mL 3-necked round-bottom flask were added 6,7-dichloro-4-(1-methylpyrazol-3-yl)-1H-indazole-3-carbaldehyde (1.0 eq, 1.2 g, 4.1 mmol) and THF (30 mL) at room temperature. To the above mixture was added MeMgBr (3.00 eq, 4.07 mL, 12.2 mmol) dropwise at −50° C. under nitrogen atmosphere. The resulting mixture was stirred for additional 1 h at −50° C. under nitrogen atmosphere. The reaction was quenched with sat. NH₄Cl (aq.) at 0° C. The aqueous layer was extracted with EtOAc (3×10 mL). The resulting mixture was concentrated under reduced pressure. The residue was purified by silica gel column chromatography, eluted with CH₂Cl₂/MeOH (5:1) to afford 1-[6,7-dichloro-4-(1-methylpyrazol-3-yl)-1H-indazol-3-yl]ethanol (770 mg, 49% yield) as a brown yellow solid. LCMS (ES, m/z)=310.90 [M+H]⁺.

Step 6: Into a 40 mL vial were added 1-[6,7-dichloro-4-(1-methylpyrazol-3-yl)-1H-indazol-3-yl]ethanol (1.00 eq, 770 mg, 2.48 mmol) and MnO₂ (56.6 eq, 7.70 g, 140.1 mmol) at room temperature. The resulting mixture was stirred for 1 h at 50° C. The solid was filtered out and the residue was washed with CH₂Cl₂ (3×10 mL). The resulting mixture was concentrated under reduced pressure. The residue was purified by silica gel column chromatography, eluted with PE/EA (1:1) to afford 1-[6,7-dichloro-4-(1-methylpyrazol-3-yl)-1H-indazol-3-yl]ethanone (700 mg, 82% yield) as a brown solid. LCMS (ES, m/z)=308.95 [M+H]⁺.

Step 7: Into a 40 mL vial were added 1-[6,7-dichloro-4-(1-methylpyrazol-3-yl)-1H-indazol-3-yl]ethanone (1.00 eq, 700 mg, 2.26 mmol), toluene (5 mL), 2-(benzylamino)ethanol (1.20 eq, 411 mg, 2.72 mmol), AcOH (2.00 eq, 272 mg, 4.53 mmol), and Ti(Oi-Pr)₄ (1.50 eq, 965 mg, 3.40 mmol) at room temperature. The resulting mixture was stirred for 5 h at 80° C. To the above mixture was added NaBH₄ (10.0 eq, 856 mg, 22.6 mmol) in portions at 0° C. The resulting mixture was stirred for additional overnight at room temperature. The reaction was quenched with water at 0° C. The aqueous layer was extracted with EtOAc (5×100 mL). The residue was purified by silica gel column chromatography, eluted with CH₂Cl₂/MeOH (5:1) to afford 2-[benzyl({1-[6,7-dichloro-4-(1-methylpyrazol-3-yl)-2H-indazol-3-yl]ethyl})amino]ethanol (600 mg, 54% yield) as an off-white solid. LCMS (ES, m/z)=444.05 [M+H]⁺.

Step 8: Into a 8 mL vial were added 2-[benzyl({1-[6,7-dichloro-4-(1-methylpyrazol-3-yl)-2H-indazol-3-yl]ethyl})amino]ethanol (1.00 eq, 700 mg, 1.58 mmol), DBAD (3.00 eq, 1.09 g, 4.72 mmol) and PPh₃ (3.00 eq, 1.24 g, 4.72 mmol) at room temperature. The resulting mixture was stirred for 1 h at room temperature. The reaction was quenched with water at room temperature. The resulting mixture was extracted with EtOAc (3×20 mL). The resulting mixture was concentrated under reduced pressure. The residue was purified by silica gel column chromatography, eluted with PE/EA (1:1) to afford 3-{2-benzyl-7,8-dichloro-1-methyl-1H,3H,4H-pyrazino[1,2-b]indazol-10-yl}-1-methylpyrazole (220 mg, 23% yield) as an off-white solid. LCMS (ES, m/z)=425.95 [M+H]⁺.

Step 9: Into a 25 mL round-bottom flask were added 3-{2-benzyl-7,8-dichloro-1-methyl-1H,3H,4H-pyrazino[1,2-b]indazol-10-yl}-1-methylpyrazole (1.0 eq., 90 mg, 0.21 mmol), EA (1.0 mL), 2-Propanol (1.0 mL), Boc₂O (19.5 eq, 900 mg, 4.12 mmol), ZnBr₂ (3.00 eq, 143 mg, 0.633 mmol) and Pd/C (8.01 eq, 180 mg, 1.69 mmol) at room temperature. The resulting mixture was stirred for 3 h at room temperature under hydrogen atmosphere. The residue was washed with MeOH (3×20 mL). The resulting mixture was concentrated under reduced pressure. The residue was purified by silica gel column chromatography, eluted with PE/EA (4:1) to afford tert-butyl 7,8-dichloro-1-methyl-10-(1-methylpyrazol-3-yl)-1H,3H,4H-pyrazino[1,2-b]indazole-2-carboxylate (30 mg, 20% yield) as a colorless solid. LCMS (ES, m/z)=436.00 [M+H]⁺.

Step 10: Into a 25 mL round-bottom flask were added tert-butyl 7,8-dichloro-1-methyl-10-(1-methylpyrazol-3-yl)-1H,3H,4H-pyrazino[1,2-b]indazole-2-carboxylate (1.0 eq, 90 mg, 0.21 mmol) and HCl(gas) in 1,4-dioxane (5.0 mL) at room temperature. The resulting mixture was stirred for 30 min at room temperature. The resulting mixture was concentrated under reduced pressure. The crude product was used in the next step directly without further purification. LCMS (ES, m/z)=335.90 [M+H]⁺.

Step 11: Into a 40 mL vial were added glycolic acid (1.20 eq, 24.4 mg, 0.322 mmol), DMF (1.0 mL), HOBT (1.50 eq, 54.3 mg, 0.402 mmol), EDCI (3.0 eq, 154 mg, 0.804 mmol), DIEA (3.00 eq, 104 mg, 0.804 mmol) and 3-{7,8-dichloro-1-methyl-1H,2H,3H,4H-pyrazino[1,2-b]indazol-10-yl}-1-methylpyrazole (1.0 eq, 90 mg, 0.27 mmol) at room temperature. The resulting mixture was stirred overnight at room temperature. The residue was purified by reverse flash chromatography with the following conditions: column, C18 silica gel; mobile phase, MeCN in water, 10% to 50% gradient in 10 min. The crude product (50 mg) was purified by Prep-HPLC with using Xselect CSH C18 OBD Column (30*150 mm 5 um) and 20-80% acetonitrile in water (0.05% TFA) as the gradient to afford 1-(7,8-dichloro-1-methyl-10-(1-methyl-1H-pyrazol-3-yl)-3,4-dihydropyrazino[1,2-b]indazol-2(1H)-yl)-2-hydroxyethan-1-one (Compound 61, rac-61) as a white solid comprising a mixture of enantiomers: (R)-1-(7,8-dichloro-1-methyl-10-(1-methyl-1H-pyrazol-3-yl)-3,4-dihydropyrazino[1,2-b]indazol-2(1H)-yl)-2-hydroxyethan-1-one (Compound 61A*) and (S)-1-(7,8-dichloro-1-methyl-10-(1-methyl-1H-pyrazol-3-yl)-3,4-dihydropyrazino[1,2-b]indazol-2(1H)-yl)-2-hydroxyethan-1-one (Compound 61B*), stereochemistry arbitrarily assigned. The mixture of enantiomers was separated by Chiral-Prep-HPLC using CHIRALPAK IG (2*25 cm, 5 um) and 50% (EtOH:DCM 1:1) in hexanes as the eluent to afford both enantiomers: Compound 61B* (chiral RT(min)=26.19, 3.3 mg, 3% yield) as a white solid, and Compound 61A* (chiral RT(min)=8.67, 3.4 mg, 3% yield) as a white solid.

Compound 61A*: LCMS (ES, m/z)=394.0 [M+H]⁺. ¹HNMR (400 MHz, DMSO-d₆) δ 7.88 (t, J=2.0 Hz, 1H), 7.34 (d, J=16.3 Hz, 1H), 6.87-6.44 (m, 2H), 5.01-4.74 (m, 1H), 4.60-4.56 (m, 2H) 4.35-4.13 (m, 3H), 4.02 (d, J=7.1 Hz, 3H), 3.80-3.51 (m, 1H), 1.03 (d, J=6.5 Hz, 1H), 0.86 (d, J=6.7 Hz, 2H).

Compound 61B*: LCMS (ES, m/z)=394.0 [M+H]⁺. ¹HNMR (400 MHz, DMSO-d₆) δ 7.88 (t, J=2.0 Hz, 1H), 7.34 (d, J=16.3 Hz, 1H), 6.87-6.44 (m, 2H), 5.01-4.74 (m, 1H), 4.60-4.56 (m, 2H) 4.35-4.13 (m, 3H), 4.02 (d, J=7.1 Hz, 3H), 3.80-3.51 (m, 1H), 1.03 (d, J=6.5 Hz, 1H), 0.86 (d, J=6.7 Hz, 2H).

### Example 12: 6,7-dichloro-1,3,4,5-tetrahydro-2H-pyrido[4,3-b]indole-2-carbohydrazide (Compound 59)

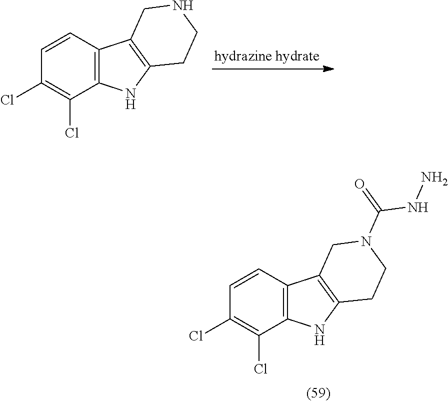 [SMILES: Clc1ccc2c3c([nH]c2c1Cl)CCNC3 ; NNC(=O)N1CCc2[nH]c3c(Cl)c(Cl)ccc3c2C1]

To a solution of 6,7-dichloro-2,3,4,5-tetrahydro-1H-pyrido[4,3-b]indole sulfuric acid (1:0.5) (Step 1 product of Example 1) (1.00 eq, 48.3 mg, 0.167 mmol) and N-ethyldiisopropylamine (2.2 eq, 65 μL, 0.37 mmol) in DMF (0.43 mL) was added carbonyldiimidazole (1.11 eq, 30.0 mg, 0.185 mmol) and the reaction mixture was stirred at room temperature for 3 h. To the reaction mixture was then added hydrazine hydrate (4.4 eq, 37 L, 0.74 mmol) and the mixture was stirred at 50° C. for 16 h. The crude mixture was concentrated in vacuo and purified by reverse-phase chromatography using a C18 column and a 10-60% MeCN/0.1% aqueous formic acid gradient to afford 6,7-dichloro-1,3,4,5-tetrahydro-2H-pyrido[4,3-b]indole-2-carbohydrazide (Compound 59) (21.3 mg, 36% yield) as a beige solid. LCMS (ES, m/z)=299.2 [M+H]⁺. ¹HNMR (400 MHz, DMSO-d₆) δ 11.43 (s, 1H), 8.00-7.61 (m, 2H), 7.38-7.31 (m, 1H), 7.21-7.12 (m, 1H), 4.53-4.48 (m, 2H), 3.76-3.60 (m, 3H), 2.78 (s, 2H).

### Example 13: (6,7-dichloro-9-(1-methyl-1H-pyrazol-3-yl)-1,3,4,5-tetrahydro-2H-pyrido[4,3-b]indol-2-yl)(5-methoxypyrimidin-2-yl)methanone (Compound 71)

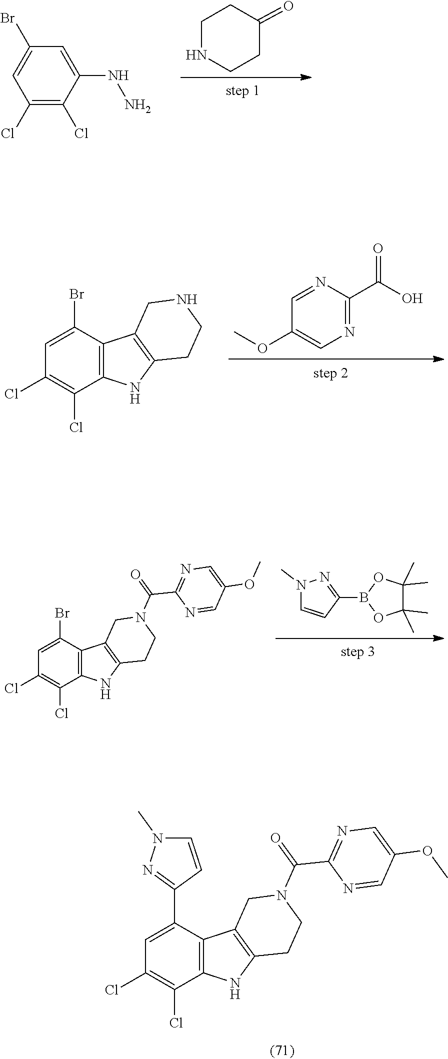

Step 1: A solution of piperidin-4-one hydrochloride (1.00 eq, 1.17 g, 8.60 mmol) and (5-bromo-2,3-dichloro-phenyl)hydrazine (1 eq, 2.20 g, 8.60 mmol) in 1,4-dioxane (28 mL) was treated with sulfuric acid (4.0 eq, 1.9 mL, 34 mmol) and stirred at 100° C. overnight. The crude mixture was cooled down to room temperature, diluted with EtOAc (40 mL), filtered, rinsed with 40% EtOH in EtOAc (30 mL) and dried in vacuo to give 2.570 g of a pale brown solid as the crude mixture. The residue was purified by automated normal phase flash chromatography using 0-100% (2% NH₄OH, 18% MeOH in DCM)/DCM as the gradient to afford 9-bromo-6,7-dichloro-2,3,4,5-tetrahydro-1H-pyrido[4,3-b]indole (1.14 g, 42% yield) as a beige solid. LCMS (ES, m/z)=320.7 [M+H]⁺. ¹HNMR (400 MHz, DMSO-d₆) δ 11.63 (s, 1H), 735 (s, 111), 4.12-4.08 (i, 2H), 3.01-2.93 (m, 2HD, 2.71-2.63 (m, 211), 2.41 (s, 1H).

Step 2: To 9-bromo-6,7-dichloro-2,3,4,5-tetrahydro-1H-pyrido[4,3-b]indole (1.0 eq, 634 mg, 1.98 mmol) in DMF (9.0 mL) was added 5-methoxypyrimidine-2-carboxylic acid (1.20 eq, 366 mg, 2.38 mmol) and diisopropylethylamine (4.0 eq, 1.4 mL, 7.9 mmol). The solution was cooled down to 0° C. and O-(7-azabenzotriazol-1-yl)-N,N,N,N-tetramethyluronium hexafluorophosphate (1.20 eq, 932 mg, 2.38 mmol) was added in one portion. The reaction mixture was stirred at room temperature overnight. The crude mixture was concentrated in vacuo and purified by automated normal phase flash chromatography using 0-40% (10% MeOH in DCM)/DCM as the gradient to afford (9-bromo-6,7-dichloro-1,3,4,5-tetrahydropyrido[4,3-b]indol-2-yl)-(5-methoxypyrimidin-2-yl)methanone (638 mg, 71% yield) as a beige solid. LCMS (ES, m/z)=455.0 [M+H]⁺. ¹HNMR (400 MHz, DMSO-d₆) δ 11.96-11.87 (m, 1H), 8.69-8.62 (m, 1H), 8.16 (s, 1H), 7.50-7.29 (m, 1H), 5.13-4.65 (m, 1H), 4.00-3.94 (m, 2H), 4.07-3.48 (m, 1H), 3.70-3.56 (m, 2H), 3.20-3.08 (m, 2H), 2.97-2.80 (m, 1H).

Step 3: (9-bromo-6,7-dichloro-1,3,4,5-tetrahydropyrido[4,3-b]indol-2-yl)-(5-methoxypyrimidin-2-yl)methanone (1.0 eq, 35 mg, 0.058 mmol), XPhos Pd G3 (0.050 eq, 2.4 mg, 0.0029 mmol), potassium phosphate (2.5 eq, 31 mg, 0.14 mmol) and 4,4,5,5-tetramethyl-2-(1-methylpyrazol-3-yl)-1,3,2-dioxaborolane (1.3 eq, 16 mg, 0.075 mmol) were combined in 1,4-dioxane (0.31 mL) and water (0.075 mL). The reaction mixture was sparged with nitrogen for 3 min and stirred at 70° C. for 1 h. The crude mixture was then diluted with DCM, filtered, concentrated in vacuo and purified reverse-phase chromatography using a C18 column and a 20-75% MeCN/0.1% aqueous formic acid gradient to afford [6,7-dichloro-9-(1-methylpyrazol-3-yl)-1,3,4,5-tetrahydropyrido[4,3-b]indol-2-yl]-(5-methoxypyrimidin-2-yl)methanone (Compound 71) (12.0 mg, 38% yield) as a pale brown solid. LCMS (ES, m/z)=457.2 [M+H]⁺. ¹HNMR (400 MHz, DMSO-d₆) δ 11.67-11.59 (m, 1H), 8.69-8.58 (m, 2H), 7.86-7.60 (m, 1H), 7.29-7.14 (m, 1H), 6.64-6.34 (m, 1H), 4.79-4.24 (m, 2H), 4.05-3.94 (m, 6H), 3.63 (s, 1H), 3.49 (t, J=5.6 Hz, 1H), 2.99-2.82 (m, 2H).

### Example 14: (6,7-dichloro-1-methyl-1,3,4,5-tetrahydro-2H-pyrido[4,3-b]indol-2-yl)(5-(2-hydroxyethoxy)pyrimidin-2-yl)methanone (Compound 77, rac-77), (R)-(6,7-dichloro-1-methyl-1,3,4,5-tetrahydro-2H-pyrido[4,3-b]indol-2-yl)(5-(2-hydroxyethoxy)pyrimidin-2-yl)methanone (Compound 77A*) and (S)-(6,7-dichloro-1-methyl-1,3,4,5-tetrahydro-2H-pyrido[4,3-b]indol-2-yl)(5-(2-hydroxyethoxy)pyrimidin-2-yl)methanone (Compound 77B*)

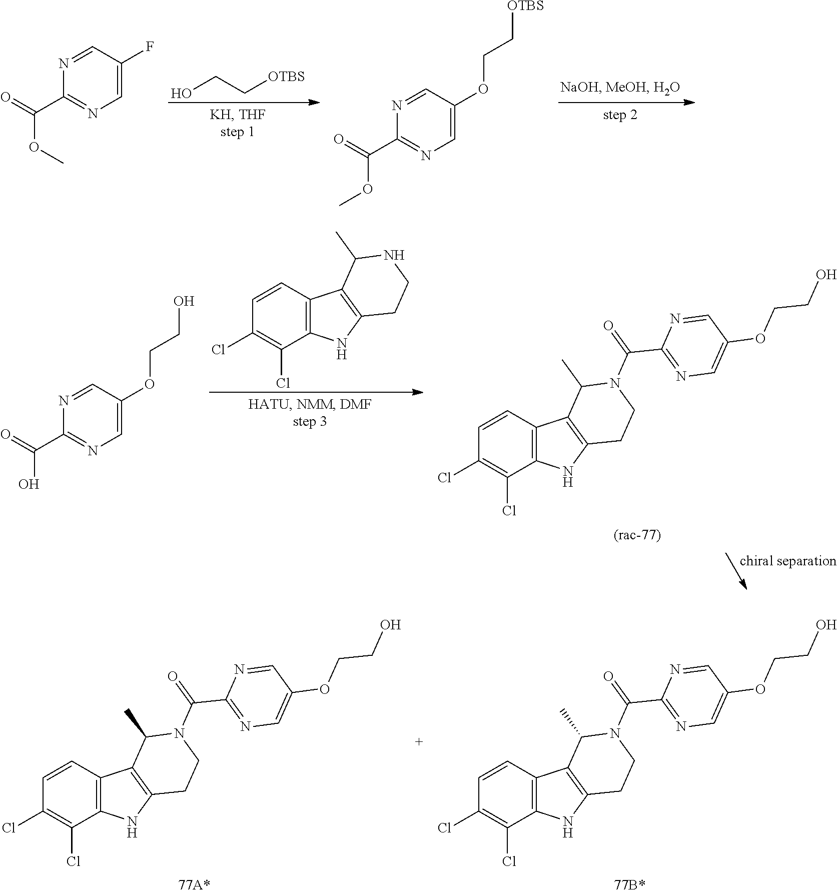

Step 1: Into a 25 mL vial were added methyl 5-fluoropyrimidine-2-carboxylate (400 mg, 2.56 mmol, 1.00 equiv), 2-[(tert-butyldimethylsilyl)oxy]ethanol (497 mg, 2.82 mmol, 1.1 equiv), potassium hydride (KH) (400 mg, 10.2 mmol, 4.00 equiv) and THF (5 mL) at room temperature. The resulting mixture was stirred for 2.5 h at room temperature. The reaction was monitored by LCMS. The resulting mixture was diluted with water and extracted with ethyl acetate (3×50 mL). The combined organic layers were dried over anhydrous MgSO₄. After filtration, the filtrate was concentrated under reduced pressure. The residue was purified by silica gel column chromatography, eluted with petroleum ether/ethyl acetate (1:1) to afford methyl 5-{2-[(tert-butyldimethylsilyl)oxy]ethoxy}pyrimidine-2-carboxylate (200 mg, 25% yield) as a white solid. LCMS (ES, m/z)=313.0 [M+1]⁺.

Step 2: Into a 25 mL vial were added methyl 5-{2-[(tert-butyldimethylsilyl)oxy]ethoxy}pyrimidine-2-carboxylate (110 mg, 0.35 mmol, 1.00 equiv), NaOH (101 mg, 2.52 mmol, 5 equiv), MeOH (5 mL) and H₂O (5 mL) at room temperature. The resulting mixture was stirred for 2.5 h at room temperature. The reaction was monitored by LCMS. The resulting mixture was diluted with water and extracted with ethyl acetate (3×50 mL). The pH value of the aqueous layer was adjusted to 7.0 with HCl (aq.) and concentrated under reduced pressure. The residue was purified by reverse flash chromatography with ACN/H₂O (10/90, v/v) to afford 5-(2-hydroxyethoxy)pyrimidine-2-carboxylic acid (54.0 mg, 48% yield) as a white solid. LCMS (ES, m/z)=185.0 [M+1]⁺.

Step 3: Into a 25 mL vial were added 5-(2-hydroxyethoxy)pyrimidine-2-carboxylic acid (40.0 mg, 0.22 mmol, 1 equiv), 6,7-dichloro-1-methyl-1H,2H,3H,4H,5H-pyrido[4,3-b]indole (Step 1 product, Example 6) (66.5 mg, 0.26 mmol, 1.20 equiv), NMM (65.9 mg, 0.65 mmol, 3.00 equiv), HATU (124 mg, 0.33 mmol, 1.50 equiv) and DMF (5 mL) at 0° C. under N₂. The resulting mixture was stirred for 16 h at room temperature under N₂. The reaction was monitored by LCMS. The resulting mixture was diluted with water and extracted with ethyl acetate (3×50 mL). The combined organic layers were dried over anhydrous MgSO₄. After filtration, the filtrate was concentrated under reduced pressure. The residue was purified by reverse flash chromatography with ACN/H₂O (50/50, v/v) to afford (6,7-dichloro-1-methyl-1,3,4,5-tetrahydro-2H-pyrido[4,3-b]indol-2-yl)(5-(2-hydroxyethoxy)pyrimidin-2-yl)methanone (Compound 77, rac-77) (17.2 mg, 19% yield) as a white solid, comprising a mixture of enantiomers: (R)-(6,7-dichloro-1-methyl-1,3,4,5-tetrahydro-2H-pyrido[4,3-b]indol-2-yl)(5-(2-hydroxyethoxy)pyrimidin-2-yl)methanone (Compound 77A*) and (S)-(6,7-dichloro-1-methyl-1,3,4,5-tetrahydro-2H-pyrido[4,3-b]indol-2-yl)(5-(2-hydroxyethoxy)pyrimidin-2-yl)methanone (Compound 77B*), stereochemistry arbitrarily assigned. LCMS (ES, m/z)=421.0 [M+1]⁺. This racemic mixture was separated by Prep-Chiral-HPLC (CHIRAL ART Cellulose-SB, 2*25 cm, 5 m; Mobile Phase A: Hexanes (0.5% 2M NH₃-MeOH), Mobile Phase B: MeOH:DCM=1:1; Flow rate: 20 mL/min; Gradient: 30% B to 30% B in 11 min; Wave Length: 220/254 nm) to afford Compound 77A* (chiral RT(min)=10.46, (4.3 mg, assumed, 5% yield) as a white solid, and Compound 77B* (chiral RT(min)=8.77, 12.5 mg, assumed, 14% yield) as a white solid.

Compound 77A*: LCMS (ES, m/z)=421.0 [M+1]⁺. ¹H NMR (400 MHz, DMSO-d₆) δ 11.54-11.50 (m, 1H), 8.65 (d, J=13.6 Hz, 2H), 7.55 (d, J=8.4 Hz, 1H), 7.31-7.08 (m, 1H), 5.77 (d, J=6.4 Hz, 1H), 4.99 (s, 1H), 4.81-4.68 (m, 1H), 4.26 (s, 2H), 3.78 (s, 2H), 3.50 (d, J=7.2 Hz, 1H), 2.89-2.69 (m, 2H), 1.54-1.47 (m, 3H).

Compound 77B*: LCMS (ES, m/z)=421.0 [M+1]⁺. ¹H NMR (400 MHz, DMSO-d₆) δ 11.54-11.50 (m, 1H), 8.65 (d, J=13.6 Hz, 2H), 7.55 (d, J=8.4 Hz, 1H), 7.31-7.08 (m, 1H), 5.79-5.76 (m, 1H), 5.01-4.97 (m, 1H), 4.81-4.68 (m, 1H), 4.27-4.23 (m, 2H), 3.80-3.76 (m, 2H), 3.50 (d, J=7.2 Hz, 1H), 2.90-2.69 (m, 2H), 1.55-1.46 (m, 3H).

### Example 15: (6,7-dichloro-1-methyl-1,3,4,5-tetrahydro-2H-pyrido[4,3-b]indol-2-yl)(4,5-dimethoxypyrimidin-2-yl)methanone (Compound 83, rac-83), (R)-(6,7-dichloro-1-methyl-1,3,4,5-tetrahydro-2H-pyrido[4,3-b]indol-2-yl)(4,5-dimethoxypyrimidin-2-yl)methanone (Compound 83A*), and (S)-(6,7-dichloro-1-methyl-1,3,4,5-tetrahydro-2H-pyrido[4,3-b]indol-2-yl)(4,5-dimethoxypyrimidin-2-yl)methanone (Compound 83B*)

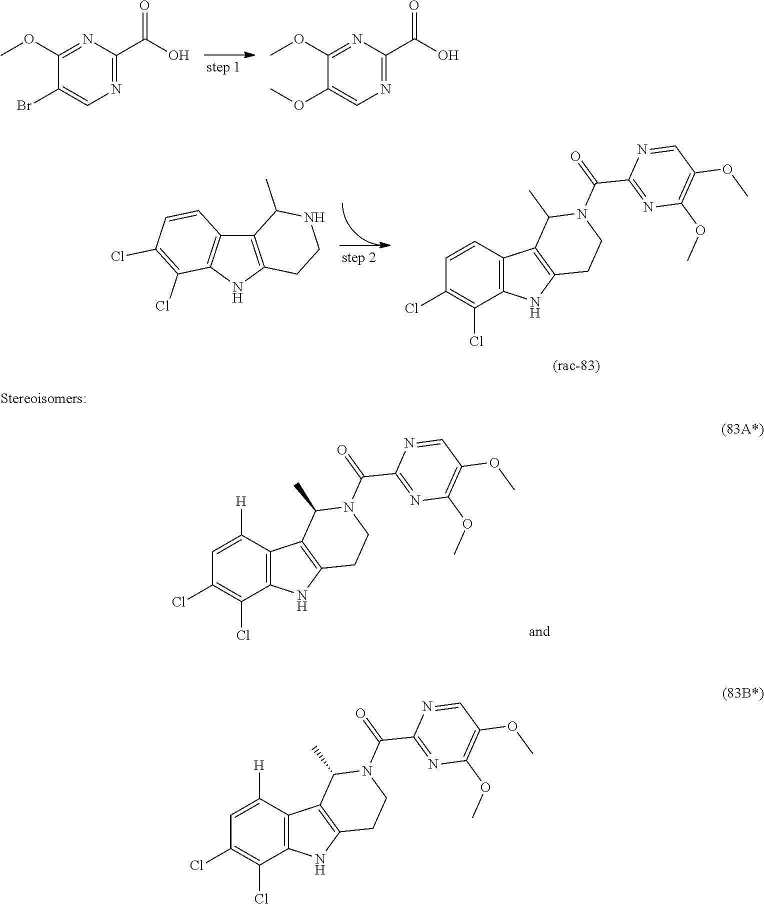 [SMILES: COc1nc(C(=O)O)ncc1Br ; COc1cnc(C(=O)O)nc1OC ; CC1NCCc2[nH]c3c(Cl)c(Cl)ccc3c21 ; COc1cnc(C(=O)N2CCc3[nH]c4c(Cl)c(Cl)ccc4c3C2C)nc1OC ; COc1cnc(C(=O)N2CCc3[nH]c4c(Cl)c(Cl)ccc4c3[C@H]2C)nc1OC ; COc1cnc(C(=O)N2CCc3[nH]c4c(Cl)c(Cl)ccc4c3[C@@H]2C)nc1OC]

Step 1: Into a 20 mL vial were added 5-bromo-4-methoxypyrimidine-2-carboxylic acid (70.0 mg, 0.30 mmol, 1 equiv), cuprous chloride (14.9 mg, 0.15 mmol, 0.5 equiv), methanol (6 mL) and sodium methoxide (81.1 mg, 1.50 mmol, 5 equiv) at room temperature. The mixture was stirred for 5 days at 80° C. under nitrogen atmosphere. The reaction was monitored by LCMS. The resulting mixture was filtered, the filtrate was concentrated under reduced pressure. The residue was purified by reverse flash chromatography (C18; mobile phase, acetonitrile in water, 13% to 15% gradient in 10 min; detector, UV 254 nm) to afford 4,5-dimethoxypyrimidine-2-carboxylic acid (20 mg, 24% yield) as a yellow solid. LCMS (ES, m/z)=185.0 [M+H]⁺.

Step 2: Into a 10 mL vial were added 4,5-dimethoxypyrimidine-2-carboxylic acid (70 mg, 0.38 mmol, 1 equiv) and thionyl chloride (2 mL) at room temperature. The mixture was stirred for 1 h at 80° C. and was concentrated under reduced pressure. Into above mixture were added 6,7-dichloro-1-methyl-1H,2H,3H,4H,5H-pyrido[4,3-b]indole (Step 1 product, Example 6) (116 mg, 0.45 mmol, 1.20 equiv), TEA (115 mg, 1.14 mmol, 3 equiv) and tetrahydrofuran (2 mL) at 0° C. and was stirred for 1 h at 0° C. The reaction was monitored by LCMS. The reaction was quenched with water (20 mL) at room temperature and was concentrated under reduced pressure. The residue was purified by reversed-phase flash chromatography (C18; mobile phase, acetonitrile in water, 30% to 32% gradient in 4 min; detector, UV 254 nm) to afford (6,7-dichloro-1-methyl-1,3,4,5-tetrahydro-2H-pyrido[4,3-b]indol-2-yl)(4,5-dimethoxypyrimidin-2-yl)methanone (Compound 83, rac-83) (10.0 mg) as a white solid, comprising a mixture of enantiomers (R)-(6,7-dichloro-1-methyl-1,3,4,5-tetrahydro-2H-pyrido[4,3-b]indol-2-yl)(4,5-dimethoxypyrimidin-2-yl)methanone (Compound 83A*) and (S)-(6,7-dichloro-1-methyl-1,3,4,5-tetrahydro-2H-pyrido[4,3-b]indol-2-yl)(4,5-dimethoxypyrimidin-2-yl)methanone (Compound 83B*), stereochemistry arbitrarily assigned. The crude product was further purified by Prep-HPLC (Xselect CSH F-Phenyl OBD column, 19*250 mm, 5 μm; Mobile Phase A: Water (0.05% TFA), Mobile Phase B: MeOH—HPLC; Flow rate: 20 mL/min; Gradient: 67% B to 72% B in 8 min, 72% B; Wave Length: 254 nm; RT(min): 11) to afford Compound 83 (rac-83) (1.90 mg, 1.2% yield) as a white solid. LCMS (ES, m/z)=421.0 [M+H]⁺. ¹H NMR (400 MHz, Methanol-d₄) δ 8.23 (s, 1H), 7.19 (s, 1H), 7.07 (s, 1H), 5.84 (s, 1H), 4.59 (s, 1H), 4.15-4.07 (d, J=3.2 Hz, 2H), 3.83 (s, 1H), 3.68-3.57 (t, J=4.4 Hz, 3H), 3.86 (s, 1H), 3.65 (s, 1H), 3.50 (s, 1H), 3.14 (s, 1H), 2.96-2.75 (t, J=9.8 Hz, 3H), 1.70-1.66 (t, J=6.5 Hz, 3H).

### Example 16: (6,7-dichloro-1-methyl-1,3,4,5-tetrahydro-2H-pyrido[4,3-b]indol-2-yl)(4-(2-(dimethylamino)ethoxy)pyrimidin-2-yl)methanone (Compound 88, rac-88), (R)-(6,7-dichloro-1-methyl-1,3,4,5-tetrahydro-2H-pyrido[4,3-b]indol-2-yl)(4-(2-(dimethylamino)ethoxy)pyrimidin-2-yl)methanone (Compound 88A*), and (S)-(6,7-dichloro-1-methyl-1,3,4,5-tetrahydro-2H-pyrido[4,3-b]indol-2-yl)(4-(2-(dimethylamino)ethoxy)pyrimidin-2-yl)methanone (Compound 88B*)

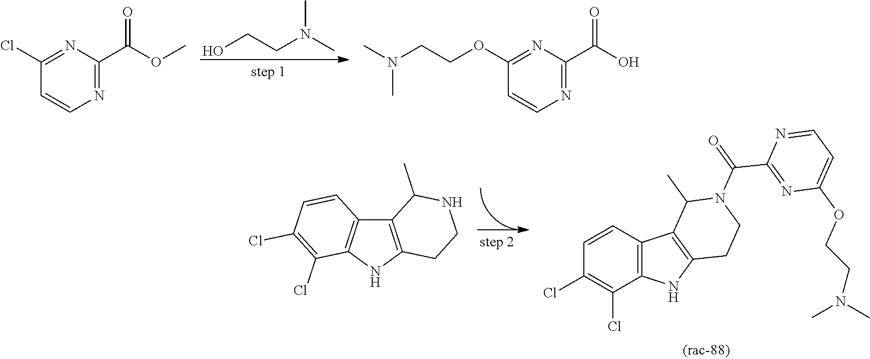

### Stereoisomers:

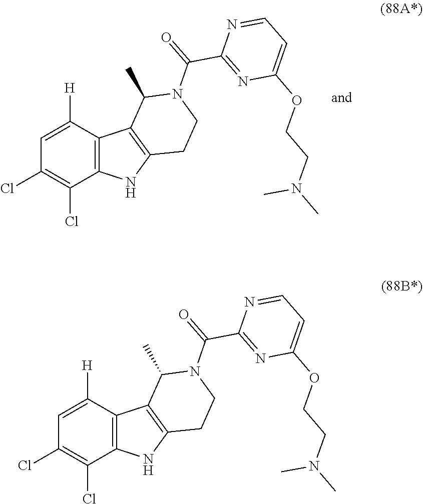 [SMILES: C[C@@H]1c2c([nH]c3c(Cl)c(Cl)ccc23)CCN1C(=O)c1nccc(OCCN(C)C)n1 ; C[C@H]1c2c([nH]c3c(Cl)c(Cl)ccc23)CCN1C(=O)c1nccc(OCCN(C)C)n1]

Step 1: To a solution of dimethylaminoethanol (103 mg, 1.16 mmol, 10 equiv) in THF was added sodium hydride (60% in oil, 40 mg) at 0° C. The mixture was stirred for 60 min. Methyl 4-chloropyrimidine-2-carboxylate (20.0 mg, 0.12 mmol, 1.00 equiv) was added and the mixture was allowed to warm to RT and stirred for 1 h. The reaction was monitored by LCMS. The reaction mixture was quenched by HCl (1M). The mixture was acidified to pH ˜4 with HCl (1 M). The residue was purified by trituration with CH₂Cl₂/MeOH (5×10 mL). The resulting mixture was concentrated under reduced pressure. This resulted in 4-[2-(dimethylamino)ethoxy]pyrimidine-2-carboxylic acid (30 mg) as a yellow oil. LCMS (ES, m/z)=212.0 [M+1]⁺.

Step 2: Into a 8 mL vial were added 4-[2-(dimethylamino)ethoxy]pyrimidine-2-carboxylic acid (30 mg, 0.14 mmol, 1 equiv) and thionyl chloride (0.50 mL). The resulting mixture was stirred for 1.5 h at 80° C. under air atmosphere. The reaction was monitored by LCMS. The resulting mixture was concentrated under reduced pressure. Into the mixture were added 6,7-dichloro-1-methyl-1H,2H,3H,4H,5H-pyrido[4,3-b]indole (Step 1 product, Example 6) (10.9 mg, 0.040 mmol, 0.30 equiv), THF (1.0 mL) and TEA (80.1 mg, 0.79 mmol, 5.6 equiv) at 0° C. The resulting mixture was stirred for 2 h at room temperature under air atmosphere. The reaction was monitored by LCMS. The resulting mixture was concentrated under reduced pressure. The residue was purified by reverse flash chromatography with the following conditions: (ACN:H₂O=1:4). The resulting mixture was concentrated under reduced pressure to provide (6,7-dichloro-1-methyl-1,3,4,5-tetrahydro-2H-pyrido[4,3-b]indol-2-yl)(4-(2-(dimethylamino)ethoxy)pyrimidin-2-yl)methanone (Compound 88, rac-88) (20 mg) as a mixture of enantiomers (R)-(6,7-dichloro-1-methyl-1,3,4,5-tetrahydro-2H-pyrido[4,3-b]indol-2-yl)(4-(2-(dimethylamino)ethoxy)pyrimidin-2-yl)methanone (Compound 88A*) and (S)-(6,7-dichloro-1-methyl-1,3,4,5-tetrahydro-2H-pyrido[4,3-b]indol-2-yl)(4-(2-(dimethylamino)ethoxy)pyrimidin-2-yl)methanone (Compound 88B*), stereochemistry arbitrarily assigned. The crude residue was further purified by Prep-HPLC (C18 silica gel; mobile phase, MeCN in water (10 mmol/L NH₄HCO₃), 10% to 30% gradient in 10 min; detector, UV 254 nm) to provide Compound 88 (rac-88) (2.4 mg, 4% yield) as a light yellow solid. LCMS (ES, m/z)=448.0 [M+1]⁺. ¹H NMR (400 MHz, DMSO-d₆) δ 11.53 (d, J=12.8 Hz, 1H), 8.67-8.56 (m, 1H), 7.60-7.32 (m, 1H), 7.23-7.07 (m, 1H), 7.07-7.00 (m, 1H), 5.80-4.69 (m, 1H), 4.53-4.35 (m, 2H), 3.52 (q, J=3.7 Hz, 2H), 2.96-2.84 (m, 2H), 2.81-2.70 (m, 2H), 2.28 (d, J=34.8 Hz, 6H), 1.52 (t, J=6.3 Hz, 3H).

### Example 17: 1-[6,7-dichloro-1-methyl-1H,3H,4H,5H-pyrido[4,3-b]indole-2-carbonyl]-3H-imidazole-4-carbonitrile (Compound 95, rac-95), 1-[(1R)-6,7-dichloro-1-methyl-1H,3H,4H,5H-pyrido[4,3-b]indole-2-carbonyl]-3H-imidazole-4-carbonitrile (Compound 95A*) and 1-[(1S)-6,7-dichloro-1-methyl-1H,3H,4H,5H-pyrido[4,3-b]indole-2-carbonyl]-3H-imidazole-4-carbonitrile (Compound 95B*)

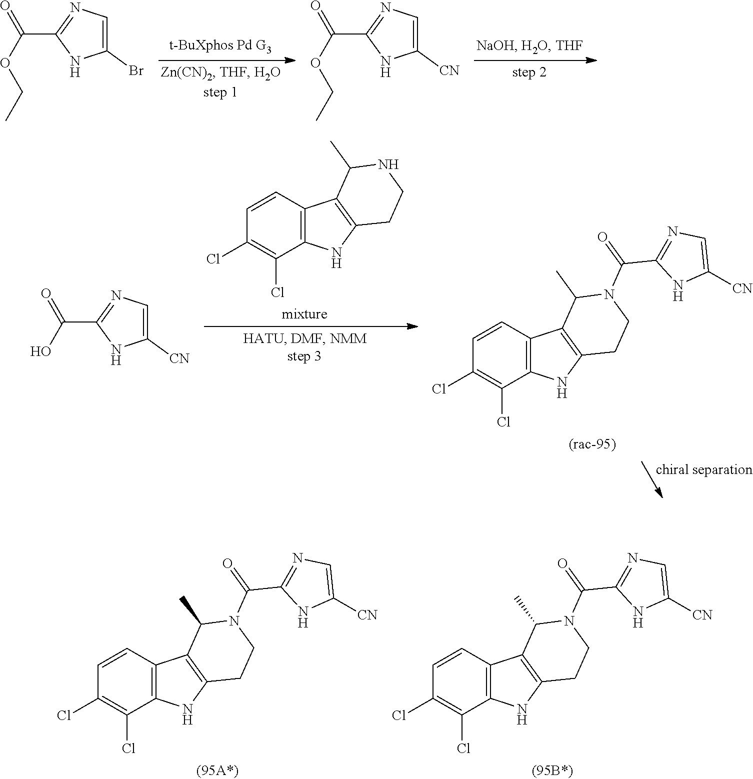

Step 1: To a mixture of ethyl 5-bromo-1H-imidazole-2-carboxylate (2 g, 9.13 mmol) and Zn(CN)₂ (3.20 g, 27.4 mmol) in H₂O/THF (4 mL/20 mL) were added t-BuXPhos Pd G3 (1.6 mg, 0.010 mmol) at room temperature under N₂. The resulting mixture was stirred at 80° C. for 4 h under N₂. After the reaction was completed, the resulting mixture was diluted with H₂O and extracted with ethyl acetate. The combined organic layer was washed with brine, dried over anhydrous sodium sulfate and filtered. The filtrate was concentrated under vacuum. The residue was purified by flash column chromatography with PE/EA (1/1, v/v) to afford ethyl 4-cyano-3H-imidazole-2-carboxylate (200 mg, 13% yield) as a white solid. LCMS (ES, m/z)=166.1 [M+1]*.

Step 2: To a mixture of ethyl 4-cyano-3H-imidazole-2-carboxylate (400 mg, 2.42 mmol) in H₂O/THF (4 mL/6 mL) were added NaOH (485 mg, 12.1 mmol) at room temperature. The resulting mixture was stirred at room temperature for 16 h. After the reaction was completed, the resulting mixture was diluted with water. The pH value of the mixture was adjusted to 7.0 with HCl (aq.). The mixture was extracted with ethyl acetate. The combined organic layer was washed with brine, dried over anhydrous sodium sulfate and filtered. The filtrate was concentrated under reduced pressure. The residue was purified by flash column chromatography with petroleum ether/ethyl acetate (1/1, v/v) to afford 4-cyano-3H-imidazole-2-carboxylic acid (150 mg, 45%) as a white solid. LCMS (ES, m/z)=138.0 [M+H]⁺.

Step 3: To a mixture of 4-cyano-3H-imidazole-2-carboxylic acid (80.0 mg, 0.58 mmol) and 6,7-dichloro-1-methyl-1H,2H,3H,4H,5H-pyrido[4,3-b]indole (Step 1 product, Example 6) (178.6 mg, 0.70 mmol) in DMF (5.0 mL) were added HATU (333 mg, 0.87 mmol) and NMM (178 mg, 1.75 mmol). The resulting mixture was stirred at room temperature for 12 h. After the reaction was completed, the reaction was monitored by LCMS. The resulting mixture was extracted with ethyl acetate. The combined organic layers were dried over anhydrous NaSO₄. After filtration, the filtrate was concentrated under reduced pressure. The residue was purified by flash column chromatography with petroleum ether/ethyl acetate (1/1, v/v) to afford 1-[6,7-dichloro-1-methyl-1H,3H,4H,5H-pyrido[4,3-b]indole-2-carbonyl]-3H-imidazole-4-carbonitrile (20 mg, 23% yield, LCMS (ES, m/z)=374.0 [M−H]⁺) as a white solid, comprising a mixture of enantiomers: 1-[(1R)-6,7-dichloro-1-methyl-1H,3H,4H,5H-pyrido[4,3-b]indole-2-carbonyl]-3H-imidazole-4-carbonitrile (Compound 95A*) and 1-[(1S)-6,7-dichloro-1-methyl-1H,3H,4H,5H-pyrido[4,3-b]indole-2-carbonyl]-3H-imidazole-4-carbonitrile (Compound 95B*), stereochemistry arbitrarily assigned. The racemic mixture was separated by Prep-Chiral-HPLC (Column: CHIRALPAK IG, 2×25 cm, 5 m; Mobile Phase A: Hexanes (0.1% TFA), Mobile Phase B: EtOH; Flow rate: 20 mL/min; Gradient: 25% B to 25% B in 10 min; Wave Length: 254/220 nm) to afford Compound 95A* (chiral RT(min)=23.04, 2.0 mg, 1% yield) as a white solid, and Compound 95B* (chiral RT(min)=15.64, 4.7 mg, 2% yield) as a white solid.

Compound 95A*: LCMS (ES, m/z)=374.0 [M−H]⁺. ¹H NMR (400 MHz, Methanol-d₄) δ 7.98 (d, J=7.6 Hz, 1H), 7.41 (d, J=8.4 Hz, 1H), 7.16 (d, J=8.4 Hz, 1H), 5.83-5.33 (m, 1H), 3.70-3.58 (m, 1H), 3.50-3.45 (m, 1H), 3.33-3.23 (m, 1H), 3.20-3.00 (m, 1H), 2.99-2.90 (m, 1H), 1.77 (d, J=6.0 Hz, 1H), 1.63 (d, J=6.8 Hz, 2H), 1.49-1.32 (m, 2H).

Compound 95B*: LCMS (ES, m/z)=374.0 [M−H]⁺. ¹H NMR (400 MHz, Methanol-d₄) δ 7.98 (d, J=7.6 Hz, 1H), 7.40 (d, J=8.0 Hz, 1H), 7.16 (d, J=8.8 Hz, 1H), 5.83-5.63 (m, 1H), 3.71-3.64 (m, 1H), 3.43-3.18 (m, 1H), 3.17-2.94 (m, 1H), 2.93-2.88 (m, 1H), 1.76 (d, J=6.8 Hz, 1H), 1.63 (d, J=6.4 Hz, 2H), 1.35-1.31 (m, 2H).

### Example 18: 6,7-dichloro-N-hydroxy-1-methyl-1,3,4,5-tetrahydro-2H-pyrido[4,3-b]indole-2-carboxamide (Compound 97, rac-97), (R)-6,7-dichloro-N-hydroxy-1-methyl-1,3,4,5-tetrahydro-2H-pyrido[4,3-b]indole-2-carboxamide (Compound 97A*), and (S)-6,7-dichloro-N-hydroxy-1-methyl-1,3,4,5-tetrahydro-2H-pyrido[4,3-b]indole-2-carboxamide (Compound 97B*)

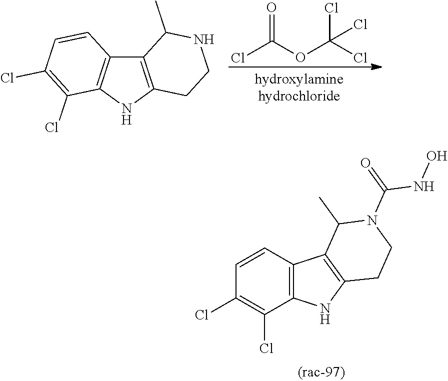

### Stereoisomers:

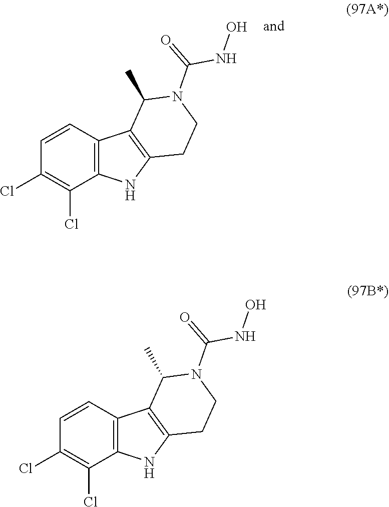 [SMILES: C[C@@H]1c2c([nH]c3c(Cl)c(Cl)ccc23)CCN1C(=O)NO ; C[C@H]1c2c([nH]c3c(Cl)c(Cl)ccc23)CCN1C(=O)NO]

To a mixture of 6,7-dichloro-1-methyl-1H,2H,3H,4H,5H-pyrido[4,3-b]indole (Step 1 product, Example 6) (50 mg, 0.20 mmol) and hydroxylamine hydrochloride (16.4 mg, 0.250 mmol) in CH₂C12 (1 mL) were added DIEA (0.10 mL, 0.58 mmol) and trichloromethyl carbonochloridate (58.5 mg, 0.290 mmol). The resulting mixture was stirred at room temperature for 12 h. After the reaction was completed, the reaction was monitored by LCMS. The resulting mixture was extracted with ethyl acetate. The combined organic layers were dried over anhydrous NaSO₄. After filtration, the filtrate was concentrated under reduced pressure. The crude product was purified Prep-HPLC (YMC-Actus Triart C18, 30*250 5 um; Mobile Phase A: Water (0.1% NH₄HCO₃), Mobile Phase B: MeOH; Flow rate: 60 mL/min; Gradient: 34% to 44% in 10 min. Detector, UV 254 nm) to afford 6,7-dichloro-N-hydroxy-1-methyl-1H,3H,4H,5H-pyrido[4,3-b]indole-2-carboxamide (Compound 97, rac-97) (1.2 mg, 2% yield) as a white solid, which comprises a mixture of enantiomers (R)-6,7-dichloro-N-hydroxy-1-methyl-1,3,4,5-tetrahydro-2H-pyrido[4,3-b]indole-2-carboxamide (Compound 97A*), and (S)-6,7-dichloro-N-hydroxy-1-methyl-1,3,4,5-tetrahydro-2H-pyrido[4,3-b]indole-2-carboxamide (Compound 97B*), stereochemistry arbitrarily assigned. LCMS (ESI, m/z)=314.0 [M+H]⁺. ¹H NMR (400 MHz, Methanol-d₄) δ 7.38-7.32 (m, 1H), 7.14 (d, J=8.4 Hz, 1H), 5.30-5.22 (m, 1H), 4.28-4.24 (m, 1H), 3.48 (s, 1H), 2.98-2.91 (m, 1H), 2.78-2.74 (m, 1H), 1.64 (d, J=12.8 Hz, 3H).

### Example 19: (6,7-dichloro-1-methyl-9-(methylthio)-1,3,4,5-tetrahydro-2H-pyrido[4,3-b]indol-2-yl)(5-methoxypyrimidin-2-yl)methanone (Compound 101, Rac-101), (R)-(6,7-dichloro-1-methyl-9-(methylthio)-1,3,4,5-tetrahydro-2H-pyrido[4,3-b]indol-2-yl)(5-methoxypyrimidin-2-yl)methanone (Compound 101A*), (S)-(6,7-dichloro-1-methyl-9-(methylthio)-1,3,4,5-tetrahydro-2H-pyrido[4,3-b]indol-2-yl)(5-methoxypyrimidin-2-yl)methanone (Compound 101B*), (9-bromo-6,7-dichloro-1-methyl-1,3,4,5-tetrahydro-2H-pyrido[4,3-b]indol-2-yl)(5-methoxypyrimidin-2-yl)methanone (Compound 156, rac-156), (R)-(9-bromo-6,7-dichloro-1-methyl-1,3,4,5-tetrahydro-2H-pyrido[4,3-b]indol-2-yl)(5-methoxypyrimidin-2-yl)methanone (Compound 156A*), and (S)-(9-bromo-6,7-dichloro-1-methyl-1,3,4,5-tetrahydro-2H-pyrido[4,3-b]indol-2-yl)(5-methoxypyrimidin-2-yl)methanone (Compound 156B*)

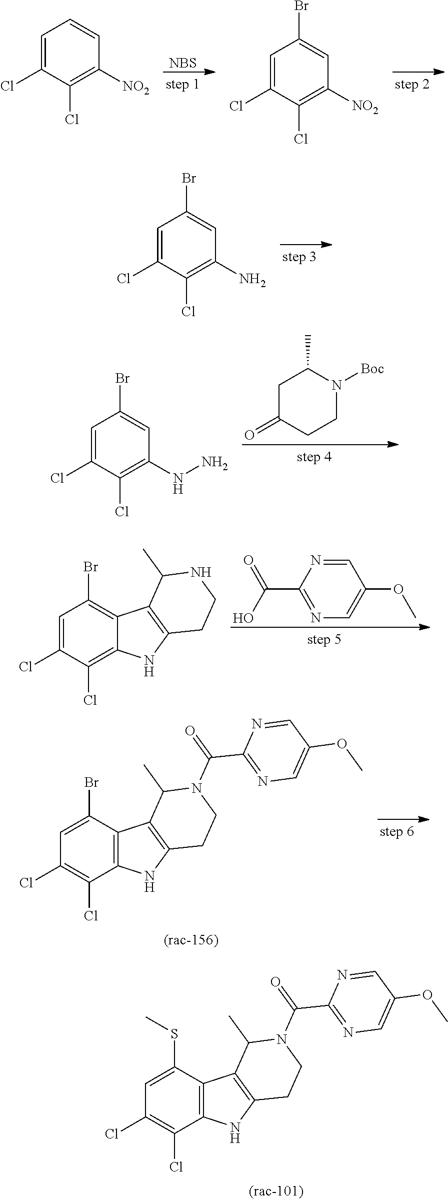

### Stereoisomers:

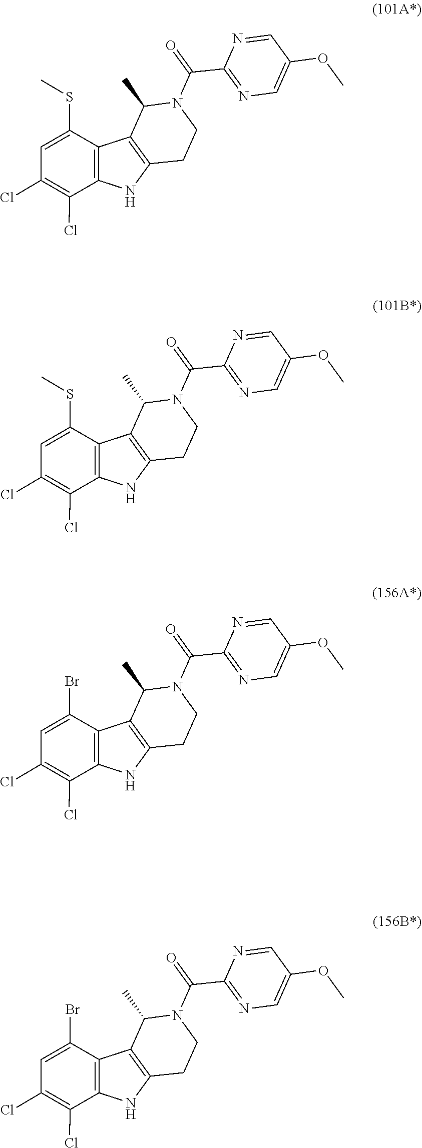 [SMILES: COc1cnc(C(=O)N2CCc3[nH]c4c(Cl)c(Cl)cc(SC)c4c3[C@H]2C)nc1 ; COc1cnc(C(=O)N2CCc3[nH]c4c(Cl)c(Cl)cc(SC)c4c3[C@@H]2C)nc1 ; COc1cnc(C(=O)N2CCc3[nH]c4c(Cl)c(Cl)cc(Br)c4c3[C@H]2C)nc1 ; COc1cnc(C(=O)N2CCc3[nH]c4c(Cl)c(Cl)cc(Br)c4c3[C@@H]2C)nc1]

Step 1: Into a 250 mL round-bottom flask were added 2,3-dichloronitrobenzene (25.0 g, 130 mmol, 1 equiv), NBS (25.5 g, 143 mmol, 1.10 equiv) and H₂SO₄ (80 mL) at 60° C. The resulting mixture was stirred for 2 h at 60° C. The reaction was monitored by LCMS. The reaction was quenched with water (200 mL) at 0° C. The resulting mixture was extracted with ethyl acetate (3×200 mL). The combined organic layers were washed with brine (50 mL), dried over anhydrous Na₂SO₄. After filtration, the filtrate was concentrated under reduced pressure. The residue was purified by silica gel column chromatography, eluted with petroleum ether/ethyl acetate (20:1) to afford 5-bromo-1,2-dichloro-3-nitrobenzene (15 g, 36% yield) as a yellow solid. LCMS (ES, m/z)=270.0 [M+H]⁺. ¹H NMR (400 MHz, DMSO-d₆) δ 7.75 (s, J=2.0, 1.0 Hz, 1H), 7.67 (s, J=2.0 Hz, 1H).

Step 2: Into a 100 mL round-bottom flask were added 5-bromo-1,2-dichloro-3-nitrobenzene (15.0 g, 55.4 mmol, 1 equiv), iron (15.5 g, 277 mmol, 5 equiv) and AcOH (20 mL) at 80° C. The resulting mixture was stirred for 2 h at 80° C. The reaction was monitored by LCMS. The reaction was quenched with water (100 mL) at 0° C. The resulting mixture was extracted with ethyl acetate (3×100 mL). The combined organic layers were washed with brine (1×50 mL), dried over anhydrous Na₂SO₄. After filtration, the filtrate was concentrated under reduced pressure. The residue was purified by silica gel column chromatography, eluted with petroleum ether/ethyl acetate (10:1) to afford 5-bromo-2,3-dichloroaniline (7 g, 42% yield) as a white solid. LCMS (ES, m/z)=240.0 [M+H]⁺. ¹H NMR (400 MHz, DMSO-d₆) δ 7.40 (s, J=8.5, 1.6 Hz, 1H), 6.94 (s, J=1.1 Hz, 1H), 6.01 (s, 2H).

Step 3: A suspension of 5-bromo-2,3-dichloroaniline (7 g, 29.0 mmol, 1 equiv) in HCl (30 mL, 12 M) was added NaNO₂ (4.01 g, 58.1 mmol, 2 equiv) in H₂O (30.00 mL) at 0° C., and then the resulting solution was stirred for 3 h at 0° C. in a water/ice bath. Then SnCl₂ (16.7 g, 87.2 mmol, 3 equiv) in HCl (30 mL, 12 M) was added in, and then the resulting solution was reacted for another 12 h at 0° C. in a water/ice bath. The solids were collected by filtration, washed with 3×20 mL of ethyl acetate. The crude product was purified by reverse phase flash with the following conditions (ACN:H₂O=1:1) to afford (5-bromo-2,3-dichlorophenyl)hydrazine (1.50 g, 17% yield) as a white solid. LCMS (ES, m/z)=255.0 [M+H]⁺. ¹H NMR (400 MHz, DMSO-d6) δ 9.01 (s, J=2.0, 1.0 Hz, 1H), δ 7.75 (s, J=2.0, 1.0 Hz, 1H), 7.67 (s, J=2.0 Hz, 1H), δ 4.59 (s, J=2.0, 1.0 Hz, 2H).

Step 4: Into a 20 mL vial were added (5-bromo-2,3-dichlorophenyl)hydrazine (1.50 g, 5.86 mmol, 1 equiv), tert-butyl (S)-2-methyl-4-oxopiperidine-1-carboxylate (1.25 g, 5.86 mmol, 1 equiv), concentrated H₂SO₄ (5.76 g, 58.6 mmol, 10 equiv) and dioxane (4.0 mL) at 80° C. The resulting mixture was stirred for 12 h at 110° C. The reaction was monitored by LCMS. The reaction was quenched by the addition of water (10 mL) at 0° C. The residue was neutralized to pH 7 with NaOH (10 M). The resulting mixture was extracted with ethyl acetate (3×20 mL). The combined organic layers were washed with brine (1×20 mL), dried over anhydrous Na₂SO₄. After filtration, the filtrate was concentrated under reduced pressure. The crude product was purified by reverse phase flash with the following conditions (ACN:H₂O=1:1) to afford 9-bromo-6,7-dichloro-1-methyl-1H,2H,3H,4H,5H-pyrido[4,3-b]indole (300 mg, 12% yield) as a white solid, which comprises a mixture of R/S enantiomers. The crude product (300 mg) was purified Prep-HPLC (XSelect CSH Prep C18 OBD Column, 19*250 mm, 5 m; Mobile Phase A: Water(0.05% TFA), Mobile Phase B: MeOH—-HPLC; Flow rate: 25 mL/min; Gradient: 50% B to 56% B in 8 min, 56% B; Wave Length: 254 nm; RT1 (min): 10) to afford purified 9-bromo-6,7-dichloro-1-methyl-1H,2H,3H,4H,5H-pyrido[4,3-b]indole (80 mg, 8% yield) as a white solid. LCMS (ES, m/z)=333.0 [M+H]⁺. ¹H NMR (400 MHz, DMSO-d₆). ¹H NMR (400 MHz, DMSO-d₆) δ 12.11 (s, 1H), 9.14 (s, 1H), 7.53 (s, 1H), 5.11 (q, J=6.6 Hz, 1H), 3.53 (d, J=6.6 Hz, 2H), 3.08-2.98 (m, 2H), 1.67 (d, J=6.6 Hz, 3H).

Step 5: Into a 20 mL vial were added 9-bromo-6,7-dichloro-1-methyl-1H,2H,3H,4H,5H-pyrido[4,3-b]indole (80.0 mg, 0.240 mmol, 1 equiv), 5-methoxypyrimidine-2-carboxylic acid (37.1 mg, 0.240 mmol, 1.10 equiv), HATU (109 mg, 0.280 mmol, 1.2 equiv), NMM (73 mg, 0.71 mmol, 3 equiv) and DMF (3.00 mL) at room temperature. The resulting mixture was stirred for 2 h at room temperature. The reaction was monitored by LCMS. The reaction was quenched with water at room temperature. The resulting mixture was washed with 1×10 mL of water. The resulting mixture was extracted with ethyl acetate (3×10 mL). The combined organic layers were washed with brine (1×10 mL), dried over anhydrous Na₂SO₄. After filtration, the filtrate was concentrated under reduced pressure. The crude product was purified by reverse phase flash with the following conditions (ACN:H₂O=8:2) to afford (9-bromo-6,7-dichloro-1-methyl-1,3,4,5-tetrahydro-2H-pyrido[4,3-b]indol-2-yl)(5-methoxypyrimidin-2-yl)methanone (Compound 156, rac-156) (70 mg, 56% yield) as a white solid, comprising a mixture of enantiomers, Compound 156A* and Compound 156B*. LCMS (ES, m/z)=469.0 [M+H]⁺. ¹H NMR (400 MHz, DMSO-d).

Step 6: Into a 10 mL pressure tank reactor was added (9-bromo-6,7-dichloro-1-methyl-1,3,4,5-tetrahydro-2H-pyrido[4,3-b]indol-2-yl)(5-methoxypyrimidin-2-yl)methanone (rac-156) (50.0 mg, 0.106 mmol, 1 equiv), Cul (6.1 mg, 0.032 mmol, 0.30 equiv), DABCO (6.9 mg, 0.022 mmol, 2.0 equiv) and DMSO (16.6 mg, 0.212 mmol, 2 equiv) at room temperature. The final reaction mixture was irradiated with microwave radiation for 3 h at 150° C. The reaction was monitored by LCMS. The reaction was quenched by the addition of water (2.0 mL) at room temperature. The resulting mixture was extracted with ethyl acetate (3×10 mL). The combined organic layers were washed with brine (1×10.0 mL), dried over anhydrous Na₂SO₄. After filtration, the filtrate was concentrated under reduced pressure. The crude product was purified by reverse phase flash chromatography ((R,R) WHELK-O1, 4.6*100 mm, 3.5 m; Flow rate: 2 mL/min; Gradient: isocratic 0% B; Wave Length: 220 nm) to afford (6,7-dichloro-1-methyl-9-(methylthio)-1,3,4,5-tetrahydro-2H-pyrido[4,3-b]indol-2-yl)(5-methoxypyrimidin-2-yl)methanone (Compound 101, rac-101) (2.0 mg, 4.3% yield) as a white solid, which comprises a mixture of enantiomers, Compound 101A* and Compound 101B*. LCMS (ES, m/z)=437.0 [M+1]⁺. ¹H NMR (400 MHz, DMSO-d6) δ 11.61 (s, 1H), 8.65 (d, J=2.2 Hz, 2H), 6.98 (s, 1H), 6.15 (q, J=6.4 Hz, 1H), 4.97-4.76 (q, J=6.4 Hz, 1H), 3.98 (d, J=3.7 Hz, 3H), 3.58 (s, 1H), 2.94-2.80 (m, 1H), 2.72 (dd, J=16.4, 4.2 Hz, 1H), 2.62 (s, 2H), 2.39 (s, 1H), 1.60 (dd, J=13.7, 6.5 Hz, 3H).

### Example 20: (6,7-dichloro-1,9-dimethyl-1,3,4,5-tetrahydro-2H-pyrido[4,3-b]indol-2-yl)(5-methoxypyrimidin-2-yl)methanone (Compound 103, rac-103), (R)-(6,7-dichloro-1,9-dimethyl-1,3,4,5-tetrahydro-2H-pyrido[4,3-b]indol-2-yl)(5-methoxypyrimidin-2-yl)methanone (Compound 103A*), and (S)-(6,7-dichloro-1,9-dimethyl-1,3,4,5-tetrahydro-2H-pyrido[4,3-b]indol-2-yl)(5-methoxypyrimidin-2-yl)methanone (Compound 103B*)

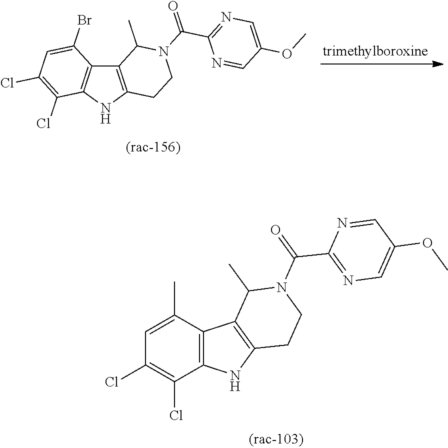 [SMILES: COc1cnc(C(=O)N2CCc3[nH]c4c(Cl)c(Cl)cc(Br)c4c3C2C)nc1 ; COc1cnc(C(=O)N2CCc3[nH]c4c(Cl)c(Cl)cc(C)c4c3C2C)nc1]

### Stereoisomers:

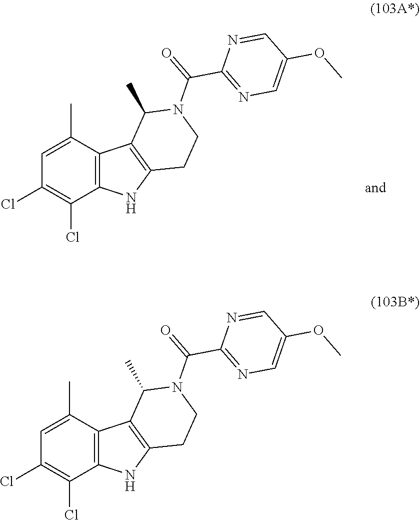 [SMILES: COc1cnc(C(=O)N2CCc3[nH]c4c(Cl)c(Cl)cc(C)c4c3[C@H]2C)nc1 ; COc1cnc(C(=O)N2CCc3[nH]c4c(Cl)c(Cl)cc(C)c4c3[C@@H]2C)nc1]

(9-bromo-6,7-dichloro-1-methyl-1,3,4,5-tetrahydro-2H-pyrido[4,3-b]indol-2-yl)(5-methoxypyrimidin-2-yl)methanone (rac-156, Step 5 product, Example 19) (1.0 eq, 40 mg, 0.085 mmol), potassium phosphate tribasic monohydrate (4.0 eq, 78 mg, 0.34 mmol), trimethylboroxine (0.80 eq, 0.0096 mL, 0.068 mmol) and 1,1′-bis(di-tert-butylphosphino)ferrocene palladium dichloride (0.10 eq, 5.5 mg, 0.0085 mmol) were combined in 1,4-dioxane (0.41 mL) and water (0.068 mL). The reaction mixture was sparged with nitrogen for 3 minutes and stirred at 90° C. for 2 h. The crude mixture was purified by reversed phase chromatography using a RediSep C18 column and a 15-65% MeCN/0.1% aqueous formic acid gradient to afford (6,7-dichloro-1,9-dimethyl-1,3,4,5-tetrahydro-2H-pyrido[4,3-b]indol-2-yl)(5-methoxypyrimidin-2-yl)methanone (Compound 103, rac-103) (8.0 mg, 23% yield) of as a beige solid, which comprised a mixture of enantiomers, Compound 103A* and Compound 103B*, stereochemistry arbitrarily assigned. LCMS (ES, m/z)=404.9 [M+1]⁺. ¹H NMR (400 MHz, DMSO-d6) δ 11.47 (d, J=19.3 Hz, 1H), 8.67-8.61 (m, 2H), 7.04-6.87 (m, 1H), 5.43 (dq, J=460.7, 6.6 Hz, 1H), 4.80-4.71 (m, OH), 4.00-3.94 (m, 3H), 3.64-3.53 (m, 1H), 3.43 (dt, J=14.6, 7.7 Hz, 1H), 2.94-2.80 (m, 2H), 2.77-2.68 (m, 1H), 2.61 (s, 2H), 1.58-1.48 (m, 3H).

### Example 21: 2-amino-1-(7,8-dichloro-1-methyl-3,4-dihydropyrazino[1,2-b]indazol-2(1H)-yl)ethan-1-one (Compound 120, rac-120), (R)-2-amino-1-(7,8-dichloro-1-methyl-3,4-dihydropyrazino[1,2-b]indazol-2(1H)-yl)ethan-1-one (Compound 120A*) and (S)-2-amino-1-(7,8-dichloro-1-methyl-3,4-dihydropyrazino[1,2-b]indazol-2(1H)-yl)ethan-1-one (Compound 120B*)

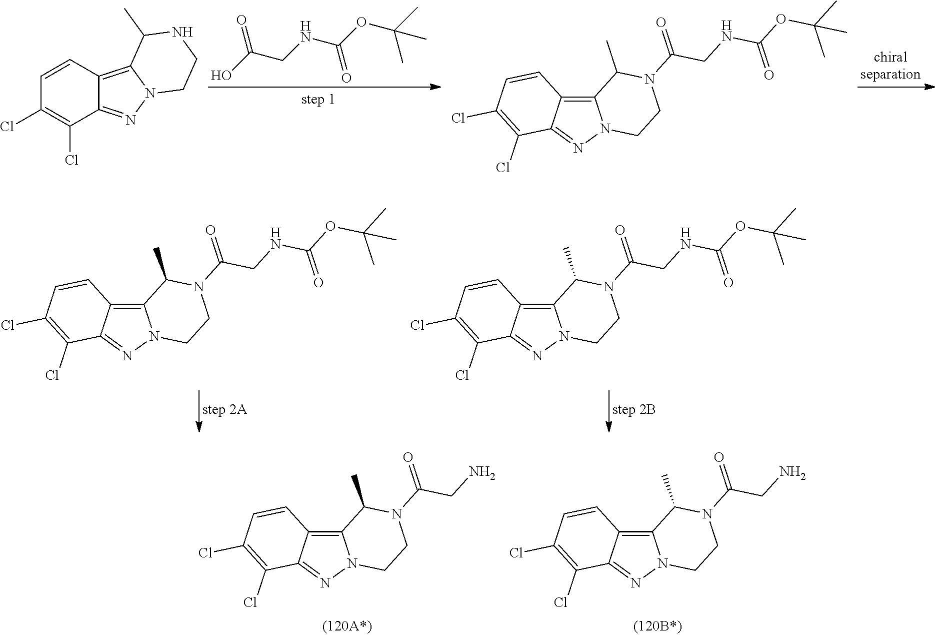

Step 1: Into a 8 mL round-bottom flask were added 7,8-dichloro-1-methyl-1H,2H,3H,4H-pyrazino[1,2-b]indazole hydrochloride (40.0 mg, 0.150 mmol, 1 equiv), [(tert-butoxycarbonyl)amino]acetic acid (32.8 mg, 0.190 mmol, 1.2 equiv), and NMM (47.4 mg, 0.470 mmol, 3.00 equiv) at room temperature. HATU (45.2 mg, 0.190 mmol, 1.20 equiv) was added in one portion. The resulting mixture was stirred for 2 h at room temperature. The reaction was monitored by LCMS and was quenched with water (15 mL) at room temperature. The resulting mixture was extracted with ethyl acetate (3×15 mL). The organic layers were washed with water (3×15 mL), dried over anhydrous Na₂SO₄ and concentrated under reduced pressure. The crude product was purified by reverse phase flash with the following conditions (acetonitrile:H₂O=1:1) to afford tert-butyl (2-(7,8-dichloro-1-methyl-3,4-dihydropyrazino[1,2-b]indazol-2(1H)-yl)-2-oxoethyl)carbamate (43.0 mg) as a white solid, which was further purified by Prep-HPLC (XBridge Prep Phenyl OBD Column, 19*250 mm, 5 μm; Mobile Phase A: Water (0.1% TFA), Mobile Phase B: acetonitrile; Flow rate: 25 mL/min; Gradient: 40% B to 50% B in 10 min, 50% B; Wave Length: 254 nm; RT1 (min): 9) to afford tert-butyl (2-(7,8-dichloro-1-methyl-3,4-dihydropyrazino[1,2-b]indazol-2(1H)-yl)-2-oxoethyl)carbamate (33 mg) as a white solid, which comprises a mixture of R/S enantiomers. The mixture of enantiomers was separated by chiral HPLC with the following conditions (Column: CHIRAL ART Cellulose-SC, 2*25 cm, 5 m; Mobile Phase A: Hex(0.50% 2.00M NH3-MeOH)—HPLC, Mobile Phase B: MeOH:DCM=1:1—HPLC; Flow rate: 20 mL/min; Gradient: 25% B to 25% B in 18 min; Wave Length: 254/220 nm; RT1 (min): 12.15; RT2 (min): 15.39; Sample Solvent: MeOH:DCM=1:1—HPLC) to afford tert-butyl N-{2-[(1R)-7,8-dichloro-1-methyl-1H,3H,4H-pyrazino[1,2-b]indazol-2-yl]-2-oxoethyl}carbamate (chiral RT(min)=12.15, 25 mg, LCMS (ES, m/z)=413.0 [M+1]⁺) as a white solid, and tert-butyl N-{2-[(1S)-7,8-dichloro-1-methyl-1H,3H,4H-pyrazino[1,2-b]indazol-2-yl]-2-oxoethyl}carbamate (chiral RT(min)=15.39, 20 mg, LCMS (ES, m/z)=413.0 [M+1]⁺) as a white solid.

Step 2A: Into a 8 mL vial was added tert-butyl N-{2-[(1R)-7,8-dichloro-1-methyl-1H,3H,4H-pyrazino[1,2-b]indazol-2-yl]-2-oxoethyl}carbamate (25 mg, 0.060 mmol, 1 equiv) and DCM (1.0 mL) at room temperature. The resulting mixture was stirred for 1 h at room temperature under hydrogen chloride(g) atmosphere. The reaction was monitored by LCMS. The resulting mixture was concentrated under reduced pressure. The product was lyophilized to afford (R)-2-amino-1-(7,8-dichloro-1-methyl-3,4-dihydropyrazino[1,2-b]indazol-2(1H)-yl)ethan-1-one hydrochloride (Compound 120A*) (21.1 mg, 91% yield) as a white solid. LCMS (ES, m/z)=313.0 [M+1]⁺. ¹H NMR (400 MHz, DMSO-d₆) δ 8.27 (s, 3H), 7.91 (d, J=8.9 Hz, 1H), 7.22 (m, J=15.5, 8.8 Hz, 1H), 6.08 (q, J=6.8 Hz, 1H), 4.84 (m, J=13.8, 4.4 Hz, 1H), 4.62-4.54 (m, 1H), 4.33-4.16 (m, 1H), 4.12 (t, J=5.7 Hz, 2H), 3.89 (m, J=15.3, 9.7, 5.9 Hz, 1H), 1.65 (d, J=6.7 Hz, 1H), 1.56 (d, J=6.8 Hz, 2H).

Step 2B: Into a 8 mL vial was added tert-butyl N-{2-[(1S)-7,8-dichloro-1-methyl-1H,3H,4H-pyrazino[1,2-b]indazol-2-yl]-2-oxoethyl}carbamate (30 mg, 0.072 mmol, 1 equiv) and DCM (3.0 mL) at room temperature. The resulting mixture was stirred for 1 h at room temperature under hydrogen chloride(g) atmosphere. The reaction was monitored by LCMS. The resulting mixture was concentrated under reduced pressure. The product was lyophilized to afford (R)-2-amino-1-(7,8-dichloro-1-methyl-3,4-dihydropyrazino[1,2-b]indazol-2(1H)-yl)ethan-1-one hydrochloride (Compound 120B*, 20.2 mg, 87% yield) as a white solid. LCMS (ES, m/z)=313.0 [M+1]⁺. ¹H NMR (400 MHz, DMSO-d₆) δ 8.27 (s, 3H), 7.91 (d, J=8.9 Hz, 1H), 7.22 (m, J=15.5, 8.8 Hz, 1H), 6.08 (q, J=6.8 Hz, 1H), 4.84 (m, J=13.8, 4.4 Hz, 1H), 4.62-4.54 (m, 1H), 4.33-4.16 (m, 1H), 4.12 (t, J=5.7 Hz, 2H), 3.89 (m, J=15.3, 9.7, 5.9 Hz, 1H), 1.65 (d, J=6.7 Hz, 1H), 1.56 (d, J=6.8 Hz, 2H).

### Example 22: (6,7-dichloro-9-methoxy-1-methyl-1,3,4,5-tetrahydro-2H-pyrido[4,3-b]indol-2-yl)(5-methoxypyrimidin-2-yl)methanone (Compound 132, rac-132), (R)-(6,7-dichloro-9-methoxy-1-methyl-1,3,4,5-tetrahydro-2H-pyrido[4,3-b]indol-2-yl)(5-methoxypyrimidin-2-yl)methanone (Compound 132A*), and (S)-(6,7-dichloro-9-methoxy-1-methyl-1,3,4,5-tetrahydro-2H-pyrido[4,3-b]indol-2-yl)(5-methoxypyrimidin-2-yl)methanone (Compound 132B*)

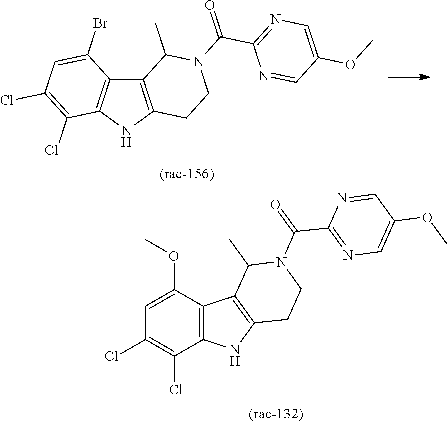 [SMILES: COc1cnc(C(=O)N2CCc3[nH]c4c(Cl)c(Cl)cc(Br)c4c3C2C)nc1 ; COc1cnc(C(=O)N2CCc3[nH]c4c(Cl)c(Cl)cc(OC)c4c3C2C)nc1]

### Stereoisomers:

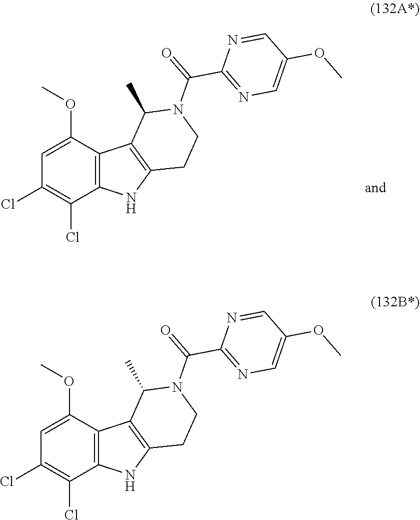 [SMILES: COc1cnc(C(=O)N2CCc3[nH]c4c(Cl)c(Cl)cc(OC)c4c3[C@H]2C)nc1 ; COc1cnc(C(=O)N2CCc3[nH]c4c(Cl)c(Cl)cc(OC)c4c3[C@@H]2C)nc1]

Into an 8-mL vial tube was placed (9-bromo-6,7-dichloro-1-methyl-1,3,4,5-tetrahydro-2H-pyrido[4,3-b]indol-2-yl)(5-methoxypyrimidin-2-yl)methanone (rac-156, Step 5 product, Example 19) (40 mg, 0.085 mmol, 1 equiv), MeONa (23.0 mg, 0.425 mmol, 5 equiv), potassium iodide (14.1 mg, 0.0850 mmol, 1 equiv) and EPhos Pd G4 (3.9 mg, 0.0040 mmol, 0.05 equiv) in 1,4-dioxane (2 mL) under N₂ atmosphere. The resulting mixture was stirred for 4 h at room temperature. Dimethylacetamide (0.5 mL) was added in above reaction mixture. The crude mixture was filtered and the filtrate was purified by Prep-HPLC (Welch Xtimate C18 30*150 mm, 10 um; Mobile phase A: 0.05% NH₃H₂O/Mobile phase B: acetonitrile; Gradient: B % 35-55 7 min; Flow rate: 35 ml/min) to afford (6,7-dichloro-9-methoxy-1-methyl-1,3,4,5-tetrahydro-2H-pyrido[4,3-b]indol-2-yl)(5-methoxypyrimidin-2-yl)methanone (Compound 132, rac-132) (3.0 mg, 8% yield) as a beige solid, which comprises a mixture of enantiomers, Compound 132A* and Compound 132B*. LCMS (ES, m/z)=421.1 [M+1]⁺.

Compounds provided in the below Table A were, or may be, synthesized following the Procedures as described with reference to the Examples. LC-MS data, and optionally ¹H NMR data, is provided for compounds synthesized. “Rac-X” signifies a mixture of stereoisomers: Compounds “XA” and “XB”.

TABLE B
Additional Compounds

| | | | m/z | |
|---|---|---|---|---|
| # | Compound | ¹H NMR | [M + H]⁺ | Preparation Method |
| 2 | (6,7-dichloro-1,3,4,5- | ¹H NMR (400 MHz, | 347.2 | The compound was |
| | tetrahydro-2H- | DMSO-d6) δ 11.60-11.50 | | synthesized following Ex 1 |
| | pyrido[4,3-b]indol-2- | (m, 1H), 8.92 (dd, J = 17.6, | | |
| | yl)(pyrimidin-2- | 5.0 Hz, 2H), 7.63 (dt, J = | | |
| | yl)methanone | 10.4, 5.0 Hz, 1H), 7.53 (d, | | |
| | | J = 8.4 Hz, 1H), 7.29-7.04 | | |
| | | (m, 1H), 4.92-4.32 (m, | | |
| | | 2H), 4.12-3.44 (m, 2H), | | |
| | | 3.03-2.75 (m, 2H). | | |
| 3 | (6,7-dichloro-1,3,4,5- | ¹H NMR (400 MHz, | 349.3 | The compound was |
| | tetrahydro-2H- | DMSO-d6) δ 11.53 (s, 1H), | | synthesized following Ex 1 |
| | pyrido[4,3-b]indol-2- | 7.49 (d, J = 8.4 Hz, 1H), | | |
| | yl)(1-methyl-1H- | 7.37-7.27 (m, 1H), 7.22- | | |
| | imidazol-2-yl)methanone | 7.09 (m, 1H), 7.03 (d, J = | | |
| | | 1.0 Hz, 1H), 5.13-4.77 (m, | | |
| | | 2H), 4.09 (dt, J = 58.6, 5.7 | | |
| | | Hz, 2H), 3.80-3.73 (m, | | |
| | | 3H), 3.00-2.88 (m, 2H). | | |
| 4 | (6,7-dichloro-1,3,4,5- | ¹H NMR (400 MHz, | 377.2 | The compound was |
| | tetrahydro-2H- | DMSO-d6) δ 11.59-11.51 | | synthesized following Ex 1 |
| | pyrido[4,3-b]indol-2- | (m, 1H), 8.65-8.54 (m, | | |
| | yl)(4-methoxypyrimidin- | 1H), 7.56-7.28 (m, 1H), | | |
| | 2-yl)methanone | 7.23-7.07 (m, 1H), 7.07- | | |
| | | 7.00 (m, 1H), 4.86-4.80 | | |
| | | (m, 1H), 4.43 (s, 1H), 4.03 | | |
| | | (t, J = 5.8 Hz, 1H), 3.98- | | |
| | | 3.87 (m, 3H), 3.54 (t, J = | | |
| | | 5.7 Hz, 1H), 2.97-2.81 (m, | | |
| | | 2H). | | |
| 5 | (6,7-dichloro-1,3,4,5- | ¹H NMR (400 MHz, | 335.2 | The compound was |
| | tetrahydro-2H- | DMSO-d6) δ 13.00-12.95 | | synthesized following Ex 1 |
| | pyrido[4,3-b]indol-2- | (m, 1H), 11.56-11.49 (m, | | |
| | yl)(1H-imidazol-2- | 1H), 7.50 (d, J = 8.4 Hz, | | |
| | yl)methanone | 1H), 7.37 (d, J = 8.4 Hz, | | |
| | | 0H), 7.31-7.24 (m, 1H), | | |
| | | 7.18 (d, J = 8.2 Hz, 1H), | | |
| | | 7.13 (s, 1H), 5.62 (s, 1H), | | |
| | | 4.82 (s, 1H), 4.73 (t, J = 5.6 | | |
| | | Hz, 1H), 4.06-3.99 (m, | | |
| | | 1H), 3.01-2.93 (m, 1H), | | |
| | | 2.91 (t, J = 5.8 Hz, 1H). | | |
| 6 | (6,7-dichloro-1,3,4,5- | ¹H NMR (400 MHz, | 336.1 | The compound was |
| | tetrahydro-2H- | DMSO-d6) δ 13.66 (br. s, | | synthesized following Ex 1 |
| | pyrido[4,3-b]indol-2- | 1H), 11.55 (s, 1H), 8.54- | | |
| | yl)(1H-1,2,4-triazol-3- | 8.24 (m, 1H), 7.55-7.27 | | |
| | yl)methanone | (m, 1H), 7.23-7.09 (m, | | |
| | | 1H), 5.06-4.81 (m, 2H), | | |
| | | 4.15-4.00 (m, 2H), 3.01- | | |
| | | 2.84 (m, 2H). | | |
| 7 | (6,7-dichloro-1,3,4,5- | ¹H NMR (400 MHz, | 337.1 | The compound was |
| | tetrahydro-2H- | DMSO-d6) δ 11.62-11.53 | | synthesized following Ex 1 |
| | pyrido[4,3-b]indol-2- | (m, 1H), 7.56-7.44 (m, | | |
| | yl)(1H-tetrazol-5- | 1H), 7.39-7.11 (m, 1H), | | |
| | yl)methanone (tautomer | 7.19 (d, J = 8.4 Hz, 1H), | | |
| | 1) | 5.11-4.85 (m, 2H), 4.26- | | |
| | (6,7-dichloro-1,3,4,5- | 4.04 (m, 2H), 3.02-2.91 | | |
| | tetrahydro-2H- | (m, 2H). | | |
| | pyrido[4,3-b]indol-2- | | | |
| | yl)(2H-tetrazol-5- | | | |
| | yl)methanone (tautomer | | | |
| | 2) | | | |
| 8 | (6,7-dichloro-1,3,4,5- | ¹H NMR (400 MHz, | 361.2 | The compound was |
| | tetrahydro-2H- | DMSO-d6) δ 11.60-11.51 | | synthesized following Ex 1 |
| | pyrido[4,3-b]indol-2- | (m, 1H), 8.79-8.69 (m, | | |
| | yl)(4-methylpyrimidin-2- | 1H), 7.56-7.51 (m, 1H), | | |
| | yl)methanone | 7.51-7.24 (m, 1H), 7.22- | | |
| | | 7.05 (m, 1H), 4.87-4.34 | | |
| | | (m, 2H), 4.08-3.44 (m, | | |
| | | 2H), 2.97-2.78 (m, 2H), | | |
| | | 2.56-2.50 (m, 3H). | | |
| 9 | (6,7-dichloro-1,3,4,5- | ¹H NMR (400 MHz, | 350.0 | The compound was |
| | tetrahydro-2H- | DMSO-d6) δ 11.57-11.51 | | synthesized following Ex 1 |
| | pyrido[4,3-b]indol-2- | (m, 1H), 8.64-8.57 (m, | | |
| | yl)(1-methyl-1H-1,2,4- | 1H), 7.50 (d, J = 8.3 Hz, | | |
| | triazol-3-yl)methanone | 1H), 7.22-7.08 (m, 1H), | | |
| | | 4.83-4.75 (m, 2H), 4.05- | | |
| | | 3.88 (m, 2H), 3.96-3.91 | | |
| | | (m, 3H), 2.90 (t, J = 5.7 Hz, | | |
| | | 2H). | | |
| 10 | (6,7-dichloro-1,3,4,5- | ¹H NMR (400 MHz, | 335.2 | The compound was |
| | tetrahydro-2H- | DMSO-d6) δ 12.75-12.55 | | synthesized following Ex 1 |
| | pyrido[4,3-b]indol-2- | (m, 1H), 11.52 (s, 1H), 7.53- | | |
| | yl)(5-methyl-1H- | 7.32 (m, 1H), 7.21-7.12 | | |
| | imidazol-2-yl)methanone | (m, 1H), 7.00-6.80 (m, | | |
| | | 1H), 5.61 (s, 1H), 4.84- | | |
| | | 4.72 (m, 2H), 4.05-3.73 | | |
| | | (m, 1H), 3.01-2.86 (m, | | |
| | | 2H), 2.22-2.16 (m, 3H). | | |
| 11 | (6,7-dichloro-1,3,4,5- | ¹H NMR (400 MHz, | 336.1 | The compound was |
| | tetrahydro-2H- | DMSO-d6) δ 11.54 (s, 1H), | | synthesized following Ex 1 |
| | pyrido[4,3-b]indol-2- | 8.69-8.59 (m, 1H), 8.56 (s, | | |
| | yl)(oxazol-2- | 1H), 7.58-7.07 (m, 2H), | | |
| | yl)methanone | 5.09-4.74 (m, 2H), 4.21- | | |
| | | 3.96 (m, 2H), 2.92 (d, J = | | |
| | | 22.0 Hz, 2H). | | |
| 12 | (6,7-dichloro-1,3,4,5- | ¹H NMR (400 MHz, | 361.2 | The compound was |
| | tetrahydro-2H- | DMSO-d6) δ 11.55 (d, J = | | synthesized following Ex 1 |
| | pyrido[4,3-b]indol-2- | 14.4 Hz, 1H), 8.78 (s, 1H), | | |
| | yl)(5-methylpyrimidin-2- | 7.52 (d, J = 8.4 Hz, 1H), | | |
| | yl)methanone | 7.22-7.04 (m, 1H), 4.84 (s, | | |
| | | 1H), 4.38 (s, 1H), 4.06- | | |
| | | 3.47 (m, 2H), 2.96-2.78 | | |
| | | (m, 3H), 2.37-2.31 (m, | | |
| | | 3H). | | |
| 13 | 5-(6,7-dichloro-2,3,4,5- | ¹H NMR (400 MHz, | 352.1 | The compound was |
| | tetrahydro-1H- | DMSO-d6) δ 11.99 (br. s, | | synthesized following Ex 1 |
| | pyrido[4,3-b]indole-2- | 2H), 11.55 (s, 1H), 7.43 (dd, | | |
| | carbonyl)-2,4-dihydro- | J = 51.1, 8.4 Hz, 1H), 7.17 | | |
| | 3H-1,2,4-triazol-3-one | (t, J = 9.0 Hz, 1H), 5.24- | | |
| | | 4.75 (m, 2H), 4.34-3.95 | | |
| | | (m, 2H), 2.98-2.86 (m, | | |
| | | 2H). | | |
| 14 | (4-aminopyrimidin-2- | ¹H NMR (400 MHz, | 362.2 | The compound was |
| | yl)(6,7-dichloro-1,3,4,5- | DMSO-d6) δ 11.56-11.49 | | synthesized following Ex 1 |
| | tetrahydro-2H- | (m, 1H), 8.13-8.05 (m, | | |
| | pyrido[4,3-b]indol-2- | 1H), 7.54-7.34 (m, 1H), | | |
| | yl)methanone | 7.32-7.17 (m, 1H), 7.17 | | |
| | | 7.05 (m, 1H), 6.48-6.39 | | |
| | | (m, 1H), 4.80-4.35 (m, | | |
| | | 2H), 4.00-3.47 (m, 2H), | | |
| | | 2.91-2.78 (m, 1H), 0.96- | | |
| | | 0.79 (m, 2H). | | |
| 16 | (6,7-dichloro-1,3,4,5- | ¹H NMR (400 MHz, | 363.1 | The compound was |
| | tetrahydro-2H- | DMSO-d6) δ 11.56-11.49 | | synthesized following Ex 1 |
| | pyrido[4,3-b]indol-2- | (m, 1H), 8.40-8.30 (m, | | |
| | yl)(5-hydroxypyrimidin- | 2H), 7.55-7.25 (m, 1H), | | |
| | 2-yl)methanone | 7.22-7.04 (m, 1H), 4.84- | | |
| | | 4.40 (m, 2H), 4.06-3.51 | | |
| | | (m, 2H), 2.94-2.79 (m, | | |
| | | 2H), 2.14-1.89 (s, 1H). | | |
| 18 | (6,7-dichloro-5-(2- | ¹H NMR (400 MHz, | 483.2 | The compound was |
| | (methylsulfonyl)ethyl)- | DMSO-d6) δ 8.68-8.59 | | synthesized following Ex 3 |
| | 1,3,4,5-tetrahydro-2H- | (m, 2H), 7.61-7.15 (m, | | |
| | pyrido[4,3-b]indol-2- | 1H), 7.30 (d, J = 8.4 Hz, | | |
| | yl)(5-methoxypyrimidin- | 1H), | | |
| | 2-yl)methanone | 4.88-4.37 (m, 4H), 4.08- | | |
| | | 3.62 (m, 2H), 4.01-3.94 | | |
| | | (m, 3H), 3.61 (dt, J = 19.9, | | |
| | | 6.7 Hz, 2H), 3.09-3.04 (m, | | |
| | | 3H), 3.04-2.89 (m, 2H). | | |
| 20 | (6,7-dichloro-5- | ¹H NMR (400 MHz, | 455.1 | The compound was |
| | (methylsulfonyl)-1,3,4,5- | DMSO-d6) δ 8.70-8.65 | | synthesized following Ex 3 |
| | tetrahydro-2H- | (m, 2H), 7.70-7.46 (m, | | |
| | pyrido[4,3-b]indol-2- | 2H), 4.86-4.79 (m, 2H), | | |
| | yl)(5-methoxypyrimidin- | 4.00-3.97 (m, 3H), 3.93- | | |
| | 2-yl)methanone | 3.87 (m, 3H), 3.57-3.38 | | |
| | | (m, 2H), 3.17-3.04 (m, | | |
| | | 2H). | | |
| 21 | 3-(6,7-dichloro-2-(5- | ¹H NMR (400 MHz, | 449.2 | The compound was |
| | methoxypyrimidine-2- | DMSO-d6) δ 11.53 (s, 1H), | | synthesized following Ex 4 |
| | carbonyl)-1,2,3,4- | 8.69-8.59 (m, 2H), 7.56- | | |
| | tetrahydro-5H- | 7.11 (m, 1H), 7.30-7.22 | | |
| | pyrido[4,3-b]indol-5- | (m, 1H), 4.86-4.37 (m, | | |
| | yl)propanoic acid | 2H), 4.61-4.52 (m, 2H), | | |
| | | 4.00-3.94 (m, 3H), 4.07- | | |
| | | 3.52 (m, 2H), 3.04-2.87 | | |
| | | (m, 2H), 2.64-2.57 (m, | | |
| | | 2H). | | |
| 22 | (6,7-dichloro-5-(2- | ¹H NMR (400 MHz, | 421.1 | The compound was |
| | hydroxyethyl)-1,3,4,5- | DMSO-d6) δ 8.64 (d, J = | | synthesized following Ex 3 |
| | tetrahydro-2H- | 19.6 Hz, 2H), 7.58-7.27 | | |
| | pyrido[4,3-b]indol-2- | (m, 1H), 7.27-7.10 (m, | | |
| | yl)(5-methoxypyrimidin- | 1H), 4.95 (br. s, 1H), 4.85- | | |
| | 2-yl)methanone | 4.37 (m, 2H), 4.46 (dt, J = | | |
| | | 10.9, 5.7 Hz, 2H), 4.01- | | |
| | | 3.94 (m, 3H), 4.08-3.53 | | |
| | | (m, 2H), 3.75-3.66 (m, | | |
| | | 2H), 3.03-2.88 (m, 2H). | | |
| 23 | (6,7-dichloro-5-((2- | ¹H NMR (400 MHz, | 465.2 | The compound was |
| | methoxyethoxy)methyl)- | DMSO-d6) δ 8.71-8.59 | | synthesized following Ex 3 |
| | 1,3,4,5-tetrahydro-2H- | (m, 2H), 7.62-7.29 (m, | | |
| | pyrido[4,3-b]indol-2- | 1H), 7.32 (dd, J = 10.2, 8.4 | | |
| | yl)(5-methoxypyrimidin- | Hz, 1H), 5.90-5.75 (m, | | |
| | 2-yl)methanone | 2H), 4.86-4.40 (m, 2H), | | |
| | | 3.98 (d, J = 6.8 Hz, 3H), | | |
| | | 4.11-3.57 (m, 2H), 3.56- | | |
| | | 3.45 (m, 2H), 3.45-3.35 | | |
| | | (m, 2H), 3.25-3.14 (m, | | |
| | | 3H), 3.02-2.85 (m, 2H). | | |
| 24 | (6,7-dichloro-5-(3- | — | 435.1 | The compound was |
| | hydroxypropyl)-1,3,4,5- | | | synthesized following Ex 3 |
| | tetrahydro-2H- | | | |
| | pyrido[4,3-b]indol-2- | | | |
| | yl)(5-methoxypyrimidin- | | | |
| | 2-yl)methanone | | | |
| 25 | 4-(6,7-dichloro-2-(5- | ¹HNMR(400 MHz, DMSO- | 463.3 | The compound was |
| | methoxypyrimidine-2- | d6) δ 12.44 (br. s, 1H), | | synthesized following Ex 4 |
| | carbonyl)-1,2,3,4- | 8.71-8.59 (m, 2H), 7.58-7.26 | | |
| | tetrahydro-5H- | (m, 1H), 7.26-7.11 (m, | | |
| | pyrido[4,3-b]indol-5- | 1H), 4.85-4.40 (m, 2H), | | |
| | yl)butanoic acid | 4.40-4.34 (m, 2H), 4.01- | | |
| | | 3.93 (m, 3H), 4.10-3.54 | | |
| | | (m, 2H), 2.98-2.83 (m, | | |
| | | 2H), 2.32-2.22 (m, 2H), | | |
| | | 1.93-1.83 (m, 2H). | | |
| 26 | methyl 4-(6,7-dichloro- | ¹HNMR(400 MHz, DMSO- | 477.2 | The compound was |
| | 2-(5- | d6) δ 8.69-8.59 (m, 2H), | | synthesized following Ex 3 |
| | methoxypyrimidine-2- | 7.58-7.27 (m, 1H), 7.27- | | |
| | carbonyl)-1,2,3,4- | 7.12 (m, 1H), 4.85-4.41 | | |
| | tetrahydro-5H- | (m, 2H), 4.41-4.35 (m, | | |
| | pyrido[4,3-b]indol-5- | 2H), 4.01-3.92 (m, 3H), | | |
| | yl)butanoate | 4.10-3.58 (m, 2H), 3.58- | | |
| | | 3.53 (m, 3H), 2.99-2.81 | | |
| | | (m, 2H), 2.45-2.35 (m, | | |
| | | 2H), 2.00-1.86 (m, 2H). | | |
| 27 | 1-(6,7-dichloro-2-(5- | ¹H NMR (400 MHz, | 419.2 | The compound was |
| | methoxypyrimidine-2- | DMSO-d6) δ 8.69-8.60 | | synthesized following Ex 3 |
| | carbonyl)-1,2,3,4- | (m, 2H), 7.68 (d, J = 8.3 Hz, | | |
| | tetrahydro-5H- | 1H), 7.53 (d, J = 8.4 Hz, | | |
| | pyrido[4,3-b]indol-5- | 1H), 7.41 (d, J = 0.9 Hz, | | |
| | yl)ethan-1-one | 1H), 4.87-4.38 (m, 1H), | | |
| | | 4.01-3.93 (m, 3H), 3.80 | | |
| | | (dt, J = 188.8, 5.7 Hz, 2H), | | |
| | | 3.09-2.92 (m, 2H), 2.67- | | |
| | | 2.59 (m, 3H). | | |
| 28 | 2-(6,7-dichloro-2-(5- | — | 416.2 | The compound was |
| | methoxypyrimidine-2- | | | synthesized following Ex 3 |
| | carbonyl)-1,2,3,4- | | | |
| | tetrahydro-5H- | | | |
| | pyrido[4,3-b]indol-5- | | | |
| | yl)acetonitrile | | | |
| 30 | (5-aminopyrimidin-2- | ¹H NMR (400 MHz, | 362.2 | The compound was |
| | yl)(6,7-dichloro-1,3,4,5- | DMSO-d6) δ 11.59-11.48 | | synthesized following Ex 1 |
| | tetrahydro-2H- | (m, 1H), 8.19-8.10 (m, | | |
| | pyrido[4,3-b]indol-2- | 1H), 7.51-7.27 (m, 1H), | | |
| | yl)methanone | 7.26-7.18 (m, 1H), 5.93 | | |
| | | 5.86 (m, 2H), 4.81-4.43 | | |
| | | (m, 2H), 4.02-3.55 (m, | | |
| | | 2H), 3.01-2.78 (m, 3H). | | |
| 31 | (6,7-dichloro-1,3,4,5- | ¹H NMR (400 MHz, | 432.3 | The compound was |
| | tetrahydro-2H- | DMSO-d6) δ 11.58-11.50 | | synthesized following Ex 5 |
| | pyrido[4,3-b]indol-2- | (m, 1H), 8.60-8.51 (m, | | |
| | yl)(5- | 2H), 7.55-7.24 (m, 1H), | | |
| | morpholinopyrimidin-2- | 7.22-7.04 (m, 1H), 4.85- | | |
| | yl)methanone | 4.43 (m, 2H), 4.36 (s, 1H), | | |
| | | 4.06-3.51 (m, 2H), 3.82- | | |
| | | 3.70 (m, 4H), 3.44 (q, J = | | |
| | | 7.0 Hz, 1H), 3.36-3.30 (m, | | |
| | | 2H), 2.95-2.80 (m, 2H). | | |
| 32 | (6,7-dichloro-1,3,4,5- | ¹H NMR (400 MHz, | 420.2 | The compound was |
| | tetrahydro-2H- | DMSO-d6) δ 11.57-11.50 | | synthesized following Ex 5 |
| | pyrido[4,3-b]indol-2- | (m, 1H), 8.36-8.27 (m, | | |
| | yl)(5-((2- | 2H), 7.54-7.23 (m, 1H), | | |
| | hydroxyethyl)(methyl)ami- | 7.22-7.05 (m, 1H), 4.83- | | |
| | no)pyrimidin-2- | 4.47 (m, 2H), 4.01 (t, J = | | |
| | yl)methanone | 5.7 Hz, 1H), 3.65-3.56 (m, | | |
| | | 3H), 3.56-3.48 (m, 2H), | | |
| | | 3.44 (q, J = 6.6 Hz, 1H), | | |
| | | 3.06-3.00 (m, 3H), 2.95- | | |
| | | 2.82 (m, 2H). | | |
| 33 | (6,7-dichloro-1,3,4,5- | ¹H NMR (400 MHz, | 447.2 | The compound was |
| | tetrahydro-2H- | DMSO-d6) δ 11.82-11.75 | | synthesized following Ex 5 |
| | pyrido[4,3-b]indol-2- | (m, 1H), 8.61-8.52 (m, | | |
| | yl)(5-((2- | 2H), 8.44 (s, 1H), 7.80- | | |
| | (dimethylamino)ethyl)(meth- | 7.49 (m, 1H), 7.47-7.30 | | |
| | yl)amino)pyrimidin- | (m, 1H), 5.09-4.72 (m, | | |
| | 2-yl)methanone | 2H), 4.30-3.84 (m, 2H), | | |
| | | 3.84-3.64 (m, 2H), 3.30- | | |
| | | 3.21 (m, 3H), 3.20-3.08 | | |
| | | (m, 2H), 2.76-2.71 (m, | | |
| | | 1H), 2.52-2.43 (m, 6H). | | |
| 34 | (6,7-dichloro-1,3,4,5- | ¹H NMR (400 MHz, | 475.3 | The compound was |
| | tetrahydro-2H- | DMSO-d6) δ 11.57-11.50 | | synthesized following Ex 5 |
| | pyrido[4,3-b]indol-2- | (m, 1H), 8.60-8.49 (m, | | |
| | yl)(5-(4-(2- | 2H), 8.19 (s, 1H), 7.55- | | |
| | hydroxyethyl)piperazin- | 7.25 (m, 1H), 7.22-7.05 | | |
| | 1-yl)pyrimidin-2- | (m, 1H), 4.85-4.44 (m, | | |
| | yl)methanone | 2H), 4.01 (t, J = 5.7 Hz, | | |
| | | 1H), 3.61-3.50 (m, 4H), | | |
| | | 3.44 (q, J = 7.0 Hz, 1H), | | |
| | | 3.37-3.31 (m, 2H), 2.95- | | |
| | | 2.80 (m, 2H), 2.62-2.54 | | |
| | | (m, 4H), 2.49-2.41 (m, | | |
| | | 2H). | | |
| 35 | (R)-(6,7-dichloro- | ¹H NMR (400 MHz, | 446.2 | The compound was |
| | 1,3,4,5-tetrahydro-2H- | DMSO-d6) δ 11.57-11.50 | | synthesized following Ex 5 |
| | pyrido[4,3-b]indol-2- | (m, 1H), 8.28-8.17 (m, | | |
| | yl)(5-(2- | 2H), 7.54-7.23 (m, 1H), | | |
| | (hydroxymethyl)pyrrolidin- | 7.22-7.05 (m, 1H), 4.84- | | |
| | 1-yl)pyrimidin-2- | 4.49 (m, 2H), 4.90-4.30 | | |
| | yl)methanone | (m, 1H), 4.05-3.85 (m, | | |
| | | 2H), 3.61 (t, J = 5.6 Hz, | | |
| | | 1H), 3.55-3.11 (m, 5H), | | |
| | | 2.95-2.82 (m, 2H), 2.04- | | |
| | | 1.89 (m, 3H). | | |
| 36 | (S)-(6,7-dichloro-1,3,4,5- | ¹H NMR (400 MHz, | 446.2 | The compound was |
| | tetrahydro-2H- | DMSO-d6) δ 11.57-11.50 | | synthesized following Ex 5 |
| | pyrido[4,3-b]indol-2- | (m, 1H), 8.28-8.18 (m, | | |
| | yl)(5-(2- | 2H), 7.54-7.23 (m, 1H), | | |
| | (hydroxymethyl)pyrrolidin- | 7.21-7.05 (m, 1H), 4.90- | | |
| | 1-yl)pyrimidin-2- | 4.30 (m, 1H), 4.83-4.47 | | |
| | yl)methanone | (m, 2H), 4.06-3.85 (m, | | |
| | | 2H), 3.61 (t, J = 5.6 Hz, | | |
| | | 1H), 3.54-3.09 (m, 5H), | | |
| | | 2.95-2.82 (m, 2H), 2.06- | | |
| | | 1.89 (m, 3H). | | |
| 37 | (6,7-dichloro-1,3,4,5- | ¹H NMR (400 MHz, | 390.1 | The compound was |
| | tetrahydro-2H- | DMSO-d6) δ 11.57-11.50 | | synthesized following Ex 5 |
| | pyrido[4,3-b]indol-2- | (m, 1H), 8.38-8.28 (m, | | |
| | yl)(5- | 2H), 7.55-7.23 (m, 1H), | | |
| | (dimethylamino)pyrimidin- | 7.22-7.04 (m, 1H), 4.83- | | |
| | 2-yl)methanone | 4.45 (m, 2H), 4.06-3.55 | | |
| | | (m, 2H), 3.07-2.98 (m, | | |
| | | 6H), 2.95-2.82 (m, 2H). | | |
| 38 | (6,7-dichloro-1,3,4,5- | ¹H NMR (400 MHz, | 376.1 | The compound was |
| | tetrahydro-2H- | DMSO-d6) δ 11.57-11.49 | | synthesized following Ex 5 |
| | pyrido[4,3-b]indol-2- | (m, 1H), 8.18-8.09 (m, | | |
| | yl)(5- | 2H), 7.54-7.24 (m, 1H), | | |
| | (methylamino)pyrimidin- | 7.22-7.03 (m, 1H), 6.49 (q, | | |
| | 2-yl)methanone | J = 4.9 Hz, 1H), 4.84-4.45 | | |
| | | (m, 2H), 4.04-3.55 (m, | | |
| | | 2H), 2.94-2.81 (m, 2H), | | |
| | | 2.78 (d, J = 5.1 Hz, 3H). | | |
| 39 | (S)-(6,7-dichloro-1,3,4,5- | ¹H NMR (400 MHz, | 432.2 | The compound was |
| | tetrahydro-2H- | DMSO-d6) δ 11.56-11.49 | | synthesized following Ex 5 |
| | pyrido[4,3-b]indol-2- | (m, 1H), 8.20-8.11 (m, | | |
| | yl)(5-(3- | 2H), 7.55-7.24 (m, 1H), | | |
| | hydroxypyrrolidin-1- | 7.22-7.05 (m, 1H), 5.10- | | |
| | yl)pyrimidin-2- | 4.42 (m, 2H), 4.82-4.47 | | |
| | yl)methanone | (m, 2H), 4.05-3.56 (m, | | |
| | | 2H), 3.47-3.14 (m, 4H), | | |
| | | 2.95-2.82 (m, 2H), 2.11- | | |
| | | 1.90 (m, 2H). | | |
| 40 | (R)-(6,7-dichloro- | ¹H NMR (400 MHz, | 432.2 | The compound was |
| | 1,3,4,5-tetrahydro-2H- | DMSO-d6) δ 11.56-11.49 | | synthesized following Ex 5 |
| | pyrido[4,3-b]indol-2- | (m, 1H), 8.20-8.11 (m, | | |
| | yl)(5-(3- | 2H), 7.55-7.24 (m, 1H), | | |
| | hydroxypyrrolidin-1- | 7.22-7.04 (m, 1H), 5.10- | | |
| | yl)pyrimidin-2- | 4.42 (m, 2H), 4.84-4.47 | | |
| | yl)methanone | (m, 2H), 4.05-3.56 (m, | | |
| | | 2H), 3.46-3.14 (m, 4H), | | |
| | | 2.95-2.82 (m, 2H), 2.08- | | |
| | | 1.89 (m, 2H). | | |
| 41 | (6,7-dichloro-1,3,4,5- | ¹H NMR (400 MHz, | 416.1 | The compound was |
| | tetrahydro-2H- | DMSO-d6) δ 11.56-11.49 | | synthesized following Ex 5 |
| | pyrido[4,3-b]indol-2- | (m, 1H), 8.20-8.11 (m, | | |
| | yl)(5-(pyrrolidin-1- | 2H), 7.55-7.23 (m, 1H), | | |
| | yl)pyrimidin-2- | 7.23-7.05 (m, 1H), 4.82- | | |
| | yl)methanone | 4.46 (m, 2H), 3.80 (dt, J = | | |
| | | 164.1, 5.7 Hz, 2H), 3.38- | | |
| | | 3.30 (m, 4H), 2.95-2.82 | | |
| | | (m, 2H), 2.04-1.93 (m, | | |
| | | 4H). | | |
| 42 | (6,7-dichloro-1,3,4,5- | ¹H NMR (400 MHz, | 418.3 | The compound was |
| | tetrahydro-2H- | DMSO-d6) δ 11.57-11.49 | | synthesized following Ex 5 |
| | pyrido[4,3-b]indol-2- | (m, 1H), 8.13-8.03 (m, | | |
| | yl)(5-(3- | 2H), 7.55-7.23 (m, 1H), | | |
| | hydroxyazetidin-1- | 7.21-7.04 (m, 1H), 5.78 | | |
| | yl)pyrimidin-2- | (br. s, 1H), 4.82-4.42 (m, | | |
| | yl)methanone | 2H), 4.70-4.33 (m, 1H), | | |
| | | 4.30-3.69 (m, 4H), 4.05- | | |
| | | 3.52 (m, 2H), 2.94-2.80 | | |
| | | (m, 2H). | | |
| 43 | (6,7-dichloro-1,3,4,5- | ¹H NMR (400 MHz, | 420.2 | The compound was |
| | tetrahydro-2H- | DMSO-d6) δ 11.56-11.48 | | synthesized following Ex 5 |
| | pyrido[4,3-b]indol-2- | (m, 1H), 8.25-8.16 (m, | | |
| | yl)(5-((2- | 2H), 7.54-7.24 (m, 1H), | | |
| | methoxyethyl)ami- | 7.21-7.05 (m, 1H), 6.51 (q, | | |
| | no)pyrimidin-2- | J = 5.4 Hz, 1H), 4.81-4.45 | | |
| | yl)methanone | (m, 2H), 4.02-3.55 (m, | | |
| | | 2H), 3.48-3.38 (m, 1H), | | |
| | | 3.34-3.32 (m, 2H), 3.32- | | |
| | | 3.28 (m, 4H), 2.92-2.81 | | |
| | | (m, 2H). | | |
| Rac-46 | (6,7-dichloro-1-methyl- | ¹H NMR (400 MHz, | 390.2 | The compound was |
| | 1,3,4,5-tetrahydro-2H- | DMSO-d6) δ 11.55-11.47 | | synthesized following Ex 6 |
| | pyrido[4,3-b]indol-2- | (m, 1H), 8.18-8.10 (m, | | |
| | yl)(5- | 2H), 7.56-7.26 (m, 1H), | | |
| | (methylamino)pyrimidin- | 7.21-7.04 (m, 1H), 6.52- | | |
| | 2-yl)methanone | 6.40 (m, 1H), 5.77-4.76 | | |
| 46A* | (R)-(6,7-dichloro-1- | (m, 1H), 3.67-3.58 (m, | | |
| | methyl-1,3,4,5- | 1H), 3.51-3.21 (m, 1H), | | |
| | tetrahydro-2H- | 2.98-2.82 (m, 1H), 2.82- | | |
| | pyrido[4,3-b]indol-2- | 2.74 (m, 3H), 2.74-2.65 | | |
| | yl)(5- | (m, 1H), 1.53-1.45 (m, | | |
| | (methylamino)pyrimidin- | 3H). | | |
| | 2-yl)methanone | | | |
| 46B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5- | | | |
| | (methylamino)pyrimidin- | | | |
| | 2-yl)methanone | | | |
| Rac-47 | (6,7-dichloro-1-methyl- | ¹H NMR (400 MHz, | 461.3 | The compound was |
| | 1,3,4,5-tetrahydro-2H- | DMSO-d6) δ 11.56-11.47 | | synthesized following Ex 6 |
| | pyrido[4,3-b]indol-2- | (m, 1H), 8.36-8.28 (m, | | |
| | yl)(5-((2- | 2H), 7.57-7.25 (m, 1H), | | |
| | (dimethylamino)ethyl)(meth- | 7.23-7.04 (m, 1H), 5.73 (q, | | |
| | yl)amino)pyrimidin- | J = 6.5 Hz, 1H), 4.86-4.71 | | |
| | 2-yl)methanone | (m, 1H), 3.70-3.56 (m, | | |
| 47A* | (R)-(6,7-dichloro-1- | 2H), 3.53-3.41 (m, 1H), | | |
| | methyl-1,3,4,5- | 3.04-2.99 (m, 3H), 2.97- | | |
| | tetrahydro-2H- | 2.83 (m, 1H), 2.76-2.66 | | |
| | pyrido[4,3-b]indol-2- | (m, 1H), 2.62-2.51 (m, | | |
| | yl)(5-((2- | 2H), 2.33-2.28 (m, 6H), | | |
| | (dimethylamino)ethyl)(meth- | 1.50 (dd, J = 6.6, 2.3 Hz, | | |
| | yl)amino)pyrimidin- | 3H). | | |
| | 2-yl)methanone | | | |
| 47B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-((2- | | | |
| | (dimethylamino)ethyl)(meth- | | | |
| | yl)amino)pyrimidin- | | | |
| | 2-yl)methanone | | | |
| Rac-48 | (6,7-dichloro-1-methyl- | ¹H NMR (400 MHz, | 459.2 | The compound was |
| | 1,3,4,5-tetrahydro-2H- | DMSO-d6) δ 11.56-11.47 | | synthesized following Ex 6 |
| | pyrido[4,3-b]indol-2- | (m, 2H), 8.56 (s, 2H), 8.54- | | |
| | yl)(5-(4- | 8.12 (m, 2H), 7.57-7.27 | | |
| | methylpiperazin-1- | (m, 1H), 7.23-7.05 (m, | | |
| | yl)pyrimidin-2- | 1H), 5.73 (q, J = 6.7 Hz, | | |
| | yl)methanone | 1H), 4.81-4.71 (m, 1H), | | |
| 48A* | (R)-(6,7-dichloro-1- | 3.61-3.14 (m, 3H), 2.97- | | |
| | methyl-1,3,4,5- | 2.83 (m, 2H), 2.75-2.65 | | |
| | tetrahydro-2H- | (m, 1H), 2.29-2.23 (m, | | |
| | pyrido[4,3-b]indol-2- | 4H), 1.54-1.44 (m, 4H). | | |
| | yl)(5-(4- | | | |
| | methylpiperazin-1- | | | |
| | yl)pyrimidin-2- | | | |
| | yl)methanone | | | |
| 48B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-(4- | | | |
| | methylpiperazin-1- | | | |
| | yl)pyrimidin-2- | | | |
| | yl)methanone | | | |
| Rac-49 | (6,7-dichloro-1-methyl- | ¹H NMR (400 MHz, | 501.3 | The compound was |
| | 1,3,4,5-tetrahydro-2H- | DMSO-d6) δ 11.56-11.47 | | synthesized following Ex 6 |
| | pyrido[4,3-b]indol-2- | (m, 1H), 8.56 (s, 1H), 8.35 | | |
| | yl)(5-(4-(oxetan-3- | (d, J = 144.0 Hz, 2H), 7.57- | | |
| | yl)piperazin-1- | 7.27 (m, 1H), 7.23-7.05 | | |
| | yl)pyrimidin-2- | (m, 1H), 5.73 (q, J = 6.6 Hz, | | |
| | yl)methanone | 1H), 4.80-4.72 (m, 1H), | | |
| 49A* | (R)-(6,7-dichloro-1- | 4.60-4.44 (m, 4H), 3.62- | | |
| | methyl-1,3,4,5- | 3.20 (m, 3H), 2.96-2.65 | | |
| | tetrahydro-2H- | (m, 3H), 2.46-2.37 (m, | | |
| | pyrido[4,3-b]indol-2- | 4H), 1.54-1.45 (m, 4H). | | |
| | yl)(5-(4-(oxetan-3- | | | |
| | yl)piperazin-1- | | | |
| | yl)pyrimidin-2- | | | |
| | yl)methanone | | | |
| 49B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-(4-(oxetan-3- | | | |
| | yl)piperazin-1- | | | |
| | yl)pyrimidin-2- | | | |
| | yl)methanone | | | |
| Rac-50 | (6,7-dichloro-1-methyl- | ¹H NMR (400 MHz, | 521.2 | The compound was |
| | 1,3,4,5-tetrahydro-2H- | DMSO-d6) δ 11.58-11.47 | | synthesized following Ex 6 |
| | pyrido[4,3-b]indol-2- | (m, 1H), 9.06-8.98 (m, | | |
| | yl)(5-((S)-7,7- | 1H), 8.60-8.52 (m, 1H), | | |
| | difluorohexahydropyrrolo[1,2- | 7.58-7.26 (m, 1H), 7.23- | | |
| | a]pyrazin-2(1H)- | 7.05 (m, 1H), 5.82-5.68 | | |
| | yl)pyrimidin-2- | (m, 1H), 4.82-4.72 (m, | | |
| | yl)methanone | 1H), 4.12-3.89 (m, 2H), | | |
| 50A* | ((R)-6,7-dichloro-1- | 3.62-3.22 (m, 3H), 3.08- | | |
| | methyl-1,3,4,5- | 2.81 (m, 3H), 2.75-2.52 | | |
| | tetrahydro-2H- | (m, 3H), 2.44-1.90 (m, | | |
| | pyrido[4,3-b]indol-2- | 2H), 1.56-1.42 (m, 4H). | | |
| | yl)(5-((S)-7,7- | | | |
| | difluorohexahydropyrrolo[1,2- | | | |
| | a]pyrazin-2(1H)- | | | |
| | yl)pyrimidin-2- | | | |
| | yl)methanone | | | |
| 50B* | ((S)-6,7-dichloro-1- | | | |
| | methyl-1,3,4,5- | | | |
| | tetrahydro-2H- | | | |
| | pyrido[4,3-b]indol-2- | | | |
| | yl)(5-((S)-7,7- | | | |
| | difluorohexahydropyrrolo[1,2- | | | |
| | a]pyrazin-2(1H)- | | | |
| | yl)pyrimidin-2- | | | |
| | yl)methanone | | | |
| 54 | (R)-2-amino-1-(6,7- | ¹H NMR (400 MHz, | 328.1 | The compound was |
| | dichloro-1,3,4,5- | DMSO-d6) δ 11.55-11.48 | | synthesized following Ex 2 |
| | tetrahydro-2H- | (m, 1H), 8.28 (br. s, 2H), | | |
| | pyrido[4,3-b]indol-2-yl)- | 7.50-7.41 (m, 1H), 7.23- | | |
| | 3-hydroxypropan-1-one | 7.13 (m, 1H), 4.86-4.71 | | |
| | | (m, 1H), 4.71-4.59 (m, | | |
| | | 1H), 4.19-4.14 (m, 1H), | | |
| | | 4.06-3.73 (m, 1H), 3.91- | | |
| | | 3.85 (m, 2H), 3.63-3.57 | | |
| | | (m, 2H), 2.95-2.78 (m, | | |
| | | 2H). | | |
| 55 | (6,7-dichloro-1,3,4,5- | ¹H NMR (400 MHz, | 377.0 | The compound was |
| | tetrahydro-2H- | DMSO-d6) δ 11.53 (s, 1H), | | synthesized following Ex 1 |
| | pyrido[4,3-b]indol-2- | 8.56-8.46 (m, 1H), 8.39- | | |
| | yl)(5-methoxypyrazin-2- | 8.32 (m, 1H), 7.55-7.25 | | |
| | yl)methanone | (m, 1H), 7.21-7.07 (m, | | |
| | | 1H), 4.86-4.69 (m, 2H), | | |
| | | 4.00-3.94 (m, 3H), 4.05- | | |
| | | 3.76 (m, 2H), 2.95-2.90 | | |
| | | (m, 2H). | | |
| 56 | (6,7-dichloro-1,3,4,5- | ¹H NMR (400 MHz, | 377.0 | The compound was |
| | tetrahydro-2H- | DMSO-d6) δ 11.58-11.52 | | synthesized following Ex 1 |
| | pyrido[4,3-b]indol-2- | (m, 1H), 7.90-7.77 (m, | | |
| | yl)(6-methoxypyridazin- | 1H), 7.56-7.27 (m, 1H), | | |
| | 3-yl)methanone | 7.41-7.32 (m, 1H), 7.23- | | |
| | | 7.07 (m, 1H), 4.89-4.70 | | |
| | | (m, 2H), 4.13-4.09 (m, | | |
| | | 3H), 4.09-3.78 (m, 2H), | | |
| | | 2.99-2.86 (m, 2H). | | |
| 57 | 2-(6,7-dichloro-1,3,4,5- | ¹H NMR (400 MHz, | 312.1 | The compound was |
| | tetrahydro-2H- | DMSO-d6) δ 11.57-11.50 | | synthesized following Ex 1 |
| | pyrido[4,3-b]indol-2-yl)- | (m, 1H), 8.21-8.07 (m, | | |
| | 2-oxoacetamide | 1H), 7.79-7.67 (m, 1H), | | |
| | | 7.52-7.40 (m, 1H), 7.21- | | |
| | | 7.12 (m, 1H), 4.69-4.63 | | |
| | | (m, 2H), 3.91-3.74 (m, | | |
| | | 2H), 2.96-2.82 (m, 2H). | | |
| 58 | (R)-2-amino-1-(6,7- | ¹H NMR (400 MHz, | 312.2 | The compound was |
| | dichloro-1,3,4,5- | DMSO-d6) δ 11.57-11.50 | | synthesized following Ex 2 |
| | tetrahydro-2H- | (m, 1H), 8.21-8.07 (m, | | |
| | pyrido[4,3-b]indol-2- | 1H), 7.79-7.67 (m, 1H), | | |
| | yl)propan-1-one | 7.52-7.40 (m, 1H), 7.21- | | |
| | | 7.12 (m, 1H), 4.69-4.63 | | |
| | | (m, 2H), 3.91-3.74 (m, | | |
| | | 2H), 2.96-2.82 (m, 2H). | | |
| 64 | 1-(6,7-dichloro-1,3,4,5- | ¹H NMR (400 MHz, | 312.2 | The compound was |
| | tetrahydro-2H- | DMSO-d6) δ 11.58-11.49 | | synthesized following Ex 2 |
| | pyrido[4,3-b]indol-2-yl)- | (m, 1H), 8.21 (br. s, 1H), | | |
| | 2-(methylamino)ethan-1- | 7.52-7.39 (m, 1H), 7.24- | | |
| | one | 7.13 (m, 1H), 4.74-4.59 | | |
| | | (m, 2H), 4.07-3.95 (m, | | |
| | | 2H), 3.94-3.68 (m, 2H), | | |
| | | 3.37 (br. s, 1H), 3.46-2.86 | | |
| | | (m, 3H), 2.86-2.75 (m, | | |
| | | 1H) | | |
| 65 | 2-amino-1-(5,6-dichloro- | ¹H NMR (400 MHz, | 284.1 | The compound was |
| | 3,4-dihydropyrrolo[3,4- | DMSO-d6) δ 11.84 (s, 1H), | | synthesized following Ex 2 |
| | b]indol-2(1H)-yl)ethan- | 8.29 (s, 2H), 7.52-7.42 (m, | | |
| | 1-one | 1H), 7.27-7.20 (m, 1H), | | |
| | | 4.83-4.70 (m, 2H), 4.68- | | |
| | | 4.58 (m, 2H), 3.65-3.58 | | |
| | | (m, 2H). | | |
| 66 | 2-amino-1-(8,9-dichloro- | ¹H NMR (400 MHz, | 240.2 | The compound was |
| | 1,2,4,5,6,7-hexahydro- | DMSO-d6) δ 11.63-11.57 | | synthesized following Ex 2 |
| | 1,5-epiminooxocino[5,4- | (m, 1H), 8.23 (s, 1H), 7.60- | | |
| | b]indol-12-yl)ethan-1- | 7.47 (m, 1H), 7.24-7.13 | | |
| | one | (m, 1H), 5.77-5.23 (m, | | |
| | | 1H), 4.83-4.21 (m, 1H), | | |
| | | 3.95 (dd, J = 11.3, 8.8 Hz, | | |
| | | 2H), 3.89-3.70 (m, 2H), | | |
| | | 3.70-3.48 (m, 3H), 3.48- | | |
| | | 3.10 (m, 1H), 2.96-2.87 | | |
| | | (m, 1H). | | |
| Rac-67 | (6,7-dichloro-1-methyl- | ¹H NMR (400 MHz, | 435.1 | The compound was |
| | 1,3,4,5-tetrahydro-2H- | DMSO-d6) δ 11.54 (d, J = | | synthesized following Ex 7 |
| | pyrido[4,3-b]indol-2- | 14.1 Hz, 1H), 8.60 (m, J = | | |
| | yl)(4-(2- | 17.2, 5.9 Hz,1H), 7.56 (d, J = | | |
| | methoxyethoxy)pyrimidin- | 8.4 Hz, 1H), 7.13-7.02 | | |
| | 2-yl)methanone | (m, 2H), 5.74 (q, J = 6.7 Hz, | | |
| 67A* | (R)-(6,7-dichloro-1- | 1H), 4.74 (m, J = 18.9, 10.3 | | |
| | methyl-1,3,4,5- | Hz, 1H), 4.54-4.40 (m, | | |
| | tetrahydro-2H- | 2H), 3.66 (dt, J = 15.7, 4.5 | | |
| | pyrido[4,3-b]indol-2- | Hz, 2H), 3.51 (d, J = 7.9 Hz, | | |
| | yl)(4-(2- | 1H), 3.31 (d, J = 0.8 Hz, | | |
| | methoxyethoxy)pyrimidin- | 3H), 2.89 (d, J = 6.5 Hz, | | |
| | 2-yl)methanone | 1H), 2.73 (d, J = 16.3 Hz, | | |
| 67B* | (S)-(6,7-dichloro-1- | 1H), 1.52 (t, J = 6.6 Hz, | | |
| | methyl-1,3,4,5- | 3H). | | |
| | tetrahydro-2H- | | | |
| | pyrido[4,3-b]indol-2- | | | |
| | yl)(4-(2- | | | |
| | methoxyethoxy)pyrimidin- | | | |
| | 2-yl)methanone | | | |
| Rac-68 | (6,7-dichloro-1-methyl- | ¹H NMR (400 MHz, | 391.0 | The compound was |
| | 1,3,4,5-tetrahydro-2H- | DMSO-d6) δ 11.52 (d, J = | | synthesized following Ex 7 |
| | pyrido[4,3-b]indol-2- | 15.6 Hz, 1H), 8.60 (m, J = | | |
| | yl)(4-methoxypyrimidin- | 16.6, 5.6 Hz, 1H), 7.56 (d, J = | | |
| | 2-yl)methanone | 8.2 Hz, 1H), 7.35 (d, J = | | |
| 68A* | (R)-(6,7-dichloro-1- | 8.4 Hz, 1H), 7.13-7.01 (m, | | |
| | methyl-1,3,4,5- | 1H), 5.75 (d, J = 6.7 Hz, | | |
| | tetrahydro-2H- | 1H), 4.82-4.69 (m, 1H), | | |
| | pyrido[4,3-b]indol-2- | 3.94 (d, J = 13.6 Hz, 3H), | | |
| | yl)(4-methoxypyrimidin- | 3.51 (d, J = 6.9 Hz, 1H), | | |
| | 2-yl)methanone | 3.35 (s, 1H), 2.98-2.87 (m, | | |
| 68B* | (S)-(6,7-dichloro-1- | 1H), 1.53 (d, J = 6.5 Hz, | | |
| | methyl-1,3,4,5- | 3H). | | |
| | tetrahydro-2H- | | | |
| | pyrido[4,3-b]indol-2- | | | |
| | yl)(4-methoxypyrimidin- | | | |
| | 2-yl)methanone | | | |
| 69 | 1-(6,7-dichloro-1,3,4,5- | ¹H NMR (400 MHz, | 356.0 | The compound was |
| | tetrahydro-2H- | DMSO-d6) δ 11.55-11.47 | | synthesized following Ex 2 |
| | pyrido[4,3-b]indol-2-yl)- | (m, 1H), 8.20 (s, 1H), 7.50- | | |
| | 2-((2- | 7.38 (m, 1H), 7.23-7.13 | | |
| | methoxyethyl)amino)ethan- | (m, 1H), 4.71-4.59 (m, | | |
| | 1-one | 2H), 3.92-3.67 (m, 2H), | | |
| | | 3.84-3.76 (m, 2H), 3.55- | | |
| | | 3.43 (m, 2H), 3.26 (s, 3H), | | |
| | | 3.00 (q, J = 7.3 Hz, 1H), | | |
| | | 2.95-2.84 (m, 2H), 2.84- | | |
| | | 2.77 (m, 1H). | | |
| Rac-70 | (6,7-dichloro-1-methyl- | ¹H NMR (400 MHz, | 421.0 | The compound was |
| | 1,3,4,5-tetrahydro-2H- | DMSO-d6) δ 11.52 (d, J = | | synthesized following Ex 7 |
| | pyrido[4,3-b]indol-2- | 15.0 Hz, 1H), 8.59 (m, J = | | |
| | yl)(4-(2- | 18.0, 5.9 Hz, 1H), 7.35 (d, J = | | |
| | hydroxyethoxy)pyrimidin- | 8.5 Hz, 1H), 7.21 (d, J = | | |
| | 2-yl)methanone | 8.4 Hz, 1H), 7.03 (t, J = 6.2 | | |
| 70A* | (R)-(6,7-dichloro-1- | Hz, 1H), 5.75 (q, J = 6.9 Hz, | | |
| | methyl-1,3,4,5- | 1H), 4.90 (m, J = 10.6, 5.6 | | |
| | tetrahydro-2H- | Hz, 2H), 4.35 (m, J = 17.2, | | |
| | pyrido[4,3-b]indol-2- | 5.1 Hz, 2H), 3.72 (m, J = | | |
| | yl)(4-(2- | 10.2, 5.2 Hz, 2H), 3.51 (s, | | |
| | hydroxyethoxy)pyrimidin- | 1H), 2.92-2.86 (m, 1H), | | |
| | 2-yl)methanone | 2.73 (d, J = 16.1 Hz, 1H), | | |
| 70B* | (S)-(6,7-dichloro-1- | 1.52 (t, J = 6.2 Hz, 3H). | | |
| | methyl-1,3,4,5- | | | |
| | tetrahydro-2H- | | | |
| | pyrido[4,3-b]indol-2- | | | |
| | yl)(4-(2- | | | |
| | hydroxyethoxy)pyrimidin- | | | |
| | 2-yl)methanone | | | |
| 72 | (6,7-dichloro-9- | ¹H NMR (400 MHz, | 455.2 | The compound was |
| | (pyrimidin-5-yl)-1,3,4,5- | DMSO-d6) δ 11.88 (s, 1H), | | synthesized following Ex 13 |
| | tetrahydro-2H- | 9.39-9.11 (m, 1H), 9.03- | | |
| | pyrido[4,3-b]indol-2- | 8.65 (m, 2H), 8.65-8.46 | | |
| | yl)(5-methoxypyrimidin- | (m, 2H), 7.34-7.16 (m, | | |
| | 2-yl)methanone | 1H), 4.32-4.00 (m, 2H), | | |
| | | 3.99-3.93 (m, 2H), 3.52- | | |
| | | 3.39 (m, 2H), 3.68-3.14 | | |
| | | (m, 1H), 2.97-2.82 (m, | | |
| | | 2H). | | |
| 73 | (6,7-dichloro-9-(pyridin- | ¹H NMR (400 MHz, | 454.1 | The compound was |
| | 3-yl)-1,3,4,5-tetrahydro- | DMSO-d6) δ 11.83-11.77 | | synthesized following Ex 13 |
| | 2H-pyrido[4,3-b]indol-2- | (m, 1H), 8.74-8.65 (m, | | |
| | yl)(5-methoxypyrimidin- | 1H), 8.63 (s, 1H), 8.47 (s, | | |
| | 2-yl)methanone | 1H), 8.38-8.29 (m, 1H), | | |
| | | 7.98-7.58 (m, 1H), 7.57- | | |
| | | 7.20 (m, 1H), 7.18-7.04 | | |
| | | (m, 1H), 4.28-4.00 (m, | | |
| | | 3H), 3.98-3.92 (m, 2H), | | |
| | | 3.56 (s, 1H), 3.47 (t, J = 5.7 | | |
| | | Hz, 1H), 2.98-2.81 (m, | | |
| | | 2H). | | |
| Rac-76 | (6,7-dichloro-1-methyl- | — | — | The compound may be |
| | 1,3,4,5-tetrahydro-2H- | | | synthesized following Ex 14 |
| | pyrido[4,3-b]indol-2- | | | |
| | yl)(5-(2- | | | |
| | (methylamino)ethoxy)py- | | | |
| | rimidin-2-yl)methanone | | | |
| 76A* | (R)-(6,7-dichloro-1- | | | |
| | methyl-1,3,4,5- | | | |
| | tetrahydro-2H- | | | |
| | pyrido[4,3-b]indol-2- | | | |
| | yl)(5-(2- | | | |
| | (methylamino)ethoxy)py- | | | |
| | rimidin-2-yl)methanone | | | |
| 76B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-(2- | | | |
| | (methylamino)ethoxy)py- | | | |
| | rimidin-2-yl)methanone | | | |
| Rac-78 | (6,7-dichloro-1-methyl- | ¹H NMR (400 MHz, | 435.0 | The compound was |
| | 1,3,4,5-tetrahydro-2H- | DMSO-d6) δ 11.54-11.50 | | synthesized following Ex 14 |
| | pyrido[4,3-b]indol-2- | (m, 1H),8.68-8.64 (m, 2H), | | 78-rac was then subjected |
| | yl)(5-(2- | 7.55 (d, J = 8.0 Hz, 1H), | | to prep chiral HPLC using |
| | methoxyethoxy)pyrimidin- | 7.31-7.08 (m, 1H), 5.79- | | the following conditions: |
| | 2-yl)methanone | 5.74 (m, 1H), 4.80-4.65 | | Column: CHIRALPAK ID, |
| 78A* | (R)-(6,7-dichloro-1- | (m, 1H), 4.37-4.33 (m, | | 2*25 cm, 5 μm; Mobile |
| | methyl-1,3,4,5- | 2H), 3.73-3.70 (m, 2H), | | Phase A: Hex (0.1% TFA)-- |
| | tetrahydro-2H- | 3.51-3.48 (m, 1H), 3.33 (s, | | HPLC, Mobile Phase B: |
| | pyrido[4,3-b]indol-2- | 3H) 2.90-2.84 (m, 1H), | | MeOH:DCM = 1:1-- |
| | yl)(5-(2- | 2.72-2.68 (m, 1H), 1.53 (d, | | HPLC; Flow rate: 20 |
| | methoxyethoxy)pyrimidin- | J = 4.0 Hz, 2H), 1.47 (d, | | mL/min; Gradient: 20% B |
| | 2-yl)methanone | J = 8.0 Hz, 1H). | | to 20% B in 20 min |
| 78B* | (S)-(6,7-dichloro-1- | | | Compound 78A*: chiral |
| | methyl-1,3,4,5- | | | RT(min): 17.01 |
| | tetrahydro-2H- | | | Compound 78B*: chiral |
| | pyrido[4,3-b]indol-2- | | | RT(min): 12.95 |
| | yl)(5-(2- | | | |
| | methoxyethoxy)pyrimidin- | | | |
| | 2-yl)methanone | | | |
| Rac-79 | 3-((2-(6,7-dichloro-1- | — | — | The compound may be |
| | methyl-2,3,4,5- | | | synthesized following Ex 14 |
| | tetrahydro-1H- | | | |
| | pyrido[4,3-b]indole-2- | | | |
| | carbonyl)pyrimidin-5- | | | |
| | yl)oxy)propanenitrile | | | |
| 79A* | (R)-3-((2-(6,7-dichloro- | | | |
| | 1-methyl-2,3,4,5- | | | |
| | tetrahydro-1H- | | | |
| | pyrido[4,3-b]indole-2- | | | |
| | carbonyl)pyrimidin-5- | | | |
| | yl)oxy)propanenitrile | | | |
| 79B* | (S)-3-((2-(6,7-dichloro- | | | |
| | 1-methyl-2,3,4,5- | | | |
| | tetrahydro-1H- | | | |
| | pyrido[4,3-b]indole-2- | | | |
| | carbonyl)pyrimidin-5- | | | |
| | yl)oxy)propanenitrile | | | |
| Rac-80 | (6,7-dichloro-1-methyl- | — | — | The compound may be |
| | 1,3,4,5-tetrahydro-2H- | | | synthesized following Ex 14 |
| | pyrido[4,3-b]indol-2- | | | |
| | yl)(5-hydroxypyrimidin- | | | |
| | 2-yl)methanone | | | |
| 80A* | (R)-(6,7-dichloro-1- | | | |
| | methyl-1,3,4,5- | | | |
| | tetrahydro-2H- | | | |
| | pyrido[4,3-b]indol-2- | | | |
| | yl)(5-hydroxypyrimidin- | | | |
| | 2-yl)methanone | | | |
| 80B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-hydroxypyrimidin- | | | |
| | 2-yl)methanone | | | |
| Rac-81 | (6,7-dichloro-1-methyl- | — | — | The compound may be |
| | 1,3,4,5-tetrahydro-2H- | | | synthesized following Ex 14 |
| | pyrido[4,3-b]indol-2- | | | |
| | yl)(4-hydroxypyrimidin- | | | |
| | 2-yl)methanone | | | |
| 81A* | (R)-(6,7-dichloro-1- | | | |
| | methyl-1,3,4,5- | | | |
| | tetrahydro-2H- | | | |
| | pyrido[4,3-b]indol-2- | | | |
| | yl)(4-hydroxypyrimidin- | | | |
| | 2-yl)methanone | | | |
| 81B* | (S)-(6,7-dichloro-1- | | | |
| | methyl-1,3,4,5- | | | |
| | tetrahydro-2H- | | | |
| | pyrido[4,3-b]indol-2- | | | |
| | yl)(4-hydroxypyrimidin- | | | |
| | 2-yl)methanone | | | |
| Rac-82 | (6,7-dichloro-1-methyl- | — | — | The compound may be |
| | 1,3,4,5-tetrahydro-2H- | | | synthesized following Ex 15 |
| | pyrido[4,3-b]indol-2- | | | |
| | yl)(4-methoxy-5- | | | |
| | methylpyrimidin-2- | | | |
| | yl)methanone | | | |
| 82A* | (R)-(6,7-dichloro-1- | | | |
| | methyl-1,3,4,5- | | | |
| | tetrahydro-2H- | | | |
| | pyrido[4,3-b]indol-2- | | | |
| | yl)(4-methoxy-5- | | | |
| | methylpyrimidin-2- | | | |
| | yl)methanone | | | |
| 82B* | (S)-(6,7-dichloro-1- | | | |
| | methyl-1,3,4,5- | | | |
| | tetrahydro-2H- | | | |
| | pyrido[4,3-b]indol-2- | | | |
| | yl)(4-methoxy-5- | | | |
| | methylpyrimidin-2- | | | |
| | yl)methanone | | | |
| Rac-84 | (6,7-dichloro-1-methyl- | — | — | The compound may be |
| | 1,3,4,5-tetrahydro-2H- | | | synthesized following Ex |
| | pyrido[4,3-b]indol-2- | | | 15 and Ex 7 |
| | yl)(4-methoxy-5- | | | |
| | (methylamino)pyrimidin- | | | |
| | 2-yl)methanone | | | |
| 84A* | (R)-(6,7-dichloro-1- | | | |
| | methyl-1,3,4,5- | | | |
| | tetrahydro-2H- | | | |
| | pyrido[4,3-b]indol-2- | | | |
| | yl)(4-methoxy-5- | | | |
| | (methylamino)pyrimidin- | | | |
| | 2-yl)methanone | | | |
| 84B* | (S)-(6,7-dichloro-1- | | | |
| | methyl-1,3,4,5- | | | |
| | tetrahydro-2H- | | | |
| | pyrido[4,3-b]indol-2- | | | |
| | yl)(4-methoxy-5- | | | |
| | (methylamino)pyrimidin- | | | |
| | 2-yl)methanone | | | |
| Rac-85 | (5-amino-4- | — | — | The compound may be |
| | methoxypyrimidin-2- | | | synthesized following Ex |
| | yl)(6,7-dichloro-1- | | | 15 and Ex 7 |
| | methyl-1,3,4,5- | | | |
| | tetrahydro-2H- | | | |
| | pyrido[4,3-b]indol-2- | | | |
| | yl)methanone | | | |
| 85A* | (R)-(5-amino-4- | | | |
| | methoxypyrimidin-2- | | | |
| | yl)(6,7-dichloro-1- | | | |
| | methyl-1,3,4,5- | | | |
| | tetrahydro-2H- | | | |
| | pyrido[4,3-b]indol-2- | | | |
| | yl)methanone | | | |
| 85B* | (S)-(5-amino-4- | | | |
| | methoxypyrimidin-2- | | | |
| | yl)(6,7-dichloro-1- | | | |
| | methyl-1,3,4,5- | | | |
| | tetrahydro-2H- | | | |
| | pyrido[4,3-b]indol-2- | | | |
| | yl)methanone | | | |
| Rac-86 | (9-(6-aminopyridin-3- | — | — | The compound may be |
| | yl)-6,7-dichloro-1- | | | synthesized following Ex |
| | methyl-1,3,4,5- | | | 19 using Suzuki cross- |
| | tetrahydro-2H- | | | coupling reaction at the last |
| | pyrido[4,3-b]indol-2- | | | step with 5-(4,4,5,5- |
| | yl)(5-methoxypyrimidin- | | | tetramethyl-1,3,2- |
| | 2-yl)methanone | | | dioxaborolan-2-yl)pyridin- |
| 86A* | (R)-(9-(6-aminopyridin- | | | 2-amine |
| | 3-yl)-6,7-dichloro-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 86B* | (S)-(9-(6-aminopyridin- | | | |
| | 3-yl)-6,7-dichloro-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-87 | (10-(6-aminopyridin-3- | — | — | The compound may be |
| | yl)-7,8-dichloro-1- | | | synthesized following Ex |
| | methyl-3,4- | | | 7 and Ex 11 |
| | dihydropyrazino[1,2- | | | |
| | b]indazol-2(1H)-yl)(5- | | | |
| | methoxypyrimidin-2- | | | |
| | yl)methanone | | | |
| 87A* | (R)-(10-(6-aminopyridin- | | | |
| | 3-yl)-7,8-dichloro-1- | | | |
| | methyl-3,4- | | | |
| | dihydropyrazino[1,2- | | | |
| | b]indazol-2(1H)-yl)(5- | | | |
| | methoxypyrimidin-2- | | | |
| | yl)methanone | | | |
| 87B* | (S)-(10-(6-aminopyridin- | | | |
| | 3-yl)-7,8-dichloro-1- | | | |
| | methyl-3,4- | | | |
| | dihydropyrazino[1,2- | | | |
| | b]indazol-2(1H)-yl)(5- | | | |
| | methoxypyrimidin-2- | | | |
| | yl)methanone | | | |
| Rac-89 | 3-((2-(6,7-dichloro-1- | — | — | The compound may be |
| | methyl-2,3,4,5- | | | synthesized following Ex |
| | tetrahydro-1H- | | | 16 and Ex 7 |
| | pyrido[4,3-b]indole-2- | | | |
| | carbonyl)pyrimidin-4- | | | |
| | yl)oxy)propanenitrile | | | |
| 89A* | (R)-3-((2-(6,7-dichloro- | | | |
| | 1-methyl-2,3,4,5- | | | |
| | tetrahydro-1H- | | | |
| | pyrido[4,3-b]indole-2- | | | |
| | carbonyl)pyrimidin-4- | | | |
| | yl)oxy)propanenitrile | | | |
| 89B* | (S)-3-((2-(6,7-dichloro- | | | |
| | 1-methyl-2,3,4,5- | | | |
| | tetrahydro-1H- | | | |
| | pyrido[4,3-b]indole-2- | | | |
| | carbonyl)pyrimidin-4- | | | |
| | yl)oxy)propanenitrile | | | |
| Rac-90 | (5-amino-1H-imidazol- | — | — | The compound may be |
| | 2-yl)(6,7-dichloro-1- | | | synthesized following Ex 17 |
| | methyl-1,3,4,5- | | | |
| | tetrahydro-2H- | | | |
| | pyrido[4,3-b]indol-2- | | | |
| | yl)methanone | | | |
| 90A* | (R)-(5-amino-1H- | | | |
| | imidazol-2-yl)(6,7- | | | |
| | dichloro-1-methyl- | | | |
| | 1,3,4,5-tetrahydro-2H- | | | |
| | pyrido[4,3-b]indol-2- | | | |
| | yl)methanone | | | |
| 90B* | (S)-(5-amino-1H- | | | |
| | imidazol-2-yl)(6,7- | | | |
| | dichloro-1-methyl- | | | |
| | 1,3,4,5-tetrahydro-2H- | | | |
| | pyrido[4,3-b]indol-2- | | | |
| | yl)methanone | | | |
| Rac-91 | (6,7-dichloro-1-methyl- | — | — | The compound may be |
| | 1,3,4,5-tetrahydro-2H- | | | synthesized following Ex 17 |
| | pyrido[4,3-b]indol-2- | | | |
| | yl)(5-(methylamino)-1H- | | | |
| | imidazol-2-yl)methanone | | | |
| 91A* | (R)-(6,7-dichloro-1- | | | |
| | methyl-1,3,4,5- | | | |
| | tetrahydro-2H- | | | |
| | pyrido[4,3-b]indol-2- | | | |
| | yl)(5-(methylamino)-1H- | | | |
| | imidazol-2-yl)methanone | | | |
| 91B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-(methylamino)-1H- | | | |
| | imidazol-2-yl)methanone | | | |
| Rac-92 | (6,7-dichloro-1-methyl- | — | — | The compound may be |
| | 1,3,4,5-tetrahydro-2H- | | | synthesized following Ex 17 |
| | pyrido[4,3-b]indol-2- | | | |
| | yl)(5-(dimethylamino)- | | | |
| | 1H-imidazol-2- | | | |
| | yl)methanone | | | |
| 92A* | (R)-(6,7-dichloro-1- | | | |
| | methyl-1,3,4,5- | | | |
| | tetrahydro-2H- | | | |
| | pyrido[4,3-b]indol-2- | | | |
| | yl)(5-(dimethylamino)- | | | |
| | 1H-imidazol-2- | | | |
| | yl)methanone | | | |
| 92B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-(dimethylamino)- | | | |
| | 1H-imidazol-2- | | | |
| | yl)methanone | | | |
| Rac-93 | (6,7-dichloro-1-methyl- | — | — | The compound may be |
| | 1,3,4,5-tetrahydro-2H- | | | synthesized following Ex 17 |
| | pyrido[4,3-b]indol-2- | | | |
| | yl)(5-hydroxy-1H- | | | |
| | imidazol-2-yl)methanone | | | |
| 93A* | (R)-(6,7-dichloro-1- | | | |
| | methyl-1,3,4,5- | | | |
| | tetrahydro-2H- | | | |
| | pyrido[4,3-b]indol-2- | | | |
| | yl)(5-hydroxy-1H- | | | |
| | imidazol-2-yl)methanone | | | |
| 93B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-hydroxy-1H- | | | |
| | imidazol-2-yl)methanone | | | |
| Rac-94 | (6,7-dichloro-1-methyl- | — | — | The compound may be |
| | 1,3,4,5-tetrahydro-2H- | | | synthesized following Ex 17 |
| | pyrido[4,3-b]indol-2- | | | |
| | yl)(5-methoxy-1H- | | | |
| | imidazol-2-yl)methanone | | | |
| 94A* | (R)-(6,7-dichloro-1- | | | |
| | methyl-1,3,4,5- | | | |
| | tetrahydro-2H- | | | |
| | pyrido[4,3-b]indol-2- | | | |
| | yl)(5-methoxy-1H- | | | |
| | imidazol-2-yl)methanone | | | |
| 94B* | (S)-(6,7-dichloro-1- | | | |
| | methyl-1,3,4,5- | | | |
| | tetrahydro-2H- | | | |
| | pyrido[4,3-b]indol-2- | | | |
| | yl)(5-methoxy-1H- | | | |
| | imidazol-2-yl)methanone | | | |
| Rac-96 | (7-chloro-6-hydroxy-1- | — | — | The compound may be |
| | methyl-1,3,4,5- | | | synthesized following Ex |
| | tetrahydro-2H- | | | 15 using a late-stage |
| | pyrido[4,3-b]indol-2- | | | hydroxylation reaction |
| | yl)(5-methoxypyrimidin- | | | |
| | 2-yl)methanone | | | |
| 96A* | (R)-(7-chloro-6-hydroxy- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 96B* | (S)-(7-chloro-6-hydroxy- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-98 | 6,7-dichloro-N-methoxy- | ¹H NMR (400 MHz, | 328.0 | The compound was be |
| | 1-methyl-1,3,4,5- | DMSO-d6) δ 11.43 (s, 1H), | | synthesized following Ex |
| | tetrahydro-2H- | 9.79 (s, 1H), 7.41 (d, J = 8.4 | | 18 and Ex 7 |
| | pyrido[4,3-b]indole-2- | Hz, 1H), 7.17 (d, J = 8.4 Hz, | | |
| | carboxamide | 1H), 5.22 (q, J = 6.6 Hz, | | |
| 98A* | (R)-6,7-dichloro-N- | 1H), 4.07 (m, 1H), 3.56 (s, | | |
| | methoxy-1-methyl- | 3H), 3.24-3.13 (m, 1H), | | |
| | 1,3,4,5-tetrahydro-2H- | 2.81 (m, 1H), 2.71 (d, J = | | |
| | pyrido[4,3-b]indole-2- | 3.8 Hz, 1H), 1.39 (d, J = 6.5 | | |
| | carboxamide | Hz, 3H). | | |
| 98B* | (S)-6,7-dichloro-N- | | | |
| | methoxy-1-methyl- | | | |
| | 1,3,4,5-tetrahydro-2H- | | | |
| | pyrido[4,3-b]indole-2- | | | |
| | carboxamide | | | |
| Rac-99 | 6,7-dichloro-2-(5- | — | — | The compound may be |
| | methoxypyrimidine-2- | | | synthesized following Ex 6 |
| | sulfonimidoyl)-1- | | | and replacing the amide |
| | methyl-2,3,4,5- | | | coupling step with a |
| | tetrahydro-1H- | | | sulfonamide formation step |
| | pyrido[4,3-b]indole | | | using 5-methoxy |
| 99A* | (1R)-6,7-dichloro-2-(5- | | | pyrimidine-2-sulfonyl |
| | methoxypyrimidine-2- | | | chloride, followed by |
| | sulfonimidoyl)-1- | | | sulfonimidamide formation |
| | methyl-2,3,4,5- | | | following the method |
| | tetrahydro-1H- | | | described in |
| | pyrido[4,3-b]indole | | | CN108558883. |
| 99B* | (1S)-6,7-dichloro-2-(5- | | | |
| | methoxypyrimidine-2- | | | |
| | sulfonimidoyl)-1- | | | |
| | methyl-2,3,4,5- | | | |
| | tetrahydro-1H- | | | |
| | pyrido[4,3-b]indole | | | |
| Rac-100 | (6,7-dichloro-1-methyl- | — | — | The compound may be |
| | 3,4-dihydroimidazo[1,2- | | | synthesized following a |
| | a:5,4-c']dipyridin-2(1H)- | | | Pictet-Spengler approach |
| | yl)(5-methoxypyrimidin- | | | described in Heterocycles |
| | 2-yl)methanone | | | (1995), 41(1), 21-8, using |
| | | | | 3,4-dichloropyridin-2- |
| 100A* | (R)-(6,7-dichloro-1- | | | amine as the starting |
| | methyl-3,4- | | | material to form the |
| | dihydroimidazo[1,2- | | | imidazo-pyridine core and |
| | a:5,4-c']dipyridin-2(1H)- | | | following Ex 7 for the |
| | yl)(5-methoxypyrimidin- | | | amide formation step |
| | 2-yl)methanone | | | |
| 100B* | (S)-(6,7-dichloro-1- | | | |
| | methyl-3,4- | | | |
| | dihydroimidazo[1,2- | | | |
| | a:5,4-c']dipyridin-2(1H)- | | | |
| | yl)(5-methoxypyrimidin- | | | |
| | 2-yl)methanone | | | |
| Rac-102 | (6,7-dichloro-9-ethynyl- | — | — | The compound may be |
| | 1-methyl-1,3,4,5- | | | synthesized following Ex |
| | tetrahydro-2H- | | | 19 and Ex 7, using a |
| | pyrido[4,3-b]indol-2- | | | Sonogashira reaction at the |
| | yl)(5-methoxypyrimidin- | | | last step with |
| | 2-yl)methanone | | | (trimethylsilyl)acetylene |
| 102A* | (R)-(6,7-dichloro-9- | | | |
| | ethynyl-1-methyl- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| 102B* | (S)-(6,7-dichloro-9- | | | |
| | ethynyl-1-methyl- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| Rac-104 | (6,7-dichloro-1-methyl- | — | — | The compound may be |
| | 9-(prop-1-yn-1-yl)- | | | synthesized following Ex |
| | 1,3,4,5-tetrahydro-2H- | | | 19 and Ex 7, using a Stille |
| | pyrido[4,3-b]indol-2- | | | reaction at the last step |
| | yl)(5-methoxypyrimidin- | | | with tributyl-1-propyn-1- |
| | 2-yl)methanone | | | ylstannane |
| 104A* | (R)-(6,7-dichloro-1- | | | |
| | methyl-9-(prop-1-yn-1- | | | |
| | yl)-1,3,4,5-tetrahydro- | | | |
| | 2H-pyrido[4,3-b]indol-2- | | | |
| | yl)(5-methoxypyrimidin- | | | |
| | 2-yl)methanone | | | |
| 104B* | (S)-(6,7-dichloro-1- | | | |
| | methyl-9-(prop-1-yn-1- | | | |
| | yl)-1,3,4,5-tetrahydro- | | | |
| | 2H-pyrido[4,3-b]indol-2- | | | |
| | yl)(5-methoxypyrimidin- | | | |
| | 2-yl)methanone | | | |
| Rac-105 | (6,7-dichloro-9-(3- | — | — | The compound may be |
| | hydroxyprop-1-yn-1-yl)- | | | synthesized following Ex |
| | 1-methyl-1,3,4,5- | | | 19 and Ex 7, using a |
| | tetrahydro-2H- | | | Sonogashira reaction at the |
| | pyrido[4,3-b]indol-2- | | | last step with prop-2-yn-1- |
| | yl)(5-methoxypyrimidin- | | | ol |
| | 2-yl)methanone | | | |
| 105A* | (R)-(6,7-dichloro-9-(3- | | | |
| | hydroxyprop-1-yn-1-yl)- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 105B* | (S)-(6,7-dichloro-9-(3- | | | |
| | hydroxyprop-1-yn-1-yl)- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-106 | (6,7-dichloro-1-methyl- | — | — | The compound may be |
| | 1,3,4,5-tetrahydro-2H- | | | synthesized following Ex |
| | pyrido[4,3-b]indol-2- | | | 16 and Ex 7 using methyl |
| | yl)(6,7-dihydro-5H- | | | 4-chloro-5- |
| | pyrimido[4,5- | | | fluoropyrimidine-2- |
| | b][1,4]oxazin-2- | | | carboxylate and 2- |
| | yl)methanone | | | aminoethan-1-ol in step 1 |
| 106A* | (R)-(6,7-dichloro-1- | | | |
| | methyl-1,3,4,5- | | | |
| | tetrahydro-2H- | | | |
| | pyrido[4,3-b]indol-2- | | | |
| | yl)(6,7-dihydro-5H- | | | |
| | pyrimido [4,5- | | | |
| | b][1,4]oxazin-2- | | | |
| | yl)methanone | | | |
| 106B* | (S)-(6,7-dichloro-1- | | | |
| | methyl-1,3,4,5- | | | |
| | tetrahydro-2H- | | | |
| | pyrido[4,3-b]indol-2- | | | |
| | yl)(6,7-dihydro-5H- | | | |
| | pyrimido [4,5- | | | |
| | b][1,4]oxazin-2- | | | |
| | yl)methanone | | | |
| Rac-107 | (6,7-dichloro-1-methyl- | — | — | The compound may be |
| | 5-(oxetan-3-ylmethyl)- | | | synthesized following Ex |
| | 1,3,4,5-tetrahydro-2H- | | | 3 and Ex 7 |
| | pyrido[4,3-b]indol-2- | | | |
| | yl)(5-methoxypyrimidin- | | | |
| | 2-yl)methanone | | | |
| 107A* | (R)-(6,7-dichloro-1- | | | |
| | methyl-5-(oxetan-3- | | | |
| | ylmethyl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 107B* | (S)-(6,7-dichloro-1- | | | |
| | methyl-5-(oxetan-3- | | | |
| | ylmethyl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-108 | 2-(6,7-dichloro-2-(5- | — | — | The compound may be |
| | methoxypyrimidine-2- | | | synthesized following Ex |
| | carbonyl)-1-methyl- | | | 3 and Ex 7 |
| | 1,2,3,4-tetrahydro-5H- | | | |
| | pyrido[4,3-b]indol-5- | | | |
| | yl)acetonitrile | | | |
| 108A* | (R)-2-(6,7-dichloro-2-(5- | | | |
| | methoxypyrimidine-2- | | | |
| | carbonyl)-1-methyl- | | | |
| | 1,2,3,4-tetrahydro-5H- | | | |
| | pyrido[4,3-b]indol-5- | | | |
| | yl)acetonitrile | | | |
| 108B* | (S)-2-(6,7-dichloro-2-(5- | | | |
| | methoxypyrimidine-2- | | | |
| | carbonyl)-1-methyl- | | | |
| | 1,2,3,4-tetrahydro-5H- | | | |
| | pyrido[4,3-b]indol-5- | | | |
| | yl)acetonitrile | | | |
| Rac-109 | (6,7-dichloro-5-((2- | — | — | The compound may be |
| | methoxyethoxy)methyl)- | | | synthesized following Ex |
| | 1-methyl-1,3,4,5- | | | 3 and Ex 7 |
| | tetrahydro-2H- | | | |
| | pyrido[4,3-b]indol-2- | | | |
| | yl)(5-methoxypyrimidin- | | | |
| | 2-yl)methanone | | | |
| 109A* | (R)-(6,7-dichloro-5-((2- | | | |
| | methoxyethoxy)methyl)- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 109B* | (S)-(6,7-dichloro-5-((2- | | | |
| | methoxyethoxy)methyl)- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-110 | 3,4-dichloro-8-(5- | — | — | The compound may be |
| | methoxypyrimidine-2- | | | synthesized following the |
| | carbonyl)-2,9-dimethyl- | | | method described in |
| | 2,5,6,7,8,9-hexahydro- | | | Tetrahedron (1986), 42(8), |
| | 1H-pyrrolo[3,2-c:4,5- | | | 2303-9, using 5,6-dichloro- |
| | c′]dipyridin-1-one | | | 4-hydrazineylpyridin- |
| 110A* | (R)-3,4-dichloro-8-(5- | | | 2(1H)-one (made from |
| | methoxypyrimidine-2- | | | 4,5,6-trichloropyridin- |
| | carbonyl)-2,9-dimethyl- | | | 2(1H)-one via SN_{Ar}; |
| | 2,5,6,7,8,9-hexahydro- | | | Journal of the Chemical |
| | 1H-pyrrolo[3,2-c:4,5- | | | Society, Chemical |
| | c′]dipyridin-1-one | | | Communications (1972), |
| 110B* | (S)-3,4-dichloro-8-(5- | | | (9), 505-6) as the starting |
| | methoxypyrimidine-2- | | | material for the core |
| | carbonyl)-2,9-dimethyl- | | | formation and following Ex |
| | 2,5,6,7,8,9-hexahydro- | | | 7 for the amide formation |
| | 1H-pyrrolo[3,2-c:4,5- | | | step |
| | c′]dipyridin-1-one | | | |
| Rac-111 | (3,4-dichloro-9-methyl- | — | — | The compound may be |
| | 6,9- | | | synthesized following a |
| | dihydropyrido[4′,3′:4,5] | | | Pictet-Spengler approach |
| | imidazo[1,2-c]pyrimidin- | | | described in Heterocycles |
| | 8(7H)-yl)(5- | | | (1995), 41(1), 21-8, using |
| | methoxypyrimidin-2- | | | 5,6-dichloropyrimidin-4- |
| | yl)methanone | | | amine as the starting |
| 111A* | (R)-(3,4-dichloro-9- | | | material to form the |
| | methyl-6,9- | | | imidazo-pyridine core and |
| | dihydropyrido[4′,3′:4,5] | | | following Ex 7 for the |
| | imidazo[1,2-c]pyrimidin- | | | amide formation step |
| | 8(7H)-yl)(5- | | | |
| | methoxypyrimidin-2- | | | |
| | yl)methanone | | | |
| 111B* | (S)-(3,4-dichloro-9- | | | |
| | methyl-6,9- | | | |
| | dihydropyrido[4′,3′:4,5] | | | |
| | imidazo[1,2-c]pyrimidin- | | | |
| | 8(7H)-yl)(5- | | | |
| | methoxypyrimidin-2- | | | |
| | yl)methanone | | | |
| Rac-112 | (5,6-dichloro-1-methyl- | — | — | The compound may be |
| | 3,4-dihydropyrrolo[3,4- | | | synthesized following Ex 6 |
| | b]indol-2(1H)-yl)(5- | | | and Ex 7, using 2- |
| | methoxypyrimidin-2- | | | methylpiperidin-4-one |
| | yl)methanone | | | hydrochloride in the |
| 112A* | (R)-(5,6-dichloro-1- | | | Fischer indole formation |
| | methyl-3,4- | | | step |
| | dihydropyrrolo[3,4- | | | |
| | b]indol-2(1H)-yl)(5- | | | |
| | methoxypyrimidin-2- | | | |
| | yl)methanone | | | |
| 112B* | (S)-(5,6-dichloro-1- | | | |
| | methyl-3,4- | | | |
| | dihydropyrrolo[3,4- | | | |
| | b]indol-2(1H)-yl)(5- | | | |
| | methoxypyrimidin-2- | | | |
| | yl)methanone | | | |
| Rac-113 | (6,7-dichloro-1-methyl- | — | — | The compound may be |
| | 9-(1-methyl-1H-pyrazol- | | | synthesized following Ex |
| | 3-yl)-1,3,4,5-tetrahydro- | | | 19, using a Suzuki coupling |
| | 2H-pyrido[4,3-b]indol-2- | | | at the last step using 1- |
| | yl)(5-methoxypyrimidin- | | | methyl-3-(4,4,5,5- |
| | 2-yl)methanone | | | tetramethyl-1,3,2- |
| 113A* | (R)-(6,7-dichloro-1- | | | dioxaborolan-2-yl)-1H- |
| | methyl-9-(1-methyl-1H- | | | pyrazole |
| | pyrazol-3-yl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 113B* | (S)-(6,7-dichloro-1- | | | |
| | methyl-9-(1-methyl-1H- | | | |
| | pyrazol-3-yl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-114 | (6,7-dichloro-1-methyl- | — | — | The compound may be |
| | 1,3,4,5-tetrahydro-2H- | | | synthesized following Ex 6 |
| | pyrrolo[3,2-c:4,5- | | | and Ex 7, using 2,3- |
| | c′]dipyridin-2-yl)(5- | | | dichloro-4- |
| | methoxypyrimidin-2- | | | hydrazineylpyridine in the |
| | yl)methanone | | | Fischer indole formation |
| 114A* | (R)-(6,7-dichloro-1- | | | step |
| | methyl-1,3,4,5- | | | |
| | tetrahydro-2H- | | | |
| | pyrrolo[3,2-c:4,5- | | | |
| | c′]dipyridin-2-yl)(5- | | | |
| | methoxypyrimidin-2- | | | |
| | yl)methanone | | | |
| 114B* | (S)-(6,7-dichloro-1- | | | |
| | methyl-1,3,4,5- | | | |
| | tetrahydro-2H- | | | |
| | pyrrolo[3,2-c:4,5- | | | |
| | c′]dipyridin-2-yl)(5- | | | |
| | methoxypyrimidin-2- | | | |
| | yl)methanone | | | |
| Rac-115 | (6,7-dichloro-8- | — | — | The compound may be |
| | methoxy-1-methyl- | | | synthesized following Ex 6 |
| | 1,3,4,5-tetrahydro-2H- | | | and Ex 7, using (2,3- |
| | pyrido[4,3-b]indol-2- | | | dichloro-4- |
| | yl)(5-methoxypyrimidin- | | | methoxyphenyl)hydrazine |
| | 2-yl)methanone | | | in the Fischer indole |
| 115A* | (R)-(6,7-dichloro-8- | | | formation step |
| | methoxy-1-methyl- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| 115B* | (S)-(6,7-dichloro-8- | | | |
| | methoxy-1-methyl- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| Rac-116 | (6,7-dichloro-8-hydroxy- | — | — | The compound may be |
| | 1-methyl-1,3,4,5- | | | synthesized following Ex 6 |
| | tetrahydro-2H- | | | and Ex 7, using (2,3- |
| | pyrido[4,3-b]indol-2- | | | dichloro-4- |
| | yl)(5-methoxypyrimidin- | | | hydroxyphenyl)hydrazine |
| | 2-yl)methanone | | | in the Fischer indole |
| 116A* | (R)-(6,7-dichloro-8- | | | formation step |
| | hydroxy-1-methyl- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| 116B* | (S)-(6,7-dichloro-8- | | | |
| | hydroxy-1-methyl- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| Rac-117 | (6,7-dichloro-1-methyl- | — | — | The compound may be |
| | 8-(methylthio)-1,3,4,5- | | | synthesized following Ex 6 |
| | tetrahydro-2H- | | | and Ex 7, using (2,3- |
| | pyrido[4,3-b]indol-2- | | | dichloro-4- |
| | yl)(5-methoxypyrimidin- | | | (methylthio)phenyl)hydrazine |
| | 2-yl)methanone | | | in the Fischer indole |
| 117A* | (R)-(6,7-dichloro-1- | | | formation step |
| | methyl-8-(methylthio)- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| 117B* | (S)-(6,7-dichloro-1- | | | |
| | methyl-8-(methylthio)- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| Rac-118 | (7-chloro-6-fluoro-1- | — | — | The compound may be |
| | methyl-1,3,4,5- | | | synthesized following Ex 6 |
| | tetrahydro-2H- | | | and Ex 7, using (3-chloro- |
| | pyrido[4,3-b]indol-2- | | | 2-fluorophenyl)hydrazine |
| | yl)(5-methoxypyrimidin- | | | in the Fischer indole |
| | 2-yl)methanone | | | formation step |
| 118A* | (R)-(7-chloro-6-fluoro-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 118B* | (S)-(7-chloro-6-fluoro-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 119 | 6-chloro-2-(5- | — | — | The compound may be |
| | methoxypyrimidine-2- | | | synthesized following Ex 1, |
| | carbonyl)-7-methyl- | | | using 6-chloro-5- |
| | 1,2,3,4,5,7-hexahydro- | | | hydrazineyl-1- |
| | 8H-pyrrolo[2,3-c:4,5- | | | methylpyridin-2(1H)-one |
| | c']dipyridin-8-one | | | as the starting material in |
| | | | | the Fischer indole |
| | | | | formation step made via |
| | | | | SNAr on 5,6-dichloro-1- |
| | | | | methylpyridin-2(1H)-one |
| | | | | (made following the |
| | | | | procedure of Journal of |
| | | | | Medicinal Chemistry |
| | | | | (2020), 63(15) 8276-8295.) |
| Rac-121 | (7-bromo-6-chloro-1- | — | — | The compound may be |
| | methyl-1,3,4,5- | | | synthesized following Ex 6 |
| | tetrahydro-2H- | | | and Ex 7, using (3-bromo- |
| | pyrido[4,3-b]indol-2- | | | 2-chlorophenyl)hydrazine |
| | yl)(5-methoxypyrimidin- | | | in the Fischer indole |
| | 2-yl)methanone | | | formation step |
| 121A* | (R)-(7-bromo-6-chloro- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 121B* | (S)-(7-bromo-6-chloro- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 122 | 3-chloro-8-(5- | — | — | The compound may be |
| | methoxypyrimidine-2- | | | synthesized following Ex 1, |
| | carbonyl)-1-methyl-2- | | | using 3-chloro-5- |
| | oxo-2,5,6,7,8,9- | | | hydrazineyl-1-methyl-2- |
| | hexahydro-1H- | | | oxo-1,2-dihydropyridine-4- |
| | pyrrolo[3,2-b:4,5- | | | carbonitrile as the starting |
| | c']dipyridine-4- | | | material in the Fischer |
| | carbonitrile | | | indole formation step, |
| | | | | made from 4-bromo-3- |
| | | | | chloro-1-methyl-2(1H)- |
| | | | | pyridinone via aromatic |
| | | | | chlorination followed by |
| | | | | palladium-catalyzed |
| | | | | cyanation coupling reation |
| | | | | on the aryl bromide and a |
| | | | | SN_{Ar} step with hydrazine |
| Rac-123 | 7-chloro-2-(5- | — | — | The compound may be |
| | methoxypyrimidine-2- | | | synthesized following Ex 6 |
| | carbonyl)-1-methyl- | | | and Ex 7, using 2-chloro-6- |
| | 2,3,4,5-tetrahydro-1H- | | | hydrazineylbenzonitrile in |
| | pyrido[4,3-b]indole-6- | | | the Fischer indole |
| | carbonitrile | | | formation step |
| 123A* | (R)-7-chloro-2-(5- | | | |
| | methoxypyrimidine-2- | | | |
| | carbonyl)-1-methyl- | | | |
| | 2,3,4,5-tetrahydro-1H- | | | |
| | pyrido[4,3-b]indole-6- | | | |
| | carbonitrile | | | |
| 123B* | (S)-7-chloro-2-(5- | | | |
| | methoxypyrimidine-2- | | | |
| | carbonyl)-1-methyl- | | | |
| | 2,3,4,5-tetrahydro-1H- | | | |
| | pyrido[4,3-b]indole-6- | | | |
| | carbonitrile | | | |
| 124 | (7,8-dichloro-1,4,5,6- | | | The compound may be |
| | tetrahydroazepino[4,5- | | | synthesized following Ex 1 |
| | b]indol-3(2H)-yl)(5- | | | using azepan-4-one in the |
| | methoxypyrimidin-2- | | | Fischer indole formation |
| | yl)methanone | | | step |
| Rac-125 | 3-(6,7-dichloro-2-(5- | — | — | The compound may be |
| | methoxypyrimidine-2- | | | synthesized following Ex |
| | carbonyl)-1-methyl- | | | 19 using a Heck reaction at |
| | 2,3,4,5-tetrahydro-1H- | | | the last step with |
| | pyrido[4,3-b]indol-9- | | | acrylonitrile |
| | yl)propanenitrile | | | |
| 125A* | (R)-3-(6,7-dichloro-2-(5- | | | |
| | methoxypyrimidine-2- | | | |
| | carbonyl)-1-methyl- | | | |
| | 2,3,4,5-tetrahydro-1H- | | | |
| | pyrido[4,3-b]indol-9- | | | |
| | yl)propanenitrile | | | |
| 125B* | (S)-3-(6,7-dichloro-2-(5- | | | |
| | methoxypyrimidine-2- | | | |
| | carbonyl)-1-methyl- | | | |
| | 2,3,4,5-tetrahydro-1H- | | | |
| | pyrido[4,3-b]indol-9- | | | |
| | yl)propanenitrile | | | |
| Rac-126 | 2-(6,7-dichloro-2-(5- | — | — | The compound may be |
| | methoxypyrimidine-2- | | | synthesized following Ex |
| | carbonyl)-1-methyl- | | | 19 using a palladium- |
| | 2,3,4,5-tetrahydro-1H- | | | catalyzed arylation of |
| | pyrido[4,3-b]indol-9- | | | nitrile at the last step with |
| | yl)acetonitrile | | | 2- |
| 126A* | (R)-2-(6,7-dichloro-2-(5- | | | (trimethylsilyl)acetonitrile |
| | methoxypyrimidine-2- | | | |
| | carbonyl)-1-methyl- | | | |
| | 2,3,4,5-tetrahydro-1H- | | | |
| | pyrido[4,3-b]indol-9- | | | |
| | yl)acetonitrile | | | |
| 126B* | (S)-2-(6,7-dichloro-2-(5- | | | |
| | methoxypyrimidine-2- | | | |
| | carbonyl)-1-methyl- | | | |
| | 2,3,4,5-tetrahydro-1H- | | | |
| | pyrido[4,3-b]indol-9- | | | |
| | yl)acetonitrile | | | |
| Rac-127 | (6,7-dichloro-9- | — | — | The compound may be |
| | (difluoromethyl)-1- | | | synthesized following Ex |
| | methyl-1,3,4,5- | | | 19 using Ni-catalyzed |
| | tetrahydro-2H- | | | coupling at the last step |
| | pyrido[4,3-b]indol-2- | | | with bromo- |
| | yl)(5-methoxypyrimidin- | | | difluoromethane |
| | 2-yl)methanone | | | |
| 127A* | (R)-(6,7-dichloro-9- | | | |
| | (difluoromethyl)-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 127B* | (S)-(6,7-dichloro-9- | | | |
| | (difluoromethyl)-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-128 | (6,7-dichloro-9-ethyl-1- | — | — | The compound may be |
| | methyl-1,3,4,5- | | | synthesized following Ex |
| | tetrahydro-2H- | | | 19 using Negishi coupling |
| | pyrido[4,3-b]indol-2- | | | at the last step using |
| | yl)(5-methoxypyrimidin- | | | diethylzinc |
| | 2-yl)methanone | | | |
| 128A* | (R)-(6,7-dichloro-9- | | | |
| | ethyl-1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 128B* | (S)-(6,7-dichloro-9- | | | |
| | ethyl-1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-129 | (6,7-dichloro-9- | — | — | The compound may be |
| | (difluoromethoxy)-1- | | | synthesized following Ex |
| | methyl-1,3,4,5- | | | 19 using a palladium- |
| | tetrahydro-2H- | | | catalyzed hydroxylation |
| | pyrido[4,3-b]indol-2- | | | reaction at the last step, |
| | yl)(5-methoxypyrimidin- | | | followed by carbene |
| | 2-yl)methanone | | | trapping with sodium |
| 129A* | (R)-(6,7-dichloro-9- | | | chlorodifluoroacetate |
| | (difluoromethoxy)-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 129B* | (S)-(6,7-dichloro-9- | | | |
| | (difluoromethoxy)-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-130 | (6,7-dichloro-9- | — | — | The compound may be |
| | ((difluoromethyl)thio)-1- | | | synthesized following Ex |
| | methyl-1,3,4,5- | | | 19 using a palladium- |
| | tetrahydro-2H- | | | catalyzed SCF2H |
| | pyrido[4,3-b]indol-2- | | | installation at the last step |
| | yl)(5-methoxypyrimidin- | | | with 2-(2- |
| | 2-yl)methanone | | | methylphenyl)aniline; |
| 130A* | (R)-(6,7-dichloro-9- | | | palladium (2+); 2- |
| | ((difluoromethyl)thio)-1- | | | phenylaniline |
| | methyl-1,3,4,5- | | | methanesulfonic acid |
| | tetrahydro-2H- | | | |
| | pyrido[4,3-b]indol-2- | | | |
| | yl)(5-methoxypyrimidin- | | | |
| | 2-yl)methanone | | | |
| 130B* | (S)-(6,7-dichloro-9- | | | |
| | ((difluoromethyl)thio)-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-131 | (6,7-dichloro-1-methyl- | — | — | The compound may be |
| | 9-vinyl-1,3,4,5- | | | synthesized following Ex |
| | tetrahydro-2H- | | | 19 and Ex 7, using a |
| | pyrido[4,3-b]indol-2- | | | Suzuki coupling at the last |
| | yl)(5-methoxypyrimidin- | | | step with 4,4,5,5- |
| | 2-yl)methanone | | | tetramethyl-2-vinyl-1,3,2- |
| 131A* | (R)-(6,7-dichloro-1- | | | dioxaborolane |
| | methyl-9-vinyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 131B* | (S)-(6,7-dichloro-1- | | | |
| | methyl-9-vinyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-133 | 7,8-dichloro-2,3,4,9- | — | — | The compound may be |
| | tetrahydro-1H-2,4- | | | synthesized following Ex 9 |
| | epiminocarbazol-10- | | | and Ex 10, using 2,3- |
| | yl)(5-methoxypyrimidin- | | | dichloro-4-nitropyridine in |
| | 2-yl)methanone | | | the first step |
| 133A* | ((2S,4R)-7,8-dichloro- | | | |
| | 2,3,4,9-tetrahydro-1H- | | | |
| | 2,4-epiminocarbazol-10- | | | |
| | yl)(5-methoxypyrimidin- | | | |
| | 2-yl)methanone | | | |
| 133B* | ((2R,4S)-7,8-dichloro- | | | |
| | 2,3,4,9-tetrahydro-1H- | | | |
| | 2,4-epiminocarbazol-10- | | | |
| | yl)(5-methoxypyrimidin- | | | |
| | 2-yl)methanone | | | |
| Rac-134 | (3,4-dichloro-10-methyl- | — | — | The compound may be |
| | 7,8- | | | synthesized following Ex 9 |
| | dihydropyrido[4′,3′:3,4] | | | and Ex 10, using 2,3- |
| | pyrazolo[1,5-a]pyrazin- | | | dichloro-4-nitropyridine in |
| | 9(10H)-yl)(5- | | | the first step |
| | methoxypyrimidin-2- | | | |
| | yl)methanone | | | |
| 134A* | (R)-(3,4-dichloro-10- | | | |
| | methyl-7,8- | | | |
| | dihydropyrido[4′,3′:3,4] | | | |
| | pyrazolo[1,5-a]pyrazin- | | | |
| | 9(10H)-yl)(5- | | | |
| | methoxypyrimidin-2- | | | |
| | yl)methanone | | | |
| 134B* | (S)-(3,4-dichloro-10- | | | |
| | methyl-7,8- | | | |
| | dihydropyrido[4′,3′:3,4] | | | |
| | pyrazolo[1,5-a]pyrazin- | | | |
| | 9(10H)-yl)(5- | | | |
| | methoxypyrimidin-2- | | | |
| | yl)methanone | | | |
| 135 | (5,6-dichloro-1-methyl- | — | — | The compound may be |
| | 3,4-dihydropyrazolo[4,3- | | | synthesized following the |
| | b]indol-2(1H)-yl)(5- | | | procedure in Org. Biomol. |
| | methoxypyrimidin-2- | | | Chem., 2013, 11, 3288 to |
| | yl)methanone | | | provide intermediate |
| | | | | methyl 5,6-dichloro-2-(4- |
| | | | | methoxybenzyl)-3,4- |
| | | | | dihydropyrazolo[4,3- |
| | | | | b]indole-1(2H)-carboxylate |
| | | | | using 2-azido-3,4-dichloro- |
| | | | | 1-iodobenzene as the |
| | | | | starting material. |
| | | | | Compound 135 could then |
| | | | | be obtained using strong |
| | | | | acidic conditions for the |
| | | | | carbamate and PMB |
| | | | | deprotections followed by |
| | | | | methylation with Mel in |
| | | | | the presence of a base and |
| | | | | amide coupling as |
| | | | | described in Ex 1. |
| 136 | (6,7-dichloro-1-methyl- | — | — | The compound may be |
| | 1,3,4,5-tetrahydro-2H- | | | synthesized using Ex 9 for |
| | pyridazino[4,3-b]indol- | | | the synthesis of |
| | 2-yl)(5- | | | intermediate 6,7-dichloro- |
| | methoxypyrimidin-2- | | | 1H-indole, followed by |
| | yl)methanone | | | conditions of Organic |
| | | | | Letters (2011), 13(20), |
| | | | | 5564 to access intermediate |
| | | | | 2-(6,7-dichloro-1H-indol- |
| | | | | 2-yl)ethan-1-ol. |
| | | | | Intermediate 6,7-dichloro- |
| | | | | 2,3,4,5-tetrahydro-1H- |
| | | | | pyridazino[4,3-b]indole can |
| | | | | then be accessed through |
| | | | | C-3 bromination followed |
| | | | | by mesylation of the |
| | | | | alcohol, SN2 reaction using |
| | | | | hydrazine followed by |
| | | | | intramolecular palladium- |
| | | | | catalyzed C-N coupling. |
| | | | | Compound 136 could then |
| | | | | be synthesized following |
| | | | | methylation and amide |
| | | | | coupling as described in Ex |
| | | | | 1. |
| Rac-137 | (6,7-dichloro-4-phenyl- | — | — | The compound may be |
| | 1,3,4,5-tetrahydro-2H- | | | synthesized following Ex 1 |
| | pyrido[4,3-b]indol-2- | | | using 2-phenylpiperidin-4- |
| | yl)(5-methoxypyrimidin- | | | one in the Fischer indole |
| | 2-yl)methanone | | | formation step |
| 137A* | (R)-(6,7-dichloro-4- | | | |
| | phenyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 137B* | (S)-(6,7-dichloro-4- | | | |
| | phenyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-138 | (6,7-dichloro-4-(pyridin- | — | — | The compound may be |
| | 3-yl)-1,3,4,5-tetrahydro- | | | synthesized following Ex 1 |
| | 2H-pyrido[4,3-b]indol-2- | | | using 2-(pyridin-3- |
| | yl)(5-methoxypyrimidin- | | | yl)piperidin-4-one in the |
| | 2-yl)methanone | | | Fischer indole formation |
| 138A* | (R)-(6,7-dichloro-4- | | | step |
| | (pyridin-3-yl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 138B* | (S)-(6,7-dichloro-4- | | | |
| | (pyridin-3-yl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-139 | 6,7-dichloro-2-(5- | — | — | The compound may be |
| | methoxypyrimidine-2- | | | synthesized following Ex 1 |
| | carbonyl)-2,3,4,5- | | | using 4-oxopiperidine-2- |
| | tetrahydro-1H- | | | carbonitrile in the Fischer |
| | pyrido[4,3-b]indole-4- | | | indole formation step |
| | carbonitrile | | | |
| 139A* | (R)-6,7-dichloro-2-(5- | | | |
| | methoxypyrimidine-2- | | | |
| | carbonyl)-2,3,4,5- | | | |
| | tetrahydro-1H- | | | |
| | pyrido[4,3-b]indole-4- | | | |
| | carbonitrile | | | |
| 139B* | (S)-6,7-dichloro-2-(5- | | | |
| | methoxypyrimidine-2- | | | |
| | carbonyl)-2,3,4,5- | | | |
| | tetrahydro-1H- | | | |
| | pyrido[4,3-b]indole-4- | | | |
| | carbonitrile | | | |
| Rac-140 | (7,8-dichloro-1-methyl- | — | — | The compound may be |
| | 10-(1-methyl-1H- | | | synthesized following Ex |
| | pyrazol-3-yl)-3,4- | | | 7 and Ex 11 |
| | dihydropyrazino[1,2- | | | |
| | b]indazol-2(1H)-yl)(5- | | | |
| | methoxypyrimidin-2- | | | |
| | yl)methanone | | | |
| 140A* | (R)-(7,8-dichloro-1- | | | |
| | methyl-10-(1-methyl- | | | |
| | 1H-pyrazol-3-yl)-3,4- | | | |
| | dihydropyrazino[1,2- | | | |
| | b]indazol-2(1H)-yl)(5- | | | |
| | methoxypyrimidin-2- | | | |
| | yl)methanone | | | |
| 140B* | (S)-(7,8-dichloro-1- | | | |
| | methyl-10-(1-methyl- | | | |
| | 1H-pyrazol-3-yl)-3,4- | | | |
| | dihydropyrazino[1,2- | | | |
| | blindazol-2(1H)-yl)(5- | | | |
| | methoxypyrimidin-2- | | | |
| | yl)methanone | | | |
| 141 | (6,7-dichloro-5-methyl- | — | — | The compound may be |
| | 1,3,4,5-tetrahydro-2H- | | | synthesized following Ex 3 |
| | pyrido[4,3-b]indol-2- | | | |
| | yl)(5-methoxypyrimidin- | | | |
| | 2-yl)methanone | | | |
| Rac-142 | (7-bromo-6-fluoro-1- | — | — | The compound may be |
| | methyl-1,3,4,5- | | | synthesized following Ex 6 |
| | tetrahydro-2H- | | | and Ex 7, using (3-bromo- |
| | pyrido[4,3-b]indol-2- | | | 2-fluorophenyl)hydrazine |
| | yl)(5-methoxypyrimidin- | | | in the Fischer indole |
| | 2-yl)methanone | | | formation step |
| 142A* | (R)-(7-bromo-6-fluoro- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 142B* | (S)-(7-bromo-6-fluoro-1- | | | |
| | methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-143 | (3,4-dichloro-2- | — | — | The compound may be |
| | methoxy-9-methyl- | | | synthesized following Ex 6 |
| | 5,6,7,9-tetrahydro-8H- | | | and Ex 7, using 3,4- |
| | pyrrolo[3,2-b:4,5- | | | dichloro-5-hydrazineyl-2- |
| | c']dipyridin-8-yl)(5- | | | methoxypyridine in the |
| | methoxypyrimidin-2- | | | Fischer indole formation |
| | yl)methanone | | | step |
| 143A* | (R)-(3,4-dichloro-2- | | | |
| | methoxy-9-methyl- | | | |
| | 5,6,7,9-tetrahydro-8H- | | | |
| | pyrrolo[3,2-b:4,5- | | | |
| | c']dipyridin-8-yl)(5- | | | |
| | methoxypyrimidin-2- | | | |
| | yl)methanone | | | |
| 143B* | (S)-(3,4-dichloro-2- | | | |
| | methoxy-9-methyl- | | | |
| | 5,6,7,9-tetrahydro-8H- | | | |
| | pyrrolo[3,2-b:4,5- | | | |
| | c']dipyridin-8-yl)(5- | | | |
| | methoxypyrimidin-2- | | | |
| | yl)methanone | | | |
| Rac-144 | (6-chloro-7-ethynyl-1- | — | — | The compound may be |
| | methyl-1,3,4,5- | | | synthesized following Ex 6 |
| | tetrahydro-2H- | | | and Ex 7, using a late-stage |
| | pyrido[4,3-b]indol-2- | | | Sonogashira coupling with |
| | yl)(5-methoxypyrimidin- | | | ethynyltrimethylsilane |
| | 2-yl)methanone | | | |
| 144A* | (R)-(6-chloro-7-ethynyl- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 144B* | (S)-(6-chloro-7-ethynyl- | | | |
| | 1-methyl-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-145 | (3-bromo-4-chloro-2- | — | — | The compound may be |
| | methoxy-9-methyl- | | | synthesized following Ex 6 |
| | 5,6,7,9-tetrahydro-8H- | | | and Ex 7, using 3-bromo-4- |
| | pyrrolo[3,2-b:4,5- | | | chloro-5-hydrazineyl-2- |
| | c']dipyridin-8-yl)(5- | | | methoxypyridine in the |
| | methoxypyrimidin-2- | | | Fischer indole formation |
| | yl)methanone | | | step |
| 145A* | (R)-(3-bromo-4-chloro- | | | |
| | 2-methoxy-9-methyl- | | | |
| | 5,6,7,9-tetrahydro-8H- | | | |
| | pyrrolo[3,2-b:4,5- | | | |
| | c']dipyridin-8-yl)(5- | | | |
| | methoxypyrimidin-2- | | | |
| | yl)methanone | | | |
| 145B* | (S)-(3-bromo-4-chloro- | | | |
| | 2-methoxy-9-methyl- | | | |
| | 5,6,7,9-tetrahydro-8H- | | | |
| | pyrrolo[3,2-b:4,5- | | | |
| | c']dipyridin-8-yl)(5- | | | |
| | methoxypyrimidin-2- | | | |
| | yl)methanone | | | |
| Rac-146 | (6,7-dichloro-9- | — | — | The compound may be |
| | methoxy-1-methyl- | | | synthesized following Ex 6 |
| | 1,3,4,5-tetrahydro-2H- | | | and Ex 7, using 2,3- |
| | pyrrolo [3,2-c:4,5- | | | dichloro-4-hydrazineyl-6- |
| | c']dipyridin-2-yl)(5- | | | methoxypyridine in the |
| | methoxypyrimidin-2- | | | Fischer indole formation |
| | yl)methanone | | | step |
| 146A* | (R)-(6,7-dichloro-9- | | | |
| | methoxy-1-methyl- | | | |
| | 1,3,4,5-tetrahydro-2H- | | | |
| | pyrrolo[3,2-c:4,5- | | | |
| | c']dipyridin-2-yl)(5- | | | |
| | methoxypyrimidin-2- | | | |
| | yl)methanone | | | |
| 146B* | (S)-(6,7-dichloro-9- | | | |
| | methoxy-1-methyl- | | | |
| | 1,3,4,5-tetrahydro-2H- | | | |
| | pyrrolo [3,2-c:4,5- | | | |
| | c']dipyridin-2-yl)(5- | | | |
| | methoxypyrimidin-2- | | | |
| | yl)methanone | | | |
| Rac-147 | (6,7-dichloro-1- | — | — | The compound may be |
| | (fluoromethyl)-1,3,4,5- | | | synthesized following Ex 9 |
| | tetrahydro-2H- | | | and Ex 10, installing the |
| | pyrido[4,3-b]indol-2- | | | fluoride with SelectFluor |
| | yl)(5-methoxypyrimidin- | | | between step 4 and step 5 |
| | 2-yl)methanone | | | of Ex 9 |
| 147A* | (S)-(6,7-dichloro-1- | | | |
| | (fluoromethyl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 147B* | (R)-(6,7-dichloro-1- | | | |
| | (fluoromethyl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-148 | (6,7-dichloro-1- | — | — | The compound may be |
| | ((methylthio)methyl)- | | | synthesized following Ex 9 |
| | 1,3,4,5-tetrahydro-2H- | | | and Ex 10, using (sodium |
| | pyrido[4,3-b]indol-2- | | | methanethiolate as the |
| | yl)(5-methoxypyrimidin- | | | nucleophile species in step |
| | 2-yl)methanone | | | 3 of Ex 9 |
| 148A* | (S)-(6,7-dichloro-1- | | | |
| | ((methylthio)methyl)- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| 148B* | (R)-(6,7-dichloro-1- | | | |
| | ((methylthio)methyl)- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| Rac-149 | (6,7-dichloro-1- | — | — | The compound may be |
| | (difluoromethyl)-1,3,4,5- | | | synthesized following Ex 9 |
| | tetrahydro-2H- | | | and Ex 10, using |
| | pyrido[4,3-b]indol-2- | | | (difluoromethyl)trimethylsilane |
| | yl)(5-methoxypyrimidin- | | | as the nucleophile |
| | 2-yl)methanone | | | species in step 3 of Ex 9 |
| 149A* | (S)-(6,7-dichloro-1- | | | |
| | (difluoromethyl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| 149B* | (R)-(6,7-dichloro-1- | | | |
| | (difluoromethyl)-1,3,4,5- | | | |
| | tetrahydro-2H- | | | |
| | pyrido[4,3-b]indol-2- | | | |
| | yl)(5-methoxypyrimidin- | | | |
| | 2-yl)methanone | | | |
| Rac-150 | (6,7-dichloro-1- | — | — | The compound may be |
| | (methoxymethyl)- | | | synthesized following Ex 9 |
| | 1,3,4,5-tetrahydro-2H- | | | and Ex 10, performing |
| | pyrido[4,3-b]indol-2- | | | alpha-hydroxylation of the |
| | yl)(5-methoxypyrimidin- | | | ketone after step 4 of Ex 9, |
| | 2-yl)methanone | | | followed by methylation |
| 150A* | (S)-(6,7-dichloro-1- | | | |
| | (methoxymethyl)- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| 150B* | (R)-(6,7-dichloro-1- | | | |
| | (methoxymethyl)- | | | |
| | 1,3,4,5-tetrahydro-2H- | | | |
| | pyrido[4,3-b]indol-2- | | | |
| | yl)(5-methoxypyrimidin- | | | |
| | 2-yl)methanone | | | |
| Rac-151 | (6,7-dichloro-1-methyl- | — | — | The compound may be |
| | 3,4- | | | synthesized using a |
| | dihydropyrimido[1,6- | | | palladium-catalyzed C2-H |
| | a]indol-2(1H)-yl)(5- | | | activation coupling of 4,5- |
| | methoxypyrimidin-2- | | | dichloro-1H-indole with |
| | yl)methanone | | | tert-butyl (2- |
| 151A* | (R)-(6,7-dichloro-1- | | | bromoethyl)carbamate to |
| | methyl-3,4- | | | provide intermediate tert- |
| | dihydropyrimido[1,6- | | | butyl (2-(4,5-dichloro-1H- |
| | a]indol-2(1H)-yl)(5- | | | indol-2-yl)ethyl)carbamate. |
| | methoxypyrimidin-2- | | | C-3 bromination can be |
| | yl)methanone | | | performed using NBS, |
| 151B* | (S)-(6,7-dichloro-1- | | | followed by acidic Boc- |
| | methyl-3,4- | | | deprotection step and |
| | dihydropyrimido[1,6- | | | Pictet-Spengler cyclization |
| | a]indol-2(1H)-yl)(5- | | | with acetaldehyde with |
| | methoxypyrimidin-2- | | | conditions in Organic |
| | yl)methanone | | | Letters (2011), 13(20), |
| | | | | 5564 to provide |
| | | | | intermediate 6,7-dichloro- |
| | | | | 1-methyl-1,2,3,4- |
| | | | | tetrahydropyrimido[1,6- |
| | | | | a]indole. Desired |
| | | | | compound could then be |
| | | | | obtained using amide |
| | | | | coupling conditions in Ex |
| | | | | 7. |
| Rac-152 | (6,7-dichloro-1-methyl- | — | — | The compound may be |
| | 3,4-dihydropyrido[4,3- | | | synthesized using a |
| | b]indolizin-2(1H)-yl)(5- | | | cyclization between 2-(4- |
| | methoxypyrimidin-2- | | | bromo-3- |
| | yl)methanone | | | oxobutyl)isoindoline-1,3- |
| | | | | dione and 3,4-dichloro-2- |
| | | | | methylpyridine promoted |
| | | | | by DBU to provide |
| | | | | intermediate 2-(2-(7,8- |
| 152A* | (R)-(6,7-dichloro-1- | | | dimethylindolizin-2- |
| | methyl-3,4- | | | yl)ethyl)isoindoline-1,3- |
| | dihydropyrido[4,3- | | | dione. Then, phtalimide |
| | b]indolizin-2(1H)-yl)(5- | | | deprotection using |
| | methoxypyrimidin-2- | | | hydrazine followed by |
| | yl)methanone | | | Pictet-Spengler cyclization |
| | | | | using acetaldehyde |
| | | | | following procedure in |
| | | | | Organic Letters (2011), |
| 152B* | (S)-(6,7-dichloro-1- | | | 13(20), 5564 can lead to |
| | methyl-3,4- | | | intermediate 6,7-dichloro- |
| | dihydropyrido[4,3- | | | 1-methyl-1,2,3,4- |
| | b]indolizin-2(1H)-yl)(5- | | | tetrahydropyrido[4,3- |
| | methoxypyrimidin-2- | | | blindolizine. The desired |
| | yl)methanone | | | compound can then be |
| | | | | prepared following the |
| | | | | amide coupling conditions |
| | | | | in Ex 7. |
| Rac-153 | (7,8-dichloro-1-methyl- | — | — | The compound may be |
| | 3,4-dihydropyrazino[2,1- | | | synthesized using the |
| | a]isoindol-2(1H)-yl)(5- | | | procedures in J. Org. |
| | methoxypyrimidin-2- | | | Chem. 1969, 34, 1, 249 and |
| | yl)methanone | | | J. Org. Chem. 1969, 34, 6, |
| | | | | 1720 to access intermediate |
| 153A* | (R)-(7,8-dichloro-1- | | | 2-(4,5-dichloro-2H- |
| | methyl-3,4- | | | isoindol-2-yl)ethan-1- |
| | dihydropyrazino[2,1- | | | amine from 3,4-dichloro-2- |
| | a]isoindol-2(1H)-yl)(5- | | | formylbenzoyl chloride as |
| | methoxypyrimidin-2- | | | the starting material. Then, |
| | yl)methanone | | | a Pictet-Spengler |
| 153B* | (S)-(7,8-dichloro-1- | | | cyclization using |
| | methyl-3,4- | | | acetaldehyde with |
| | dihydropyrazino[2,1- | | | procedure in Organic |
| | a]isoindol-2(1H)-yl)(5- | | | Letters (2011), 13(20), |
| | methoxypyrimidin-2- | | | 5564, followed by amide |
| | yl)methanone | | | coupling using Ex 7 could |
| | | | | lead to the desired |
| | | | | compound. |
| 154 | (6,7-dichloro-1,3,4,5- | — | — | The compound may be |
| | tetrahydro-2H- | | | synthesized following Ex 1 |
| | pyrido[4,3-b]indol-2- | | | using 1-methyl-4,5- |
| | yl)(1-methyl-4,5- | | | dihydro-1H-1,2,4-triazole- |
| | dihydro-1H-1,2,4- | | | 3-carboxylic acid in the |
| | triazol-3-yl)methanone | | | amide coupling step |
| 157 | (S)-1-(6,7-dichloro-1- | ¹H NMR (400 MHz, | 313.0 | The compound may be |
| | methyl-1,3,4,5- | DMSO-d6) δ 11.45 (s, 1H), | | synthesized following Ex 7 |
| | tetrahydro-2H- | 7.47 (m, J = 14.5, 8.4 Hz, | | using glycolic acid in the |
| | pyrido[4,3-b]indol-2-yl)- | 1H), 7.18 (t, J = 8.3 Hz, | | amide coupling step |
| | 2-hydroxyethan-1-one | 1H), 5.63-5.11 (q, J = 6.5 | | |
| | | Hz, 1H), 4.75-4.66 (m, | | |
| | | 1H) 4.21 (m, 2H), 3.92 (m, J = | | |
| | | 14.0, 5.4 Hz, 1H), 3.41 | | |
| | | (m, J = 14.5, 11.8, 4.0 Hz, | | |
| | | 1H), 3.12 (m, J = 13.3, 11.6, | | |
| | | 5.6 Hz, 1H), 2.94 (m, J = | | |
| | | 17.2, 11.5, 5.4 Hz, 1H), 1.49 | | |
| | | (d, J = 6.4 Hz, 3H). | | |
| 158 | 2-amino-1-(6,7-dichloro- | ¹H NMR (400 MHz, | 298.2 | The compound may be |
| | 1,3,4,5-tetrahydro-2H- | DMSO-d6) δ 11.57-11.47 | | synthesized following Ex 2 |
| | pyrido[4,3-b]indol-2- | (m, 1H), 7.45 (dd, J = 8.5 | | |
| | yl)ethan-1-one | Hz, 1H), 7.17 (dd, J = 8.8 | | |
| | | Hz, 1H), 4.68-4.55 (m, | | |
| | | 2H), 3.90-3.67 (m, 2H), | | |
| | | 3.50-3.31 (m, 4H), 2.91- | | |
| | | 2.75 (m, 2H). | | |

### Assay Methods

heGAS Kinase-Glo Assay

Certain compounds of the present disclosure were tested for their h-cGAS inhibition activity using the methodology reported in Lama et al., “Development of human cGAS-specific small molecule inhibitors for repression of dsDNA-triggered interferon expression”, Nature Communications 10, Article number: 2261 (2019), with slight changes to some conditions as shown in Table B.

TABLE B
Summary of assay conditions

| | Lama | Present |
|---|---|---|
| | et al. 2019 | Disclosure |
| Enzyme | | |
| h-cGAS (nM) | 100 | 40 |
| Buffer | | |
| Tris-HCl pH 7.4 (mM) | 20 | 20 |
| MgCl₂ (mM) | 5 | 10 |
| NaCl (mM) | 150 | 25 |
| Tween ™-20 (%) | 0.01 | 0.01 |
| ZnCl₂ (μM) | 1 | 1 |
| DTT (mM) | 1 | 1 |
| DMSO (%) | 0.5 | 5 |
| Substrates | | |
| ATP (μM) | 100 | 100 |
| GTP (μM) | 100 | 100 |
| dsDNA (nM) | 25 | 25 |
| Assay | | |
| Plate (wells) | 384 | 384 |
| Incubation length (h) | 7 | 3 |
| Total volume (μL) | 20 | 20 |
| Kinase-Glo Max (μL) | 20 | 20 |

### Activity Data

The results of the hcGAS Kinase-Glo assay, expressed in IC₅₀ ranges, are provided in Table C with the following designations: A represents an IC₅₀ value <0.1 μM; B represents an IC₅₀ value ≥0.1 μM and <0.5 μM; C represents an IC₅₀ value ≥0.5 μM and <1.0 μM; D represents an IC₅₀ value ≥1.0 μM and <5.0 μM; and E represents an IC₅₀ value ≥5.0 μM. Raw data is provided in parentheses.

TABLE C
Activity Data

| # | hcGAS IC₅₀ |
|---|---|
| 1 | B (0.360) |
| 2 | D (1.18) |
| 3 | D (3.15) |
| 4 | C (0.513) |
| 5 | C (0.878) |
| 6 | D (1.765) |
| 7 | D (3.30) |
| 8 | D (1.801) |
| 9 | D (1.07) |
| 10 | D (3.22) |
| 11 | D (4.84) |
| 12 | C (0.513) |
| 13 | D (4.42) |
| 14 | D (1.52) |
| 15 | C (0.842) |
| 16 | B (0.463) |
| 17 | D (1.46) |
| 18 | D (2.58) |
| 19 | D (4.16) |
| 20 | D (1.17) |
| 21 | C (0.683) |
| 22 | D (4.54) |
| 23 | D (2.25) |
| 24 | D (2.52) |
| 25 | B (0.411) |
| 26 | D (3.57) |
| 27 | D (2.32) |
| 28 | D (2.54) |
| 29 | B (0.174) |
| 30 | B (0.226) |
| 31 | B (0.222) |
| 32 | B (0.156) |
| 33 | B (0.177) |
| 34 | B (0.219) |
| 35 | B (0.188) |
| 36 | B (0.236) |
| 37 | B (0.245) |
| 38 | B (0.207) |
| 39 | B (0.259) |
| 40 | B (0.209) |
| 41 | B (0.297) |
| 42 | B (0.256) |
| 43 | B (0.337) |
| Rac-44 | A (0.0685) |
| Rac-45 | A (0.0692) |
| 45A* | E (>10) |
| 45B* | A (0.0374) |
| Rac-46 | A (0.0514) |
| Rac-47 | A (0.0501) |
| Rac-48 | A (0.0520) |
| Rac-49 | B (0.102) |
| Rac-50 | A (0.0895) |
| Rac-51 | A (0.0377) |
| 51A* | E (>10) |
| 51B* | A (0.0178) |
| 52A* | E (>10) |
| 52B* | A (0.0715) |
| 53 | D (1.28) |
| 54 | D (1.45) |
| 55 | D (2.39) |
| 56 | D (4.35) |
| 57 | D (4.21) |
| 58 | D (2.08) |
| 59 | B (0.620) |
| 60A | E (>10) |
| 60B | A (0.0828) |
| 61A* | E (>10) |
| 61B* | A (0.0599) |
| 64 | D (1.49) |
| 65 | C (0.933) |
| 66 | B (0.314) |
| Rac-67 | B (0.251) |
| Rac-68 | A (0.0945) |
| 69 | C (0.678) |
| Rac-70 | A (0.0787) |
| 71 | B (0.133) |
| 72 | B (0.156) |
| 73 | B (0.224) |
| 77A* | E (>10) |
| 77B* | A (0.0320) |
| 78A* | — |
| 78B* | A (0.0904) |
| Rac-83 | B (0.206) |
| Rac-88 | B (0.110) |
| 95A* | E (>10) |
| 95B* | C (0.587) |
| Rac-97 | C (0.821) |
| Rac-98 | D (1.32) |
| Rac-101 | B (0.409) |
| Rac-103 | B (0.115) |
| 120A* | E (>10) |
| 120B* | A (0.0331) |
| Rac-132 | A (0.0455) |
| 157 | A (0.019) |
| 158 | A (0.095) |

### Comparative Data

As demonstrated herein, certain structural aspects of compounds of Formula (I) show an improvement in hcGAS activity and solubility (e.g., in phosphate buffered saline (PBS)). For example, while a ring at position R³ results in an improvement in hcGAS activity, the inclusion of a methyl group at the X⁷/R² position results dramatically improved solubility while maintaining improved hcGAS activity. Contrast the hcGAS activity and solubility of Comparative Examples A and B to Compound 157 (Table D). Further contrast the hcGAS activity and solubility of Comparative Examples C and D to Compound 60B and Compound 120B* (Table D).

TABLE D
Comparative Data

| Name/Structure | hcGAS (IC₅₀) | Solubility(uM) |
|---|---|---|
| 1-(6,7-dichloro-1,3,4,5-tetrahydro-2H-pyrido[4,3-b]indol-2- yl)-2-hydroxyethan-1-one 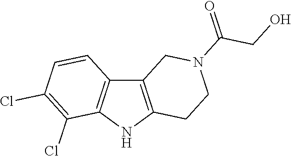 [SMILES: O=C(CO)N1CCc2[nH]c3c(Cl)c(Cl)ccc3c2C1] Comparative Example A WO 2019/153002 (TDI-005685) | B (0.1462) | 2.0 |
| 1-(6,7-dichloro-9-(1-methyl-1H-pyrazol-3-yl)-1,3,4,5- tetrahydro-2H-pyrido[4,3-b]indol-2-yl)-2-hydroxyethan-1- one 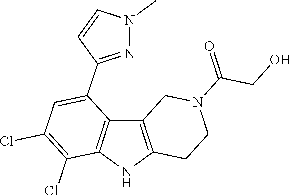 [SMILES: Cn1ccc(-c2cc(Cl)c(Cl)c3[nH]c4c(c23)CN(C(=O)CO)CC4)n1] Comparative Example B WO 2019/153002 (TDI-007635) | A (0.03524) | 8.1 |
| (S)-1-(6,7-dichloro-1-methyl-1,3,4,5-tetrahydro-2H- pyrido[4,3-b]indol-2-yl)-2-hydroxyethan-1-one - 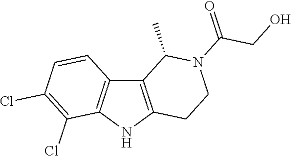 [SMILES: C[C@H]1c2c([nH]c3c(Cl)c(Cl)ccc23)CCN1C(=O)CO] (157) | A (0.019) | 42 |
| 1-(7,8-dichloro-3,4-dihydropyrazino[1,2-b]indazol-2(1H)- yl)-2-hydroxyethan-1-one 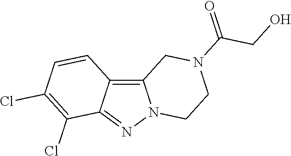 [SMILES: O=C(CO)N1CCn2nc3c(Cl)c(Cl)ccc3c2C1] Comparative Example C WO 2020/186027 (Compound 4) | E (12.84) | 76 |
| 1-(7,8-dichloro-10-(1-methyl-1H-pyrazol-3-yl)-3,4- dihydropyrazino[1,2-b]indazol-2(1H)-yl)-2-hydroxyethan-1- one 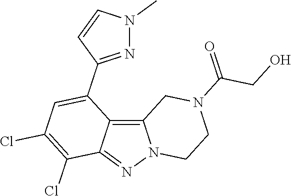 [SMILES: Cn1ccc(-c2cc(Cl)c(Cl)c3nn4c(c23)CN(C(=O)CO)CC4)n1] Comparative Example D WO 2020/186027 (Compound 67) | A (0.02728) | 53 |
| (S)-1-(7,8-dichloro-1-methyl-3,4-dihydropyrazino[1,2- b]indazol-2(1H)-yl)-2-hydroxyethan-1-one 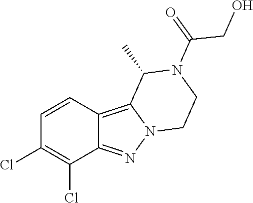 [SMILES: C[C@H]1c2c3ccc(Cl)c(Cl)c3nn2CCN1C(=O)CO] (60B) | A (0.0828) | 250 |
| (S)-2-amino-1-(7,8-dichloro-1-methyl-3,4- dihydropyrazino[1,2-b]indazol-2(1H)-yl)ethan-1-one 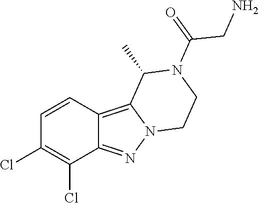 [SMILES: C[C@H]1c2c3ccc(Cl)c(Cl)c3nn2CCN1C(=O)CN] (120B*) | A (0.0331) | 313 |

As further demonstrated herein, compounds with a non-hydrogen X⁷/R² group with (S)-stereochemistry (in the “down” orientation) demonstrate an improvement in hcGAS activity. Compare Compound 60B with the (S)-methyl group (“A” activity) to Compound 60A with the (R)-methyl group (“E” activity), in contrast to Comparative Example A with no methyl group at the X⁷/R² position (“B” activity). See also pyrimindinyl compounds of Formula (IV), demonstrating a non-hydrogen group at the X⁷/R² position leads to improvement in activity: compare Compound 44 (“A” activity) to Compound 31 (“B” activity); Compound 45 (“A” activity) to Compound 1 (“B” activity); and Compound 46 (“A” activity) to Compound 38 (“B” activity).

Compounds of Formula (I), wherein Y is C, show an improvement in activity when R¹ is hydrogen. Compare Compounds 158 (“A” activity), Compound 51 (“A” activity), Compound 611B* (“A” activity), Compound 59 (“B” activity) and Compound 66 (“B” activity), where Y is C and R¹ is hydrogen, to Compounds 53, 54, 57, and 58, each where Y is C and R¹ is not hydrogen and each having “D” activity.

Compounds of the present disclosure also show an improvement in activity when R⁹ is hydrogen. Compare Compounds 157 and 158, where R⁹ is hydrogen (“A” activity) to Compound 64 (“D” activity) and Compound 69 (“D” activity), where R⁹ is not hydrogen.

Compounds of Formula (I) wherein X¹ is NR⁵, such as pyrimidinyl compounds of Formula (IV), show an improvement in activity when R¹ is hydrogen. Compare Compound 1 (“B” activity) to Compounds 17, 18, 19, 20, 22, 23, 24, 26, 27, and 28 (each having “D” activity), Compound 21 (“C” activity) and Compound 25 (“B” activity).

Pyrimidinyl compounds of Formula (IV) wherein R⁴ is a non-hydrogen group at the para position of the pyrimidinyl ring show an improvement in activity. Compare Compound 1 (“B” activity), comprising a para R⁴ substituent, to Compound 2 (“D” activity), Compound 4 (“C” activity), Compound 8 (“D” activity) and Compound 14 (“D” activity), which comprise no substituent or a meta R⁴ substituent. Furthermore, such compounds where R⁴ is a para electron donating group, such as an alkoxy or amino group, show an improvement in activity. Compare Compounds 1, 29, 30, 31, 32, 33, 34, 35, 36, 37, 38, 39, 40, 41, 42, and 43 (each having “B” activity) to Compounds 12 and 15 (each having “C” activity).

Pyrimidinyl compounds of Formula (IV) also show improved activity when X⁷ is CHR² and R² is a non-hydrogen group, including upon moving the R⁴ para substituent to the meta position of the pyrimidinyl ring. Compare, for example, moving the para —OMe substituent of Compound 1, where X⁷ is —CH₂—(“B” activity) to the meta position of Compound 4, where X⁷ is —CH₂—(“C” activity), versus moving the para —OMe substituent of Compound 45, where X⁷ is —CH(CH₃)—(“A” activity) to the meta position of Compound 68, where X⁷ is —CH(CH₃)— (“A” activity). See also Compounds 67 and 70, each having “A” activity.

Compounds of Formula (I), wherein R¹ and R⁹ combine to form a heteroaryl ring, such as pyrimidinyl compounds of Formula (IV), also show an improvement in activity moving from 5-membered heteroaryl rings to 6-membered heteroaryl rings. Compare 5-membered heteroaryl-containing Compounds 3, 6, 7, 9, 10, 11, and 13 (each having “D” activity) and 5-membered heteroaryl-containing Compound 5 (“C” activity) to pyrimidinyl-containing Compound 1 of Formula (IV) (“B” activity).

### Additional Embodiments

Additional embodiments of the disclosure are indicated by the following numbered embodiments:

Embodiment 1. A compound of Formula I

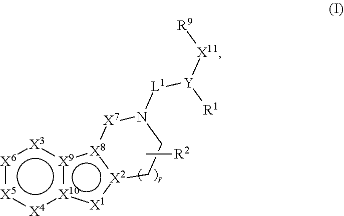

or a pharmaceutically acceptable salt, hydrate, solvate, prodrug, stereoisomer, and tautomer thereof, wherein:
L¹ is —C(O)—, —S(O), —S(O)₂—, —S(NH)(O)—;
X¹ is independent N, NR⁵, or CH;
X² are independent N or C wherein at least one of X¹ is N or NR⁵ or X² is N;
X³, X⁴, X⁵, X⁶, X⁸, X⁹, and X¹⁰ are independently CR³ or N, wherein at least one of X³, X⁴, X⁵, and X⁶ is CR³ and wherein X³, X⁴, X⁵, X⁶, X⁸, X⁹, and X¹⁰, independently, are not more than 7 N in total;
X⁷ is independently NH, NCH₃, or C(R²)₂;
X¹¹ is independently O, N or NH;
Y is NH, CH, or C
R¹ is H or C₁-C₆ alkyl, wherein the alkyl is optionally substituted with one or more R⁴; or
R¹ and R⁹ combine to form a 3- to 8-membered heterocycle, or 5- to 10-membered heteroaryl, wherein the heterocycle, or heteroaryl is optionally substituted with one or more R⁴;
R² is H, halogen, CN, OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are attached can form a C₄-C₈ cycloalkyl or 4- to 6-membered heterocycle;
each R³ is independently H, halogen, oxo, —CN, OR⁵, SR⁵, —NH₂, NH(R⁵), —N(R⁵)(R⁶), —NHC(O)R⁵, —CO(OR⁵), —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶;
R⁴ is H, halogen, —CN, OR⁵, —NH₂, NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or R₇;
each R⁵ is H, —C(O)OH, —(CH₂)ₙ—O—(CH₂)ₚ—OR⁸, —(CH₂)ₙ—OR⁸, —(CH₂)ₙ—S(O)₂R⁸, CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or more halogen, OH, CN, NH₂, N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl;
R⁶, R⁷, and R⁸ is independently, at each occurrence, H, halogen, OH, CN, NH₂, C₁-C₆ alkyl, C₁-C₆ hydroxyalkyl, C₁-C₆ alkoxy, C₂-C₆ alkenyl, C₂-C₆ alkynyl, C₃-C₈ cycloalkyl, heterocyclyl, heteroaryl, or aryl;
R⁹ is independently H or C₁-C₄ alkyl, wherein the alkyl is optionally substituted with one or more OH, halogen, CN, C₁-C₆ alkoxy, or cycloalkyl;
each n is independently an integer from 0 to 6;
each p is independently an integer from 0 to 6; and
r is an integer from 0 to 2;
provided that (1) when R¹ and R⁹ combine to form a 3- to 8-membered heterocycle, or 5- to 10-membered heteroaryl, X¹¹ is N or NH; (2) when X¹¹ is O, X¹ and X² are both N and X⁷ is —CH(R²)—; (3) when X¹¹ is NH and R⁹ is H, X⁷ is not —CH₂—; and (4) when X³ is CR³ and R³ is heteroaryl, R¹ and R⁹ form a heteroaryl; (5) R¹ and R⁹ when do not combine to form a pyrazole.

Embodiment 2. The compound of embodiment 1, wherein the compound is of Formula (I-a):

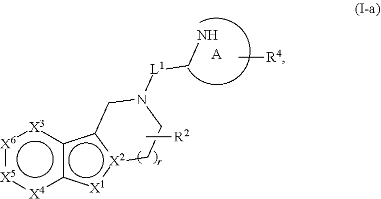

or a pharmaceutically acceptable salt, isomer, solvate, prodrug, or tautomer thereof,
wherein A represents a 3- to 8-membered heterocycle or 5- or 10-membered heteroaryl.

Embodiment 3. The compound of embodiment 1, wherein Formula (I-a) is Formula (I-a-1)

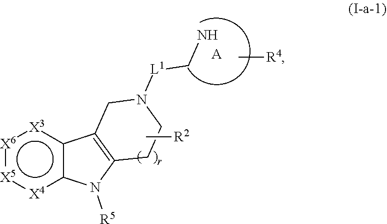

or a pharmaceutically acceptable salt, isomer, solvate, prodrug, or tautomer thereof,
wherein A represents a 3- to 8-membered heterocycle or 5- or 10-membered heteroaryl.

Embodiment 4. The compound of embodiment 1, wherein Formula (I-a) is Formula (I-a-1-i)

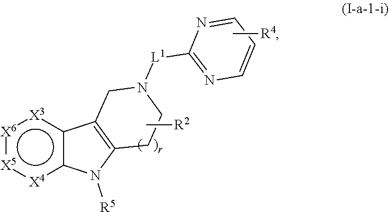

or a pharmaceutically acceptable salt, isomer, solvate, prodrug, or tautomer thereof.

Embodiment 5. The compound of embodiment 1, wherein Formula (I-a) is Formula (I-a-1-ii)

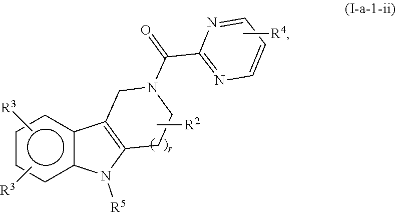

or a pharmaceutically acceptable salt, isomer, solvate, prodrug, or tautomer thereof.

Embodiment 6. The compound of embodiment 1, wherein Formula (I-a) is Formula (I-a-2)

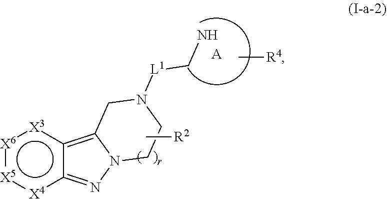

or a pharmaceutically acceptable salt, isomer, solvate, prodrug, or tautomer thereof,
wherein A represents a 3- to 8-membered heterocycle or 5- or 10-membered heteroaryl.

Embodiment 7. The compound of embodiment 1, wherein Formula (I-a) is Formula (I-a-2-i):

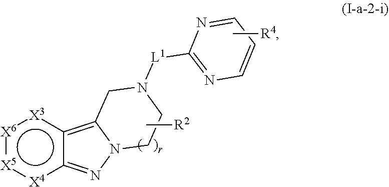

or a pharmaceutically acceptable salt, isomer, solvate, prodrug, or tautomer thereof.

Embodiment 8. The compound of embodiment 1, wherein Formula (I-a) is Formula (I-a-2-ii)

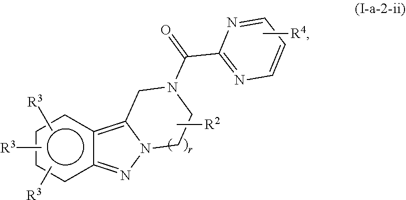

or a pharmaceutically acceptable salt, isomer, solvate, prodrug, or tautomer thereof.

Embodiment 9. The compound of embodiment 1, wherein Formula (I-a) is Formula (I-a-3) to Formula (I-a-11):

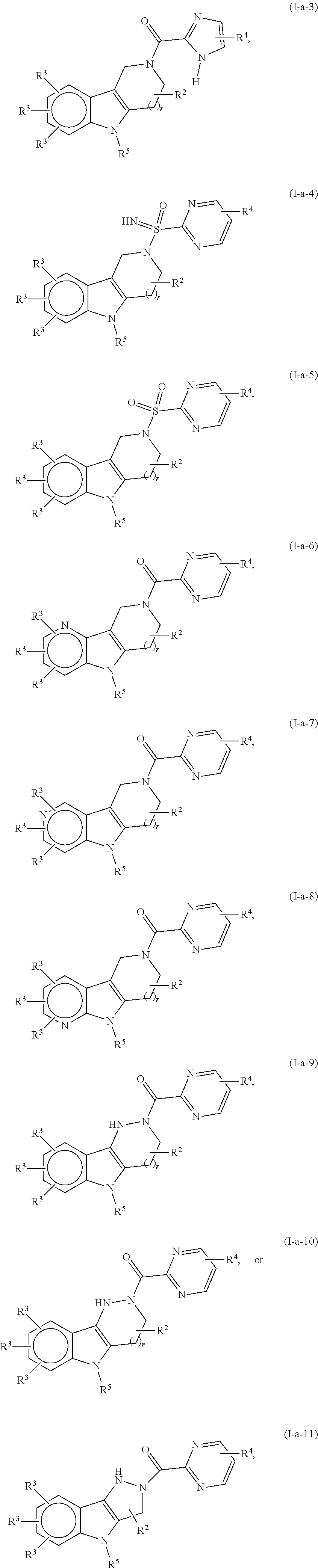

or a pharmaceutically acceptable salt, isomer, solvate, prodrug, or tautomer thereof.

Embodiment 10. The compound of embodiment 1, wherein the compound is of Formula I-b

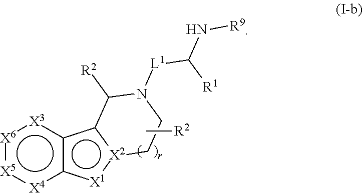

Embodiment 11. The compound of embodiment 1, wherein the compound is of Formula I-b-1

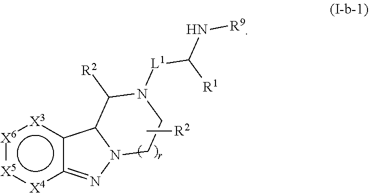

Embodiment 12. The compound of embodiment 1, wherein the compound is of Formula I-b-2

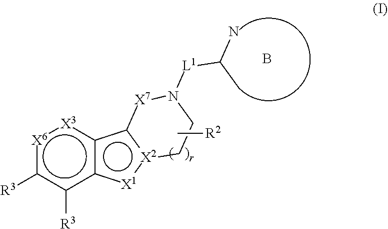

wherein ring B is:

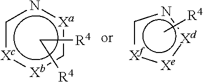

wherein X^{a}, X^{b}, and X^{c} are independently N or CH; X^{d}, X^{e}, and X are independently N, NH, or CH.

Embodiment 13. The compound of embodiment 1 wherein the compound has the formula Ic:

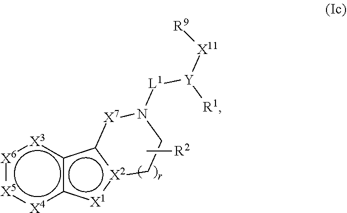

wherein Y is CH or NH; X¹¹ is O, N, or NH; R¹ and R⁹ combine to form a 5- to 10-membered heteroaryl having the formulae:

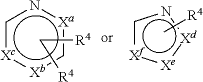

wherein X^{a}, X^{b}, and X^{c} are independently N or CH; X^{d}, X^{e}, and X^{f} are independently N, NH, or CH.

Embodiment 14. The compound of embodiment 1 wherein the compound has the formula Ic:

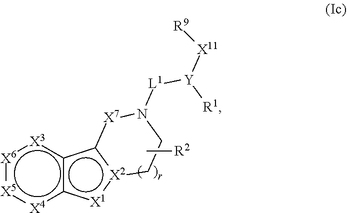

wherein Y is CH or NH; X¹¹ is O, N, or NH; R¹ and R⁹ combine to form a 5- to 10-membered heteroaryl having the formulae:

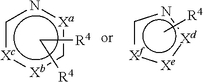

wherein X^{a}, X^{b}, and X^{c} are independently N or CH; X^{d}, X^{e}, and X^{f} are independently N, NH, or CH.

Embodiment 15. The compound of embodiment 1, wherein R¹ is H.

Embodiment 16. The compound of embodiment 11, wherein R¹ and R⁹ combine to form a 3- to 8-membered heterocycle, or 5- to 10-membered heteroaryl.

Embodiment 17. The compound of any one of the preceding embodiments, wherein R² is H.

Embodiment 18. The compound of any of the preceding embodiments wherein R² is halogen, CN, OH, C₁-C₆ alkyl, C₁-C₆ haloalkyl, C₁-C₆ haloalkoxy, —(CH₂)ₙ—SR⁸, —(CH₂)ₙ—OR⁸, aryl, or heteroaryl.

Embodiment 19. The compound of any of the preceding embodiments, wherein R⁵ is H.

Embodiment 20. The compound of any of the preceding embodiments, wherein R⁵ is —(CH₂)ₙ—OR⁸, CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl.

Embodiment 21. The compound of embodiment any of the preceding embodiments, wherein R⁵ is —C(O)OH, —(CH₂)ₙ—O—(CH₂)ₚ—OR⁸, —(CH₂)ₙ—OR⁸, or —(CH₂)ₙ—S(O)₂R⁸.

Embodiment 22. The compound of any of the preceding embodiments, wherein R⁴ is H.

Embodiment 23. The compound of any one of the preceding embodiments, wherein R⁴ is halogen, —CN, OR⁵, —NH₂, NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl.

Embodiment 24. The compound of any of the preceding embodiments, wherein R⁴ is OR⁵, —NH₂, NH(R⁵), —N(R⁵)(R⁶), —NHC(O)R⁵, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl.

Embodiment 25. The compound of embodiment 18, wherein R⁴ is —CO(OR⁵), —C(O)R⁵, —C(O)N(R⁵)₂.

Embodiment 26. The compound of any of the preceding embodiments, wherein L¹ is —S(O)₂—.

Embodiment 27. The compound of any of the preceding embodiments, wherein L¹ is —S(NH)(O)—.

Embodiment 28. The compound of any of the preceding embodiments, wherein L¹ is —S(O)—.

Embodiment 29. The compound of any of the preceding embodiments, wherein X¹ is —NR⁵—.

Embodiment 30. The compound of any of the preceding embodiments, wherein X¹ is —CH—.

Embodiment 31. The compound of any of the preceding embodiments, wherein X⁷ is —NH—.

Embodiment 32. The compound of any of the preceding embodiments, wherein X⁷ is —C(R²)₂—.

Embodiment 33. The compound of any of the preceding embodiments, wherein X⁷ is —NCH₃—.

Embodiment 34. The compound of any of the preceding embodiments, wherein two R², combined with the carbons to which they are attached, form C₄-C₈ cycloalkyl.

Embodiment 35. The compound of any of the preceding embodiments, wherein two R², combined with the carbo n to which they are attached form a 5- to 6-membered heterocycle.

Embodiment 36. The compound of any of the preceding embodiments, wherein r is 1.

Embodiment 37. The compound of any of the preceding embodiments, wherein r is 0.

Embodiment 38. A compound selected from Tables 1-5, or a pharmaceutically acceptable salt thereof.

Embodiment 39. The compound of embodiment 1 selected from the group consisting of:
(S)-(7,8-dichloro-1-methyl-3,4-dihydropyrazino[1,2-b]indazol-2(1H)-yl)(5-methoxypyrimidin-2-yl)methanone (Compound 52B*),
(6,7-dichloro-1-methyl-1,3,4,5-tetrahydro-2H-pyrido[4,3-b]indol-2-yl)(5-(methylamino)pyrimidin-2-yl)methanone (Rac-46),
(6,7-dichloro-1-methyl-1,3,4,5-tetrahydro-2H-pyrido[4,3-b]indol-2-yl)(5-morpholinopyrimidin-2-yl)methanone (Rac-44),
(6,7-dichloro-1,3,4,5-tetrahydro-2H-pyrido[4,3-b]indol-2-yl)(5-methoxypyrazin-2-yl)methanone (Compound 55),
(S)-(6,7-dichloro-1-methyl-1,3,4,5-tetrahydro-2H-pyrido[4,3-b]indol-2-yl)(5-methoxypyrimidin-2-yl)methanone (Compound 45B*),
(6,7-dichloro-5-(2-(methylsulfonyl)ethyl)-1,3,4,5-tetrahydro-2H-pyrido[4,3-b]indol-2-yl)(5-methoxypyrimidin-2-yl)methanone (Compound 18),
(6,7-dichloro-1-methyl-1,3,4,5-tetrahydro-2H-pyrido[4,3-b]indol-2-yl)(5-((2-(dimethylamino)ethyl)(methyl)amino)pyrimidin-2-yl)methanone (Rac-47),
(5-aminopyrimidin-2-yl)(6,7-dichloro-1,3,4,5-tetrahydro-2H-pyrido[4,3-b]indol-2-yl)methanone (Compound 30),
(6,7-dichloro-1,3,4,5-tetrahydro-2H-pyrido[4,3-b]indol-2-yl)(4-methylpyrimidin-2-yl)methanone (Compound 8),
(6,7-dichloro-1,3,4,5-tetrahydro-2H-pyrido[4,3-b]indol-2-yl)(5-methoxypyrimidin-2-yl)methanone (Compound 1),
(6,7-dichloro-1,3,4,5-tetrahydro-2H-pyrido[4,3-b]indol-2-yl)(4-methoxypyrimidin-2-yl)methanone (Compound 4),
(6,7-dichloro-1,3,4,5-tetrahydro-2H-pyrido[4,3-b]indol-2-yl)(oxazol-2-yl)methanone (Compound 11),
(6,7-dichloro-1,3,4,5-tetrahydro-2H-pyrido[4,3-b]indol-2-yl)(1-methyl-4,5-dihydro-1H-1,2,4-triazol-3-yl)methanone (Compound 154),
(6,7-dichloro-1,3,4,5-tetrahydro-2H-pyrido[4,3-b]indol-2-yl)(1-methyl-1H-imidazol-2-yl)methanone (Compound 3),
5-(6,7-dichloro-2,3,4,5-tetrahydro-1H-pyrido[4,3-b]indole-2-carbonyl)-2,4-dihydro-3H-1,2,4-triazol-3-one (Compound 13),
2-(6,7-dichloro-2-(5-methoxypyrimidine-2-carbonyl)-1,2,3,4-tetrahydro-5H-pyrido[4,3-b]indol-5-yl)acetic acid (Compound 17),
3-(6,7-dichloro-2-(5-methoxypyrimidine-2-carbonyl)-1,2,3,4-tetrahydro-5H-pyrido[4,3-b]indol-5-yl)propanoic acid (Compound 21)
(6,7-dichloro-1,3,4,5-tetrahydro-2H-pyrido[4,3-b]indol-2-yl)(5-hydroxypyrimidin-2-yl)methanone (Compound 16),
(6,7-dichloro-5-(3-methoxypropyl)-1,3,4,5-tetrahydro-2H-pyrido[4,3-b]indol-2-yl)(5-methoxypyrimidin-2-yl)methanone (Compound 19),
(6,7-dichloro-1,3,4,5-tetrahydro-2H-pyrido[4,3-b]indol-2-yl)(5-((2-methoxyethyl)amino)pyrimidin-2-yl)methanone (Compound 43),
(6,7-dichloro-1,3,4,5-tetrahydro-2H-pyrido[4,3-b]indol-2-yl)(5-((2-hydroxyethyl)amino)pyrimidin-2-yl)methanone (Compound 29),
(6,7-dichloro-1,3,4,5-tetrahydro-2H-pyrido[4,3-b]indol-2-yl)(1H-imidazol-2-yl)methanone (Compound 5),
(6,7-dichloro-1,3,4,5-tetrahydro-2H-pyrido[4,3-b]indol-2-yl)(1H-1,2,4-triazol-3-yl)methanone (Compound 6),
(6,7-dichloro-1,3,4,5-tetrahydro-2H-pyrido[4,3-b]indol-2-yl)(1-methyl-1H-1,2,4-triazol-3-yl)methanone (Compound 9),
(6,7-dichloro-1,3,4,5-tetrahydro-2H-pyrido[4,3-b]indol-2-yl)(2H-tetrazol-5-yl)methanone (Compound 7, tautomer 2),
(6,7-dichloro-1,3,4,5-tetrahydro-2H-pyrido[4,3-b]indol-2-yl)(5-(3-hydroxyazetidin-1-yl)pyrimidin-2-yl)methanone (Compound 42),
(S)-(6,7-dichloro-1,3,4,5-tetrahydro-2H-pyrido[4,3-b]indol-2-yl)(5-(2-(hydroxymethyl)pyrrolidin-1-yl)pyrimidin-2-yl)methanone (Compound 36),
(6,7-dichloro-1,3,4,5-tetrahydro-2H-pyrido[4,3-b]indol-2-yl)(5-(pyrrolidin-1-yl)pyrimidin-2-yl)methanone (Compound 41),
(R)-(6,7-dichloro-1,3,4,5-tetrahydro-2H-pyrido[4,3-b]indol-2-yl)(5-(3-hydroxypyrrolidin-1-yl)pyrimidin-2-yl)methanone (Compound 40),
(6,7-dichloro-1,3,4,5-tetrahydro-2H-pyrido[4,3-b]indol-2-yl)(pyrimidin-2-yl)methanone (Compound 2),
(S)-(6,7-dichloro-1,3,4,5-tetrahydro-2H-pyrido[4,3-b]indol-2-yl)(5-(3-hydroxypyrrolidin-1-yl)pyrimidin-2-yl)methanone (Compound 39),
(6,7-dichloro-5-(2-hydroxyethyl)-1,3,4,5-tetrahydro-2H-pyrido[4,3-b]indol-2-yl)(5-methoxypyrimidin-2-yl)methanone (Compound 22),
(6,7-dichloro-1,3,4,5-tetrahydro-2H-pyrido[4,3-b]indol-2-yl)(5-(methylamino)pyrimidin-2-yl)methanone (Compound 38),
(6,7-dichloro-1,3,4,5-tetrahydro-2H-pyrido[4,3-b]indol-2-yl)(5-(dimethylamino)pyrimidin-2-yl)methanone (Compound 37),
(6,7-dichloro-1,3,4,5-tetrahydro-2H-pyrido[4,3-b]indol-2-yl)(5-fluoropyrimidin-2-yl)methanone (Compound 15),
2-amino-1-(6,7-dichloro-1,3,4,5-tetrahydro-2H-pyrido[4,3-b]indol-2-yl)ethan-1-one (Compound 158),
(S)-2-amino-1-(6,7-dichloro-1-methyl-1,3,4,5-tetrahydro-2H-pyrido[4,3-b]indol-2-yl)ethan-1-one (Compound 51B*),
(R)-(6,7-dichloro-1,3,4,5-tetrahydro-2H-pyrido[4,3-b]indol-2-yl)(5-(2-(hydroxymethyl)pyrrolidin-1-yl)pyrimidin-2-yl)methanone (Compound 35),
(6,7-dichloro-1,3,4,5-tetrahydro-2H-pyrido[4,3-b]indol-2-yl)(5-(4-(2-hydroxyethyl)piperazin-1-yl)pyrimidin-2-yl)methanone (Compound 34),
(6,7-dichloro-1,3,4,5-tetrahydro-2H-pyrido[4,3-b]indol-2-yl)(5-((2-(dimethylamino)ethyl)(methyl)amino)pyrimidin-2-yl)methanone (Compound 33),
(6,7-dichloro-1,3,4,5-tetrahydro-2H-pyrido[4,3-b]indol-2-yl)(5-((2-hydroxyethyl)(methyl)amino)pyrimidin-2-yl)methanone (Compound 32),
(6,7-dichloro-5-(methylsulfonyl)-1,3,4,5-tetrahydro-2H-pyrido[4,3-b]indol-2-yl)(5-methoxypyrimidin-2-yl)methanone (Compound 20),
(6,7-dichloro-1,3,4,5-tetrahydro-2H-pyrido[4,3-b]indol-2-yl)(5-morpholinopyrimidin-2-yl)methanone (Compound 31),
(6,7-dichloro-1,3,4,5-tetrahydro-2H-pyrido[4,3-b]indol-2-yl)(5-methylpyrimidin-2-yl)methanone (Compound 12),
6,7-dichloro-1,3,4,5-tetrahydro-2H-pyrido[4,3-b]indole-2-carbohydrazide (Compound 59), (R)-2-amino-1-(6,7-dichloro-1,3,4,5-tetrahydro-2H-pyrido[4,3-b]indol-2-yl)propan-1-one (Compound 58),
2-(6,7-dichloro-1,3,4,5-tetrahydro-2H-pyrido[4,3-b]indol-2-yl)-2-oxoacetamide (Compound 57),
(6,7-dichloro-1,3,4,5-tetrahydro-2H-pyrido[4,3-b]indol-2-yl)(6-methoxypyridazin-3-yl)methanone (Compound 56),
(R)-2-amino-1-(6,7-dichloro-1,3,4,5-tetrahydro-2H-pyrido[4,3-b]indol-2-yl)-3-hydroxypropan-1-one (Compound 54),
(R)-2-amino-1-(6,7-dichloro-1,3,4,5-tetrahydro-2H-pyrido[4,3-b]indol-2-yl)-3-methoxypropan-1-one (Compound 53),
1-(6,7-dichloro-1,3,4,5-tetrahydro-2H-pyrido[4,3-b]indol-2-yl)-2-(methylamino)ethan-1-one (Compound 64),
2-amino-1-(5,6-dichloro-3,4-dihydropyrrolo[3,4-b]indol-2(1H)-yl)ethan-1-one (Compound 65),
2-amino-1-(8,9-dichloro-1,2,4,5,6,7-hexahydro-1,5-epiminooxocino[5,4-b]indol-12-yl)ethan-1-one (Compound 66),
(6,7-dichloro-1-methyl-1,3,4,5-tetrahydro-2H-pyrido[4,3-b]indol-2-yl)(4-(2-methoxyethoxy)pyrimidin-2-yl)methanone (Rac-67),
(6,7-dichloro-1-methyl-1,3,4,5-tetrahydro-2H-pyrido[4,3-b]indol-2-yl)(4-methoxypyrimidin-2-yl)methanone (Rac-68),
1-(6,7-dichloro-1,3,4,5-tetrahydro-2H-pyrido[4,3-b]indol-2-yl)-2-((2-methoxyethyl)amino)ethan-1-one (Compound 69),
(6,7-dichloro-1-methyl-1,3,4,5-tetrahydro-2H-pyrido[4,3-b]indol-2-yl)(4-(2-hydroxyethoxy)pyrimidin-2-yl)methanone (Rac-70),
(6,7-dichloro-9-(1-methyl-1H-pyrazol-3-yl)-1,3,4,5-tetrahydro-2H-pyrido[4,3-b]indol-2-yl)(5-methoxypyrimidin-2-yl)methanone (Compound 71),
(6,7-dichloro-9-(pyrimidin-5-yl)-1,3,4,5-tetrahydro-2H-pyrido[4,3-b]indol-2-yl)(5-methoxypyrimidin-2-yl)methanone (Compound 72), and
(6,7-dichloro-9-(pyridin-3-yl)-1,3,4,5-tetrahydro-2H-pyrido[4,3-b]indol-2-yl)(5-methoxypyrimidin-2-yl)methanone (Compound 73).

Embodiment 40. The compound of embodiment 1 selected from the group consisting of:
(S)-(6,7-dichloro-1-methyl-1,3,4,5-tetrahydro-2H-pyrido[4,3-b]indol-2-yl)(5-(2-(methylamino)ethoxy)pyrimidin-2-yl)methanone (Compound 76B*),
(S)-(6,7-dichloro-1-methyl-1,3,4,5-tetrahydro-2H-pyrido[4,3-b]indol-2-yl)(5-(2-methoxyethoxy)pyrimidin-2-yl)methanone (Compound 78B*),
(S)-3-((2-(6,7-dichloro-1-methyl-2,3,4,5-tetrahydro-1H-pyrido[4,3-b]indole-2-carbonyl)pyrimidin-5-yl)oxy)propanenitrile (Compound 79B*),
(S)-(6,7-dichloro-1-methyl-1,3,4,5-tetrahydro-2H-pyrido[4,3-b]indol-2-yl)(5-hydroxypyrimidin-2-yl)methanone (Compound 80B*),
(S)-(5-amino-4-methoxypyrimidin-2-yl)(6,7-dichloro-1-methyl-1,3,4,5-tetrahydro-2H-pyrido[4,3-b]indol-2-yl)methanone (Compound 85B*),
(S)-(6,7-dichloro-1-methyl-1,3,4,5-tetrahydro-2H-pyrido[4,3-b]indol-2-yl)(5-(2-hydroxyethoxy)pyrimidin-2-yl)methanone (Compound 77B*),
(S)-(6,7-dichloro-1-methyl-1,3,4,5-tetrahydro-2H-pyrido[4,3-b]indol-2-yl)(4-hydroxypyrimidin-2-yl)methanone (Compound 81B*),
(S)-(6,7-dichloro-1-methyl-1,3,4,5-tetrahydro-2H-pyrido[4,3-b]indol-2-yl)(4-methoxy-5-methylpyrimidin-2-yl)methanone (Compound 82B*),
(S)-(6,7-dichloro-1-methyl-1,3,4,5-tetrahydro-2H-pyrido[4,3-b]indol-2-yl)(4,5-dimethoxypyrimidin-2-yl)methanone (Compound 83B*),
(S)-3-((2-(6,7-dichloro-1-methyl-2,3,4,5-tetrahydro-1H-pyrido[4,3-b]indole-2-carbonyl)pyrimidin-4-yl)oxy)propanenitrile (Compound 89B*),
(S)-(6,7-dichloro-1-methyl-1,3,4,5-tetrahydro-2H-pyrido[4,3-b]indol-2-yl)(4-methoxy-5-(methylamino)pyrimidin-2-yl)methanone (Compound 84B*),
(S)-(6,7-dichloro-1-methyl-1,3,4,5-tetrahydro-2H-pyrido[4,3-b]indol-2-yl)(4-(2-hydroxyethoxy)pyrimidin-2-yl)methanone (Rac-70),
(S)-(6,7-dichloro-1-methyl-1,3,4,5-tetrahydro-2H-pyrido[4,3-b]indol-2-yl)(4-(2-methoxyethoxy)pyrimidin-2-yl)methanone (Rac-67),
(S)-(6,7-dichloro-1-methyl-1,3,4,5-tetrahydro-2H-pyrido[4,3-b]indol-2-yl)(4-(2-(dimethylamino)ethoxy)pyrimidin-2-yl)methanone (Compound 88B*),
(S)-(5-amino-1H-imidazol-2-yl)(6,7-dichloro-1-methyl-1,3,4,5-tetrahydro-2H-pyrido[4,3-b]indol-2-yl)methanone (Compound 90B*),
(S)-(6,7-dichloro-1-methyl-1,3,4,5-tetrahydro-2H-pyrido[4,3-b]indol-2-yl)(5-(dimethylamino)-1H-imidazol-2-yl)methanone (Compound 92B*),
(S)-(6,7-dichloro-1-methyl-1,3,4,5-tetrahydro-2H-pyrido[4,3-b]indol-2-yl)(5-hydroxy-1H-imidazol-2-yl)methanone (Compound 93B*),
(S)-(6,7-dichloro-1-methyl-1,3,4,5-tetrahydro-2H-pyrido[4,3-b]indol-2-yl)(5-(methylamino)-1H-imidazol-2-yl)methanone (Compound 91B*),
(S)-2-(6,7-dichloro-1-methyl-2,3,4,5-tetrahydro-1H-pyrido[4,3-b]indole-2-carbonyl)-1H-imidazole-5-carbonitrile (Compound 95B*),
(S)-(6,7-dichloro-1-methyl-1,3,4,5-tetrahydro-2H-pyrido[4,3-b]indol-2-yl)(5-methoxy-1H-imidazol-2-yl)methanone (Compound 94B*),
(S)-6,7-dichloro-N-hydroxy-1-methyl-1,3,4,5-tetrahydro-2H-pyrido[4,3-b]indole-2-carboxamide (Compound 97B*),
(S)-(7-chloro-6-hydroxy-1-methyl-1,3,4,5-tetrahydro-2H-pyrido[4,3-b]indol-2-yl)(5-methoxypyrimidin-2-yl)methanone (Compound 96B*),
(S)-(6,7-dichloro-9-ethynyl-1-methyl-1,3,4,5-tetrahydro-2H-pyrido[4,3-b]indol-2-yl)(5-methoxypyrimidin-2-yl)methanone (Compound 102B*),
(S)-6,7-dichloro-N-methoxy-1-methyl-1,3,4,5-tetrahydro-2H-pyrido[4,3-b]indole-2-carboxamide (Compound 98B*),
(1S)-6,7-dichloro-2-(5-methoxypyrimidine-2-sulfonimidoyl)-1-methyl-2,3,4,5-tetrahydro-1H-pyrido[4,3-b]indole (Compound 99B*),
(S)-(6,7-dichloro-1-methyl-3,4-dihydroimidazo[1,2-a:5,4-c′]dipyridin-2(1H)-yl)(5-methoxypyrimidin-2-yl)methanone (Compound 100B*),
(S)-(6,7-dichloro-1-methyl-9-(methylthio)-1,3,4,5-tetrahydro-2H-pyrido[4,3-b]indol-2-yl)(5-methoxypyrimidin-2-yl)methanone (Compound 101B*),
(S)-(6,7-dichloro-1-methyl-9-(prop-1-yn-1-yl)-1,3,4,5-tetrahydro-2H-pyrido[4,3-b]indol-2-yl)(5-methoxypyrimidin-2-yl)methanone (Compound 104B*),
(S)-(6,7-dichloro-1,9-dimethyl-1,3,4,5-tetrahydro-2H-pyrido[4,3-b]indol-2-yl)(5-methoxypyrimidin-2-yl)methanone (Compound 103B*),
(S)-(6,7-dichloro-9-(3-hydroxyprop-1-yn-1-yl)-1-methyl-1,3,4,5-tetrahydro-2H-pyrido[4,3-b]indol-2-yl)(5-methoxypyrimidin-2-yl)methanone (Compound 105B*),
(S)-(6,7-dichloro-1-methyl-1,3,4,5-tetrahydro-2H-pyrido[4,3-b]indol-2-yl)(6,7-dihydro-5H-pyrimido[4,5-b][1,4]oxazin-2-yl)methanone (Compound 106B*),
(S)-2-(6,7-dichloro-2-(5-methoxypyrimidine-2-carbonyl)-1-methyl-1,2,3,4-tetrahydro-5H-pyrido[4,3-b]indol-5-yl)acetonitrile (Compound 108B*),
(S)-(6,7-dichloro-1-methyl-5-(oxetan-3-ylmethyl)-1,3,4,5-tetrahydro-2H-pyrido[4,3-b]indol-2-yl)(5-methoxypyrimidin-2-yl)methanone (Compound 107B*),
(S)-(6,7-dichloro-5-((2-methoxyethoxy)methyl)-1-methyl-1,3,4,5-tetrahydro-2H-pyrido[4,3-b]indol-2-yl)(5-methoxypyrimidin-2-yl)methanone (Compound 109B*),
(S)-3,4-dichloro-8-(5-methoxypyrimidine-2-carbonyl)-2,9-dimethyl-2,5,6,7,8,9-hexahydro-1H-pyrrolo[3,2-c:4,5-c′]dipyridin-1-one (Compound 110B*),
(S)-(5,6-dichloro-1-methyl-3,4-dihydropyrrolo[3,4-b]indol-2(1H)-yl)(5-methoxypyrimidin-2-yl)methanone (Compound 112B*),
(S)-(3,4-dichloro-9-methyl-6,9-dihydropyrido[4′,3′:4,5]imidazo[1,2-c]pyrimidin-8(7H)-yl)(5-methoxypyrimidin-2-yl)methanone (Compound 111B*),
(S)-(7-chloro-6-fluoro-1-methyl-1,3,4,5-tetrahydro-2H-pyrido[4,3-b]indol-2-yl)(5-methoxypyrimidin-2-yl)methanone (Compound 118B*),
(S)-2-amino-1-(7,8-dichloro-1-methyl-3,4-dihydropyrazino[1,2-b]indazol-2(1H)-yl)ethan-1-one (Compound 120B*),
(S)-(6,7-dichloro-1-methyl-1,3,4,5-tetrahydro-2H-pyrrolo[3,2-c:4,5-c′]dipyridin-2-yl)(5-methoxypyrimidin-2-yl)methanone (Compound 114B*),
(S)-(6,7-dichloro-8-methoxy-1-methyl-1,3,4,5-tetrahydro-2H-pyrido[4,3-b]indol-2-yl)(5-methoxypyrimidin-2-yl)methanone (Compound 115B*),
(S)-(6,7-dichloro-8-hydroxy-1-methyl-1,3,4,5-tetrahydro-2H-pyrido[4,3-b]indol-2-yl)(5-methoxypyrimidin-2-yl)methanone (Compound 116B*),
(S)-(6,7-dichloro-1-methyl-8-(methylthio)-1,3,4,5-tetrahydro-2H-pyrido[4,3-b]indol-2-yl)(5-methoxypyrimidin-2-yl)methanone (Compound 117B*),
6-chloro-2-(5-methoxypyrimidine-2-carbonyl)-7-methyl-1,2,3,4,5,7-hexahydro-8H-pyrrolo[2,3-c:4,5-c′]dipyridin-8-one (Compound 119),
(S)-(7-bromo-6-chloro-1-methyl-1,3,4,5-tetrahydro-2H-pyrido[4,3-b]indol-2-yl)(5-methoxypyrimidin-2-yl)methanone (Compound 121B*),
(S)-7-chloro-2-(5-methoxypyrimidine-2-carbonyl)-1-methyl-2,3,4,5-tetrahydro-1H-pyrido[4,3-b]indole-6-carbonitrile (Compound 123B*),
(7,8-dichloro-1,4,5,6-tetrahydroazepino[4,5-b]indol-3(2H)-yl)(5-methoxypyrimidin-2-yl)methanone (Compound 124),
(S)-3-(6,7-dichloro-2-(5-methoxypyrimidine-2-carbonyl)-1-methyl-2,3,4,5-tetrahydro-1H-pyrido[4,3-b]indol-9-yl)propanenitrile (Compound 125B*),
3-chloro-8-(5-methoxypyrimidine-2-carbonyl)-1-methyl-2-oxo-2,5,6,7,8,9-hexahydro-1H-pyrrolo[3,2-b:4,5-c′]dipyridine-4-carbonitrile (Compound 122),
(S)-2-(6,7-dichloro-2-(5-methoxypyrimidine-2-carbonyl)-1-methyl-2,3,4,5-tetrahydro-1H-pyrido[4,3-b]indol-9-yl)acetonitrile (Compound 126B*),
(S)-(6,7-dichloro-9-(difluoromethyl)-1-methyl-1,3,4,5-tetrahydro-2H-pyrido[4,3-b]indol-2-yl)(5-methoxypyrimidin-2-yl)methanone (Compound 127B*),
(S)-(6,7-dichloro-9-ethyl-1-methyl-1,3,4,5-tetrahydro-2H-pyrido[4,3-b]indol-2-yl)(5-methoxypyrimidin-2-yl)methanone (Compound 128B*),
(S)-(6,7-dichloro-9-(difluoromethoxy)-1-methyl-1,3,4,5-tetrahydro-2H-pyrido[4,3-b]indol-2-yl)(5-methoxypyrimidin-2-yl)methanone (Compound 129B*),
(5,6-dichloro-1-methyl-3,4-dihydropyrazolo[4,3-b]indol-2(1H)-yl)(5-methoxypyrimidin-2-yl)methanone (Compound 135B*),
(S)-(6,7-dichloro-9-((difluoromethyl)thio)-1-methyl-1,3,4,5-tetrahydro-2H-pyrido[4,3-b]indol-2-yl)(5-methoxypyrimidin-2-yl)methanone (Compound 130B*),
(S)-(6,7-dichloro-1-methyl-9-vinyl-1,3,4,5-tetrahydro-2H-pyrido[4,3-b]indol-2-yl)(5-methoxypyrimidin-2-yl)methanone (Compound 131B*),
(S)-(6,7-dichloro-9-methoxy-1-methyl-1,3,4,5-tetrahydro-2H-pyrido[4,3-b]indol-2-yl)(5-methoxypyrimidin-2-yl)methanone (Compound 132B*),
(S)-(3,4-dichloro-10-methyl-7,8-dihydropyrido[4′,3′:3,4]pyrazolo[1,5-a]pyrazin-9(10H)-yl)(5-methoxypyrimidin-2-yl)methanone (Compound 134B*),
((2R,4S)-7,8-dichloro-2,3,4,9-tetrahydro-1H-2,4-epiminocarbazol-10-yl)(5-methoxypyrimidin-2-yl)methanone (Compound 133B*),
(6,7-dichloro-1-methyl-1,3,4,5-tetrahydro-2H-pyridazino[4,3-b]indol-2-yl)(5-methoxypyrimidin-2-yl)methanone (Compound 136),
(6,7-dichloro-4-phenyl-1,3,4,5-tetrahydro-2H-pyrido[4,3-b]indol-2-yl)(5-methoxypyrimidin-2-yl)methanone (Rac-137),
(6,7-dichloro-4-(pyridin-3-yl)-1,3,4,5-tetrahydro-2H-pyrido[4,3-b]indol-2-yl)(5-methoxypyrimidin-2-yl)methanone (Rac-138),
(S)-(7,8-dichloro-1-methyl-10-(1-methyl-1H-pyrazol-3-yl)-3,4-dihydropyrazino[1,2-b]indazol-2(1H)-yl)(5-methoxypyrimidin-2-yl)methanone (Compound 140B*),
6,7-dichloro-2-(5-methoxypyrimidine-2-carbonyl)-2,3,4,5-tetrahydro-1H-pyrido[4,3-b]indole-4-carbonitrile (Rac-139),
(6,7-dichloro-5-methyl-1,3,4,5-tetrahydro-2H-pyrido[4,3-b]indol-2-yl)(5-methoxypyrimidin-2-yl)methanone (Compound 141),
(S)-(7-bromo-6-fluoro-1-methyl-1,3,4,5-tetrahydro-2H-pyrido[4,3-b]indol-2-yl)(5-methoxypyrimidin-2-yl)methanone (Compound 142B*),
(S)-(3,4-dichloro-2-methoxy-9-methyl-5,6,7,9-tetrahydro-8H-pyrrolo[3,2-b:4,5-c′]dipyridin-8-yl)(5-methoxypyrimidin-2-yl)methanone (Compound 143B*),
(R)-(6,7-dichloro-1-(methoxymethyl)-1,3,4,5-tetrahydro-2H-pyrido[4,3-b]indol-2-yl)(5-methoxypyrimidin-2-yl)methanone (Compound 150B*),
(S)-(3-bromo-4-chloro-2-methoxy-9-methyl-5,6,7,9-tetrahydro-8H-pyrrolo[3,2-b:4,5-c′]dipyridin-8-yl)(5-methoxypyrimidin-2-yl)methanone (Compound 145B*),
(S)-(6,7-dichloro-9-methoxy-1-methyl-1,3,4,5-tetrahydro-2H-pyrrolo[3,2-c:4,5-c′]dipyridin-2-yl)(5-methoxypyrimidin-2-yl)methanone (Compound 146B*),
(R)-(6,7-dichloro-1-(fluoromethyl)-1,3,4,5-tetrahydro-2H-pyrido[4,3-b]indol-2-yl)(5-methoxypyrimidin-2-yl)methanone (Compound 147B*),
(R)-(6,7-dichloro-1-(difluoromethyl)-1,3,4,5-tetrahydro-2H-pyrido[4,3-b]indol-2-yl)(5-methoxypyrimidin-2-yl)methanone (Compound 149B*),
(R)-(6,7-dichloro-1-((methylthio)methyl)-1,3,4,5-tetrahydro-2H-pyrido[4,3-b]indol-2-yl)(5-methoxypyrimidin-2-yl)methanone (Compound 148B*),
(S)-(6-chloro-7-ethynyl-1-methyl-1,3,4,5-tetrahydro-2H-pyrido[4,3-b]indol-2-yl)(5-methoxypyrimidin-2-yl)methanone (Compound 144B*),
(6,7-dichloro-1-methyl-1,3,4,5-tetrahydro-2H-pyrido[4,3-b]indol-2-yl)(5-((S)-7,7-difluorohexahydropyrrolo[1,2-a]pyrazin-2(1H)-yl)pyrimidin-2-yl)methanone (Rac-50),
(S)-(7,8-dichloro-1-methyl-3,4-dihydropyrazino[2,1-a]isoindol-2(1H)-yl)(5-methoxypyrimidin-2-yl)methanone (Compound 153B*),
(6,7-dichloro-1-methyl-1,3,4,5-tetrahydro-2H-pyrido[4,3-b]indol-2-yl)(5-(4-methylpiperazin-1-yl)pyrimidin-2-yl)methanone (Rac-48),
(6,7-dichloro-1-methyl-1,3,4,5-tetrahydro-2H-pyrido[4,3-b]indol-2-yl)(5-(4-(oxetan-3-yl)piperazin-1-yl)pyrimidin-2-yl)methanone (Rac-49),
(S)-(6,7-dichloro-1-methyl-9-(1-methyl-1H-pyrazol-3-yl)-1,3,4,5-tetrahydro-2H-pyrido[4,3-b]indol-2-yl)(5-methoxypyrimidin-2-yl)methanone (Compound 113B*),
(S)-(9-(6-aminopyridin-3-yl)-6,7-dichloro-1-methyl-1,3,4,5-tetrahydro-2H-pyrido[4,3-b]indol-2-yl)(5-methoxypyrimidin-2-yl)methanone (Compound 86B*), and
(S)-(10-(6-aminopyridin-3-yl)-7,8-dichloro-1-methyl-3,4-dihydropyrazino[1,2-b]indazol-2(1H)-yl)(5-methoxypyrimidin-2-yl)methanone (Compound 87B*).

Embodiment 41. The compound selected from embodiment 1 selected from the group consisting of:
(S)-2-amino-1-(6,7-dichloro-1-methyl-1,3,4,5-tetrahydro-2H-pyrido[4,3-b]indol-2-yl)ethan-1-one (Compound 51B*),
(S)-(7,8-dichloro-1-methyl-3,4-dihydropyrazino[1,2-b]indazol-2(1H)-yl)(5-methoxypyrimidin-2-yl)methanone (Compound 52B*),
(S)-(6,7-dichloro-1-methyl-1,3,4,5-tetrahydro-2H-pyrido[4,3-b]indol-2-yl)(5-methoxypyrimidin-2-yl)methanone (Compound 45B*), and
(S)-2-amino-1-(7,8-dichloro-1-methyl-3,4-dihydropyrazino[1,2-b]indazol-2(1H)-yl)ethan-1-one (Compound 120B*).

Embodiment 42. The compound of any of the preceding embodiments, or a pharmaceutically acceptable salt or stereoisomer thereof.

Embodiment 43. The compound of any of the preceding embodiments, or a pharmaceutically acceptable salt thereof.

Embodiment 44. An isotopic derivative of the compound of any one of the preceding embodiments.

Embodiment 45. A pharmaceutical composition comprising the compound of any one of the preceding embodiments and one or more pharmaceutically acceptable carriers.

Embodiment 46. A method of treating or preventing a cGAS-related disease or disorder, the method comprising administering to the subject at least one therapeutically effective amount of the compound of any one of the preceding embodiments.

Embodiment 47. A method of inhibiting cGAS, the method comprising administering to the subject at least one therapeutically effective amount of the compound of any one of the preceding embodiments

Embodiment 48. The compound of any one of the preceding embodiments for use in treating or preventing a cGAS-related disease or disorder.

Embodiment 49. Use of the compound of any one of the preceding embodiments, in the manufacture of a medicament, for treating or preventing a cGAS-related disease or disorder.

Embodiment 50. The method, compound, or use of any one of the preceding embodiments, wherein the subject is a human.

Embodiment 51. The method, compound, or use of any one of the preceding embodiments, wherein the cGAS-related disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or an cGAS-related disease in a subject that has been determined to carry a germline or somatic non-silent mutation in cGAS.

Embodiment 52. The method, compound, or use of any one of the preceding embodiments, wherein the disease or disorder of the central nervous system is Parkinson's disease, Alzheimer's disease, traumatic brain injury, spinal cord injury, amyotrophic lateral sclerosis, or multiple sclerosis.

Embodiment 53. The method, compound, or use of any one of the preceding embodiments, wherein the kidney disease is an acute kidney disease, a chronic kidney disease, or a rare kidney disease.

Embodiment 54. The method, compound, or use of any one of the preceding embodiments, wherein the skin disease is psoriasis, hidradenitis suppurativa (HS), or atopic dermatitis.

Embodiment 55. The method, compound, or use of any one of the preceding embodiments, wherein the rheumatic disease is dermatomyositis, Still's disease, or juvenile idiopathic arthritis.

Embodiment 56. The method, compound, or use of any one of the preceding embodiments, wherein the cGAS-related disease in a subject that has been determined to carry a germline or somatic non-silent mutation in cGAS is cryopyrin-associated autoinflammatory syndrome.

Embodiment 57. The method, compound, or use of any one of the preceding embodiments, wherein the cryopyrin-associated autoinflammatory syndrome is familial cold autoinflammatory syndrome, Muckle-Wells syndrome, or neonatal onset multisystem inflammatory disease.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (II): 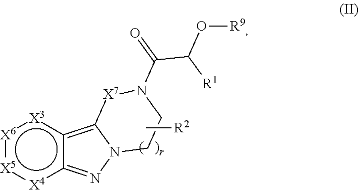
or a pharmaceutically acceptable salt, hydrate, solvate, prodrug, stereoisomer, or tautomer thereof, wherein:
X³, X⁴, X⁵, and X⁶ are each independently CR³;
R¹ is H or C₁-C₆ alkyl, wherein the alkyl is optionally substituted with one or more R⁴;
X⁷ is —CH(R²)—, wherein R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl;
the other R² is H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle;
each R³ is independently H, halogen, oxo, —CN, —OR⁵, —SR⁵, —NH₂, —NH(R⁵), —N(R⁵)(R⁶), —NHC(O)R⁵, —CO(OR⁵), —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶;
each R⁴ is independently H, halogen, —CN, —OR⁵, —NH₂, —NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or more R⁷;
each R⁵ is independently H, —C(O)OH, —(CH₂)ₙ—O—(CH₂)ₚ—OR⁸, —(CH₂)ₙ—OR⁸, —(CH₂)ₙ—S(O)₂R⁸, —CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl;
R⁶, R⁷, and R⁸ are independently, at each occurrence, H, halogen, —OH, —CN, —NH₂, C₁-C₆ alkyl, C₁-C₆ hydroxyalkyl, C₁-C₆ alkoxy, C₂-C₆ alkenyl, C₂-C₆ alkynyl, C₃-C₈ cycloalkyl, heterocyclyl, heteroaryl, or aryl;
R⁹ is independently H or C₁-C₄ alkyl, wherein the alkyl is optionally substituted with one or more halogen, —OH, —CN, C₁-C₆ alkoxy, or cycloalkyl;
each n is independently an integer from 0 to 6;
each p is independently an integer from 0 to 6; and
r is an integer from 0 to 2.

2. The compound of claim 1, wherein the compound is of Formula (II-a): 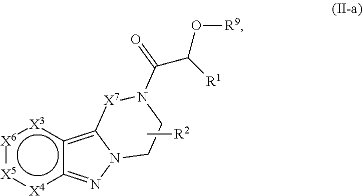
or a pharmaceutically acceptable salt, hydrate, solvate, prodrug, stereoisomer, or tautomer thereof; wherein R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl.

3. The compound of claim 1 or 2, wherein the compound is of Formula (II-b): 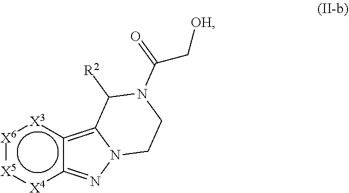
or a pharmaceutically acceptable salt, hydrate, solvate, prodrug, stereoisomer, or tautomer thereof.

4. The compound of any one of claims 1-3, wherein the compound is of Formula (II-c): 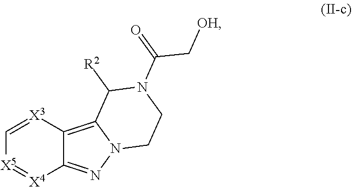
or a pharmaceutically acceptable salt, hydrate, solvate, prodrug, stereoisomer, or tautomer thereof.

5. The compound of any one of claims 1-4, wherein the compound is are of Formula (II-c): 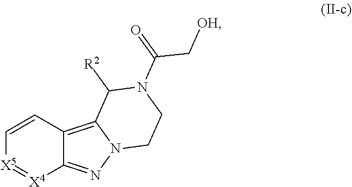
or a pharmaceutically acceptable salt, hydrate, solvate, prodrug, stereoisomer, or tautomer thereof.

6. The compound of claim 1 selected from those in Table 1, or a pharmaceutically acceptable salt, hydrate, solvate, prodrug, stereoisomer, or tautomer thereof.

7. A compound of Formula (III): 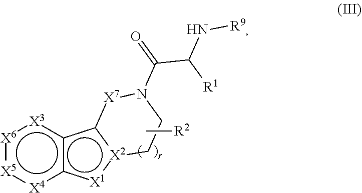
or a pharmaceutically acceptable salt, hydrate, solvate, prodrug, stereoisomer, or tautomer thereof, wherein:
X¹ is N or NR⁵, and X² is N or C;
X³, X⁴, X⁵, and X⁶ are each independently CR³
X⁷ is —CH(R²)—, wherein R² is halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl;
the other R² is hydrogen, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C4-C₈ cycloalkyl or 4- to 6-membered heterocycle;
R¹ is H or C₁-C₆ alkyl, wherein the alkyl is optionally substituted with one or more R⁴; or
each R³ is independently H, halogen, oxo, —CN, —OR⁵, —SR⁵, —NH₂, —NH(R⁵), —N(R⁵)(R⁶), —NHC(O)R⁵, —CO(OR⁵), —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶;
each R⁴ is independently H, halogen, —CN, —OR⁵, —NH₂, —NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or more R⁷;
each R⁵ is independently H, —C(O)OH, —(CH₂)ₙ—O—(CH₂)ₚ—OR⁸, —(CH₂)ₙ—OR⁸, —(CH₂)ₙ—S(O)₂R⁸, —CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl;
R⁶, R⁷, and R⁸ are independently, at each occurrence, H, halogen, —OH, —CN, —NH₂, C₁-C₆ alkyl, C₁-C₆ hydroxyalkyl, C₁-C₆ alkoxy, C₂-C₆ alkenyl, C₂-C₆ alkynyl, C₃-C₈ cycloalkyl, heterocyclyl, heteroaryl, or aryl;
R⁹ is independently H or C₁-C₄ alkyl, wherein the alkyl is optionally substituted with one or more halogen, —OH, —CN, C₁-C₆ alkoxy, or cycloalkyl;
each n is independently an integer from 0 to 6;
each p is independently an integer from 0 to 6; and
r is an integer from 0 to 2.

8. The compound of claim 7, wherein the compound is of Formula (III-a): 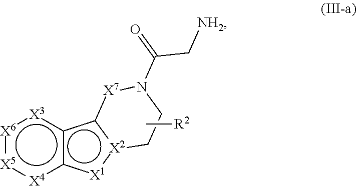
or a pharmaceutically acceptable salt, hydrate, solvate, prodrug, stereoisomer, or tautomer thereof.

9. The compound of claim 7 or 8, wherein the compound is of Formula (III-b): 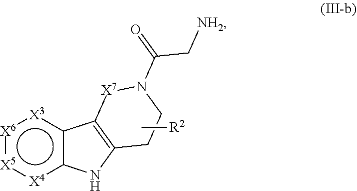
or a pharmaceutically acceptable salt, hydrate, solvate, prodrug, stereoisomer, or tautomer thereof.

10. The compound of any one of claims 7-9, wherein the compound is of Formula (III-c): 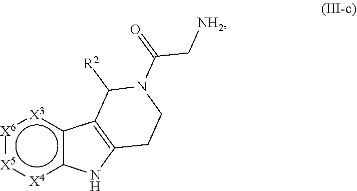
or a pharmaceutically acceptable salt, hydrate, solvate, prodrug, stereoisomer, or tautomer thereof.

11. The compound of any one of claims 7-10, wherein the compound is of Formula (III-d): 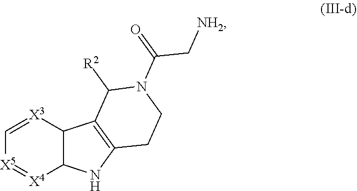
or a pharmaceutically acceptable salt, hydrate, solvate, prodrug, stereoisomer, or tautomer thereof.

12. The compound of claim 7 or 8, wherein the compound is of Formula (III-e): 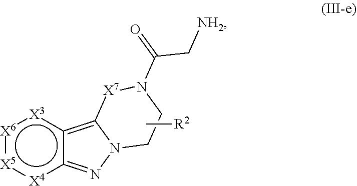
or a pharmaceutically acceptable salt, hydrate, solvate, prodrug, stereoisomer, or tautomer thereof.

13. The compound of any one of claims 7, 8, and 12, wherein the compound is of Formula (III-f): 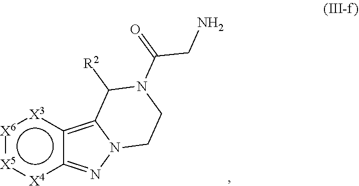
or a pharmaceutically acceptable salt, hydrate, solvate, prodrug, stereoisomer, or tautomer thereof.

14. The compound of any one of claims 7, 8, 12, or 13, wherein the compound is of Formula (III-g): 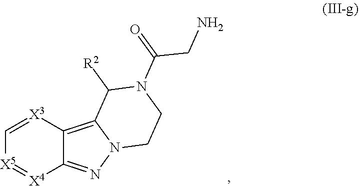
or a pharmaceutically acceptable salt, hydrate, solvate, prodrug, stereoisomer, or tautomer thereof.

15. The compound of claim 7 selected from those in Table 2, or a pharmaceutically acceptable salt, hydrate, solvate, prodrug, stereoisomer, or tautomer thereof.

16. A compound of Formula (IV): 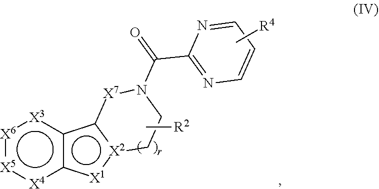
or a pharmaceutically acceptable salt, hydrate, solvate, prodrug, stereoisomer, or tautomer thereof, wherein:
X¹ is N or NR⁵, and X² is N or C;
X⁷ is —CH(R²)—;
each R² is independently H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle;
X³, X⁴, X⁵, and X⁶ are each independently CR³;
each R³ is independently H, halogen, —CN, —OR⁵, —SR⁵, —NH₂, —NH(R⁵), —N(R⁵)(R⁶), —NHC(O)R⁵, —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶;
each R⁴ is independently H, halogen, —CN, —OR⁵, —NH₂, —NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or more R⁷;
each R⁵ is independently H, —C(O)OH, —(CH₂)ₙ—O—(CH₂)ₚ—OR⁸, —(CH₂)ₙ—OR⁸, —(CH₂)ₙ—S(O)₂R⁸, —CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl;
R⁶, R⁷, and R⁸ are independently, at each occurrence, H, halogen, —OH, —CN, —NH₂, C₁-C₆ alkyl, C₁-C₆ hydroxyalkyl, C₁-C₆ alkoxy, C₂-C₆ alkenyl, C₂-C₆ alkynyl, C₃-C₈ cycloalkyl, heterocyclyl, heteroaryl, or aryl;
each n is independently an integer from 0 to 6;
each p is independently an integer from 0 to 6; and
r is an integer from 0 to 2.

17. The compound of claim 16, wherein the compound is of Formula (IV-a): 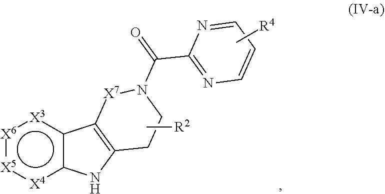
or a pharmaceutically acceptable salt, hydrate, solvate, prodrug, stereoisomer, or tautomer thereof.

18. The compound of claim 16 or 17, wherein the compound is of Formula (IV-b): 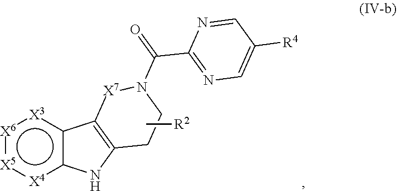
or a pharmaceutically acceptable salt, hydrate, solvate, prodrug, stereoisomer, or tautomer thereof.

19. The compound of claim 16 or 17, wherein the compound is of Formula (IV-c): 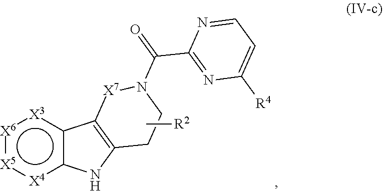
or a pharmaceutically acceptable salt, hydrate, solvate, prodrug, stereoisomer, or tautomer thereof.

20. The compound of claim 16 or 17, wherein the compound is of Formula (IV-d): 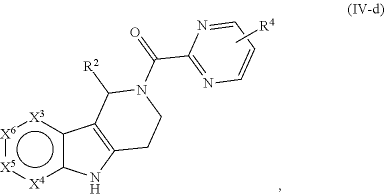
or a pharmaceutically acceptable salt, hydrate, solvate, prodrug, stereoisomer, or tautomer thereof.

21. The compound ofany one of claims 16-18 wherein the compound is of Formula (IV-e): 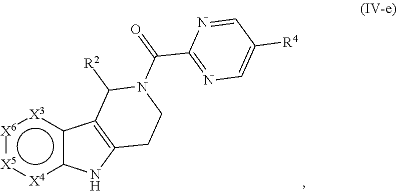
or a pharmaceutically acceptable salt, hydrate, solvate, prodrug, stereoisomer, or tautomer thereof.

22. The compound of claim 16 or 17, wherein the compound is of Formula (IV-f): 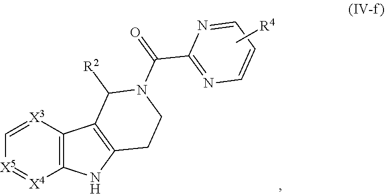
or a pharmaceutically acceptable salt, hydrate, solvate, prodrug, stereoisomer, or tautomer thereof.

23. The compound of any one of claims 16-18, wherein the compound is of Formula (IV-g): 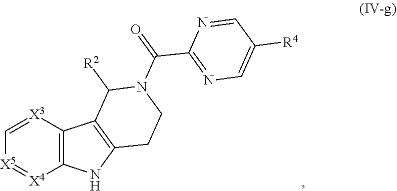
or a pharmaceutically acceptable salt, hydrate, solvate, prodrug, stereoisomer, or tautomer thereof.

24. The compound of claim 16 selected from those in Table 3, or a pharmaceutically acceptable salt, hydrate, solvate, prodrug, stereoisomer, or tautomer thereof.

25. The compound of any one of the preceding claims, or a pharmaceutically acceptable salt, hydrate, solvate, prodrug, stereoisomer, or tautomer thereof, wherein r is 1.

26. The compound of any one of the preceding claims, or a pharmaceutically acceptable salt, hydrate, solvate, prodrug, stereoisomer, or tautomer thereof, wherein R¹ is H.

27. The compound of any one of the preceding claims, or a pharmaceutically acceptable salt, hydrate, solvate, prodrug, stereoisomer, or tautomer thereof, wherein each instance of R² is independently H, C₁-C₆ alkyl, C₂-C₆ alkenyl, C₂-C₆ alkynyl, C₁-C₆ haloalkyl, —(CH₂)ₙ—SR⁸, —(CH₂)ₙ—OR⁸, aryl, or heteroaryl.

28. The compound of any one of the preceding claims, or a pharmaceutically acceptable salt, hydrate, solvate, prodrug, stereoisomer, or tautomer thereof, wherein at least one instance of R² is C₁-C₆ alkyl, C₂-C₆ alkenyl, C₂-C₆ alkynyl, C₁-C₆ haloalkyl, —(CH₂)ₙ—SR⁸, —(CH₂)ₙ—OR⁸, aryl, or heteroaryl.

29. The compound of any one of the preceding claims, or a pharmaceutically acceptable salt, hydrate, solvate, prodrug, stereoisomer, or tautomer thereof, wherein each instance of R² is independently H or C₁-C₆ alkyl.

30. The compound of any one of the preceding claims, or a pharmaceutically acceptable salt, hydrate, solvate, prodrug, stereoisomer, or tautomer thereof, wherein at least one instance of R² is C₁-C₆ alkyl.

31. The compound of any one of the preceding claims, or a pharmaceutically acceptable salt, hydrate, solvate, prodrug, stereoisomer, or tautomer thereof, wherein at least one instance of R² is methyl.

32. The compound of any one of the preceding claims, or a pharmaceutically acceptable salt, hydrate, solvate, prodrug, stereoisomer, or tautomer thereof, wherein the carbon atom bonded to R² is in the (S) configuration.

33. The compound of any one of the preceding claims, or a pharmaceutically acceptable salt, hydrate, solvate, prodrug, stereoisomer, or tautomer thereof, wherein the carbon atom bonded to R² is in the (R) configuration.

34. The compound of any one of the preceding claims, or a pharmaceutically acceptable salt, hydrate, solvate, prodrug, stereoisomer, or tautomer thereof, wherein X⁷ is of the following formula: 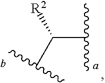
wherein a indicates the point of attachment to the amide nitrogen and b indicates the point of attachment to the heteroaryl ring.

35. The compound of any one of the preceding claims, or a pharmaceutically acceptable salt, hydrate, solvate, prodrug, stereoisomer, or tautomer thereof, wherein X⁷ is of the following formula: 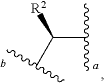
wherein a indicates the point of attachment to the amide nitrogen and b indicates the point of attachment to the heteroaryl ring.

36. The compound of any one of the preceding claims, or a pharmaceutically acceptable salt, hydrate, solvate, prodrug, stereoisomer, or tautomer thereof, wherein X⁷ is of the following formula: 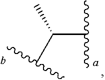
wherein a indicates the point of attachment to the amide nitrogen and b indicates the point of attachment to the heteroaryl ring.

37. The compound of any one of the preceding claims, or a pharmaceutically acceptable salt, hydrate, solvate, prodrug, stereoisomer, or tautomer thereof, wherein X⁷ is of the following formula: 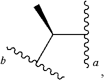
wherein a indicates the point of attachment to the amide nitrogen and b indicates the point of attachment to the heteroaryl ring.

38. The compound of any one of the preceding claims, or a pharmaceutically acceptable salt, hydrate, solvate, prodrug, stereoisomer, or tautomer thereof, wherein each R³ is independently H, halogen, C₁-C₆ alkyl, or heteroaryl, wherein each alkyl or heteroaryl is optionally substituted with one or more R⁶.

39. The compound of any one of the preceding claims, or a pharmaceutically acceptable salt, hydrate, solvate, prodrug, stereoisomer, or tautomer thereof, wherein R⁴ is H, halogen, C₁-C₆ alkyl, —OR⁵, —NH₂, —NH(R⁵), or —N(R⁵)(R⁶).

40. The compound of any one of the preceding claims, or a pharmaceutically acceptable salt, hydrate, solvate, prodrug, stereoisomer, or tautomer thereof, wherein R⁴ is —OR⁵, —NH₂, —NH(R⁵), or —N(R⁵)(R⁶).

41. The compound of any one of the preceding claims, or a pharmaceutically acceptable salt, hydrate, solvate, prodrug, stereoisomer, or tautomer thereof, wherein R⁵ is H.

42. The compound of any one of the preceding claims, or a pharmaceutically acceptable salt, hydrate, solvate, prodrug, stereoisomer, or tautomer thereof, wherein R⁹ is H.

43. The compound of any one of the preceding claims, or a pharmaceutically acceptable salt, hydrate, solvate, prodrug, stereoisomer, or tautomer thereof, wherein X¹ is NH.

44. The compound of any one of the preceding claims, or a pharmaceutically acceptable salt, hydrate, solvate, prodrug, stereoisomer, or tautomer thereof, wherein X² is N.

45. The compound of any one of the preceding claims, or a pharmaceutically acceptable salt, hydrate, solvate, prodrug, stereoisomer, or tautomer thereof, wherein X² is C.

46. The compound of any one of the preceding claims, or a pharmaceutically acceptable salt, hydrate, solvate, prodrug, stereoisomer, or tautomer thereof, wherein X³ is CR³, wherein R³ is H or heteroaryl, wherein the heteroaryl is optionally substituted with one or more R⁶.

47. The compound of any one of the preceding claims, or a pharmaceutically acceptable salt, hydrate, solvate, prodrug, stereoisomer, or tautomer thereof, wherein X⁴ is CR³, wherein R³ is halogen.

48. The compound of any one of the preceding claims, or a pharmaceutically acceptable salt, hydrate, solvate, prodrug, stereoisomer, or tautomer thereof, wherein X⁶ is CR³, wherein R³ is H.

49. The compound of any one of the preceding claims, or a pharmaceutically acceptable salt, hydrate, solvate, prodrug, stereoisomer, or tautomer thereof, wherein X⁵ is CR³, wherein R³ is halogen.

50. The compound of any one of the preceding claims, or a pharmaceutically acceptable salt, hydrate, solvate, prodrug, stereoisomer, or tautomer thereof, wherein X⁷ is —CH(R²)—, and R² is C₁-C₆ alkyl.

51. The compound of any one of the preceding claims, or a pharmaceutically acceptable salt, hydrate, solvate, prodrug, stereoisomer, or tautomer thereof, wherein X⁷ is —CH(R²)—, and R² is methyl.

52. The compound of any one of the preceding claims, or a pharmaceutically acceptable salt, hydrate, solvate, prodrug, stereoisomer, or tautomer thereof, wherein at least one instance of R⁶ is C₁-C₆ alkyl.

53. The compound of any one of the preceding claims, or a pharmaceutically acceptable salt, hydrate, solvate, prodrug, stereoisomer, or tautomer thereof, wherein at least one instance of R⁶ is methyl.

54. A compound selected from those in Table 4, or a pharmaceutically acceptable salt, hydrate, solvate, prodrug, stereoisomer, or tautomer thereof.

55. A compound selected from those in Table 5, or a pharmaceutically acceptable salt, hydrate, solvate, prodrug, stereoisomer, or tautomer thereof.

56. A compound of Formula (I): 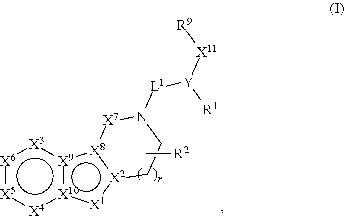
or a pharmaceutically acceptable salt, hydrate, solvate, prodrug, stereoisomer, or tautomer thereof, wherein:
L¹ is —C(O)—, —S(O), —S(O)₂—, or —S(NH)(O)—;
X¹ is independently N, NR⁵, or CH;
X² is independently N or C, provided that at least one of X¹ and X² includes N, wherein X¹ is N or NR⁵ and/or X² is N;
X³, X⁴, X⁵, X⁶, X⁸, X⁹, and X¹⁰ are independently C, CR³ or N, as valency permits, wherein at least one of X³, X⁴, X⁵, and X⁶ is CR³, and wherein X³, X⁴, X⁵, X⁶, X⁸, X⁹, and X¹⁰, independently, are not more than 7 N in total;
X⁷ is independently NH, NCH₃, or C(R²)₂;
X¹¹ is independently O, N or NH;
Y is NH, CH, or C;
R¹ is H or C₁-C₆ alkyl, wherein the alkyl is optionally substituted with one or more R⁴; or
R¹ and R⁹ combine to form a 3- to 8-membered heterocycle or 5- to 10-membered heteroaryl, wherein the heterocycle or heteroaryl is optionally substituted with one or more R⁴;
each R² is independently H, halogen, —CN, —OH, C₁-C₆ alkyl, C₂-C₆ alkenyl, C₂-C₆ alkynyl, C₁-C₆ haloalkyl, C₁-C₆ haloalkoxy, —(CH₂)ₙ—SR⁸, —(CH₂)ₙ—OR⁸, aryl, or heteroaryl; or
two R², combined with the carbon to which they are each individually attached can form a C₄-C₈ cycloalkyl or 4- to 6-membered heterocycle;
each R³ is independently H, halogen, oxo, —CN, —OR⁵, —SR⁵, —NH₂, —NH(R⁵), —N(R⁵)(R⁶), —NHC(O)R⁵, —CO(OR⁵), —C(O)R⁵, —C(O)N(R⁵)₂, C₁-C₆ alkyl, C₁-C₆ haloalkyl, C₁-C₆ haloalkoxy, C₂-C₆ alkenyl, C₂-C₆ alkynyl, C₃-C₈ cycloalkyl, heteroaryl or heterocyclyl, wherein the alkyl, alkenyl, alkynyl, cycloalkyl, heterocyclyl or heteroaryl is optionally substituted with one or more R⁶;
each R⁴ is independently H, halogen, —CN, —OR⁵, —NH₂, —NH(R⁵), —N(R⁵)(R⁶), —NHC(O)R⁵, —CO(OR⁵), —C(O)R⁵, —C(O)N(R⁵)₂, —(CH₂)ₙ—OR⁸, C₁-C₆ alkyl, C₁-C₆ alkoxy, C₂-C₆ alkenyl, C₂-C₆ alkynyl, C₃-C₃ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with one or more R⁷;
each R⁵ is independently H, —C(O)OH, —(CH₂)ₙ—O—(CH₂)ₚ—OR⁸, —(CH₂)ₙ—OR⁸, —(CH₂)ₙ—S(O)₂R⁸, —CN, C₁-C₆ alkyl, C₁-C₆ alkoxy, C₂-C₆ alkenyl, C₂-C₆ alkynyl, C₃-C₈ cycloalkyl, heterocyclyl, heteroaryl, or aryl, wherein the alkyl, alkoxy, alkenyl, alkynyl, cycloalkyl, aryl, heterocyclyl, or heteroaryl is optionally substituted with halogen, —OH, —CN, —NH₂, —N(R⁷)(R⁸), —NHC(O)OR⁸, —(CH₂)ₙ—NHC(O)R⁸, —(CH₂)ₙ—NHC(O)—(CH₂)ₚ—OR⁸, —(CH₂)ₙ—NHR⁸, —(CH₂)ₙ—NHS(O)R⁸, —(CH₂)ₙ—NHS(O)₂R⁸, —(CH₂)ₙ—C(O)R⁸, —(CH₂)ₙ—S(O)R⁸, —(CH₂)ₙ—S(O)₂R⁸, —(CH₂)ₙ—C(O)OR⁸, —(CH₂)ₙ—OR⁸, —(CH₂)ₙO(CH₂)ₙC(O)NHR⁸, C₁-C₆ alkyl, C₁-C₆ alkoxy, C₃-C₈ cycloalkyl, heteroaryl, heterocyclyl, or alkylaryl;
R⁶, R⁷, and R⁸ are independently, at each occurrence, H, halogen, —OH, —CN, —NH₂, C₁-C₆ alkyl, C₁-C₆ hydroxyalkyl, C₁-C₆ alkoxy, C₂-C₆ alkenyl, C₂-C₆ alkynyl, C₃-C₈ cycloalkyl, heterocyclyl, heteroaryl, or aryl;
R⁹ is independently H or C₁-C₄ alkyl, wherein the alkyl is optionally substituted with one or more halogen, —OH, —CN, C₁-C₆ alkoxy, or cycloalkyl;
each n is independently an integer from 0 to 6;
each p is independently an integer from 0 to 6; and
r is an integer from 0 to 2;
provided that:
(1) when R¹ and R⁹ combine to form a 3- to 8-membered heterocycle or 5- to 10-membered heteroaryl, then X¹¹ is N or NH; and/or
(2) when X¹¹ is 0, then X¹ and X² are both N and X⁷ is —CH(R²)—; and/or
(3) when X¹¹ is NH and R⁹ is H, then X⁷ is not —CH₂—; and/or
(4) when X³ is CR³ and R³ is heteroaryl, then R¹ and R⁹ form a heteroaryl;
and further provided:
(5) R¹ and R⁹ do not combine to form a pyrazole.

57. The compound of any of the preceding claims, or a pharmaceutically acceptable salt thereof.

58. An isotopic derivative of the compound of any one of the preceding claims.

59. A pharmaceutical composition comprising the compound of any one of the preceding claims, or a pharmaceutically acceptable salt, hydrate, solvate, prodrug, stereoisomer, or tautomer thereof, and one or more pharmaceutically acceptable carriers.

60. A method of treating or preventing a cGAS-related disease or disorder in a subject, the method comprising administering to the subject a therapeutically effective amount of the compound of any one of the preceding claims, or a pharmaceutically acceptable salt, hydrate, solvate, prodrug, stereoisomer, or tautomer thereof.

61. A method of inhibiting cGAS in a subject, the method comprising administering to the subject a therapeutically effective amount of the compound of any one of the preceding claims, or a pharmaceutically acceptable salt, hydrate, solvate, prodrug, stereoisomer, or tautomer thereof.

62. The compound of any one of the preceding claims, or a pharmaceutically acceptable salt, hydrate, solvate, prodrug, stereoisomer, or tautomer thereof, for use in treating or preventing a cGAS-related disease or disorder in a subject.

63. Use of the compound of any one of the preceding claims, or a pharmaceutically acceptable salt, hydrate, solvate, prodrug, stereoisomer, or tautomer thereof, in the manufacture of a medicament for treating or preventing a cGAS-related disease or disorder in a subject.

64. The method, compound, or use of any one of the preceding claims, wherein the subject is a human.

65. The method, compound, or use of any one of the preceding claims, wherein the cGAS-related disease or disorder is inflammation, an auto-immune disease, a cancer, an infection, a disease or disorder of the central nervous system, a metabolic disease, a cardiovascular disease, a respiratory disease, a kidney disease, a liver disease, an ocular disease, a skin disease, a lymphatic disease, a rheumatic disease, a psychological disease, graft versus host disease, allodynia, or a cGAS-related disease in a subject that has been determined to carry a germline or somatic non-silent mutation in cGAS.

66. The method, compound, or use of any one of the preceding claims, wherein the disease or disorder of the central nervous system is Parkinson's disease, Alzheimer's disease, traumatic brain injury, spinal cord injury, amyotrophic lateral sclerosis, or multiple sclerosis.

67. The method, compound, or use of any one of the preceding claims, wherein the kidney disease is an acute kidney disease, a chronic kidney disease, or a rare kidney disease.

68. The method, compound, or use of any one of the preceding claims, wherein the skin disease is psoriasis, hidradenitis suppurativa (HS), or atopic dermatitis.

69. The method, compound, or use of any one of the preceding claims, wherein the rheumatic disease is dermatomyositis, Still's disease, or juvenile idiopathic arthritis.

70. The method, compound, or use of any one of the preceding claims, wherein the cGAS-related disease in a subject that has been determined to carry a germline or somatic non-silent mutation in cGAS is cryopyrin-associated autoinflammatory syndrome.

71. The method, compound, or use of any one of the preceding claims, wherein the cryopyrin-associated autoinflammatory syndrome is familial cold autoinflammatory syndrome, Muckle-Wells syndrome, or neonatal onset multisystem inflammatory disease.
